(19) United States
(12) Patent Application Publication
(10) Pub. No.: US 2022/0324803 A1
(43) Pub. Date: Oct. 13, 2022

(54) PROSTAGLANDIN E2 RECEPTOR 4 ANTAGONISTS AND USES THEREOF

(71) Applicant: Teon Therapeutics, Inc., Redwood City, CA (US)

(72) Inventor: Jiwen Liu, Redwood City, CA (US)

(21) Appl. No.: 17/615,821

(22) PCT Filed: Jun. 9, 2020
(86) PCT No.: PCT/US2020/036852

§ 371 Date: Dec. 1, 2021

Related U.S. Application Data
(60) Provisional application No. 62/859,924, filed on Jun. 11, 2019.

(51) Int. Cl. C07D 209/42; C07D 231/56; C07D 471/04; C07D 401/06; A61K 45/06

(52) U.S. Cl. CPC C07D 209/42; C07D 231/56; C07D 471/04; C07D 401/06; A61K 45/06

(57) ABSTRACT

Disclosed herein are compounds, compositions, and methods for modulating the prostaglandin E2 receptor 4 (EP4) with the compounds and compositions disclosed herein. Also described are methods of treating diseases or disorders that are mediated by the action of prostaglandin E2 (PGE2) at the prostaglandin E2 receptor 4 (EP4), such as cancer, with EP4 antagonists.

### CROSS-REFERENCE

This application claims benefit of U.S. Provisional Application No. 62/859,924, filed on Jun. 11, 2019, which is herein incorporated by reference in its entirety.

### FIELD OF THE INVENTION

Described herein are compounds, methods of making such compounds, pharmaceutical compositions and medicaments comprising such compounds, and methods of using such compounds for the treatment of conditions, diseases, or disorders that would benefit from reduction or inhibition of prostaglandin E₂ receptor 4 (EP₄) activity.

### BACKGROUND OF THE INVENTION

Prostaglandin E2 (PGE₂) is a potent inflammatory mediator that is produced from arachidonic acid by cyclooxygenase 2 (COX2) and other metabolic enzymes. Elevated-levels of COX2 and PGE₂ are found in numerous cancers, and are associated with tumor development and progression. Prostaglandin E₂ receptor 4 (EP₄) is one of the four identified EP receptors for PGE₂, and it has been implicated in various physiological and pathological diseases or conditions such as cancer, inflammation, pain, and migraine. EP₄ is one of the main downstream targets of COX2 pathways involved in tumor promotion and immune suppression. EP₄ antagonists have demonstrated efficacy in numerous clinical and safety trials, such as tumor models and anti-tumor immunity.

### SUMMARY OF THE INVENTION

In one aspect, described herein is a compound of Formula (I), or a pharmaceutically acceptable salt or solvate thereof.

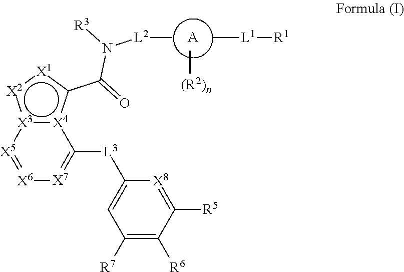

wherein,
R¹ is —CO₂H, —CO₂(C₁-C₆alkyl), —C(═O)NHSO₂R¹², —C(═O)N(R¹³)₂, tetrazolyl, or a carboxylic acid bioisostere;
L¹ is absent, C₁-C₄alkylene, or C₃-C₆cycloalkylene;
ring A is a phenyl, naphthyl, C₃-C₁₂cycloalkyl, C₂-C₁₀heterocycloalkyl, or heteroaryl;
L² is absent, C₁-C₄alkylene, or C₃-C₆cycloalkylene;
each R² is independently selected from H, halogen, —OH, —CN, —NH₂, —NH(C₁-C₆alkyl), —N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R³ is H or C₁-C₆alkyl;
or R³ and L² are taken together with the N-atom to which they are attached to form a N-containing C₂-C₆heterocycloalkyl that is unsubstituted or substituted with 1, 2, 3 or 4
R².
or R³ and ring A are taken together with the intervening atoms to which they are attached to form a substituted or unsubstituted N-containing heterocycloalkyl or a substituted or unsubstituted N-containing heteroaryl, wherein if the ring is substituted then the ring is substituted with 1-4 R²;
or R³ and one R² on ring A are taken together with the intervening atoms to which they are attached to form a form a substituted or unsubstituted fused ring with ring A that is a substituted or unsubstituted fused N-containing heterocycloalkyl or a substituted or unsubstituted fused 5-membered or 6-membered heteroaryl, wherein if the fused ring is substituted then the fused ring is substituted with 1-4 R²;
X¹ is N, C—R^{a}, or N—R^{c};
X² is N, C—R^{b}, or N—R^{c};
   R^{a} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
   R^{b} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
   R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂;
X³ is C or N;
X⁴ is C or N; provided that both X³ and X⁴ are not N at the same time;
L³ is C₁-C₄alkylene, —O—C₁-C₄alkylene-, —NR^{d}—C₁-C₄alkylene-, or —NR^{d}—;
   R^{d} is H, C₁-C₆alkyl, C₁-C₆fluoroalkyl, or C₁-C₆heteroalkyl;
X⁵ is C—R⁸ or N;
X⁶ is C—R⁹ or N;
X⁷ is C—R¹⁰ or N;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², —CO₂R¹³;
or R⁵ and R⁶ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
or R⁶ and R⁷ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
each R⁸, R⁹, R¹⁰, and R¹¹ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, 2, 3, or 4.

In one aspect, provided herein is a pharmaceutical composition comprising a compound disclosed herein, or a pharmaceutically acceptable salt, or solvate thereof, and at least one pharmaceutically acceptable excipient.

In some embodiments, the compounds disclosed herein, or a pharmaceutically acceptable salt thereof, are formulated for administration to a mammal by intravenous administration, subcutaneous administration, oral administration, inhalation, nasal administration, dermal administration, or ophthalmic administration. In some embodiments, the compound disclosed herein, or a pharmaceutically acceptable salt thereof, is in the form of a tablet, a pill, a capsule, a liquid, a suspension, a gel, a dispersion, a solution, an emulsion, an ointment, or a lotion.

In one aspect, described herein is a method of modulating the activity of prostaglandin E2 receptor 4 (EP4) in a mammal comprising administering to the mammal a compound described herein, or any pharmaceutically acceptable salt or solvate thereof.

In another aspect, described herein is a method of treating a disease or disorder in a mammal that is mediated by the action of prostaglandin E2 (PGE2) at prostaglandin E2 receptor 4 (EP4) comprising administering to the mammal a compound of Formula (I), or a pharmaceutically acceptable salt or solvate thereof. In some embodiments, the disease or disorder is cancer.

In another aspect, described herein is a method for treating cancer in a mammal, the method comprising administering to the mammal a compound of Formula (I), or a pharmaceutically acceptable salt or solvate thereof.

In some embodiments, the cancer is a solid tumor. In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer. In some embodiments, the cancer is prostate cancer, breast cancer, colon cancer, or lung cancer. In some embodiments, the cancer is a sarcoma, carcinoma, or lymphoma.

In some embodiments of the methods of treatment described herein are further embodiments that include the co-administration of at least one additional therapy to the mammal in addition to the compound of Formula (I), or a pharmaceutically acceptable salt or solvate thereof.

In some embodiments, the mammal is a human.

In any of the aforementioned aspects are further embodiments in which an effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal.

In any of the aforementioned aspects are further embodiments comprising single administrations of an effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

Articles of manufacture, which include packaging material, a formulation within the packaging material (e.g. a formulation suitable for topical administration), and a label that indicates that the compound or composition, or pharmaceutically acceptable salt, or solvate thereof, is used for reducing or inhibiting EP4 activity, or for the treatment, prevention or amelioration of one or more symptoms of a disease or disorder that is associated with EP4 activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Prostaglandin E2 (PGE₂) is a potent inflammatory mediator that is produced from arachidonic acid by cyclooxygenase 2 (COX2) and other metabolic enzymes. In some instances, PGE₂ is produced from arachidonic acid by COX1 and COX2 cyclooxygenases and the cytosolic (cPGES1) and microsomal (mPGES1 and mPGES2) prostaglandin E synthases.

The prostaglandin E2 (PGE2) receptors are G protein-coupled receptors that bind and are activated by prostaglandin E2. They are members of the prostaglandin receptors class of receptors and include the following isoforms: prostaglandin E2 receptor 1 (EP1), prostaglandin E2 receptor 2 (EP2), prostaglandin E2 receptor 3 (EP3), and prostaglandin E2 receptor 4 (EP4)

PGE₂ acts on all members of the prostaglandin E₂ receptor family (EP₁-EP₄), but it has the highest affinity for EP₃ and EP₄. Modulation of the prostaglandin E₂ receptor family has been implicated in a variety of therapeutic indications, such as cancer, ulcerative colitis, Alzheimer's disease, pulmonary fibrosis, and cardiovascular disease.

Prostaglandin E₂ receptor 4 (EP₄, PTGER4 gene) is a major effector of various signaling pathways vital for cellular functions, such as cell proliferation, cellular differentiation, cellular survival, and angiogenesis.

Elevated COX2 and PGE₂ levels are found in numerous cancers and are associated with tumor development and progression. Also, certain genetic variations in the PTGER4 gene have been associated with increased incidence of diseases, such as inflammatory bowel disease, Ankylosing spondylitis, and primary graft dysfunction. In some instances, EP₄ is over-expressed in human prostate cancer tissue. EP₄ has been implicated in various important physiological and pathological signaling pathways.

In some instances, EP₄ activity increases various hallmarks of cancer, such as inflammation, cell motility, cell migration, angiogenesis, proliferation, invasion, and aggressiveness of tumors. In some instances, EP4 is upregulated in numerous tumor lineages, such as brain, thyroid, pancreas, breast, renal, lung, liver, bone, cardiovascular, glioblastoma, ovarian and prostate cancer. In some instances, EP₄ overexpression is observed in malignant tumor tissues, such as breast cancer, renal cancer, colorectal cancer, prostate cancer, and lung cancer.

Among the four PGE2 receptors, EP3 and EP4 are the most widely expressed in the body. By contrast, the distribution of the EP1 receptor is restricted to a few organs in humans. All EP receptor subtypes are present on the plasma membrane. EP3 and EP4 are also expressed on the cell nuclei membranes.

### Cancer

EP₄ antagonists have been examined as treatments for various cancers, such as, but not limited to, prostate cancer, lung cancer, breast cancer, B cell lymphoma, and colorectal cancer. In addition, EP₄ overexpression is frequently observed in malignant tumor tissues, such as breast cancer, renal cancer, colorectal cancer, prostate cancer, and lung cancer.

For example, it has been noted that the overexpression of EP4 led to the progression of prostate cancer in a mouse xenograft disease model and treatment with the EP4 antagonist ONO-AE3-208 suppressed castration-resistant prostate cancer progression (Terada, N. et al. (2010) Cancer Res. 70, 1606-1615). Consistent with these findings, EP4 expression was found to be upregulated in prostate cancer patients (Jain, S. et al. (2008) Cancer Res. 68, 7750-7759).

PGE2 plays an important role in lung cancer. PGE2 is abundantly expressed in lung tissue and the inhibition of its signalling suppressed tumorigenic effects in animal models of lung cancer. These observations have been corroborated by subsequent findings that show that: (i) PGE2 promotes human NSCLC growth in vitro and its tumorigenic effects are successfully inhibited by pharmacologically blocking EP4 activity with the EP4 antagonist AH-23848; and (ii) pulmonary metastasis of lung carcinoma cells injected intravenously in mice was facilitated via the EP4 receptor. In agreement with the role of EP4 in prostate and lung cancer, studies evaluating the role of EP4 in breast cancer showed that pharmacological blockade of EP4 activity using the EP4 antagonist GW-627368X selectively inhibited both proliferation and invasion of human inflammatory breast cancer cells (Robertson, F. M. et al. (2008). J. Exp. Ther. Oncol. 7, 299-312). In addition, the EP4 receptor antagonists AH-23848 and ONO-AE3-208 reduced metastasis of murine mammary tumour cells.

The beneficial effects of blocking EP4 signalling in colorectal cancer have been confirmed in several in vivo studies as well as by a pharmacological blockade of the EP4 receptor. For example, the EP4 −/− mice treated with carcinogens had reduced development of aberrant crypt foci (precursors of colorectal tumours) relative to wild-type mice treated with the same carcinogens (Sonoshita, M. et al. (2001) Nat. Med. 7, 1048-1051). The administration of the EP4 antagonist ONO-AE2-227 reduced the formation of aberrant crypt foci and intestinal polyps in a mouse disease model (Mutoh, M. et al. (2002) Cancer Res. 62, 28-32).

In some embodiments, disclosed herein are methods of treating cancer with a compound of Formula (I), or a pharmaceutically acceptable salt or solvate thereof.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

In some embodiments, a mammal treated with a compound described herein has a disease or disorder that is or is associated with a cancer or tumor. Thus, in some embodiments, the mammal is a human that is an oncology patient. Such diseases and disorders and cancers include carcinomas, sarcomas, benign tumors, primary tumors, tumor metastases, solid tumors, non-solid tumors, blood tumors, leukemias and lymphomas, and primary and metastatic tumors.

In some embodiments, the EP4 receptor antagonists described herein are used in the treatment of solid tumours. A solid tumor is a n abnormal mass of tissue that usually does not contain cysts or liquid areas. Solid tumors may be benign (not cancer), or malignant (cancer). Different types of solid tumors are named for the type of cells that form them. Examples of solid tumors are carcinomas, sarcomas, and lymphomas.

Carcinomas include, but are not limited to, esophageal carcinoma, hepatocellular carcinoma, basal cell carcinoma, squamous cell carcinoma, bladder carcinoma, bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, renal cell carcinoma, Wilm's tumor, cervical carcinoma, uterine carcinoma, testicular carcinoma, osteogenic carcinoma, epithelial carcinoma, and nasopharyngeal carcinoma.

Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Leukemias include, but are not limited to, a) chronic myeloproliferative syndromes (neoplastic disorders of multipotential hematopoietic stem cells); b) acute myelogenous leukemias; c) chronic lymphocytic leukemias (CLL), including B-cell CLL, T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Benign tumors include, e.g., hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Primary and metastatic tumors include, e.g., lung cancer; breast cancer; colorectal cancer; anal cancer; pancreatic cancer; prostate cancer; ovarian carcinoma; liver and bile duct carcinoma; esophageal carcinoma; bladder carcinoma; carcinoma of the uterus; glioma, glioblastoma, medulloblastoma, and other tumors of the brain; kidney cancers; cancer of the head and neck; cancer of the stomach; multiple myeloma; testicular cancer; germ cell tumor; neuroendocrine tumor; cervical cancer; carcinoids of the gastrointestinal tract, breast, and other organs.

In one aspect, a compound of Formula (I), or a pharmaceutically acceptable salt or solvate thereof, reduces, ameliorates or inhibits cell proliferation associated with cancers.

### Compounds

Compounds described herein, including pharmaceutically acceptable salts, prodrugs, active metabolites and solvates thereof, are prostaglandin E₂ receptor 4 (EP₄) modulators. In some embodiments, the EP₄ modulators are EP₄ antagonists.

In one aspect, described herein is a compound of Formula (I), or a pharmaceutically acceptable salt or solvate thereof:

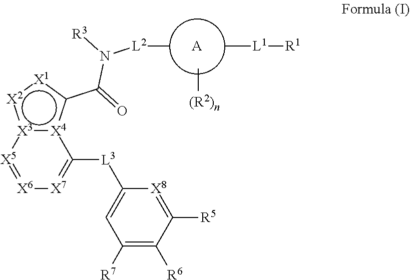

wherein,
R¹ is —CO₂H, —CO₂(C₁-C₆alkyl), —C(═O)NHSO₂R¹², —C(═O)N(R¹³)₂, tetrazolyl, or a carboxylic acid bioisostere;
L¹ is absent, C₁-C₄alkylene, or C₃-C₆cycloalkylene;
ring A is a phenyl, naphthyl, C₃-C₁₂cycloalkyl, C₂-C₁₀heterocycloalkyl, or heteroaryl;
L² is absent, C₁-C₄alkylene, or C₃-C₆cycloalkylene;
each R² is independently selected from H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R³ is H or C₁-C₆alkyl;
or R³ and L² are taken together with the N-atom to which they are attached to form a N-containing C₂-C₆heterocycloalkyl that is unsubstituted or substituted with 1, 2, 3 or 4 R².
or R³ and ring A are taken together with the intervening atoms to which they are attached to form a substituted or unsubstituted N-containing heterocycloalkyl or a substituted or unsubstituted N-containing heteroaryl, wherein if the ring is substituted then the ring is substituted with 1-4 R²;
or R³ and one R² on ring A are taken together with the intervening atoms to which they are attached to form a form a substituted or unsubstituted fused ring with ring A that is a substituted or unsubstituted fused N-containing heterocycloalkyl or a substituted or unsubstituted fused 5-membered or 6-membered heteroaryl, wherein if the fused ring is substituted then the fused ring is substituted with 1-4 R²;
X¹ is N, C—R^{a}, or N—R^{c};
X² is N, C—R^{b}, or N—R^{c};
   R^{a} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
   R^{b} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂;
X³ is C or N;
X⁴ is C or N; provided that both X³ and X⁴ are not N at the same time;
L³ is C₁-C₄alkylene, —O—C₁-C₄alkylene-, —O—, —NR^{d}—C₁-C₄alkylene-, or —NR^{d}—;
   R^{d} is H, C₁-C₆alkyl, C₁-C₆fluoroalkyl, or C₁-C₆heteroalkyl;
X⁵ is C—R⁸ or N;
X⁶ is C—R⁹ or N;
X⁷ is C—R¹⁰ or N;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
or R⁵ and R⁶ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
or R⁶ and R⁷ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
each R⁸, R⁹, R¹⁰, and R¹¹ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, 2, 3, or 4.

For any and all of the embodiments, substituents are selected from among a subset of the listed alternatives. For example, in some embodiments, R¹ is —CO₂H, —CO₂(C₁-C₆alkyl), —C(═O)NHSO₂R¹², —C(═O)N(R¹³)₂, tetrazolyl, or a carboxylic acid bioisostere. In other embodiments, R¹ is —CO₂H, —CO₂(C₁-C₆alkyl), —C(═O)NHSO₂R¹², or —C(═O)N(R¹³)₂. In other embodiments, R¹ is —CO₂H, tetrazolyl, or a carboxylic acid bioisostere. In some embodiments, X is R¹ is —CO₂H or —CO₂(C₁-C₆alkyl). In some other embodiments, R¹ is —CO₂H.

In another aspect, described herein is a compound of Formula (I), or a pharmaceutically acceptable salt or solvate thereof:

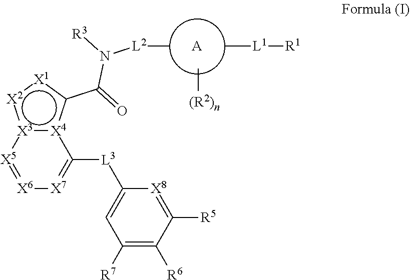

wherein,
R¹ is —CO₂H, —CO₂(C₁-C₆alkyl), —C(═O)NHSO₂R¹², —C(═O)N(R¹³)₂, tetrazolyl, or a carboxylic acid bioisostere;
L¹ is absent, C₁-C₄alkylene, or C₃-C₆cycloalkylene;
ring A is a phenyl, naphthyl, C₃-C₁₂cycloalkyl, C₂-C₁₀heterocycloalkyl, or heteroaryl;
L² is absent, C₁-C₄alkylene, or C₃-C₆cycloalkylene;
each R² is independently selected from H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R³ is H or C₁-C₆alkyl;
or R³ and L² are taken together with the N-atom to which they are attached to form a N-containing C₂-C₆heterocycloalkyl that is unsubstituted or substituted with 1, 2, 3 or 4 R²
or R³ and ring A are taken together with the intervening atoms to which they are attached to form a substituted or unsubstituted N-containing heterocycloalkyl or a substituted or unsubstituted N-containing heteroaryl, wherein if the ring is substituted then the ring is substituted with 1-4 R²;
or R³ and one R² on ring A are taken together with the intervening atoms to which they are attached to form a form a substituted or unsubstituted fused ring with ring A that is a substituted or unsubstituted fused N-containing heterocycloalkyl or a substituted or unsubstituted fused 5-membered or 6-membered heteroaryl, wherein if the fused ring is substituted then the fused ring is substituted with 1-4 R²;
X¹ is N, C—R^{a}, or N—R^{c};
X² is N, C—R^{b}, or N—R^{c};
   R^{a} is H, halogen, —OH, —CN, —NH₂, —NH(C₁-C₆alkyl), —N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
   R^{b} is H, halogen, —OH, —CN, —NH₂, —NH(C₁-C₆alkyl), —N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
   R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂;
X³ is C or N;
X⁴ is C or N; provided that both X³ and X⁴ are not N at the same time;
L³ is C₁-C₄alkylene;
   R^{d} is H, C₁-C₆alkyl, C₁-C₆fluoroalkyl, or C₁-C₆heteroalkyl;
X⁵ is C—R⁸ or N;
X⁶ is C—R⁹ or N;
X⁷ is C—R¹⁰ or N;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
or R⁵ and R⁶ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
or R⁶ and R⁷ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
each R⁸, R⁹, R¹⁰, and R¹¹ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, or substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, 2, 3, or 4.

In some embodiments, at least one of X³ or X⁴ is N. In some embodiments, X³ is N; and X⁴ is C. In some embodiments, X³ is C; and X⁴ is N.

In some embodiments, X¹ is C—R^{a}; X² is N or C—R^{b}; X³ is N; and X⁴ is C; or X¹ is C—R^{a}; X² is N—R^{c}; X³ is C; and X⁴ is C or N; or X¹ is N; X² is C—R^{b}; X³ is N; and X⁴ is C; or X¹ is N or N—R^{c}; X² is C—R^{b} or N; X³ is C; and X⁴ is C or N. In some embodiments, X¹ is C—R^{a}; X² is N or C—R^{b}; X³ is N; and X⁴ is C.

In some embodiments, X¹ is C—R^{a}; X² is N or C—R^{b}; X³ is C or N; and X⁴ is C or N; or X¹ is N; X² is N—R, or C—R^{b}; X³ is C or N; and X⁴ is C or N. In some embodiments, X¹ is N; X² is N—R^{c}; X³ is C; and X⁴ is C. In some embodiments, X¹ is C—R^{a}; X² is N; X³ is C; and X⁴ is C.

In some embodiments, X¹ is C—R^{a}; X² is N; X³ is N; and X⁴ is C; or X¹ is N; X² is C—R^{b}; X³ is N; and X⁴ is C; or X¹ is N; X² is C—R^{b}; X³ is C; and X⁴ is N; or X¹ is N; X² is N—R^{c}; X³ is C; and X⁴ is C; or X¹ is C—R^{a}; X² is N—R^{c}; X³ is C; and X⁴ is C; or X¹ is C—R^{a}; X² is N—R^{c}; X³ is C; and X⁴ is N; or X¹ is C—R^{a}; X² is C—R^{b}; X³ is N; and X⁴ is C; or X¹ is N—R^{c}; X² is N; X³ is C; and X⁴ is C. In some embodiments, X¹ is N; X² is C—R^{b}; X³ is N; and X⁴ is C.

In some embodiments, X⁵ is N; X⁶ is C—R⁹; and X⁷ is C—R¹⁰; or X⁵ is C—R⁸; X⁶ is N; and X⁷ is C—R¹⁰; or X⁵ is C—R⁸; X⁶ is C—R⁹; and X⁷ is N; or X⁵ is N; X⁶ is C—R⁹; and X⁷ is N.

In some embodiments, X¹ is C—R^{a}; X² is N; X³ is C; X⁴ is C; X⁵ is N X⁶ is C—R⁹; and X⁷ is C—R¹⁰.

In some embodiments,

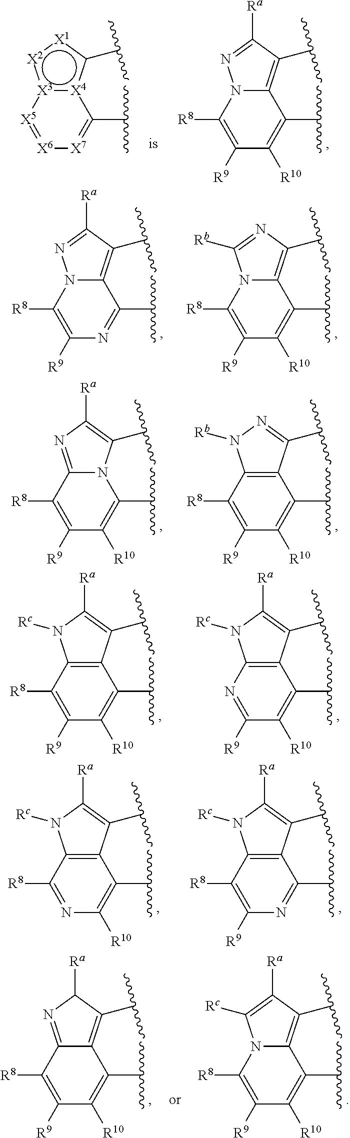

In some embodiments,

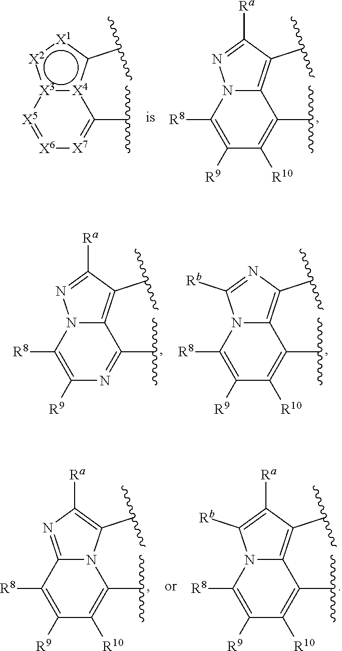

In some embodiments,

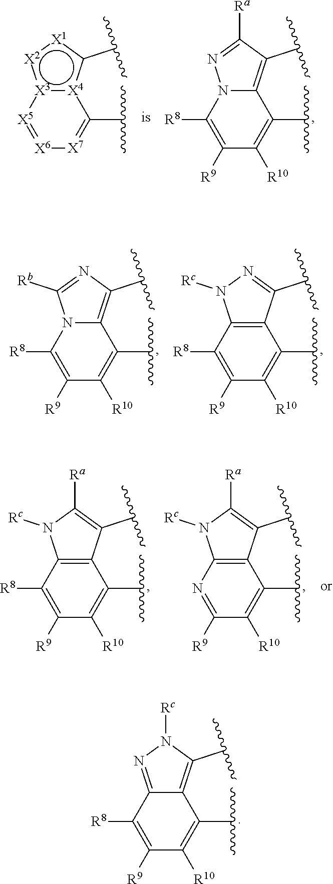

In some embodiments, R¹ is —CO₂H, or —CO₂(C₁-C₆alkyl).

In some embodiments, R¹ is —CO₂H.

In some embodiments, the compound of Formula (I) has the structure of Formula (II), Formula (III), Formula (IV), or a pharmaceutically acceptable salt or solvate thereof:

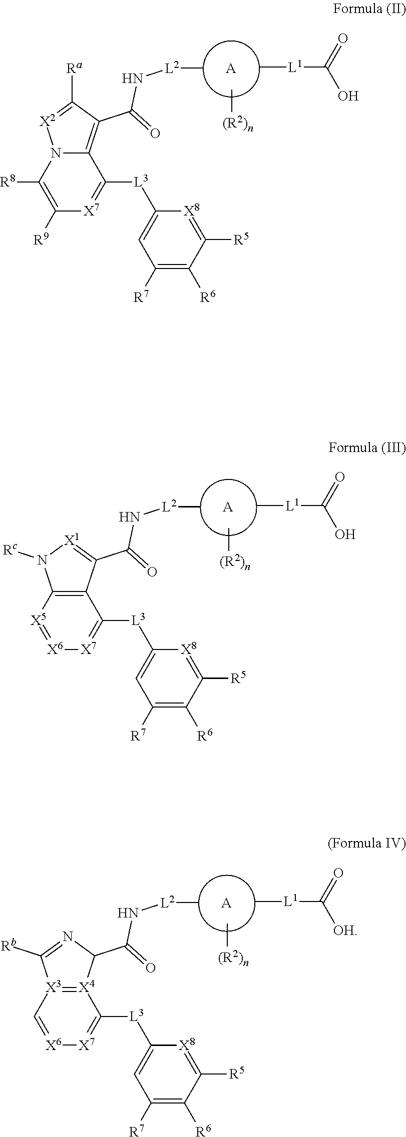

In some embodiments, the compound of Formula (I) has the structure of Formula (V), Formula (VI), Formula (VII), Formula (VIII), or a pharmaceutically acceptable salt or solvate thereof:

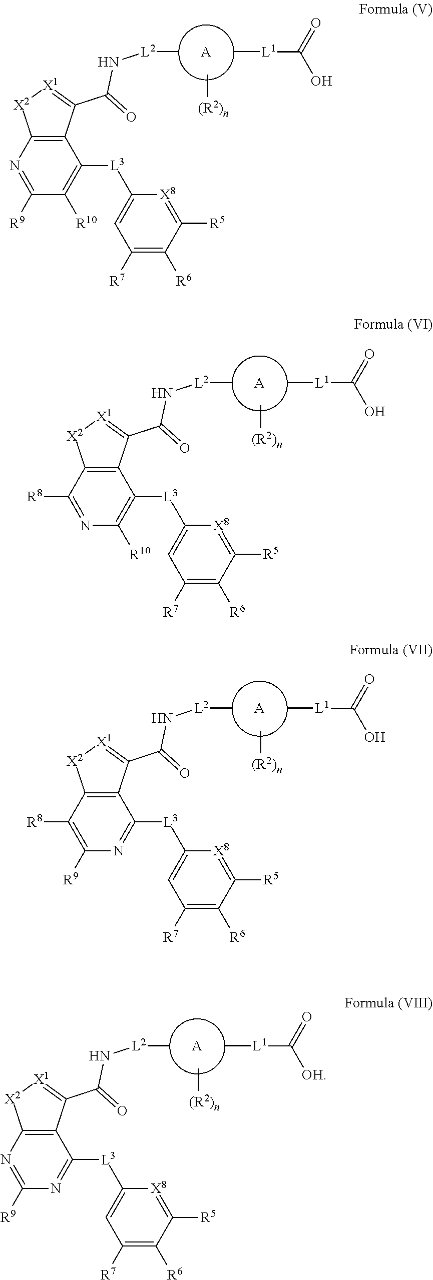

In some embodiments, L³ is C₁-C₄alkylene, —O—C₁-C₄alkylene-, —NR^{d}—C₁-C₄alkylene-, or —NR^{d}—. In some embodiments, L³ is C₁-C₄alkylene, —NR^{d}—C₁-C₄alkylene-, or —NR^{d}—. In some embodiments, L³ is C₁-C₄alkylene.

In some embodiments, L³ is —CH₂—, —CH₂CH₂—, —CH₂CH₂CH₂—, —CH(CH₃)—, —C(CH₃)₂—, —O—, —OCH₂—, —OCH₂CH₂—, —OCH₂CH₂CH₂—, —OCH(CH₃)—, —OC(CH₃)₂—, —NH—, —NHCH₂—, —NHCH₂CH₂—, —NHCH₂CH₂CH₂—, —NHCH(CH₃)—, or —NHC(CH₃)₂—. In some embodiments, L³ is —CH₂—, —CH₂CH₂—, —CH₂CH₂CH₂—, —CH(CH₃)—, —C(CH₃)₂—, —OCH₂—, —OCH₂CH₂—, —OCH₂CH₂CH₂—, —OCH(CH₃)—, —OC(CH₃)₂—, —NH—, —NHCH₂—, —NHCH₂CH₂—, —NHCH₂CH₂CH₂—, —NHCH(CH₃)—, or —NHC(CH₃)₂—. In some embodiments, L³ is —CH₂—, —CH₂CH₂—, —CH₂CH₂CH₂—, —CH(CH₃)—, —C(CH₃)₂—, —NH—, —NHCH₂—, —NHCH₂CH₂—, —NHCH₂CH₂CH₂—, —NHCH(CH₃)—, or —NHC(CH₃)₂—.

In some embodiments, L³ is —CH₂—, —CH₂CH₂—, —CH(CH₃)—, —O—, —OCH₂—, —OCH₂CH₂—, —OCH(CH₃)—, —NH—, —NHCH₂—, —NHCH₂CH₂—, or —NHCH(CH₃)—. In some embodiments, L³ is —CH₂—, —CH₂CH₂—, —CH(CH₃)—, —OCH₂—, —OCH₂CH₂—, —OCH(CH₃)—, —NH—, —NHCH₂—, —NHCH₂CH₂—, or —NHCH(CH₃)—. In some embodiments, L³ is —CH₂—, —CH₂CH₂—, —CH(CH₃)—, —NH—, —NHCH₂—, —NHCH₂CH₂—, or —NHCH(CH₃)—. In some embodiments, L³ is —CH₂—, —CH₂CH₂—, or —CH(CH₃)—.

In some embodiments, L³ is —CH₂—.

In some embodiments, the compound of Formula (I) has the structure of Formula (IX), or a pharmaceutically acceptable salt or solvate thereof:

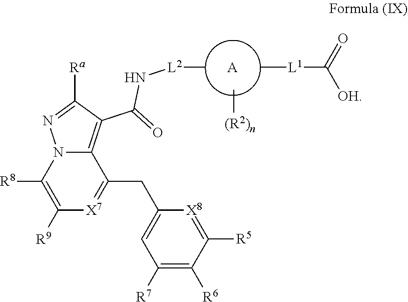

In some embodiments, the compound of Formula (I) has the structure of Formula (X), or a pharmaceutically acceptable salt or solvate thereof:

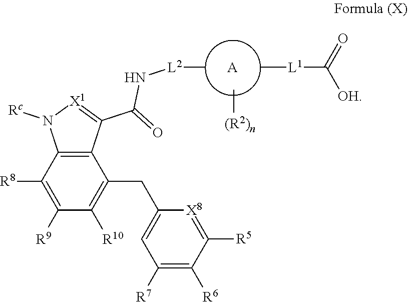

In some embodiments, the compound of Formula (I) has the structure of Formula (Xa), or a pharmaceutically acceptable salt or solvate thereof:

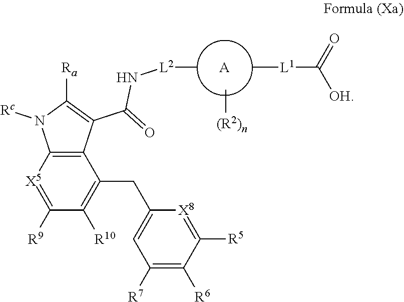

In some embodiments, the compound of Formula (I) has the structure of Formula (XI), or a pharmaceutically acceptable salt or solvate thereof:

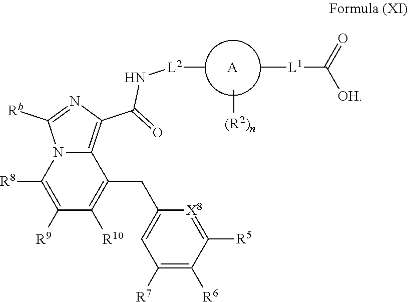

In some embodiments, L¹ is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl; L² is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl.

In some embodiments, L¹ is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl; L² is absent, —CH₂—, —CH₂CH₂—, —CH₂CH₂CH₂—, or —CH(CH₃)—.

In some embodiments, L¹ is absent, —CH₂—, —CH(CH₃)—, —C(CH₃)₂—, or cyclopropyl-1,1-diyl; and L² is absent, —CH₂—, —CH(CH₃)—, —C(CH₃)₂—, or cyclopropyl-1,1-diyl. In some embodiments, L¹ and L² are each independently —CH₂—, —CH(CH₃)—, —C(CH₃)₂—, or cyclopropyl-1,1-diyl. In some embodiments, L¹ and L² are each absent.

In some embodiments

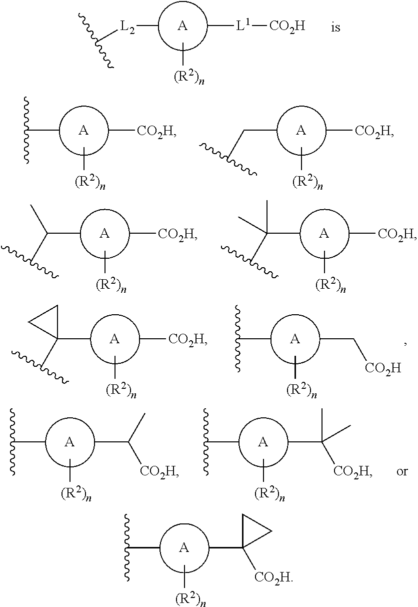

In some embodiments,

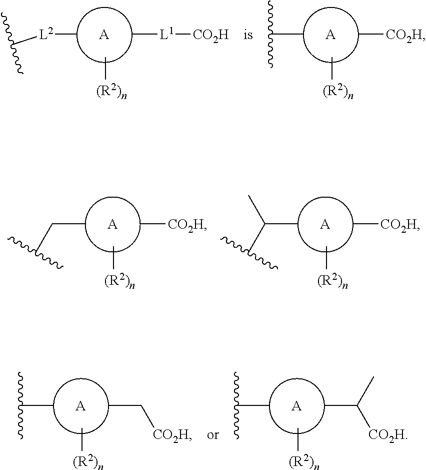

In some embodiments,

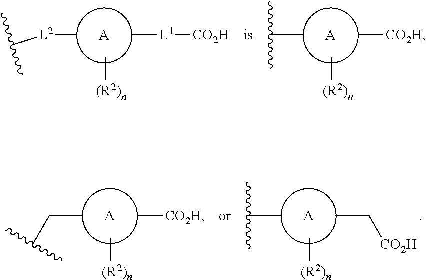

In some embodiments, ring A is a phenyl.

In some embodiments,

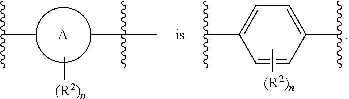

In some embodiments, ring A is a monocyclic C₃-C₈cycloalkyl, or bicyclic C₇-C₁₂cycloalkyl.

In some embodiments, ring A is a monocyclic C₃-C₈cycloalkyl; or ring A is a bicyclic C₇-C₁₂cycloalkyl that is a fused bicyclic C₇-C₁₂cycloalkyl, bridged bicyclic C₇-C₁₂cycloalkyl, or spiro bicyclic C₇-C₁₂cycloalkyl.

In some embodiments, ring A is cyclobutyl, cyclopentyl, or cyclohexyl; or ring A is a bicyclic C₇-C₁₂cycloalkyl that is a spiro[2.2]pentanyl, spiro[3.3]heptanyl, spiro[4.3]octanyl, spiro[3.4]octanyl, spiro[3.5]nonanyl, spiro[4.4]nonanyl, spiro[4.5]decanyl, spiro[5.4]decanyl, spiro[5.5]undecanyl, bicyclo[1.1.1]pentanyl, bicyclo[2.2.2]octanyl, bicyclo[2.2.1]heptanyl, adamantyl, or decalinyl.

In some embodiments, ring A is cyclobutyl, cyclopentyl, or cyclohexyl; or ring A is spiro[3.3]heptanyl, bicyclo[1.1.1]pentanyl, or bicyclo[2.2.2]octanyl.

In some embodiments, ring A is phenyl, cyclohexyl, spiro[3.3]heptanyl, bicyclo[1.1.1]pentanyl, or bicyclo[2.2.2]octanyl. In some embodiments, ring A is cyclohexyl, spiro[3.3]heptanyl, bicyclo[1.1.1]pentanyl, or bicyclo[2.2.2]octanyl.

In some embodiments,

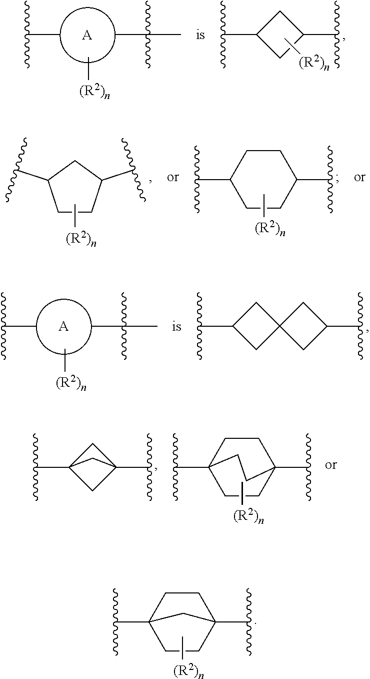

In some embodiments,

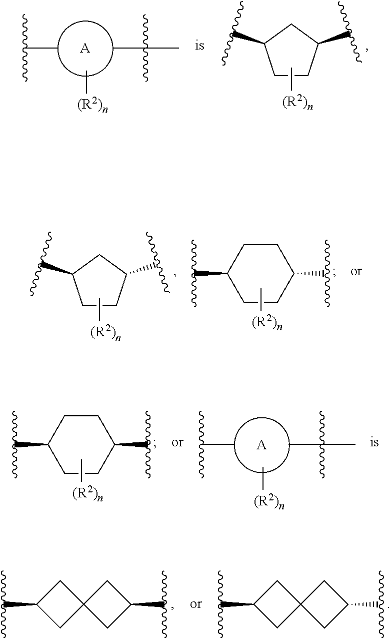

In some embodiments,

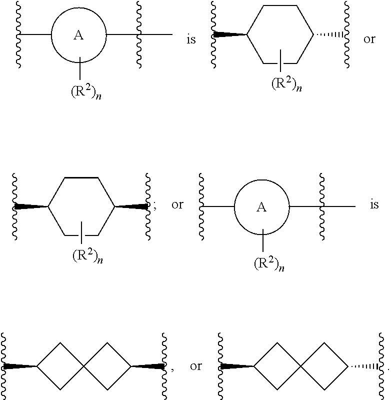

In some embodiments,

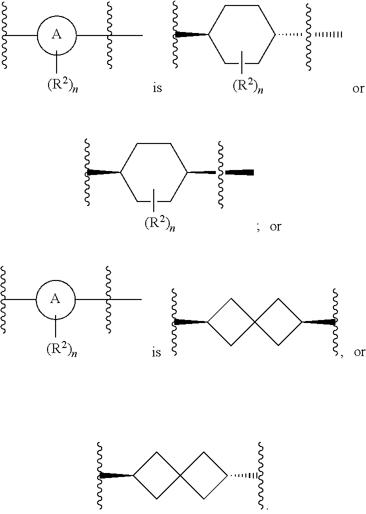

In some embodiments,

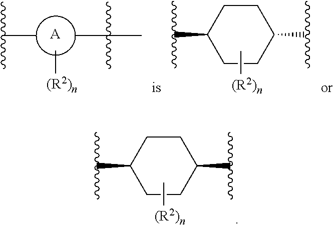

In some embodiments,

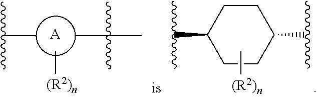

In some embodiments,

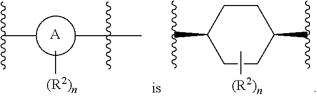

In some embodiments,

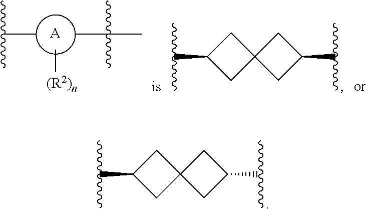

In some embodiments,

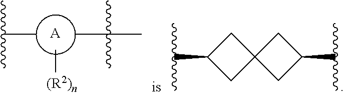

In some embodiments,

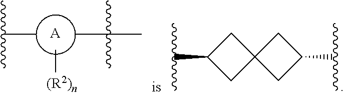

In some embodiments,

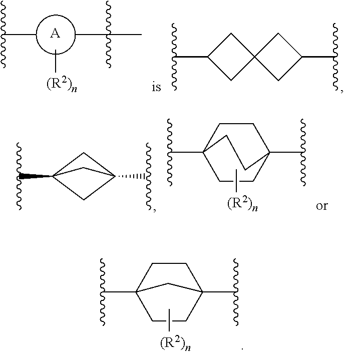

In some embodiments,

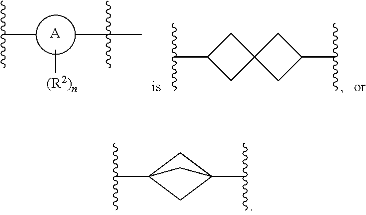

In some embodiments,

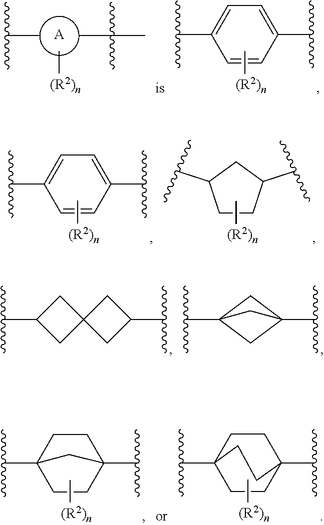

In some embodiments,

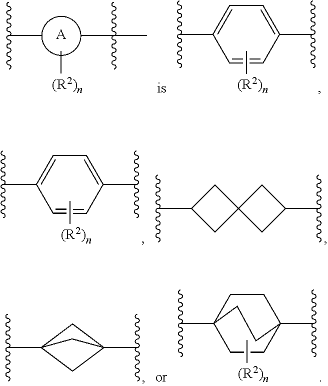

In some embodiments, ring A is a monocyclic C₂-C₆heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

In some embodiments, ring A is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

In some embodiments, ring A is a monocyclic heteroaryl.

In some embodiments, ring A is a monocyclic heteroaryl selected from furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, and triazinyl.

In some embodiments,

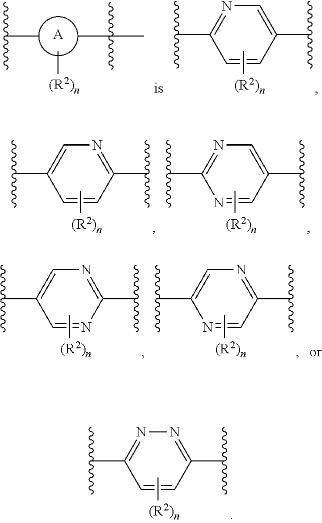

In some embodiments, ring A is a bicyclic heteroaryl selected from indolyl, benzofuranyl, benzothienyl, benzoxazolyl, benzisoxazolyl, benzimidazolyl, imidazopyrdinyl, imidazopyridazinyl, purinyl, quinolinyl, quinazolinyl, and pyridopyrimidinyl.

In some embodiments, each R² is independently selected from H, F, Cl, Br, —OH, —CN, —NH₂, —NH(CH₃), —N(CH₃)₂, —CH₃, —OCH₃, —CF₃, and —OCF₃; n is 0, 1, or 2.

In some embodiments,

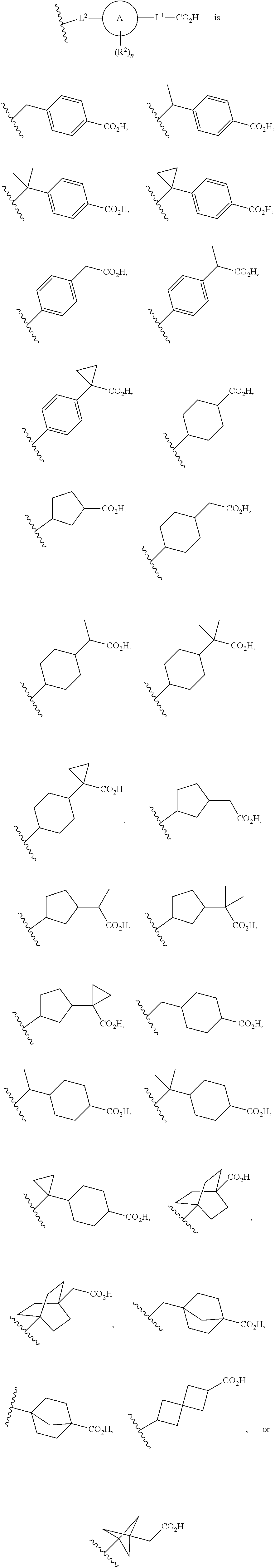

In some embodiments,

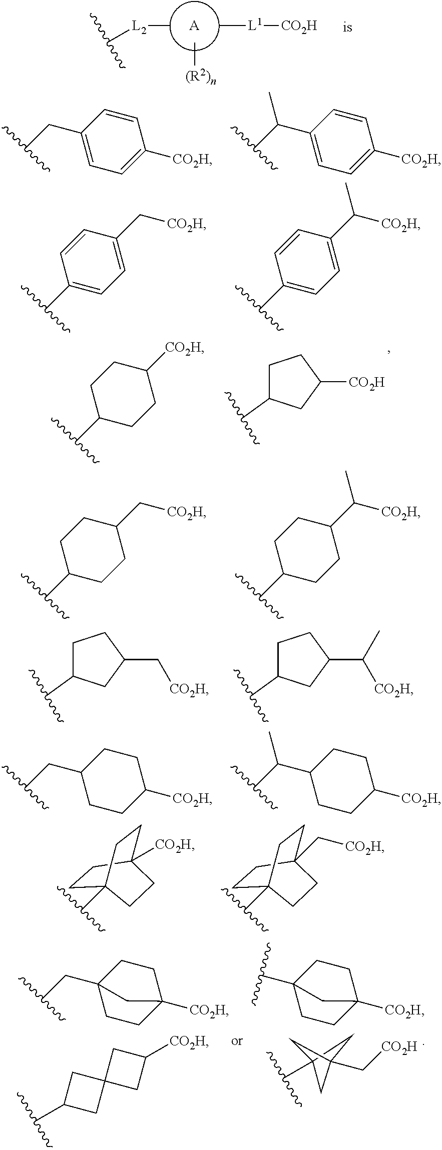

In some embodiments,

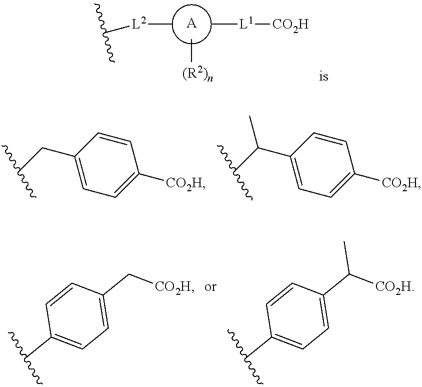

In some embodiments,

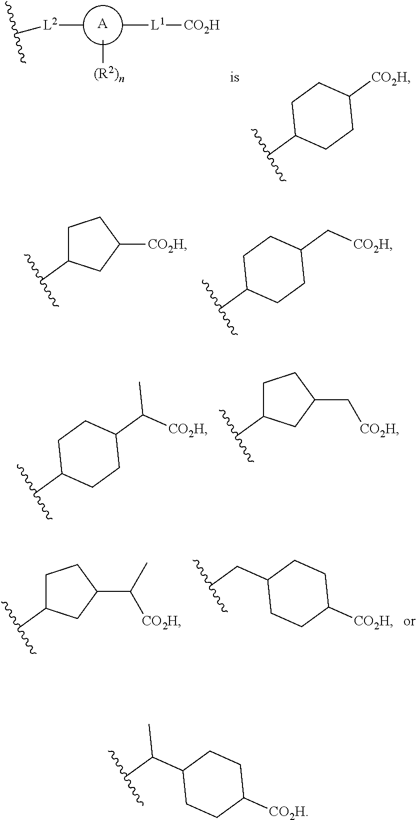

In some embodiments,

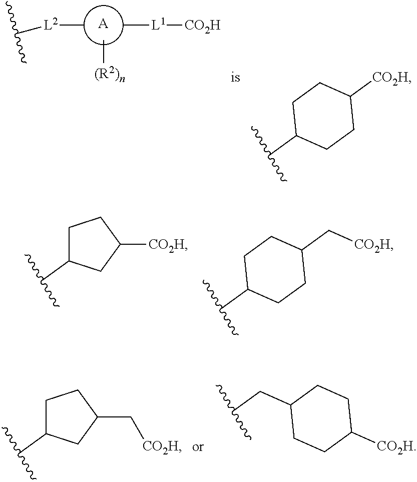

In some embodiments,

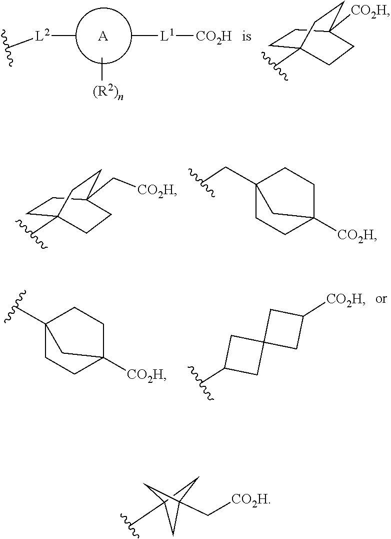

In some embodiments, L¹ is absent, —CH₂—,

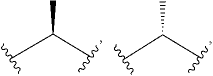

or cyclopropyl-1,1-diyl; L² is absent, —CH₂—,

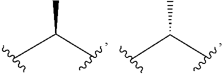

or cyclopropyl-1,1-diyl; and L³ is —CH₂—.

In some embodiments, X¹ is N, C—R^{a}, or N—R^{c}; R^{a} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl; and R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂. In some embodiments, R^{a} is H. In some embodiments, R is H, C₁-C₆alkyl, C₁-C₆alkoxy, C₁-C₆fluoroalkyl, C₁-C₆fluoroalkoxy, or C₁-C₆heteroalkyl. In some embodiments, R^{c} is H, C₁-C₆alkyl or C₁-C₆fluoroalkyl. In some embodiments, R^{c} is C₂-C₄alkyl. In some embodiments, R^{c} is C₂-C₄fluoroalkyl. In some embodiments, the C₂-C₄fluoroalkyl comprises one, two, or three fluoro substituents.

In some embodiments, X² is N, C—R^{b}, or N—R^{c}; R^{b} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl; and R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂. In some embodiments, R^{b} is H. In some embodiments, R is H, C₁-C₆alkyl, C₁-C₆alkoxy, C₁-C₆fluoroalkyl, C₁-C₆fluoroalkoxy, or C₁-C₆heteroalkyl. In some embodiments, R^{c} is H, C₁-C₆alkyl or C₁-C₆fluoroalkyl. In some embodiments, R^{c} is C₂-C₄alkyl. In some embodiments, R^{c} is C₂-C₄fluoroalkyl. In some embodiments, the C₂-C₄fluoroalkyl comprises one, two, or three fluoro substituents. In some embodiments, R^{c} is H.

In some embodiments, X⁸ is C—R⁴ or N; R⁴ is selected from H, F, C₁, Br, —OH, —CN, —NH₂, —NH(CH₃), —N(CH₃)₂, —CH₃, —OCH₃, —CF₃, and —OCF₃; R⁵ is selected from H, F, Cl, Br, —OH, —CN, —NH₂, —NH(CH₃), —N(CH₃)₂, —CH₃, —OCH₃, —CF₃, and —OCF₃; R⁶ is selected from H, F, Cl, Br, —OH, —CN, —NH₂, —NH(CH₃), —N(CH₃)₂, —CH₃, —OCH₃, —CF₃, and —OCF₃; or R⁵ and R⁶ are taken together with the intervening atoms to which they are attached to form a substituted or unsubstituted fused phenyl, wherein if the fused phenyl is substituted then the fused phenyl is substituted with 1-4 R¹¹; R⁷ is selected from H, F, Cl, Br, —OH, —CN, —NH₂, —NH(CH₃), —N(CH₃)₂, —CH₃, —OCH₃, —CF₃, and —OCF₃; or R⁶ and R⁷ are taken together with the intervening atoms to which they are attached to form a substituted or unsubstituted fused phenyl, wherein if the fused phenyl is substituted then the fused ring is substituted with 1-4 R¹¹.

In some embodiments, X⁸ is C—R⁴; R⁴ is H; R⁵ is selected from H, F, C₁, Br, —OH, —CN, —CH₃, —OCH₃, —CF₃, and —OCF₃; R⁶ is selected from H, F, Cl, Br, —OH, —CN, —CH₃, —OCH₃, —CF₃, and —OCF₃; R⁷ is H.

In some embodiments, X⁸ is N; R⁵ and R⁶ are taken together with the intervening atoms to which they are attached to form a substituted or unsubstituted fused phenyl, wherein if the fused phenyl is substituted then the fused phenyl is substituted with 1-4 R¹¹; R⁷ is H.

In some embodiments, X⁸ is N; R⁵ is H; R⁶ and R⁷ are taken together with the intervening atoms to which they are attached to form a substituted or unsubstituted fused phenyl, wherein if the fused phenyl is substituted then the fused phenyl is substituted with 1-4 R¹¹.

In some embodiments, each R¹, R⁹ and R¹⁰ is independently selected from H, F, C₁, Br, —OH, —CN, —NH₂, —NH(CH₃), —N(CH₃)₂, —CH₃, —OCH₃, —CF₃, and —OCF₃. In some embodiments, each R⁸, R⁹ and R¹⁰ is H.

In some embodiments, X¹ is N or C—R^{a}; X² is N, C—R^{b}, or N—R^{c}; each R^{a}, R^{b}, and R^{c} is independently H, halogen, C₁-C₆alkyl, C₁-C₆alkoxy, C₁-C₆fluoroalkyl, or C₁-C₆fluoroalkoxy; X³ is C or N; X⁴ is C; X⁵ is C—H or N; X⁶ is C—H; X⁷ is C—H; X⁸ is C—H; R⁵ is selected from H, F, Cl, Br, —OH, —CN, —CH₃, —OCH₃, —CF₃, and —OCF₃; and R⁶ is selected from H, F, Cl, Br, —OH, —CN, —CH₃, —OCH₃, —CF₃, and —OCF₃.

In some embodiments, R^{a} is H or C₁-C₆alkyl. In some embodiments, R^{b} is H. In some embodiments, R^{c} is H or C₁-C₆alkyl. In some embodiments, R^{a} is H or C₁-C₆alkyl; R^{b} is H; and R^{c} is H or C₁-C₆alkyl. In some embodiments, the C₁-C₆alkyl of any one of R^{a} or R^{c} is methyl, ethyl, propyl, isopropyl, butyl, isobutyl, sec-butyl, or tert-butyl.

In some embodiments, X¹ is C—H; X² is N; X³ is N; X⁴ is C; X⁵, X⁶, X⁷, and X⁸ are each C—H; R⁵ is H or —CF₃; R⁶ is H or —CF₃; and R⁷ is H.

In some embodiments, X¹ is N; X² is C—H; X³ is N; X⁴ is C; X⁵, X⁶, X⁷, and X⁸ are each C—H; R⁵ is H or —CF₃; R⁶ is H or —CF₃; and R⁷ is H.

In some embodiments, X¹ is N; X² is N—R; R^{c} is H or C₁-C₆alkyl; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is N; X² is N—R^{c}; R^{c} is H; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is N; X² is N—R; R is C₁-C₆alkyl; X³ is C; X⁴ is C; X⁵, X⁶, X⁷, and X⁸ are each C—H; R⁵ is H or —CF₃; R⁶ is H or —CF₃; and R⁷ is H.

In some embodiments, X¹ is N—R^{c}; X² is N; R^{c} is H or C₁-C₆alkyl; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is N—R^{c}; X² is N; R is H; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is N—R^{c}; X² is N; R is C₁-C₆alkyl; X³ is C; X⁴ is C; X⁵, X⁶, X⁷, and X⁸ are each C—H; R⁵ is H or —CF₃; R⁶ is H or —CF₃; and R⁷ is H.

In some embodiments, X¹ is C—H; X² is N—R; R^{c} is H or C₁-C₆alkyl; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is C—H; X² is N—R; R is H; X³ is C; X⁴ is C; X⁵, X⁶, X⁷, and X⁸ are each C—H; R⁵ is H or —CF₃; R⁶ is H or —CF₃; and R⁷ is H. In some embodiments, X¹ is C—H; X² is N—R; R is C₁-C₆alkyl; X³ is C; X⁴ is C; X⁵, X⁶, X⁷, and X⁸ are each C—H; R⁵ is H or —CF₃; R⁶ is H or —CF₃; and R⁷ is H.

In some embodiments, X¹ is C—H; X² is N—R; R^{c} is H or C₁-C₆alkyl; X³ is C; X⁴ is C; X⁵ is N; X⁶, X⁷, and X⁸ are each C—H; R⁵ is H or —CF₃; R⁶ is H or —CF₃; and R⁷ is H. In some embodiments, X¹ is C—H; X² is N—R^{c}; R^{c} is H; X³ is C; X⁴ is C; X⁵ is N; X⁶, X⁷, and X⁸ are each C—H; R⁵ is H or —CF₃; R⁶ is H or —CF₃; and R⁷ is H. In some embodiments, X¹ is C—H; X² is N—R^{c}; R^{c} is C₁-C₆alkyl; X³ is C; X⁴ is C; X⁵ is N; X⁶, X⁷, and X⁸ are each C—H; R⁵ is H or —CF₃; R⁶ is H or —CF₃; and R⁷ is H.

In some embodiments, R^{c} is H. In some embodiments, R^{c} is C₁-C₆alkyl. In some embodiments, the C₁-C₆alkyl of R^{c} is methyl, ethyl, propyl, isopropyl, butyl, isobutyl, sec-butyl, or tert-butyl.

In some embodiments, R⁵ is H or —CF₃. In some embodiments, R⁶ is H or —CF₃. In some embodiments, R⁵ is H and R⁶ is —CF₃. In some embodiments, R⁵ is —CF₃ and R⁶ is H.

In some embodiments, L³ is —CH₂—, —CH₂CH₂—, —CH(CH₃)—, —C(CH₃)₂—, —O—, —OCH₂—, —OCH₂CH₂—, —OCH(CH₃)—, —OC(CH₃)₂—, —NH—, —NHCH₂—, —NHCH₂CH₂—, —NHCH(CH₃)—, or —NHC(CH₃)₂—.

In some embodiments, L³ is —CH₂—, —O—, or —NH—.

In some embodiments, L³ is —CH₂—.

In some embodiments

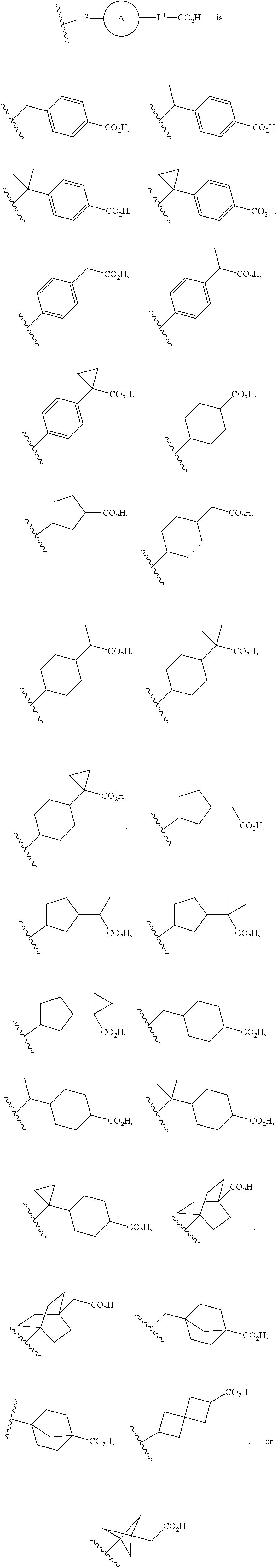

In another aspect, described herein is a compound of Formula (XII), or a pharmaceutically acceptable salt or solvate thereof:

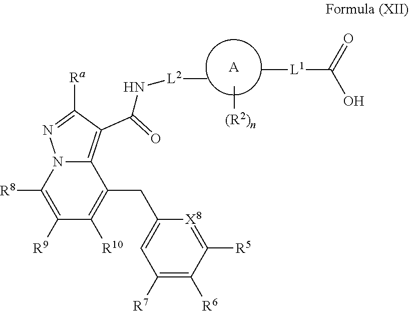

wherein,
L¹ is absent, C₁-C₄alkylene, or C₃-C₆cycloalkylene;
ring A is a phenyl, or C₃-C₁₂cycloalkyl;
L² is absent, C₁-C₄alkylene, or C₃-C₆cycloalkylene;
each R² is independently selected from H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R^{a} is H, halogen, C₁-C₆alkyl, C₁-C₆alkoxy, C₁-C₆fluoroalkyl, C₁-C₆fluoroalkoxy, or C₁-C₆heteroalkyl;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R⁸, R⁹, and R¹⁰ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, or 2.

In some embodiments, L¹ is absent or C₁-C₄alkylene. In some embodiments, L² is absent or C₁-C₄alkylene. In some embodiments, L¹ is absent or C₁-C₄alkylene and L² is absent or C₁-C₄alkylene. In some embodiments, L¹ is absent and L² is C₁-C₄alkylene.

In some embodiments, L¹ is absent, —CH₂—,

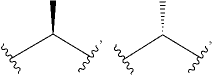

or cyclopropyl-1,1-diyl. In some embodiments, L² is absent, —CH₂—,

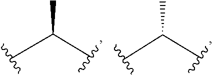

or cyclopropyl-1,1-diyl. In some embodiments, L¹ is absent and L² is —CH₂—

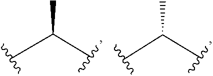

or cyclopropyl-1,1-diyl. In some embodiments, L¹ is absent and L² is —CH₂—.

In some embodiments, L¹ is absent; L² is —CH₂—; and ring A is C₃-C₁₂cycloalkyl or C₂-C₁₀heterocycloalkyl.

In some embodiments,

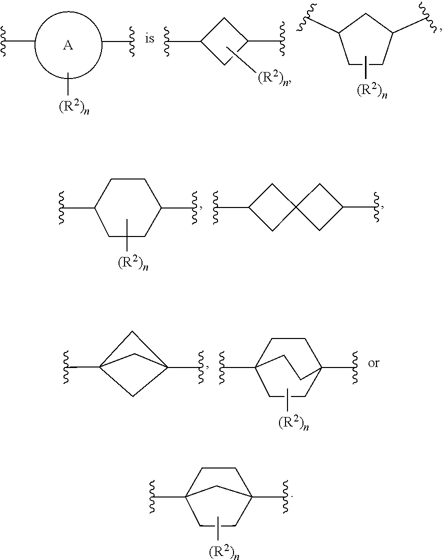

In some embodiments,

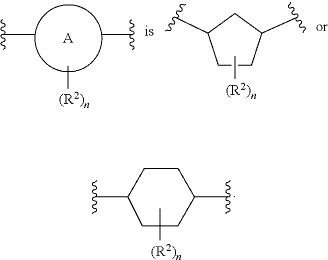

In some embodiments, n is 0.

In some embodiments,

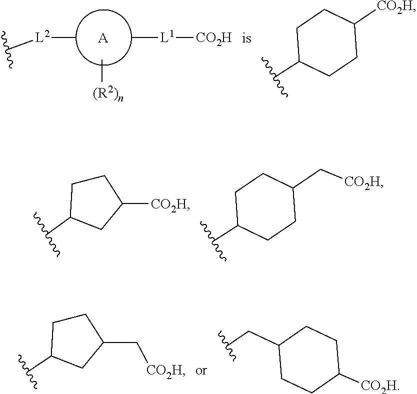

In some embodiments,

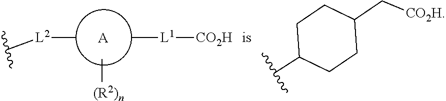

In some embodiments,

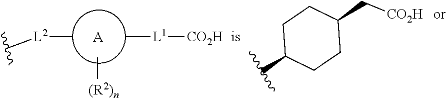

In some embodiments,

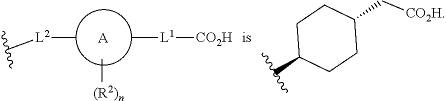

In some embodiments, R^{a} is H, halogen, C₁-C₆alkyl, C₁-C₆alkoxy, C₁-C₆fluoroalkyl, or C₁-C₆fluoroalkoxy. In some embodiments, R^{a} is H, halogen, or C₁-C₄alkyl. In some embodiments, R^{a} is H.

In some embodiments, each R¹, R⁹ and R¹⁰ is independently selected from H, F, Cl, Br, —OH, —CN, —NH₂, —NH(CH₃), —N(CH₃)₂, —CH₃, —OCH₃, —CF₃, and —OCF₃. In some embodiments, each R⁸, R⁹ and R¹⁰ is independently selected from H, F, Cl, Br, —CH₃, —OCH₃, —CF₃, and —OCF₃. In some embodiments, R⁸, R⁹ and R¹⁰ are each H.

In some embodiments, X⁸ is C—R⁴; R⁴ is H; R⁵ is selected from H, F, Cl, Br, —OH, —CN, —CH₃, —OCH₃, —CF₃, and —OCF₃; R⁶ is selected from H, F, Cl, Br, —OH, —CN, —CH₃, —OCH₃, —CF₃, and —OCF₃; and R⁷ is H.

In some embodiments, R^{a} is H; X⁸ is C—R⁴; R⁴ is H; R⁵ is H or —CF₃; R⁶ is H or —CF₃; R⁷ is H; and R⁸, R⁹ and R¹⁰ are each H.

In some embodiments, R^{a} is H; X⁸ is C—R⁴; R⁴ is H; R⁵ is H or —CF₃; R⁶ is H or —CF₃; R⁷ is H; R⁸, R⁹ and R¹⁰ are each H; and

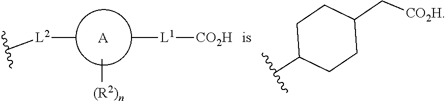

Any combination of the groups described above for the various variables is contemplated herein. Throughout the specification, groups and substituents thereof are chosen by one skilled in the field to provide stable moieties and compounds.

Representative compounds of Formula (I) include, but are not limited to:

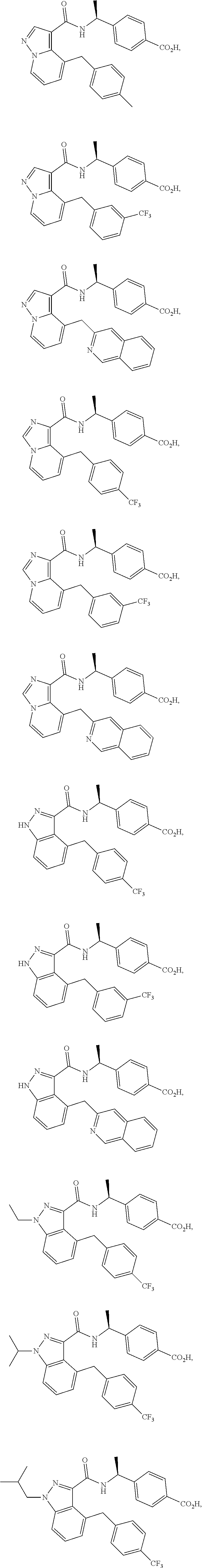

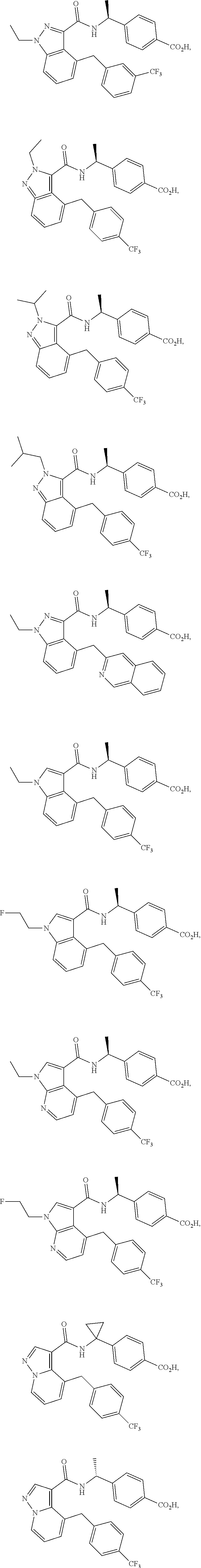

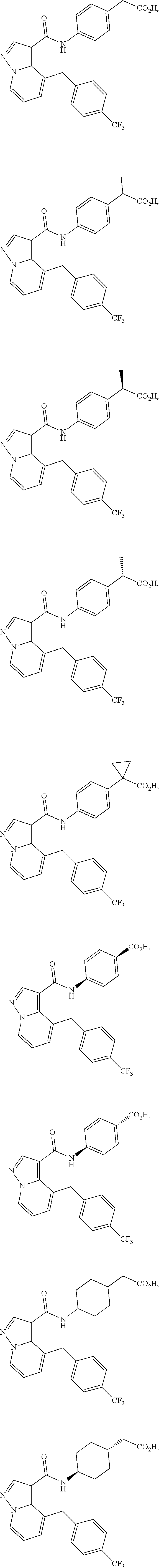

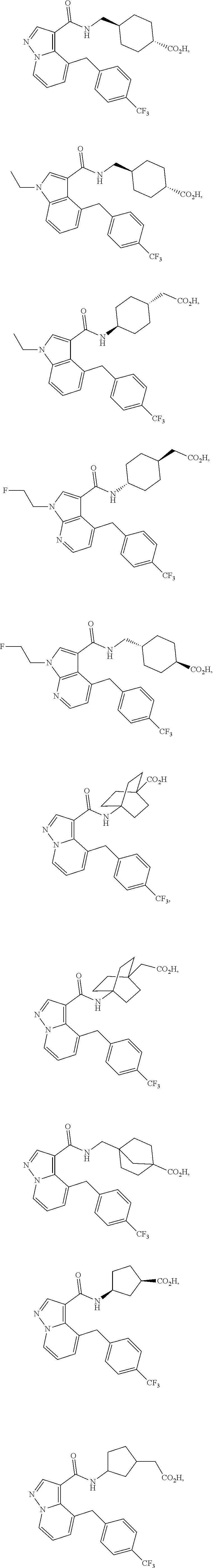

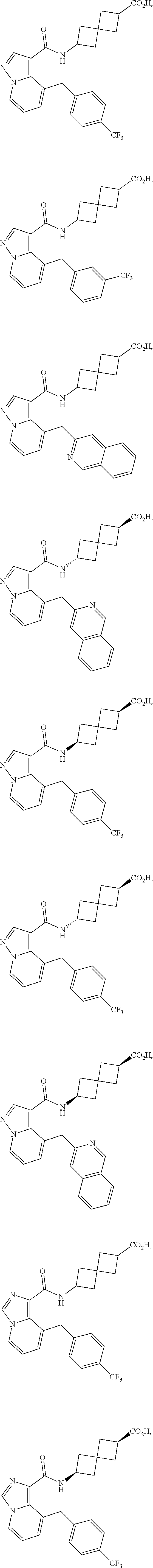

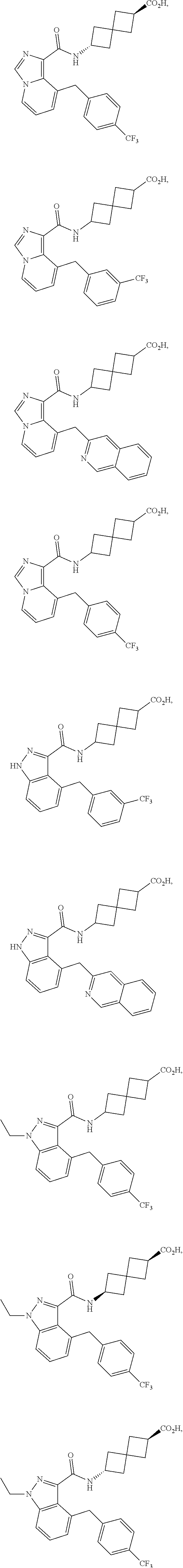

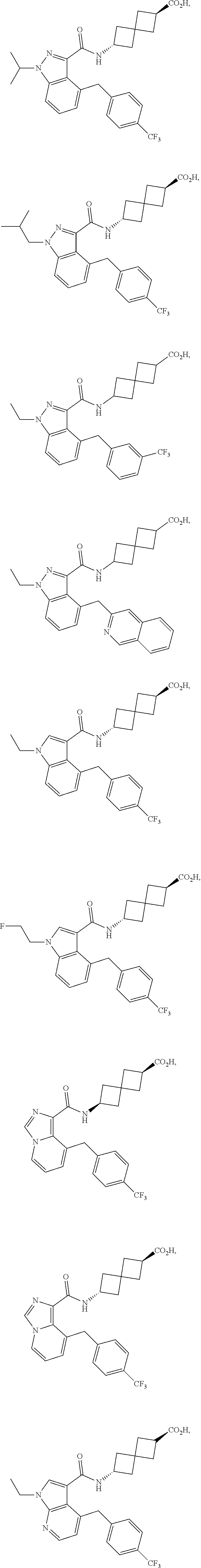

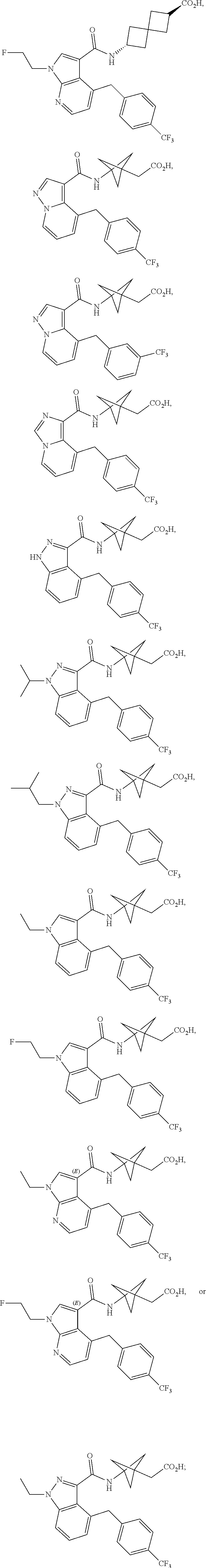

In some embodiments, compounds include, but are not limited to:
4-[(1S)-1-[[4-[[4-(trifluoromethyl)phenyl]methyl]pyrazolo[1,5-a]pyridine-3-carbonyl]amino]ethyl]benzoic acid;
2-[[4-[[4-(trifluoromethyl)phenyl]methyl]pyrazolo[1,5-a]pyridine-3-carbonyl]amino]spiro[3.3]heptane-6-carboxylic acid;
4-[(1S)-1-[[4-[[3-(trifluoromethyl)phenyl]methyl]pyrazolo[1,5-a]pyridine-3-carbonyl]amino]ethyl]benzoic acid;
2-[[4-[[3-(trifluoromethyl)phenyl]methyl]pyrazolo[1,5-a]pyridine-3-carbonyl]amino]spiro[3.3]heptane-6-carboxylic acid;
4-[(1S)-1-[[4-(3-isoquinolylmethyl) pyrazolo[1,5-a]pyridine-3-carbonyl]amino]ethyl]benzoic acid;
cis-2-[[4-(3-isoquinolylmethyl) pyrazolo[1,5-a]pyridine-3-carbonyl]amino]spiro[3.3]heptane-6-carboxylic acid;
trans-2-[[4-(3-isoquinolylmethyl) pyrazolo[1,5-a]pyridine-3-carbonyl]amino]spiro[3.3]heptane-6-carboxylic acid;
cis-6-[[4-[[4-(trifluoromethyl)phenyl]methyl]pyrazolo[1,5-a]pyridine-3-carbonyl]amino]spiro[3.3]heptane-2-carboxylic acid;
trans-2-[[4-[[4-(trifluoromethyl)phenyl]methyl]pyrazolo[1,5-a]pyridine-3-carbonyl]amino]spiro[3.3]heptane-6-carboxylic acid;
2-[3-[[4-[[4-(trifluoromethyl)phenyl]methyl]pyrazolo[1,5-a]pyridine-3-carbonyl]amino]-1-bicyclo[1.1.1]pentanyl]acetic acid;
2-[3-[[4-[[3-(trifluoromethyl)phenyl]methyl]pyrazolo[1,5-a]pyridine-3-carbonyl]amino]-1-bicyclo[1.1.1]pentanyl]acetic acid;
4-[1-[[4-[[4-(trifluoromethyl)phenyl]methyl]pyrazolo[1,5-a]pyridine-3-carbonyl]amino]cyclopropyl]benzoic acid;
4-[(1R)-1-[[4-[[4-(trifluoromethyl)phenyl]methyl]pyrazolo[1,5-a]pyridine-3-carbonyl]amino]ethyl]benzoic acid;
2-[4-[[4-[[4-(trifluoromethyl)phenyl]methyl]pyrazolo[1,5-a]pyridine-3-carbonyl]amino]phenyl]acetic acid;
1-[4-[[4-[[4-(trifluoromethyl)phenyl]methyl]pyrazolo[1,5-a]pyridine-3-carbonyl]amino]phenyl]cyclopropanecarboxylic acid;
(1s,4s)-4-[[4-[[4-(trifluoromethyl)phenyl]methyl]pyrazolo[1,5-a]pyridine-3-carbonyl]amino]cyclohexanecarboxylic acid;
(1r,4r)-4-[[4-[[4-(trifluoromethyl)phenyl]methyl]pyrazolo[1,5-a]pyridine-3-carbonyl]amino]cyclohexanecarboxylic acid;
2-[4-[[4-[[4-(trifluoromethyl)phenyl]methyl]pyrazolo[1,5-a]pyridine-3-carbonyl]amino]cyclohexyl]acetic acid;
2-(4-(4-(4-(trifluoromethyl)benzyl)pyrazolo[1,5-a]pyridine-3-carboxamido)bicyclo[2.2.2]octan-1-yl)acetic acid;
4-[[4-[[4-(trifluoromethyl)phenyl]methyl]pyrazolo[1,5-a]pyridine-3-carbonyl]amino]bicyclo[2.2.2]octane-1-carboxylic acid;
2-[3-[[4-[[4-(trifluoromethyl)phenyl]methyl]pyrazolo[1,5-a]pyridine-3-carbonyl]amino]cyclopentyl]acetic acid;
2-((1r,4r)-4-(4-(4-(trifluoromethyl)benzyl)pyrazolo[1,5-a]pyridine-3-carboxamido)cyclohexyl)acetic acid;
(1R,3S)-3-[[4-[[4-(trifluoromethyl)phenyl]methyl]pyrazolo[1,5-a]pyridine-3-carbonyl]amino]cyclopentanecarboxylic acid;
(1r,4r)-4-[[[4-[[4-(trifluoromethyl)phenyl]methyl]pyrazolo[1,5-a]pyridine-3-carbonyl]amino]methyl]cyclohexanecarboxylic acid;
2-[4-[[4-[[4-(trifluoromethyl)phenyl]methyl]pyrazolo[1,5-a]pyridine-3-carbonyl]amino]phenyl]propanoic acid;
4-[[[4-[[4-(trifluoromethyl)phenyl]methyl]pyrazolo[1,5-a]pyridine-3-carbonyl]amino]methyl]norbornane-1-carboxylic acid.

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Ztrich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the compound of Formula (I) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound of Formula (I) with a base. In some embodiments, the compound of Formula (I) is acidic and is reacted with a base. In such situations, an acidic proton of the compound of Formula (I) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (I), as well as active metabolites of these compounds having the same type of activity.

In some embodiments, sites on the organic radicals (e.g. alkyl groups, aromatic rings) of compounds of Formula (I) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogens of the compounds of Formula (I) are replaced with deuterium.

In some embodiments, the compounds of Formula (I) possess one or more stereocenters and each stereocenter exists independently in either the R or S configuration. In some embodiments, the compound of Formula (I) exists in the R configuration. In some embodiments, the compound of Formula (I) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formula (I) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds of Formula (I) described herein are synthesized using standard synthetic techniques or using methods known in the art in combination with methods described herein. Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are synthesized as outlined in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to —NH(alkyl), or —N(alkyl)₂.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, adamantyl, norbornyl, and decalinyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, and —CH₂SCH₃.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclcic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target. In some embodiments, “modulate” means to interact with a target either directly or indirectly so as to decrease or inhibit receptor activity,

The term “modulator” as used herein, refers to a molecule that interacts with a target either directly or indirectly. The interactions include, but are not limited to, the interactions of an agonist, partial agonist, an inverse agonist, antagonist, or combinations thereof. In some embodiments, a modulator is an antagonist. Receptor antagonists are inhibitors of receptor activity. Antagonists mimic ligands that bind to a receptor and prevent receptor activation by a natural ligand. Preventing activation may have many effects. If a natural agonist binding to a receptor leads to an increase in cellular function, an antagonist that binds and blocks this receptor decreases the function.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

It should be understood that in addition to the ingredients particularly mentioned above, the compounds and compositions described herein may include other agents conventional in the art having regard to the type of formulation in question, for example those suitable for oral administration may include flavoring agents.

### Methods of Treatment, Dosing and Treatment Regimens

The compounds disclosed herein, or pharmaceutically acceptable salts, solvates, or stereoisomers thereof, are useful for the modulation of prostaglandin receptors. In some embodiments, the prostaglandin receptor modulated by the compounds and methods is prostaglandin E₂ receptor 4 (EP₄).

Provided herein are antagonists of prostaglandin E₂ receptor 4 (EP₄) that useful for treating one or more diseases or disorders associated with EP₄ activity.

In some embodiments, described herein are methods for treating a disease or disorder, wherein the disease or disorder is cancer, a hyperproliferative disorder, an autoimmune disorder, or inflammatory disorder.

In one embodiment, the compounds described herein, or a pharmaceutically acceptable salt thereof, are used in the preparation of medicaments for the treatment of diseases or conditions in a mammal that would benefit from inhibition or reduction of EP4 activity. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In certain embodiments wherein a mammal's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like.

Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, radiation therapy, monoclonal antibodies, or combinations thereof.

Chemotherapy includes the use of anti-cancer agents.

### EXAMPLES

The following examples are provided for illustrative purposes only and not to limit the scope of the claims provided herein.

### Example 1—Synthesis of 4-[(1S)-1-[[4-[[4-(trifluoromethyl)phenyl]methyl]pyrazolo[1,5-a]pyridine-3-carbonyl]amino]ethyl]benzoic acid (Compound 1)

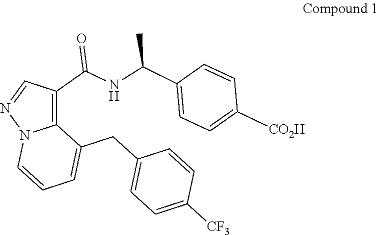 [SMILES: C[C@H](NC(=O)c1cnn2cccc(Cc3ccc(C(F)(F)F)cc3)c12)c1ccc(C(=O)O)cc1]

### Step 1: Preparation of methyl4-[[4-(trifluoromethyl)phenyl]methyl]pyrazolo[1,5-a]pyridine-3-carboxylate

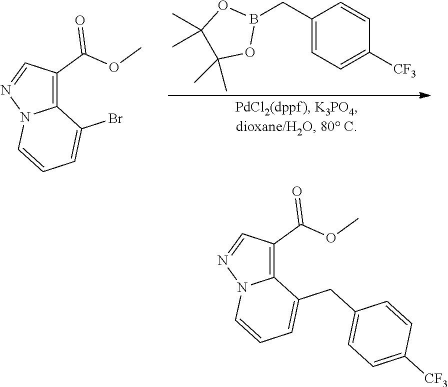

To a solution of methyl4-bromopyrazolo[1,5-a]pyridine-3-carboxylate (708 mg, 2.77 mmol, 1 eq) and 4,4,5,5-tetramethyl-2-[[4-(trifluoromethyl)phenyl]methyl]-1,3,2-dioxaborolane (794.12 mg, 2.77 mmol, 1 eq) in dioxane (5 mL) and H₂O (1 mL) was added Pd(dppf)Cl₂ (507.75 mg, 2.77 mmol, 1 eq) and K₃PO₄ (697.2 mg, 2.77 mmol, 1 eq). The mixture was stirred at 80° C. for 2 hr.

The solvent was removed to afford the crude product. The residue was purified by column chromatography (SiO₂, Petroleum ether/Ethyl acetate=100/1 to 0/1) to provide methyl4-[[4-(trifluoromethyl)phenyl]methyl]pyrazolo[1,5-a]pyridine-3-carboxylate (270 mg, 807.68 μmol, 29.10% yield) as a white solid. LCMS for product (ESI): m/z 355.2 [M+H]⁺.

¹H NMR (400 MHz, CDCl₃) δ=8.51-8.48 (m, 2H), 7.58 (d, J=8.0 Hz, 2H), 7.33 (d, J=8.0 Hz, 2H), 7.07 (d, J=7.2 Hz, 1H), 6.96-6.92 (m, 1H), 4.87 (s, 2H), 3.87 (s, 3H)

### Step 2: Preparation of 4-[[4-(trifluoromethyl)phenyl]methyl]pyrazolo[1,5-a]pyridine-3-carboxylic acid

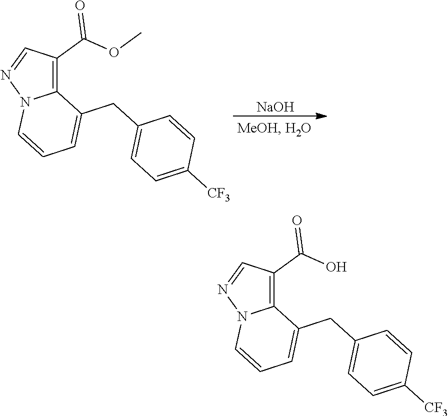 [SMILES: COC(=O)c1cnn2cccc(Cc3ccc(C(F)(F)F)cc3)c12 ; "NaOH" ; O=C(O)c1cnn2cccc(Cc3ccc(C(F)(F)F)cc3)c12]

To a solution of methyl 4-[[4-(trifluoromethyl) phenyl]methyl]pyrazolo[1,5-a]pyridine-3-carboxylate (270 mg, 807.68 μmol, 1 eq) in MeOH (0.5 mL) was added NaOH (32.31 mg, 807.68 μmol, 1 eq) and H₂O (0.5 mL). The mixture was stirred at 40° C. for 4 hr

The solvent was removed to afford the crude product. The mixture was adjusted to pH 4 with 1N HCl. The mixture was filtered and compound 4-[[4-(trifluoromethyl) phenyl]methyl]pyrazolo[1,5-a]pyridine-3-carboxylic acid (180 mg, 562.03 μmol, 69.59% yield) was obtained as a white solid. ¹H NMR (400 MHz, DMSO-d₆) δ=12.3 (br s, 1H), 8.77 (d, J=6.8 Hz, 1H), 8.44 (s, 1H), 7.62 (d, J=8.4 Hz, 2H), 7.38-7.34 (m, 3H), 7.12 (t, J=6.8 Hz, 1H), 4.84 (s, 2H)

### Step 3: Preparation of methyl4-[(1S)-1-[[4-[[4-(trifluoromethyl)phenyl]methyl]pyrazolo[1,5-a]pyridine-3-carbonyl]amino]ethyl]benzoate

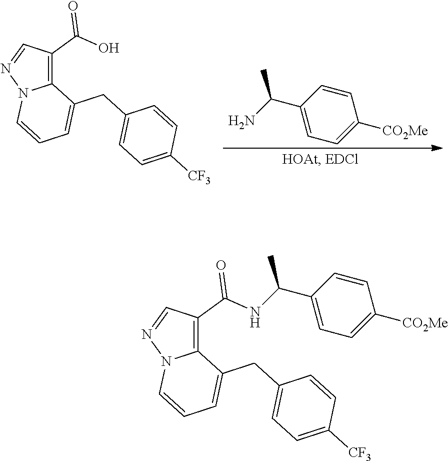 [SMILES: O=C(O)c1cnn2cccc(Cc3ccc(C(F)(F)F)cc3)c12 ; COC(=O)c1ccc([C@H](C)N)cc1 ; COC(=O)c1ccc([C@H](C)NC(=O)c2cnn3cccc(Cc4ccc(C(F)(F)F)cc4)c23)cc1]

To a solution of 4-[[4-(trifluoromethyl) phenyl]methyl]pyrazolo[1,5-a]pyridine-3-carboxylic acid (90 mg, 281.02 μmol, 1 eq) and methyl 4-[(1S)-1-aminoethyl]benzoate (75.54 mg, 421.53 μmol, 1.5 eq) in DCM (8 mL) was added HOAt (7.65 mg, 56.20 μmol, 7.86 μL, 0.2 eq), Et₃N (142.18 mg, 1.41 mmol, 195.57 μL, 5 eq) and EDCI (109.06 mg, 702.54 μmol, 124.36 μL, 2.5 eq). The mixture was stirred at 25° C. for 16 hr.

The solvent was removed to afford the crude product. The residue was purified by column chromatography (SiO₂, Petroleum ether/Ethyl acetate=100/1 to 0/1). Compound methyl 4-[(1S)-1-[[4-[[4-(trifluoromethyl)phenyl]methyl]pyrazolo[1,5-a]pyridine-3-carbonyl]amino]ethyl]benzoate (50 mg, 103.85 μmol, 36.95% yield) was obtained as a white solid, which was used into next step directly without further purification. LCMS for product (ESI): m/z 482.3 [M+H]⁺.

### Step 4: Preparation of Compound 1,4-[(1S)-1-[[4-[[4-(trifluoromethyl)phenyl]methyl]pyrazolo[1,5-a]pyridine-3-carbonyl]amino]ethyl]benzoic acid

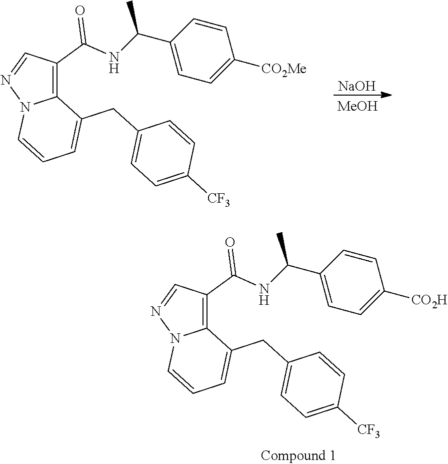 [SMILES: "MeOH" ; COC(=O)c1ccc([C@H](C)NC(=O)c2cnn3cccc(Cc4ccc(C(F)(F)F)cc4)c23)cc1 ; "NaOH" ; C[C@H](NC(=O)c1cnn2cccc(Cc3ccc(C(F)(F)F)cc3)c12)c1ccc(C(=O)O)cc1]

To a solution of methyl 4-[(1S)-1-[[4-[[4-(trifluoromethyl)phenyl]methyl]pyrazolo[1,5-a]pyridine-3-carbonyl]amino]ethyl]benzoate (50 mg, 103.85 μmol, 1 eq) in MeOH (0.5 mL) was added NaOH (4.15 mg, 103.85 μmol, 1 eq) and H₂O (0.5 mL). The mixture was stirred at 40° C. for 2 hr.

The solvent was removed to afford the crude product. The mixture was adjusted to pH 4 with 1N HCl. The mixture was filtered and Compound 1, 4-[(1S)-1-[[4-[[4-(trifluoromethyl)phenyl]methyl]pyrazolo[1,5-a]pyridine-3-carbonyl]amino]ethyl]benzoic acid (10 mg, 19.68 μmol, 18.95% yield, 92% purity), was obtained as a white solid. LCMS for product (ESI): m/z 468.1 [M+H]⁺. ¹H NMR (400 MHz, DMSO-d₆) δ=8.72-8.68 (m, 2H), 8.33 (s, 1H), 7.91 (d, J=8.4 Hz, 2H), 7.50 (d, J=8.4 Hz, 2H), 7.39-7.32 (m, 3H), 7.19 (d, J=8.4 Hz, 2H), 7.04 (t, J=6.8 Hz, 1H), 5.17 (t, J=7.2 Hz, 1H), 4.69 (d, J=14.4 Hz, 1H), 4.49 (d, J=14.4 Hz, 1H), 1.41 (d, J=7.2 Hz, 3H)

### Example 2—Synthesis of 2-[[4-[[4-(trifluoromethyl)phenyl]methyl]pyrazolo[1,5-a]pyridine-3-carbonyl]amino]spiro[3.3]heptane-6-carboxylic acid (Compound 2)

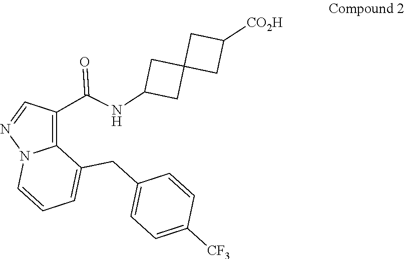 [SMILES: O=C(NC1CC2(C1)CC(C(=O)O)C2)c1cnn2cccc(Cc3ccc(C(F)(F)F)cc3)c12]

### Step 1: Preparation of ethyl 2-[[4-[[4-(trifluoromethyl)phenyl]methyl]pyrazolo[1,5-a]pyridine-3-carbonyl]amino]spiro[3.3]heptane-6-carboxylate

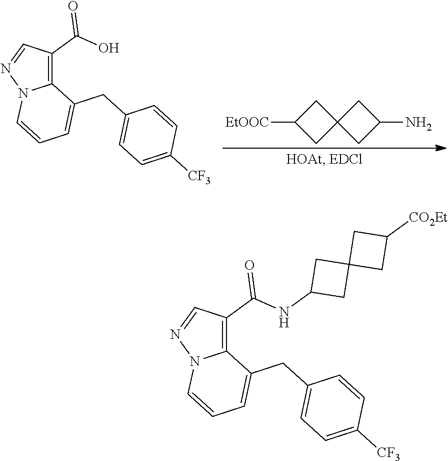 [SMILES: O=C(O)c1cnn2cccc(Cc3ccc(C(F)(F)F)cc3)c12 ; CCOC(=O)C1CC2(CC(N)C2)C1 ; CCOC(=O)C1CC2(CC(NC(=O)c3cnn4cccc(Cc5ccc(C(F)(F)F)cc5)c34)C2)C1]

To a solution of 4-[[4-(trifluoromethyl) phenyl]methyl]pyrazolo[1,5-a]pyridine-3-carboxylic acid (90 mg, 281.02 μmol, 1 eq) and ethyl 2-aminospiro[3.3]heptane-6-carboxylate (77.24 mg, 421.53 μmol, 1.5 eq) in DCM (8 mL) was added HOAt (7.65 mg, 56.20 μmol, 7.86 μL, 0.2 eq), Et₃N (142.18 mg, 1.41 mmol, 195.57 μL, 5 eq) and EDCI (109.06 mg, 702.54 μmol, 2.5 eq). The mixture was stirred at 25° C. for 16 hr.

The solvent was removed to afford the crude product. The residue was purified by column chromatography (SiO₂, Petroleum ether/Ethyl acetate=100/1 to 0/1) to provide ethyl 2-[[4-[[4-(trifluoromethyl) phenyl]methyl]pyrazolo[1,5-a]pyridine-3-carbonyl]amino]spiro[3.3]heptane-6-carboxylate (60 mg, 123.58 μmol, 43.98% yield) as a white solid, which was used into next step directly without further purification.

### Step 2: Preparation of Compound 2,2-[[4-[[4-(trifluoromethyl)phenyl]methyl]pyrazolo[1,5-a]pyridine-3-carbonyl]amino]spiro[3.3]heptane-6-carboxylic acid

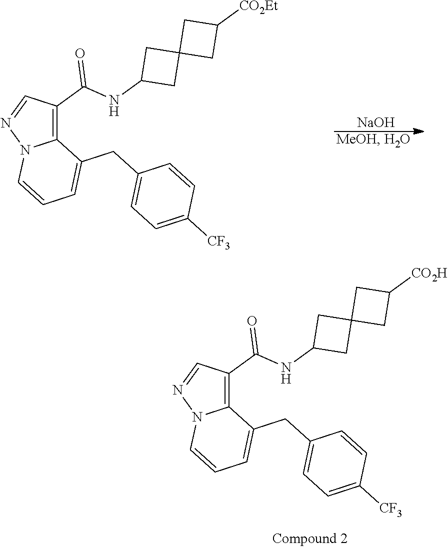

To a solution of ethyl 2-[[4-[[4-(trifluoromethyl) phenyl]methyl]pyrazolo[1,5-a]pyridine-3-carbonyl]amino]spiro[3.3]heptane-6-carboxylate (60 mg, 123.58 μmol, 1 eq) in MeOH (0.6 mL) was added NaOH (4.94 mg, 123.58 μmol, 1 eq) and H₂O (0.6 mL). The mixture was stirred at 40° C. for 2 hr.

The solvent was removed to afford the crude product. The mixture was adjusted to pH 4 with 1N HCl. The mixture was filtered and Compound 2, 2-[[4-[[4-(trifluoromethyl) phenyl]methyl]pyrazolo[1,5-a]pyridine-3-carbonyl]amino]spiro[3.3]heptane-6-carboxylic acid (40 mg, 84.82 μmol, 68.63% yield, 97% purity) was obtained as a white solid. LCMS for product (ESI): m/z 458.2 [M+H]⁺. ¹H NMR (400 MHz, DMSO-d₆) δ=12.06 (brs, 1H), 8.67 (d, J=6.8 Hz, 1H), 8.31 (d, J=7.6 Hz, 1H), 8.19 (s, 1H), 7.56 (d, J=8.0 Hz, 2H), 7.34-7.27 (m, 3H), 7.03 (t, J=6.8 Hz, 1H), 4.63 (s, 2H), 4.25-4.16 (m, 1H), 2.96-2.50 (m, 1H), 2.43-2.36 (m, 1H), 2.35-2.17 (m, 3H), 2.16-2.03 (m, 2H), 1.96-1.84 (m, 2H)

### Example 3—Synthesis of 4-[(1S)-1-[[4-[[3-(trifluoromethyl)phenyl]methyl]pyrazolo[1,5-a]pyridine-3-carbonyl]amino]ethyl]benzoic acid (Compound 3)

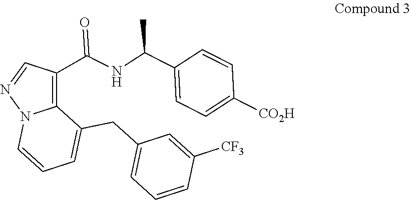 [SMILES: C[C@H](NC(=O)c1cnn2cccc(Cc3cccc(C(F)(F)F)c3)c12)c1ccc(C(=O)O)cc1]

### Step 1: Preparation of methyl4-[[3-(trifluoromethyl)phenyl]methyl]pyrazolo[1,5-a]pyridine-3-carboxylate

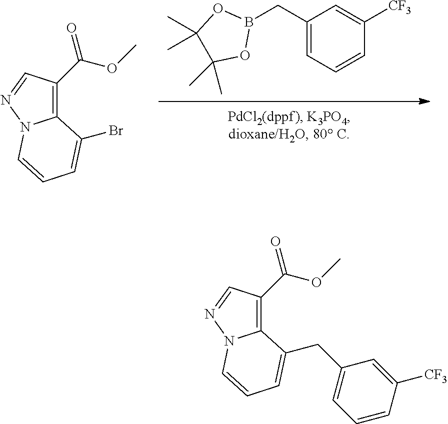

To a solution of methyl 4-bromopyrazolo[1,5-a]pyridine-3-carboxylate (800 mg, 3.136 mmol, 1 eq) and 4,4,5,5-tetramethyl-2-[[3-(trifluoromethyl) phenyl]methyl]-1,3,2-dioxaborolane (897.32 mg, 3.316 mmol, 1 eq) in dioxane (10 mL) and H₂O (2 mL) was added K₃PO₄ (665.76 mg, 3.136 mmol, 1 eq) and Pd(dppf)Cl₂ (688.48 mg, 940.92 μmol, 0.3 eq). The mixture was stirred at 80° C. for 2 hr.

The solvent was removed to afford the crude product. The residue was purified by column chromatography (SiO₂, Petroleum ether/Ethyl acetate=100/1 to 0/1) to provide methyl4-[[3-(trifluoromethyl)phenyl]methyl]pyrazolo[1,5-a]pyridine-3-carboxylate (300 mg, 897.42 μmol, 28.61% yield) as a white solid. ¹H NMR (400 MHz, DMSO-d₆) δ=8.47 (d, J=6.8 Hz, 1H), 8.44 (s, 1H), 7.49-4.46 (m, 2H), 7.39-7.33 (m, 2H), 7.05 (d, J=7.2 Hz, 1H), 6.91 (t, J=6.8 Hz, 1H), 4.82 (s, 2H), 3.84 (s, 3H)

### Step 2: Preparation of 4-[[3-(trifluoromethyl) phenyl]methyl]pyrazolo[1,5-a]pyridine-3-carboxylic acid

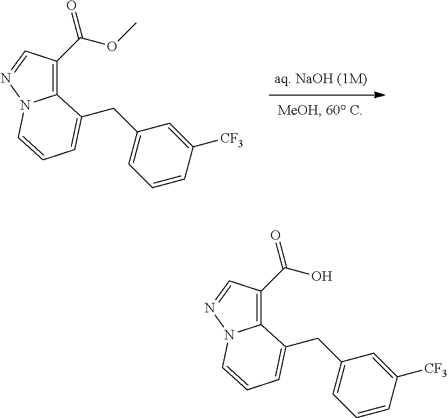 [SMILES: COC(=O)c1cnn2cccc(Cc3cccc(C(F)(F)F)c3)c12 ; O=C(O)c1cnn2cccc(Cc3cccc(C(F)(F)F)c3)c12]

To a solution of methyl 4-[[3-(trifluoromethyl) phenyl]methyl]pyrazolo[1,5-a]pyridine-3-carboxylate (290 mg, 867.51 μmol, 1 eq) in MeOH (1 mL) was added NaOH (34.70 mg, 867.51 μmol, 1 eq) and H₂O (2 mL). The mixture was stirred at 40° C. for 12 hr.

The solvent was removed to afford the crude product. The mixture was adjusted to pH 4 with 1N HCl. The mixture was filtered and 4-[[3-(trifluoromethyl) phenyl]methyl]pyrazolo[1,5-a]pyridine-3-carboxylic acid (140 mg, 437.14 μmol, 50.39% yield) was obtained as a white solid.

LCMS for product (ESI): m/z 321.2 [M+H]⁺.

### Step 3: Preparation of methyl 4-[(1S)-1-[[4-[[3-(trifluoromethyl) phenyl]methyl]pyrazolo[1,5-a]pyridine-3-carbonyl]amino]ethyl]benzoate

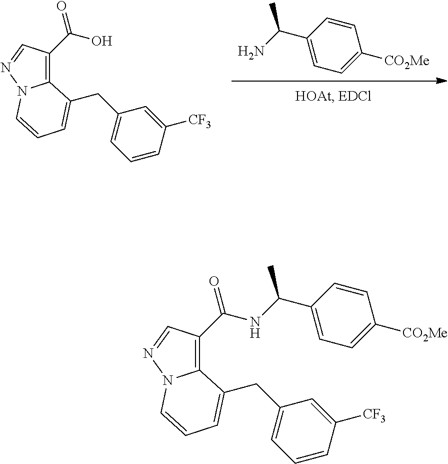 [SMILES: O=C(O)c1cnn2cccc(Cc3cccc(C(F)(F)F)c3)c12 ; COC(=O)c1ccc([C@H](C)N)cc1 ; COC(=O)c1ccc([C@H](C)NC(=O)c2cnn3cccc(Cc4cccc(C(F)(F)F)c4)c23)cc1]

To a solution of 4-[[3-(trifluoromethyl) phenyl]methyl]pyrazolo[1,5-a]pyridine-3-carboxylic acid (65.00 mg, 202.96 μmol, 1 eq) and methyl 4-[(1S)-1-aminoethyl]benzoate (72.75 mg, 405.91 μmol, 2 eq) in DCM (1 mL) was added HOAt (5.52 mg, 40.59 μmol, 5.68 μL, 0.2 eq), Et3N (102.69 mg, 1.01 mmol, 141.25 μL, 5 eq) and EDCI (63.01 mg, 405.91 μmol, 71.85 μL, 2 eq). The mixture was stirred at 25° C. for 16 hr.

The solvent was removed to afford the crude product. The residue was purified by prep-TLC. Compound methyl 4-[(1S)-1-[[4-[[3-(trifluoromethyl) phenyl]methyl]pyrazolo[1,5-a]pyridine-3-carbonyl]amino]ethyl]benzoate (50 mg, 103.85 μmol, 51.17% yield) was obtained as a white solid, which was used into the next step directly without further purification. LCMS for product (ESI): m/z 482.2 [M+H]⁺.

### Step 4: Preparation of Compound 3,4-[(1S)-1-[[4-[[3-(trifluoromethyl)phenyl]methyl]pyrazolo[1,5-a]pyridine-3-carbonyl]amino]ethyl]benzoic acid

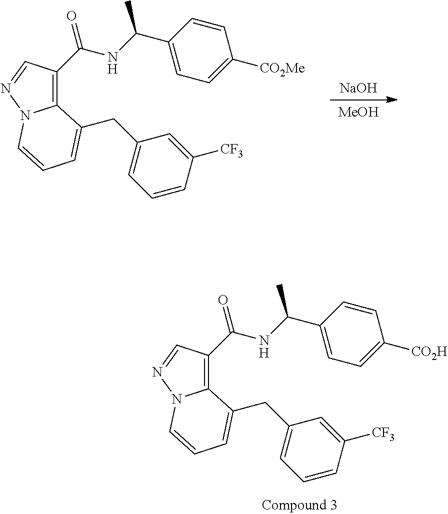 [SMILES: "MeOH" ; COC(=O)c1ccc([C@H](C)NC(=O)c2cnn3cccc(Cc4cccc(C(F)(F)F)c4)c23)cc1 ; "NaOH" ; C[C@H](NC(=O)c1cnn2cccc(Cc3cccc(C(F)(F)F)c3)c12)c1ccc(C(=O)O)cc1]

To a solution of methyl4-[(1S)-1-[[4-[[3-(trifluoromethyl)phenyl]methyl]pyrazolo[1,5-a]pyridine-3-carbonyl]amino]ethyl]benzoate (45 mg, 93.46 μmol, 1 eq) in MeOH (0.5 mL) was added NaOH (3.74 mg, 93.46 μmol, 1 eq) and H₂O (0.5 mL). The mixture was stirred at 40° C. for 16 hr.

The solvent was removed to afford the crude product. The mixture was adjusted to pH 4 with 1N HCl. The mixture was filtered and Compound 3, 4-[(1S)-1-[[4-[[3-(trifluoromethyl)phenyl]methyl]pyrazolo[1,5-a]pyridine-3-carbonyl]amino]ethyl]benzoic acid (30 mg, 62.90 μmol, 67.29% yield, 98% purity), was obtained as a white solid. LCMS for product (ESI): m/z 468.2 [M+H]⁺. ¹H NMR (400 MHz, DMSO-d₆) δ=8.73-8.68 (m, 2H), 8.38 (s, 1H), 7.88 (d, J=8.0 Hz, 2H), 7.51-7.45 (m, 4H), 7.38-7.29 (m, 3H), 7.04 (t, J=6.8 Hz, 1H), 5.19-5.11 (m, 1H), 4.70 (d, J=14.4 Hz, 1H), 4.56 (d, J=14.4 Hz, 1H), 1.40 (d, J=7.2 Hz, 3H).

### Example 4—Synthesis of 2-[[4-[[3-(trifluoromethyl)phenyl]methyl]pyrazolo[1,5-a]pyridine-3-carbonyl]amino]spiro[3.3]heptane-6-carboxylic acid (Compound 4)

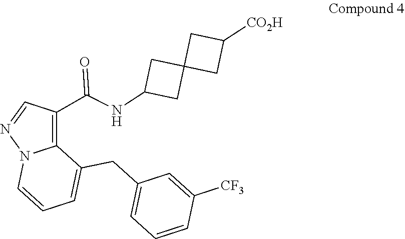 [SMILES: O=C(NC1CC2(C1)CC(C(=O)O)C2)c1cnn2cccc(Cc3cccc(C(F)(F)F)c3)c12]

### Step 1: Preparation of ethyl 2-[[4-[[3-trifluoromethyl)phenyl]methyl]-pyrazolo[1,5-a]pyridine-3-carbonyl]amino]-spiro[3.3]heptane-6-carboxylate

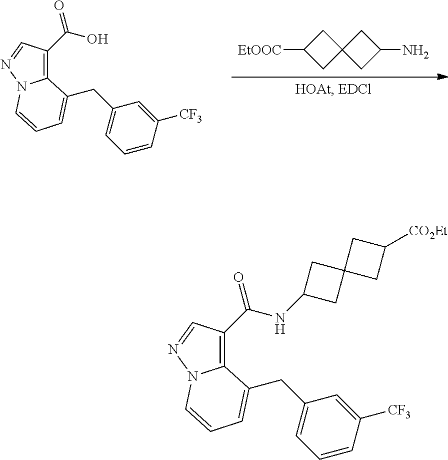

To a solution of 4-[[3-(trifluoromethyl) phenyl]methyl]pyrazolo[1,5-a]pyridine-3-carboxylic acid (65.00 mg, 202.96 μmol, 1 eq) and ethyl 2-aminospiro[3.3]heptane-6-carboxylate (37.19 mg, 202.96 μmol, 1 eq) in DCM (1 mL) was added HOAt (5.52 mg, 40.59 μmol, 5.68 μL, 0.2 eq), Et₃N (102.69 mg, 1.01 mmol, 141.25 μL, 5 eq) and EDCI (63.01 mg, 405.91 μmol, 71.85 μL, 2 eq). The mixture was stirred at 25° C. for 16 hr.

The solvent was removed to afford the crude product. The residue was purified by prep-TLC to provide ethyl 2-[[4-[[3-(trifluoromethyl)phenyl]methyl]pyrazolo[1,5-a]pyridine-3-carbonyl]amino]spiro[3.3]heptane-6-carboxylate (35 mg, 72.69 μmol, 35.82% yield) as a white solid, which was used into the next step directly without further purification. LCMS for product (ESI): m/z 486.2 [M+H]⁺.

### Step 2: Preparation of Compound 4,2-[[4-[[3-(trifluoromethyl)phenyl]methyl]pyrazolo[1,5-a]pyridine-3-carbonyl]amino]spiro[3.3]heptane-6-carboxylic acid

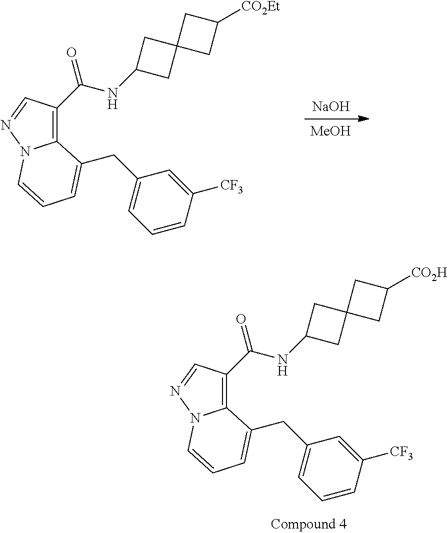 [SMILES: "MeOH" ; CCOC(=O)C1CC2(CC(NC(=O)c3cnn4cccc(Cc5cccc(C(F)(F)F)c5)c34)C2)C1 ; "NaOH" ; O=C(NC1CC2(C1)CC(C(=O)O)C2)c1cnn2cccc(Cc3cccc(C(F)(F)F)c3)c12]

To a solution of ethyl 2-[[4-[[3-(trifluoromethyl)phenyl]methyl]pyrazolo[1,5-a]pyridine-3-carbonyl]amino]spiro[3.3]heptane-6-carboxylate (30 mg, 63.63 μmol, 1 eq) in MeOH (0.5 mL) was added NaOH (2.55 mg, 63.63 μmol, 1 eq) and H₂O (0.5 mL). The mixture was stirred at 40° C. for 16 hr.

The solvent was removed to afford the crude product. The mixture was adjusted to pH 4 with 1N HCl. The mixture was filtered and Compound 4, 2-[[4-[[3-(trifluoromethyl)phenyl]methyl]pyrazolo[1,5-a]pyridine-3-carbonyl]amino]spiro[3.3]heptane-6-carboxylic acid (28 mg, 60.60 μmol, 95.23% yield, 99% purity) was obtained as a white solid. LCMS for product (ESI): m/z 458.2 [M+H]⁺. ¹HNMR (400 MHz, DMSO-d₆) δ=12.03 (brs, 1H), 8.67 (d, J=6.8 Hz, 1H), 8.36 (d, J=7.8 Hz, 1H), 8.21 (s, 1H), 7.54-7.32 (m, 5H), 7.03 (t, J=6.8 Hz, 1H), 4.65 (s, 2H), 4.27-4.13 (m, 1H), 2.94-2.92 (m, 1H), 2.43-2.35 (m, 1H), 2.34-2.26 (m, 1H), 2.24-2.17 (m, 2H), 2.16-2.02 (m, 2H), 2.00-1.87 (m, 2H).

### Example 5—Synthesis of 4-[(1S)-1-[[8-[[4-(trifluoromethyl) phenyl]methyl]imidazo[1,5-a]pyridine-1-carbonyl]amino]ethyl]benzoic acid (Compound 5)

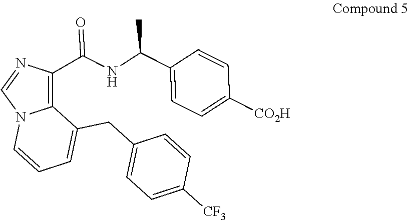 [SMILES: C[C@H](NC(=O)c1ncn2cccc(Cc3ccc(C(F)(F)F)cc3)c12)c1ccc(C(=O)O)cc1]

### Step 1: Preparation of methyl 8-[[4-(trifluoromethyl) phenyl]methyl]imidazo[1,5-a]pyridine-1-carboxylate

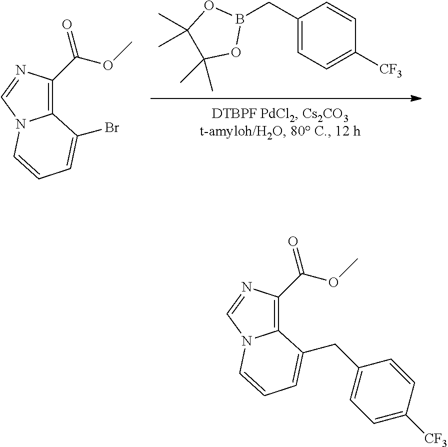

To a solution of 4,4,5,5-tetramethyl-2-[[4-(trifluoromethyl) phenyl]methyl]-1,3,2-dioxaborolane (560.83 mg, 1.96 mmol, 1 eq) and methyl 8-bromoimidazo[1,5-a]pyridine-1-carboxylate (500 mg, 1.96 mmol, 1 eq) in H₂O (1 mL) and t-amyloh (1 mL) was added ditert-butyl (cyclopentyl) phosphane; dichloropalladium; iron (127.76 mg, 196.03 μmol, 0.1 eq) and Cs₂CO₃ (1.28 g, 3.92 mmol, 2 eq) under nitrogen. The mixture was stirred at 80° C. for 16 hr.

The reaction mixture was diluted with H₂O 15 mL and extracted with DCM 30 mL (10 mL×3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=50/1 to 1/1) to provide methyl 8-[[4-(trifluoromethyl) phenyl]methyl]imidazo[1,5-a]pyridine-1-carboxylate (262 mg, 40% yield) as a white solid. LCMS for crude product (ESI): m/z 335.0 [M+H]⁺.

### Step 2: Preparation of 8-[[4-(trifluoromethyl) phenyl]methyl]imidazo[1,5-a]pyridine-1-carboxylic acid

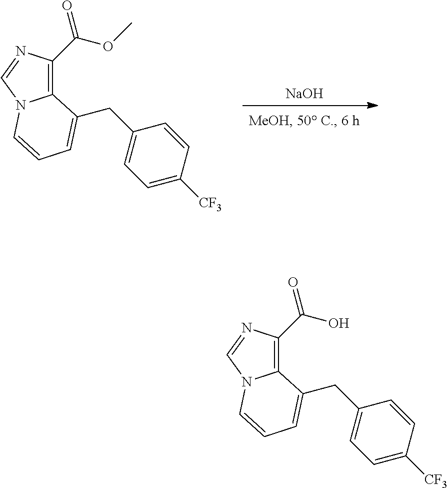 [SMILES: COC(=O)c1ncn2cccc(Cc3ccc(C(F)(F)F)cc3)c12 ; "NaOH" ; O=C(O)c1ncn2cccc(Cc3ccc(C(F)(F)F)cc3)c12]

To a solution of methyl 8-[[4-(trifluoromethyl) phenyl]methyl]imidazo[1,5-a]pyridine-1-carboxylate (150 mg, 448.7 μmol, 1 eq) in H₂O (1 mL) and MeOH (1 mL) was added NaOH (28.00 mg, 699.99 μmol, 3 eq). The mixture was stirred at 50° C. for 16 hr.

The reaction mixture was concentrated under reduced pressure to remove MeOH. The mixture was adjusted to pH 4 with 1N HCl. The mixture was filtered and compound 8-[[4-(trifluoromethyl) phenyl]methyl]imidazo[1,5-a]pyridine-1-carboxylic acid (120 mg, 374.68 μmol, 80% yield) was obtained as a white solid.

### Step 3: Preparation of methyl 4-[(1S)-1-[[8-[[4-(trifluoromethyl) phenyl]methyl]imidazo[1,5-a]pyridine-1-carbonyl]amino]ethyl]benzoate

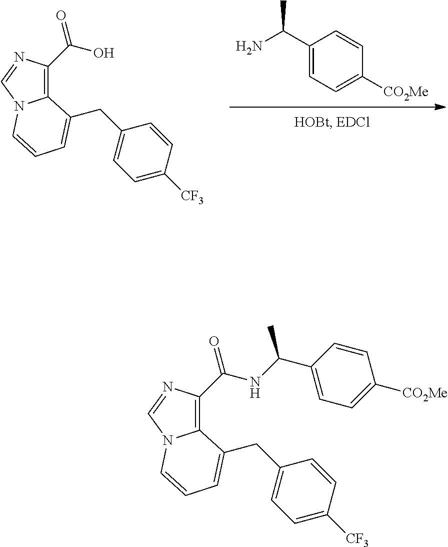 [SMILES: O=C(O)c1ncn2cccc(Cc3ccc(C(F)(F)F)cc3)c12 ; COC(=O)c1ccc([C@H](C)N)cc1 ; COC(=O)c1ccc([C@H](C)NC(=O)c2ncn3cccc(Cc4ccc(C(F)(F)F)cc4)c23)cc1]

To a solution of 8-[[4-(trifluoromethyl) phenyl]methyl]imidazo[1,5-a]pyridine-1-carboxylic acid (40 mg, 124.90 μmol, 1 eq) in DCM (2 mL) was added HOBt (3.38 mg, 24.98 μmol, 0.2 eq), Et₃N (63.19 mg, 624.48 μmol, 86.92 μL, 5 eq) and EDCI (47.89 mg, 249.79 μmol, 2 eq) at 0° C. The mixture was stirred at 0° C. for 0.5 hr. Then methyl 4-[(1S)-1-aminoethyl]benzoate (33.57 mg, 187.34 μmol, 1.5 eq) was added at 0° C. The mixture was stirred at 25° C. for 16 hr. The reaction mixture was diluted with H₂O 5 mL and extracted with DCM 30 mL (10 mL×3). The combined organic layers were washed with brine 5 mL, dried over Na₂SO₄, filtered and concentrated under reduced pressure to give a residue. The residue was purified by prep-TLC (SiO₂, PE: EA=1:1). Compound methyl 4-[(1S)-1-[[8-[[4-(trifluoromethyl) phenyl]methyl]imidazo[1,5-a]pyridine-1-carbonyl]amino]ethyl]benzoate (35 mg, 72.69 μmol, 58.20% yield) was obtained as a white solid, which was used into the next step directly without further purification. LCMS for product (ESI): m/z 482.2 [M+H]⁺.

### Step 4: Preparation of Compound 5,4-[(1S)-1-[[8-[[4-(trifluoromethyl) phenyl]methyl]imidazo[1,5-a]pyridine-1-carbonyl]amino]ethyl]benzoic acid

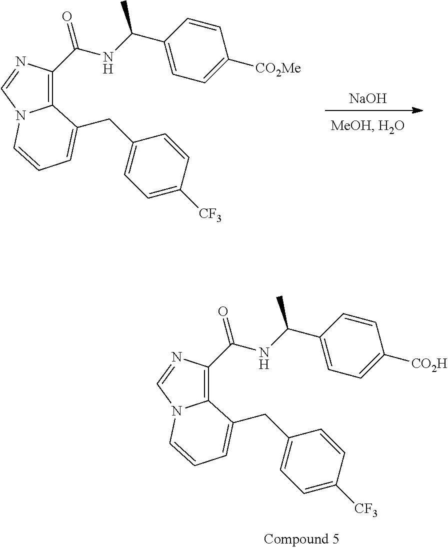 [SMILES: COC(=O)c1ccc([C@H](C)NC(=O)c2ncn3cccc(Cc4ccc(C(F)(F)F)cc4)c23)cc1 ; "NaOH" ; C[C@H](NC(=O)c1ncn2cccc(Cc3ccc(C(F)(F)F)cc3)c12)c1ccc(C(=O)O)cc1]

To a solution of methyl 4-[(1S)-1-[[8-[[4-(trifluoromethyl) phenyl]methyl]imidazo[1,5-a]pyridine-1-carbonyl]amino]ethyl]benzoate (35 mg, 72.69 μmol, 1 eq) in H₂O (1 mL) and MeOH (1 mL) was added NaOH (8.72 mg, 218.08 μmol, 3 eq). The mixture was stirred at 50° C. for 16 hr.

The reaction mixture was concentrated under reduced pressure to remove MeOH. The mixture was adjusted to pH 4 with 1N HCl. The mixture was filtered and Compound 5, 4-[(1S)-1-[[8-[[4-(trifluoromethyl) phenyl]methyl]imidazo[1,5-a]pyridine-1-carbonyl]amino]ethyl]benzoic acid (21 mg, 44.92 μmol, 60% yield, 99.13% purity), was obtained as a white solid. LCMS for product (ESI): m/z 468.2 [M+H]⁺. ¹H NMR (400 MHz, DMSO-d₆) δ ppm 8.57 (d, J=8.4 Hz, 1H), 8.52 (s, 1H), 8.38 (d, J=6.4 Hz, 1H), 7.90 (d, J=8.0 Hz, 2H), 7.51 (d, J=8.0 Hz, 2H), 7.40 (d, J=8.0 Hz, 2H), 7.28 (d, J=8.0 Hz, 2H), 6.96 (d, J=6.4 Hz, 1H), 6.83 (t, J=6.8 Hz, 1H), 5.23-5.16 (m, 1H), 4.86 (d, J=14.8 Hz, 1H), 4.67 (d, J=14.8 Hz, 1H), 1.49 (d, J=7.2 Hz, 3H)

### Example 6—Synthesis of cis-2-[[8-[[4-(trifluoromethyl) phenyl]methyl]imidazo[1,5-a]pyridine-1-carbonyl]amino]spiro[3.3]heptane-6-carboxylic acid (Compound 6A)

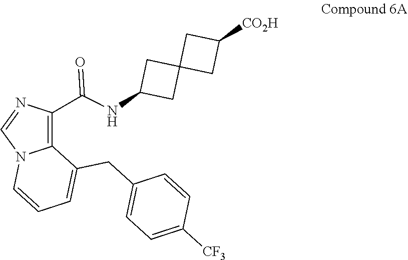 [SMILES: O=C(N[C@H]1CC2(C1)C[C@H](C(=O)O)C2)c1ncn2cccc(Cc3ccc(C(F)(F)F)cc3)c12]

### Step 1: Preparation of (S)-1-phenylethyl 6-oxospiro[3.3]heptane-2-carboxylate

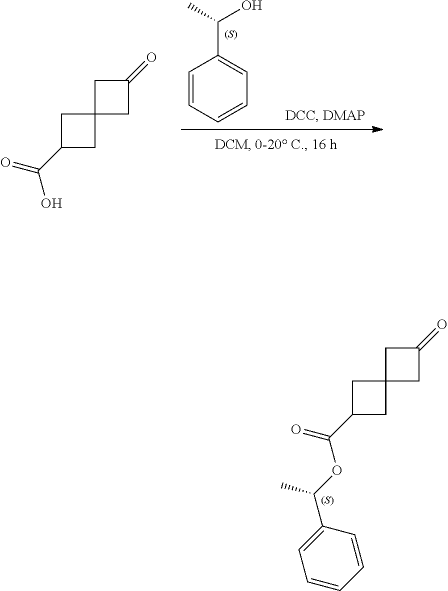

To a mixture of compound 6-oxospiro[3.3]heptane-2-carboxylic acid (10 g, 64.87 mmol, 1 eq) and DMAP (8.72 g, 71.35 mmol, 1.1 eq) in DCM (100 mL) was added DCC (14.72 g, 71.35 mmol, 14.43 mL, 1.1 eq) in DCM (50 mL). Then to the mixture was added (1S)-1-phenylethanol (7.53 g, 61.62 mmol, 7.45 mL, 0.95 eq) in DCM (100 mL) at 0° C. The reaction mixture was stirred at 20° C. for 16 hr.

Three reactions were combined for workup and purification. The mixture was filtered through celite. The filtrate was concentrated under vacuo to give a crude residue. The residue was purified by column chromatography (SiO2, Petroleum ether/Ethyl acetate=20/1 to 5/1) to give compound (S)-1-phenylethyl 6-oxospiro[3.3]heptane-2-carboxylate (40 g, 154.85 mmol, 79.57% yield) as colorless oil.

¹H NMR (ET26855-102-P1, 400 MHz, chloroform-d) δ ppm=1.54 (d, J=6.63 Hz, 3H), 2.40-2.61 (m, 4H), 3.00-3.23 (m, 5H), 5.89 (q, J=6.59 Hz, 1H), 7.27-7.39 (m, 5H).

### Step 2: Preparation of (S)-1-phenylethyl 6-(((S)-tert-butylsulfinyl)imino)spiro[3.3]heptane-2-carboxylate

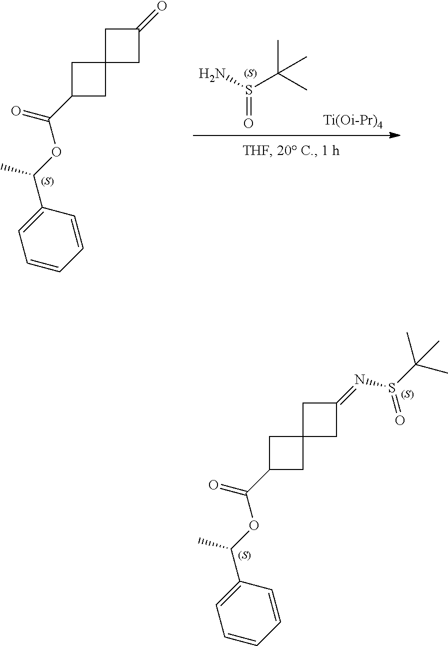

A mixture of Ti(Oi-Pr)₄ (11.00 g, 38.71 mmol, 11.43 mL, 1 eq) and compound (S)-1-phenylethyl 6-oxospiro[3.3]heptane-2-carboxylate (10 g, 38.71 mmol, 1 eq) in THF (90 mL) was stirred for 10 minutes. Then 2-methylpropane-2-sμLfinamide (5.63 g, 46.46 mmol, 1.2 eq) was added and the reaction mixture was stirred at 20° C. for 1 h.

Four reactions were combined for workup and purification. The mixture was concentrated under vacuo to give a residue. The residue was dissolved in EtOAc (500 mL). The solution was washed with saturated NaHCO₃ (500 mL). The mixture was filtered through celite. Then the mixture was separated layers. The organic layer was dried over Na₂SO₄ and concentrated under vacuo to give crude compound (S)-1-phenylethyl 6-(((S)-tert-butylsulfinyl)imino)spiro[3.3]heptane-2-carboxylate (30 g, 74.69 mmol, 48.23% yield) as colorless oil. The crude product was used for next step without any further purification.

¹H NMR (ET26855-134-P1, 400 MHz, chloroform-d) δ=1.16 (s, 9H), 1.44 (d, J=6.38 Hz, 2H), 2.25-2.52 (m, 4H), 2.93-3.12 (m, 4H), 4.88-5.87 (m, 1H), 7.20-7.36 (m, 4H).

### Step 3: Preparation of B4_cis and B4_trans

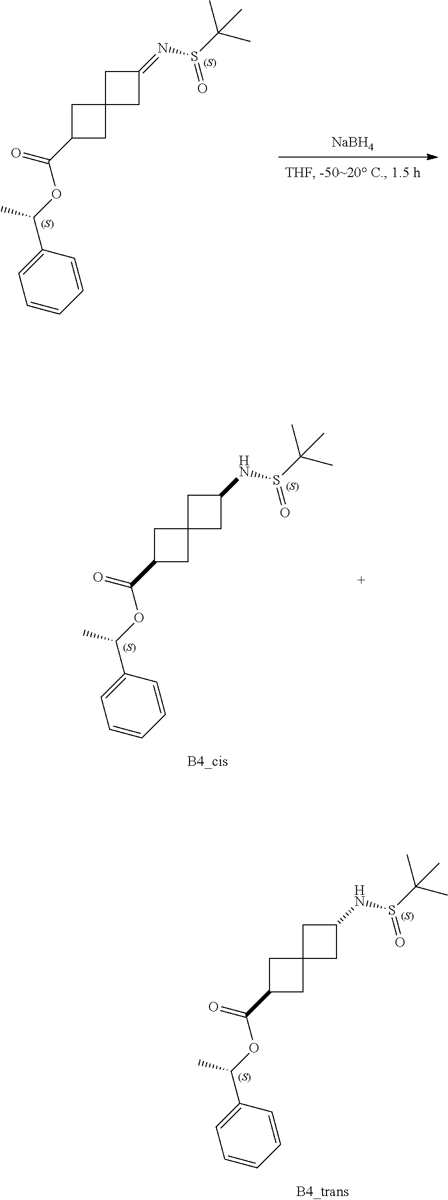

A mixture of compound (S)-1-phenylethyl 6-(((S)-tert-butylsulfinyl)imino)spiro[3.3]heptane-2-carboxylate (10 g, 27.66 mmol, 1 eq) in THE (3 mL) was added NaBH₄ (3.14 g, 82.99 mmol, 3 eq) at −50° C. The mixture was stirred at −50° C. for 30 minutes, then rise to 20° C. and stirred at 20° C. for 1 hour.

Three reactions were combined for workup and purification. The mixture was concentrated under vacuo to remove THF. The residue was extracted with DCM (200 mL). The organic layer was concentrated to give a crude product. The crude product was purified by pre-HPLC (column: Waters Xbridge BEH C18 100*30 mm*10 um; mobile phase: [water (10 mM NH₄HCO₃)-ACN]; B %: 55%-75%, 10 min) to give two diastereoisomer compound B4_cis (6.6 g, 16.34 mmol, 19.69% yield, 90% purity) as white solid and compound B4_trans (6.5 g, 16.99 mmol, 20.47% yield, 95% purity) as white solid.

HPLC of compound B4_cis (ET26855-136-P1A, retention time 13.558 min). HPLC of compound B4_trans (ET26855-136-P2A, retention time 13.438 min). ¹H NMR (B4-cis ET26855-136-P1, 400 MHz, chloroform-d) δ=1.11 (s, 9H), 1.45 (d, J=6.63 Hz, 3H), 1.78-1.94 (m, 2H), 2.03-2.13 (m, 1H), 2.17-2.31 (m, 4H), 2.35-2.45 (m, 1H), 2.97 (quin, J=8.41 Hz, 1H), 3.19 (br d, J=6.25 Hz, 1H), 3.64-3.78 (m, 1H), 5.79 (q, J=6.55 Hz, 1H), 7.20-7.31 (m, 5H). ¹H NMR (B4-trans ET26855-136-P2, 400 MHz, chloroform-d) δ=1.11 (s, 9H), 1.44 (d, J=6.50 Hz, 3H), 1.76-1.95 (m, 2H), 2.04-2.15 (m, 1H), 2.17-2.30 (m, 4H), 2.39-2.49 (m, 1H), 2.97 (quin, J=8.44 Hz, 1H), 3.18 (br d, J=6.50 Hz, 1H), 3.68 (sxt, J=7.50 Hz, 1H), 5.79 (q, J=6.59 Hz, 1H), 7.20-7.30 (m, 5H).

The absolute configuration of B4 is not determined and cis and trans designations are randomly assigned. After deprotection for the amino and carboxyl, B_cis and B4_trans become enantiomers to each other. cis and trans designations are used to differentiate the two sets of molecules. All the cis spiro[3.3]heptane molecules below come from B4_cis, and all the trans spiro[3.3]heptane moleculeds are from B4_trans.

### Step 4: Preparation of cis-(S)-1-phenylethyl 2-aminospiro[3.3]heptane-6-carboxylate

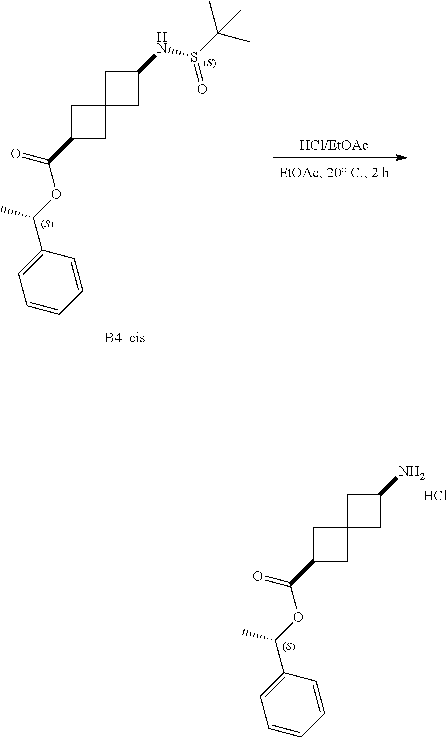

To the mixture of compound B4_cis (6.6 g, 18.16 mmol, 1 eq) in EtOAc (120 mL) was added HCl/EtOAc (4 M, 120 mL, 26.44 eq) at 20° C. The mixture was stirred at 20° C. for 2 h.

The mixture was concentrated under vacuo to give crude product. The crude product was triturated in MTBE (30 mL) and filtered. The filter cake was dried under vacuo to give cis-(S)-1-phenylethyl 2-aminospiro[3.3]heptane-6-carboxylate (5.1 g, 17.24 mmol, 94.96% yield, HCl) as white solid.

¹H NMR (400 MHz, DMSO-d₆) δ ppm=1.44 (d, J=6.60 Hz, 3H), 2.11-2.20 (m, 6H), 2.26-2.37 (m, 2H), 3.00-3.10 (m, 1H), 3.48 (quin, J=7.89 Hz, 1H), 5.77 (q, J=6.60 Hz, 1H), 7.23-7.43 (m, 5H), 8.30 (br s, 3H).

### Step 5: Preparation of trans-(S)-1-phenylethyl 2-aminospiro[3.3]heptane-6-carboxylate

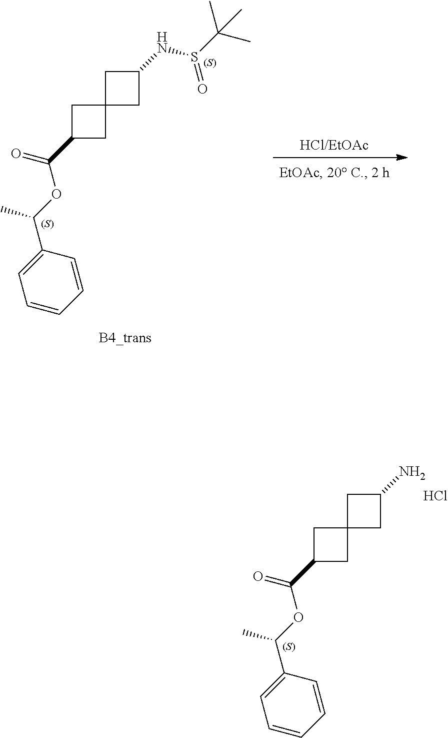

To the mixture of compound B4_trans (6.5 g, 17.88 mmol, 1 eq) in EtOAc (120 mL) was added HCl/EtOAc (4 M, 120 mL, 26.84 eq) at 20° C. The mixture was stirred at 20° C. for 2 h.

The crude product was triturated in MTBE (30 mL) and filtered. The filter cake was dried under vacuo to give trans-(S)-1-phenylethyl 2-aminospiro[3.3]heptane-6-carboxylate (5 g, 16.90 mmol, 94.53% yield, HCl) as white solid.

¹H NMR (400 MHz, DMSO-d₆) δ ppm=1.44 (d, J=6.60 Hz, 3H), 2.07-2.21 (m, 6H), 2.26-2.38 (m, 2H), 3.06 (quin, J=8.34 Hz, 1H), 3.49 (quin, J=8.04 Hz, 1H), 5.77 (q, J=6.60 Hz, 1H), 7.23-7.41 (m, 5H), 8.26 (br s, 3H).

### Step 6: Preparation of cis-(S)-1-phenylethyl 2-[[8-[[4-(trifluoromethyl) phenyl]methyl]imidazo[1,5-a]pyridine-1-carbonyl]amino]spiro[3.3]heptane-6-carboxylate

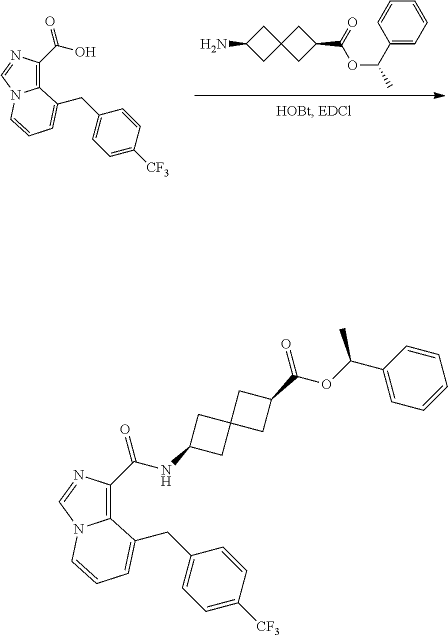 [SMILES: O=C(O)c1ncn2cccc(Cc3ccc(C(F)(F)F)cc3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3ncn4cccc(Cc5ccc(C(F)(F)F)cc5)c34)C2)C1)c1ccccc1]

To a solution of 8-[[4-(trifluoromethyl) phenyl]methyl]imidazo[1,5-a]pyridine-1-carboxylic acid (40 mg, 124.90 μmol, 1 eq) in DCM (1 mL) was added EDCI (47.89 mg, 249.79 μmol, 2 eq), Et3N (63.19 mg, 624.48 μmol, 86.92 μL, 5 eq) and HOBt (3.38 mg, 24.98 μmol, 0.2 eq) at 0° C. The mixture was stirred at 0° C. for 0.5 hr. Then cis-(S)-1-phenylethyl 2-aminospiro[3.3]heptane-6-carboxylate (64.78 mg, 249.79 μmol, 2 eq) was added at 0° C. The mixture was stirred at 25° C. for 16 hr.

The reaction mixture was diluted with H₂O 15 mL and extracted with DCM 30 mL (10 mL×3). The combined organic layers were washed with brine 5 mL, dried over Na₂SO₄, filtered and concentrated under reduced pressure to give a residue. The residue was purified by prep-TLC (SiO2, PE: EA=1:1). Compound cis-(S)-1-phenylethyl 2-[[8-[[4-(trifluoromethyl) phenyl]methyl]imidazo[1,5-a]pyridine-1-carbonyl]amino]spiro[3.3]heptane-6-carboxylate (30 mg, 53.42 μmol, 75% yield) was obtained as a white solid, which was used into the next step directly without further purification. LCMS for product (ESI): m/z 562.3 [M+H]⁺.

### Step 7: Preparation of Compound 6A, cis-2-[[8-[[4-(trifluoromethyl) phenyl]methyl]imidazo[1,5-a]pyridine-1-carbonyl]amino]spiro[3.3]heptane-6-carboxylic acid

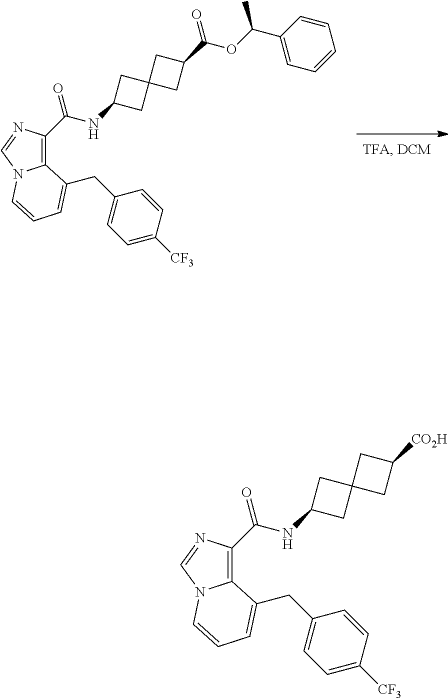 [SMILES: C[C@H](OC(=O)[C@H]1CC2(C[C@H](NC(=O)c3ncn4cccc(Cc5ccc(C(F)(F)F)cc5)c34)C2)C1)c1ccccc1 ; O=C(N[C@H]1CC2(C1)C[C@H](C(=O)O)C2)c1ncn2cccc(Cc3ccc(C(F)(F)F)cc3)c12]

To a solution of cis-(S)-1-phenylethyl 2-[[8-[[4-(trifluoromethyl) phenyl]methyl]imidazo[1,5-a]pyridine-1-carbonyl]amino]spiro[3.3]heptane-6-carboxylate (30 mg, 53.42 μmol, 1 eq) in DCM (1 mL) and TFA (1 mL). The mixture was stirred at 50° C. for 3 hr.

The reaction mixture was concentrated under reduced pressure to remove solvent. The residue was dissolved by 1 N NaOH (10 mL) and filtered. The fitrate was adjusted to PH 4 with 1N HCl. The mixture was filtered. Compound 6A, cis-2-[[8-[[4-(trifluoromethyl) phenyl]methyl]imidazo[1,5-a]pyridine-1-carbonyl]amino]spiro[3.3]heptane-6-carboxylic acid (23 mg, 50.28 μmol, 76.67% yield, 98.06% purity) was obtained as a white solid. LCMS for product (ESI): m/z 458.2 [M+H]⁺. ¹H NMR (400 MHz, DMSO-d₆) δ ppm 12.01 (br s, 1H), 8.48 (s, 1H), 8.36 (d, J=7.2 Hz, 1H), 8.24 (d, J=8.0 Hz, 1H), 7.57 (d, J=8.4 Hz, 2H), 7.38 (d, J=8.4 Hz, 2H), 6.91 (d, J=6.4 Hz, 1H), 6.83-6.79 (m, 1H), 4.80 (s, 2H), 4.29-4.23 (m, 1H), 2.97-2.95 (m, 1H), 2.40-2.36 (m, 1H), 2.28-2.27 (m, 1H), 2.27-2.22 (m, 2H), 2.20-2.08 (m, 4H).

### Example 7—Synthesis of trans-2-[[8-[[4-(trifluoromethyl) phenyl]methyl]imidazo[1,5-a]pyridine-1-carbonyl]amino]spiro[3.3]heptane-6-carboxylic acid (Compound 6B)

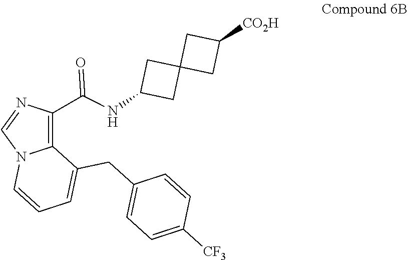 [SMILES: O=C(N[C@H]1CC2(C1)C[C@H](C(=O)O)C2)c1ncn2cccc(Cc3ccc(C(F)(F)F)cc3)c12]

### Step 1: Preparation of trans-(S)-1-phenylethyl 2-[[8-[[4-(trifluoromethyl)phenyl]methyl]imidazo[1,5-a]pyridine-1-carbonyl]amino]spiro[3.3]heptane-6-carboxylate

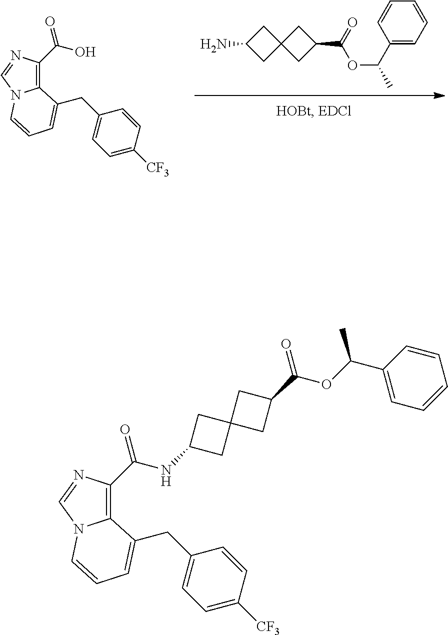 [SMILES: O=C(O)c1ncn2cccc(Cc3ccc(C(F)(F)F)cc3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3ncn4cccc(Cc5ccc(C(F)(F)F)cc5)c34)C2)C1)c1ccccc1]

To a solution of 8-[[4-(trifluoromethyl) phenyl]methyl]imidazo[1,5-a]pyridine-1-carboxylic acid (40 mg, 124.90 μmol, 1 eq) in DCM (1 mL) was added EDCI (47.89 mg, 249.80 μmol, 2 eq), Et₃N (63.19 mg, 624.50 μmol, 86.92 μL, 5 eq) and HOBt (3.38 mg, 24.98 μmol, 0.2 eq) at 0° C. The mixture was stirred at 0° C. for 0.5 hr. Then trans-(S)-1-phenylethyl 2-aminospiro[3.3]heptane-6-carboxylate (64.78 mg, 249.80 μmol, 2 eq) was added at 0° C. The mixture was stirred at 25° C. for 16 hr.

The reaction mixture was diluted with H₂O 5 mL and extracted with DCM 15 mL (5 mL×3). The combined organic layers were washed with brine 5 mL, dried over Na₂SO₄, filtered and concentrated under reduced pressure to give a residue. The residue was purified by prep-TLC (SiO₂, PE:EA=1:1) to provide trans-(S)-1-phenylethyl 2-[[8-[[4-(trifluoromethyl)phenyl]methyl]imidazo[1,5-a]pyridine-1-carbonyl]amino]spiro[3.3]heptane-6-carboxylate (29 mg, 51.63 μmol, 72.5% yield) as a white solid, which was used into the next step directly without further purification. LCMS for product (ESI): m/z 562.3 [M+H]⁺.

### Step 2: Preparation of Compound 6B, trans-2-[[8-[[4-(trifluoromethyl) phenyl]methyl]imidazo[1,5-a]pyridine-1-carbonyl]amino spiro[3.3]heptane-6-carboxylic acid

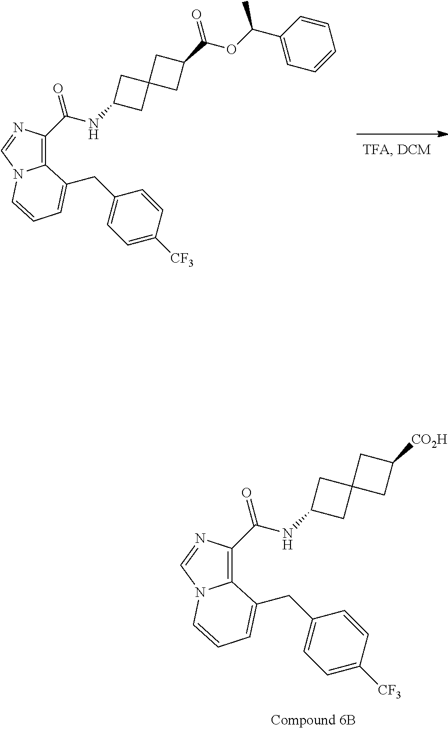 [SMILES: C[C@H](OC(=O)[C@H]1CC2(C[C@H](NC(=O)c3ncn4cccc(Cc5ccc(C(F)(F)F)cc5)c34)C2)C1)c1ccccc1 ; O=C(N[C@H]1CC2(C1)C[C@H](C(=O)O)C2)c1ncn2cccc(Cc3ccc(C(F)(F)F)cc3)c12]

To a solution of trans-(S)-1-phenylethyl 2-[[8-[[4-(trifluoromethyl)phenyl]methyl]imidazo[1,5-a]pyridine-1-carbonyl]amino]spiro[3.3]heptane-6-carboxylate (29 mg, 51.63 μmol, 1 eq) in DCM (1 mL) and TFA (1 mL). The mixture was stirred at 50° C. for 3 hr.

The reaction mixture was concentrated under reduced pressure to remove solvent. The residue was dissolved by 1 N NaOH (10 mL) and filtered. The fitrate was adjusted to PH 4 with 1N HCl. The mixture was filtered. Compound 6B, trans-2-[[8-[[4-(trifluoromethyl) phenyl]methyl]imidazo[1,5-a]pyridine-1-carbonyl]amino]spiro[3.3]heptane-6-carboxylic acid (19 mg, 41.53 μmol, 65.52% yield, 96.29% purity), was obtained as a white solid. LCMS for product (ESI): m/z 458.2 [M+H]⁺. ¹H NMR (400 MHz, DMSO-d₆) δ=12.00 (br s, 1H), 8.48 (s, 1H), 8.37 (d, J=6.8 Hz, 1H), 8.24 (d, J=8.4 Hz, 1H), 7.58 (d, J=8.0 Hz, 2H), 7.38 (d, J=8.0 Hz, 2H), 6.91 (d, J=6.8 Hz, 1H), 6.85-6.79 (m, 1H), 4.80 (s, 2H), 4.35-4.19 (m, 1H), 2.94 (t, J=8.4 Hz, 1H), 2.39-2.36 (m, 1H), 2.28-2.22 (m, 2H), 2.11-2.06 (m, 3H), 2.03-2.00 (m, 1H).

### Example 8—Synthesis of 4-[(1S)-1-[[4-[[4-(trifluoromethyl)phenyl]methyl]-1H-indazole-3-carbonyl]amino]ethyl]benzoic acid (Compound 7)

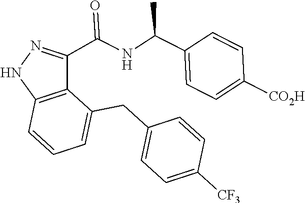 [SMILES: C[C@H](NC(=O)c1n[nH]c2cccc(Cc3ccc(C(F)(F)F)cc3)c12)c1ccc(C(=O)O)cc1]

### Compound 7

### Step 1: Preparation of methyl 4-bromo-1-tetrahydropyran-2-yl-indazole-3-carboxylate

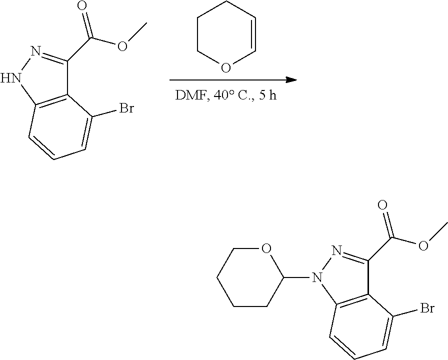 [SMILES: COC(=O)c1n[nH]c2cccc(Br)c12 ; C1=COCCC1 ; COC(=O)c1nn(C2CCCCO2)c2cccc(Br)c12]

A mixture of methyl 4-bromo-1H-indazole-3-carboxylate (1 g, 3.92 mmol, 1 eq), 3,4-dihydro-2H-pyran (1.65 g, 19.60 mmol, 1.79 mL, 5 eq) in DCM (10 mL) was added 4-methylbenzenesulfonic acid;pyridine (98.52 mg, 392.05 μmol, 0.1 eq). The mixture was degassed and purged with N₂ for 3 times, and then the mixture was stirred at 40° C. for 12 hr under N₂ atmosphere.

The residue was diluted with H₂O 30 mL and extracted with DCM 90 mL (30 mL×3). The combined organic layers were washed with brine 30 mL, dried over Na₂SO₄, filtered and concentrated under reduced pressure to give a residue. The crude product was used into the next step without further purification to provide methyl 4-bromo-1-tetrahydropyran-2-yl-indazole-3-carboxylate (1.1 g, crude) as a yellow solid, which was used into the next step directly without further purification.

### Step 2: Preparation of methyl 1-tetrahydropyran-2-yl-4-[[4-(trifluoromethyl)phenyl]methyl]indazole-3-carboxylate

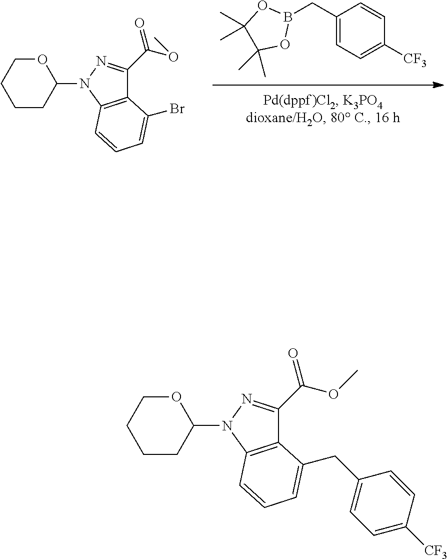 [SMILES: COC(=O)c1nn(C2CCCCO2)c2cccc(Br)c12 ; CC1(C)OB(Cc2ccc(C(F)(F)F)cc2)OC1(C)C ; COC(=O)c1nn(C2CCCCO2)c2cccc(Cc3ccc(C(F)(F)F)cc3)c12]

To a solution of methyl 4-bromo-1-tetrahydropyran-2-yl-indazole-3-carboxylate (350 mg, 1.03 mmol, 1 eq) and 4,4,5,5-tetramethyl-2-[[4-(trifluoromethyl)phenyl]methyl]-1,3,2-dioxaborolane (295.38 mg, 1.03 mmol, 1 eq) in dioxane (2 mL) and H₂O (1 mL) was added Pd(dppf)Cl₂ (323.59 mg, 442.24 μmol, 0.3 eq) and K₃PO₄ (312.91 mg, 1.47 mmol, 1 eq) under nitrogen. The mixture was stirred at 80° C. for 12 hr.

The reaction mixture was concentrated under reduced pressure to remove dioxane. The residue was diluted with H₂O 20 mL and extracted with EA 450 mL (15 mL×3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=50/1 to 1/1). Compound methyl 1-tetrahydropyran-2-yl-4-[[4-(trifluoromethyl)phenyl]methyl]indazole-3-carboxylate (180 mg, 430.20 μmol, 42% yield) was obtained as a white solid. LCMS for crude product (ESI): m/z 419.3 [M+H]⁺.

### Step 3: Preparation of 1-tetrahydropyran-2-yl-4-[[4-(trifluoromethyl)phenyl]methyl]indazole-3-carboxylic acid

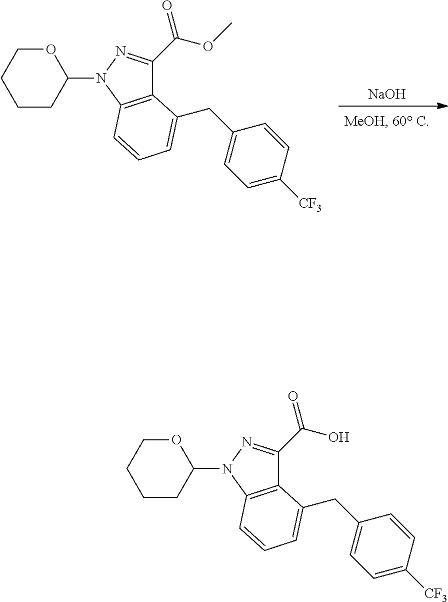 [SMILES: COC(=O)c1nn(C2CCCCO2)c2cccc(Cc3ccc(C(F)(F)F)cc3)c12 ; "NaOH" ; O=C(O)c1nn(C2CCCCO2)c2cccc(Cc3ccc(C(F)(F)F)cc3)c12]

To a solution of methyl 1-tetrahydropyran-2-yl-4-[[4-(trifluoromethyl)phenyl]methyl]indazole-3-carboxylate (180 mg, 430.20 μmol, 1 eq) in H₂O (5 mL) and MeOH (3 mL) was added NaOH (51.62 mg, 1.29 mmol, 3 eq). The mixture was stirred at 50° C. for 16 hr.

The reaction mixture was concentrated under reduced pressure to remove MeOH. The mixture was adjusted to PH 4 with 1N HCl. The mixture was filtered to give 1-tetrahydropyran-2-yl-4-[[4-(trifluoromethyl)phenyl]methyl]indazole-3-carboxylic acid (150 mg, 370.93 μmol, 86.70% yield) as a white solid.

### Step 4: Preparation of methyl 4-[(1S)-1-[[1-tetrahydropyran-2-yl-4-[[4-(trifluoromethyl)phenyl]methyl]indazole-3-carbonyl]amino]ethyl]benzoate

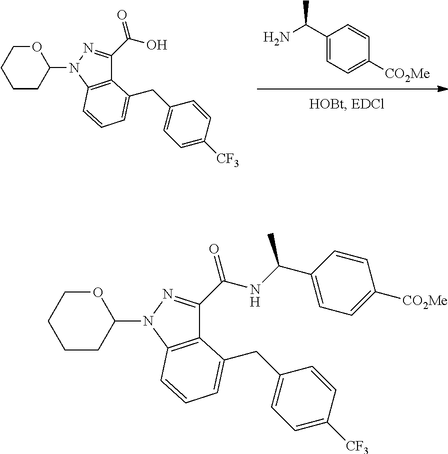 [SMILES: O=C(O)c1nn(C2CCCCO2)c2cccc(Cc3ccc(C(F)(F)F)cc3)c12 ; COC(=O)c1ccc([C@H](C)N)cc1 ; COC(=O)c1ccc([C@H](C)NC(=O)c2nn(C3CCCCO3)c3cccc(Cc4ccc(C(F)(F)F)cc4)c23)cc1]

To a solution of 1-tetrahydropyran-2-yl-4-[[4-(trifluoromethyl)phenyl]methyl]indazole-3-carboxylic acid (40 mg, 98.92 μmol, 1 eq) in DCM (2 mL) was added HOBt (2.67 mg, 19.78 μmol, 0.2 eq), EDCI (37.92 mg, 197.83 μmol, 2 eq) and Et₃N (50.05 mg, 494.58 μmol, 68.84 μL, 5 eq) at 0° C. The mixture was stirred at 0° C. for 0.5 hr. Then methyl 4-[(1S)-1-aminoethyl]benzoate (17.73 mg, 98.92 μmol, 1 eq) was added at 0° C. The mixture was stirred at 25° C. for 16 hr.

The reaction mixture was diluted with H₂O 10 mL and extracted with DCM 30 mL (10 mL×3). The combined organic layers were washed with brine 10 mL, dried over Na₂SO₄, filtered and concentrated under reduced pressure to give a residue. Compound methyl 4-[(1S)-1-[[1-tetrahydropyran-2-yl-4-[[4-(trifluoromethyl)phenyl]methyl]indazole-3-carbonyl]amino]ethyl]benzoate (35 mg, 61.88 μmol, 62.56% yield) was obtained as a white solid, which was used into the next step directly without further purification. LCMS for product (ESI): m/z 566.3 [M+H]⁺.

### Step 5: Preparation of 4-[(1S)-1-[[4-[[4-(trifluoromethyl)phenyl]methyl]-1H-indazole-3-carbonyl]amino]ethyl]benzoate

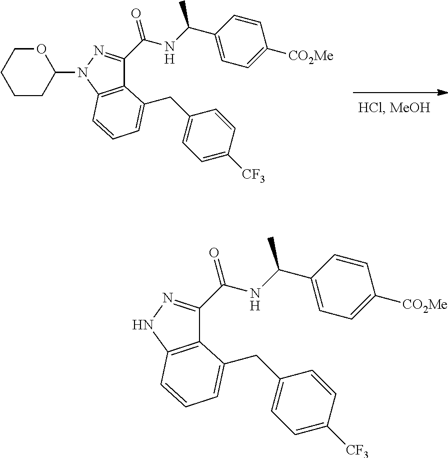 [SMILES: COC(=O)c1ccc([C@H](C)NC(=O)c2nn(C3CCCCO3)c3cccc(Cc4ccc(C(F)(F)F)cc4)c23)cc1 ; COC(=O)c1ccc([C@H](C)NC(=O)c2n[nH]c3cccc(Cc4ccc(C(F)(F)F)cc4)c23)cc1]

A solution of methyl 4-[(1S)-1-[[1-tetrahydropyran-2-yl-4-[[4-(trifluoromethyl)phenyl]methyl]indazole-3-carbonyl]amino]ethyl]benzoate (35 mg, 61.88 μmol, 1 eq) in HCl/MeOH (3 mL) was stirred at 50° C. for 16 hr.

The reaction mixture was concentrated by reduced pressure to give a residue. The crude product was used into the next step without further purification to give methyl 4-[(1S)-1-[[4-[[4-(trifluoromethyl)phenyl]methyl]-1H-indazole-3-carbonyl]amino]ethyl]benzoate (30 mg, 62.31 μmol, 97.89% yield) as a white solid, which was used into the next step directly without further purification. LCMS for product (ESI): m/z 482.2 [M+H]⁺.

### Step 6: Preparation of Compound 7,4-[(1S)-1-[[4-[[4-(trifluoromethyl)phenyl]methyl]-1H-indazole-3-carbonyl]amino]ethyl]benzoic acid

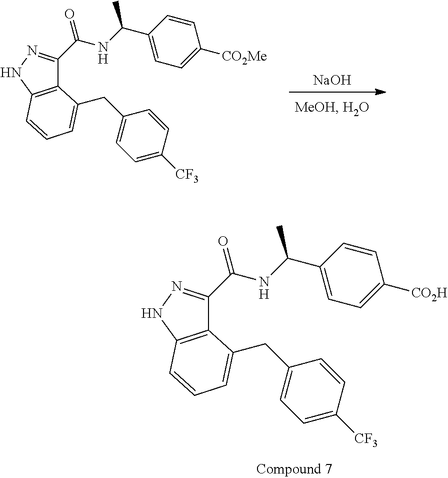 [SMILES: COC(=O)c1ccc([C@H](C)NC(=O)c2n[nH]c3cccc(Cc4ccc(C(F)(F)F)cc4)c23)cc1 ; "NaOH" ; C[C@H](NC(=O)c1n[nH]c2cccc(Cc3ccc(C(F)(F)F)cc3)c12)c1ccc(C(=O)O)cc1]

To a solution of methyl 4-[(1S)-1-[[4-[[4-(trifluoromethyl)phenyl]methyl]-1H-indazole-3-carbonyl]amino]ethyl]benzoate (30 mg, 62.31 μmol, 1 eq) in H₂O (4 mL) and MeOH (1.5 mL) was added NaOH (7.48 mg, 186.93 μmol, 3 eq). The mixture was stirred at 50° C. for 16 hr.

The reaction mixture was concentrated under reduced pressure to remove MeOH. The mixture was adjusted to PH 4 with 1N HCl. The mixture was filtered to provide Compound 7, 4-[(1S)-1-[[4-[[4-(trifluoromethyl)phenyl]methyl]-1H-indazole-3-carbonyl]amino]ethyl]benzoic acid (26 mg, 52.28 μmol, 83.90% yield, 93.99% purity) as a white solid. LCMS for product (ESI): m/z 468.2 [M+H]⁺. ¹H NMR (400 MHz, DMSO-d₆) δ ppm 13.56 (s, 1H), 8.96 (d, J=8.0 Hz, 1H), 7.91 (d, J=8.0 Hz, 2H), 7.56 (d, J=8.4 Hz, 2H), 7.50 (d, J=8.4 Hz, 1H), 7.41-7.34 (m, 3H), 7.22 (d, J=8.0 Hz, 2H), 7.11 (d, J=6.8 Hz, 1H), 5.27-5.23 (m, 1H), 4.74 (d, J=14.4 Hz, 1H), 4.56 (d, J=14.4 Hz, 1H), 1.47 (d, J=7.2 Hz, 3H).

### Example 9—Synthesis of 2-[[4-[[4-(trifluoromethyl)phenyl]methyl]-1H-indazole-3-carbonyl]amino]spiro[3.3]heptane-6-carboxylic acid (Compound 8)

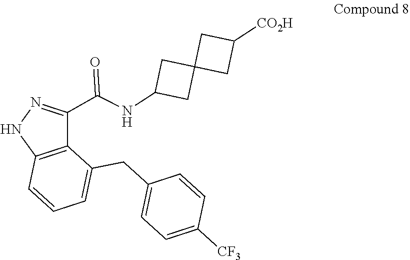 [SMILES: O=C(NC1CC2(C1)CC(C(=O)O)C2)c1n[nH]c2cccc(Cc3ccc(C(F)(F)F)cc3)c12]

### Step 1: Preparation of ethyl 2-[[1-tetrahydropyran-2-yl-4-[[4-(trifluoromethyl)phenyl]methyl]indazole-3-carbonyl]amino]spiro[3.3]heptane-6-carboxylate

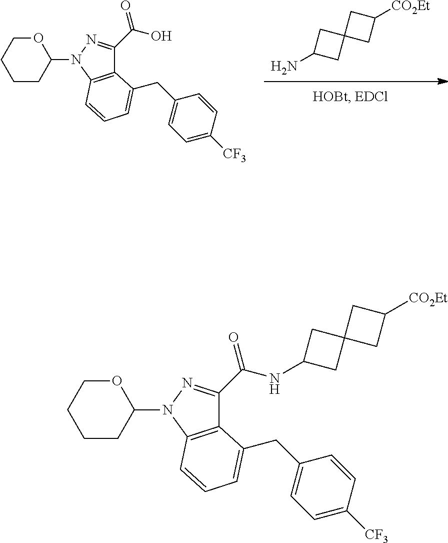 [SMILES: O=C(O)c1nn(C2CCCCO2)c2cccc(Cc3ccc(C(F)(F)F)cc3)c12 ; CCOC(=O)C1CC2(CC(N)C2)C1 ; CCOC(=O)C1CC2(CC(NC(=O)c3nn(C4CCCCO4)c4cccc(Cc5ccc(C(F)(F)F)cc5)c34)C2)C1]

To a solution of 1-tetrahydropyran-2-yl-4-[[4-(trifluoromethyl)phenyl]methyl]indazole-3-carboxylic acid (50 mg, 123.65 μmol, 1 eq) in DCM (1 mL) was added EDCI (38.39 mg, 247.29 μmol, 43.77 μL, 2 eq), Et₃N (62.56 mg, 618.23 μmol, 86.05 μL, 5 eq) and HOBt (3.34 mg, 24.73 μmol, 0.2 eq). The mixture was stirred at 0° C. for 0.5 hr. Then was added ethyl 2-aminospiro[3.3]heptane-6-carboxylate (33.99 mg, 185.47 μmol, 1.5 eq) at 0° C., the mixture was stirred at 25° C. for 12 hr.

The residue was diluted with H₂O 10 mL and extracted with DCM 15 mL (5 mL*3). The combined organic layers were washed with brine 15 mL (5 mL*3), dried over Na₂SO₄, filtered and concentrated under reduced pressure to give a residue. The residue was purified by prep-TLC (SiO₂, PE:EA=2:1) to provide ethyl 2-[[1-tetrahydropyran-2-yl-4-[[4-(trifluoromethyl)phenyl]methyl]indazole-3-carbonyl]amino]spiro[3.3]heptane-6-carboxylate (30 mg, 52.67 μmol, 42.60% yield) as a white solid, which was used into the next step directly without further purification. LCMS for product (ESI): m/z 570.4 [M+H]⁺.

### Step 2: Preparation of ethyl 6-(4-(4-(trifluoromethyl)benzyl)-1H-indazole-3-carboxamido)spiro[3.3]heptane-2-carboxylate

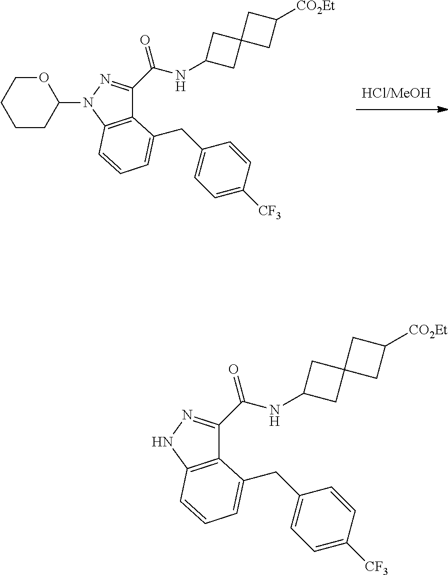 [SMILES: CCOC(=O)C1CC2(CC(NC(=O)c3nn(C4CCCCO4)c4cccc(Cc5ccc(C(F)(F)F)cc5)c34)C2)C1 ; CCOC(=O)C1CC2(CC(NC(=O)c3n[nH]c4cccc(Cc5ccc(C(F)(F)F)cc5)c34)C2)C1]

To a solution of ethyl 2-[[1-tetrahydropyran-2-yl-4-[[4-(trifluoromethyl)phenyl]methyl]indazole-3-carbonyl]amino]spiro[3.3]heptane-6-carboxylate (30 mg, 52.67 μmol, 1 eq) in HCl/MeOH (3 mL). The mixture was stirred at 25° C. for 1 hr.

The reaction mixture was concentrated under reduced pressure to remove HCl/MeOH. The crude product was used into the next step without further purification and compound ethyl 6-(4-(4-(trifluoromethyl)benzyl)-1H-indazole-3-carboxamido)spiro[3.3]heptane-2-carboxylate (25 mg, 51.49 μmol, 97.7% yield) was obtained as a white solid.

### Step 3: Preparation of Compound 8,2-[[4-[[4-(trifluoromethyl)phenyl]methyl]-1H-indazole-3-carbonyl]amino]spiro[3.3]heptane-6-carboxylic acid

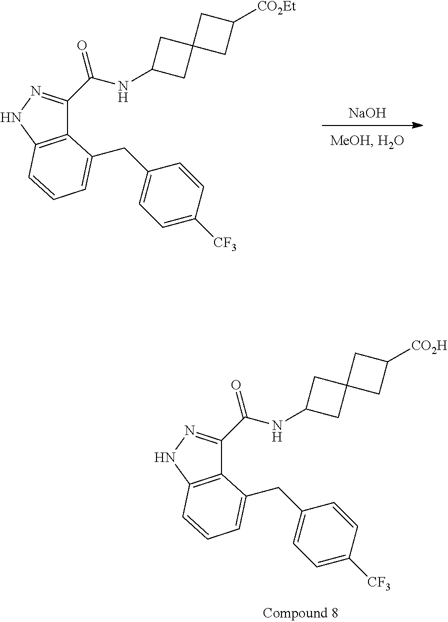 [SMILES: CCOC(=O)C1CC2(CC(NC(=O)c3n[nH]c4cccc(Cc5ccc(C(F)(F)F)cc5)c34)C2)C1 ; "NaOH" ; O=C(NC1CC2(C1)CC(C(=O)O)C2)c1n[nH]c2cccc(Cc3ccc(C(F)(F)F)cc3)c12]

To a solution of methyl ethyl 6-(4-(4-(trifluoromethyl)benzyl)-1H-indazole-3-carboxamido)spiro[3.3]heptane-2-carboxylate (25 mg, 51.49 μmol, 42% yield) in H₂O (1.5 mL) and MeOH (1.5 mL) was added NaOH (7.48 mg, 186.93 μmol, 3 eq). The mixture was stirred at 50° C. for 16 hr.

The reaction mixture was concentrated under reduced pressure to remove MeOH. The mixture was adjusted to pH 4 with 1N HCl. The mixture was filtered and Compound 8, 2-[[4-[[4-(trifluoromethyl)phenyl]methyl]-1H-indazole-3-carbonyl]amino]spiro[3.3]heptane-6-carboxylic acid (20 mg, 41.97 μmol, 79.69% yield, 96% purity), was obtained as a white solid. LCMS for product (ESI): m/z 458.1 [M+H]⁺. ¹H NMR (400 MHz, DMSO-d₆) δ=13.50 (s, 1H), 12.01 (brs, 1H), 8.54 (d, J=8.0 Hz, 1H), 7.54 (d, J=8.0 Hz, 2H), 7.47 (d, J=8.4 Hz, 1H), 7.38-7.29 (m, 3H), 7.07 (d, J=7.2 Hz, 1H), 4.68 (s, 2H), 4.31-4.23 (m, 1H), 2.93 (t, J=8.4 Hz, 1H), 2.41-2.34 (m, 1H), 2.32-2.25 (m, 1H), 2.23-2.16 (m, 2H), 2.15-2.02 (m, 3H), 2.01-1.94 (m, 1H).

### Example 10—Synthesis of 4-[(1S)-1-[[1-ethyl-4-[[4-(trifluoromethyl) phenyl]methyl]indazole-3-carbonyl]amino]ethyl]benzoic acid (Compound 9)

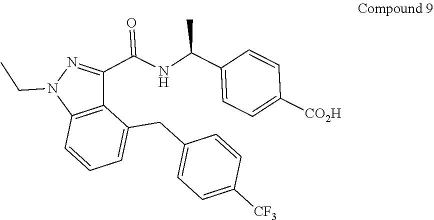 [SMILES: CCn1nc(C(=O)N[C@@H](C)c2ccc(C(=O)O)cc2)c2c(Cc3ccc(C(F)(F)F)cc3)cccc21]

### Step 1: Preparation of methyl 4-bromo-1-ethyl-indazole-3-carboxylate and methyl 4-bromo-2-ethyl-indazole-3-carboxylate

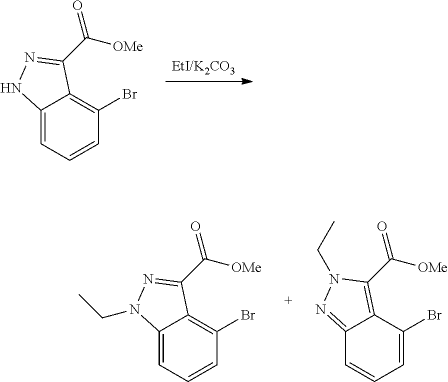 [SMILES: COC(=O)c1n[nH]c2cccc(Br)c12 ; "EtI/K2CO3" ; CCn1nc(C(=O)OC)c2c(Br)cccc21 ; CCn1nc2cccc(Br)c2c1C(=O)OC]

To a solution of methyl 4-bromo-1H-indazole-3-carboxylate (1 g, 3.92 mmol, 1 eq) and iodoethane (3.06 g, 19.60 mmol, 1.57 mL, 5 eq) in DMF (3 mL) was added K₂CO₃ (1.08 g, 7.84 mmol, 2 eq). The mixture was stirred at 25° C. for 12 hr.

The reaction mixture was concentrated under reduced pressure to remove DMF. The residue was diluted with H₂O 30 mL and extracted with EA 30 mL (10 mL×3). The combined organic layers were washed with brine 15 mL, dried over Na₂SO₄, filtered and concentrated under reduced pressure to give a residue. The residue was purified by column chromatography (SiO₂, Petroleum ether/Ethyl acetate=50/1 to 1/1) to provide methyl 4-bromo-1-ethyl-indazole-3-carboxylate (700 mg, 2.47 mmol, 63.06% yield) and methyl 4-bromo-2-ethyl-indazole-3-carboxylate (300 mg, 1.06 mmol, 27.03% yield) as a yellow oil.

Methyl 4-bromo-1-ethyl-indazole-3-carboxylate: ¹H NMR (400 MHz, DMSO-d₆) δ=7.87 (d, J=8.4 Hz, 1H), 7.55 (d, J=7.2 Hz, 1H), 7.42-7.38 (m, 1H), 4.53 (q, J=7.2 Hz, 2H), 3.91 (s, 3H), 1.42 (t, J=7.2 Hz, 3H).

Methyl 4-bromo-2-ethyl-indazole-3-carboxylate:

¹H NMR (400 MHz, DMSO-d₆) δ=7.77 (d, J=8.4 Hz, 1H), 7.49 (d, J=7.2 Hz, 1H), 7.27-7.23 (m, 1H), 4.60 (q, J=7.2 Hz, 2H), 4.00 (s, 3H), 1.48 (t, J=7.2 Hz, 3H).

### Step 2: Preparation of methyl 1-ethyl-4-[[4-(trifluoromethyl) phenyl]methyl]indazole-3-carboxylate

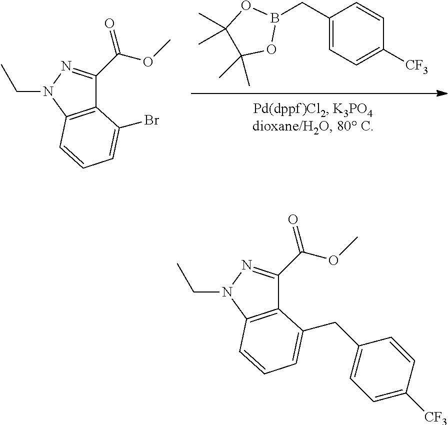

To a solution of methyl 4-bromo-1-ethyl-indazole-3-carboxylate (200 mg, 706.41 μmol, 1 eq) and 4,4,5,5-tetramethyl-2-[[4-(trifluoromethyl) phenyl]methyl]-1,3,2-dioxaborolane (202.10 mg, 706.41 μmol, 1 eq) in dioxane (1.5 mL) and H₂O (0.5 mL) was added Pd(dppf)Cl₂ (155.07 mg, 211.92 μmol, 0.3 eq) and K₃PO₄ (149.95 mg, 706.41 μmol, 1 eq) under nitrogen. The mixture was stirred at 80° C. for 12 hr.

The reaction mixture was concentrated under reduced pressure to remove dioxane. The residue was diluted with H2O 10 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO2, PE: EA=2:1). Compound methyl 1-ethyl-4-[[4-(trifluoromethyl) phenyl]methyl]indazole-3-carboxylate (130 mg, 358.77 μmol, 50.79% yield) was obtained as a white solid. HPLC showed the product was clean.

LCMS for product (ESI): m/z 331.3 [M+H]⁺.

### Step 3: Preparation of 1-ethyl-4-[[4-(trifluoromethyl) phenyl]methyl]indazole-3-carboxylic acid

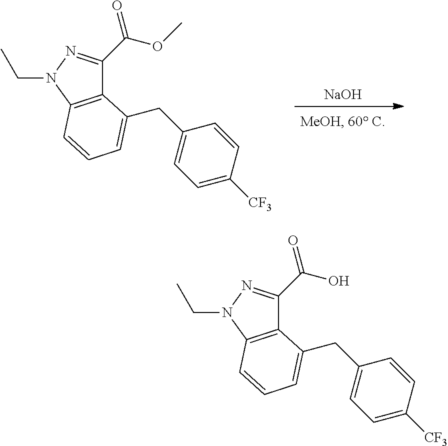 [SMILES: CCn1nc(C(=O)OC)c2c(Cc3ccc(C(F)(F)F)cc3)cccc21 ; "NaOH" ; CCn1nc(C(=O)O)c2c(Cc3ccc(C(F)(F)F)cc3)cccc21]

To a solution of methyl 1-ethyl-4-[[4-(trifluoromethyl) phenyl]methyl]indazole-3-carboxylate (120 mg, 331.18 μmol, 1 eq) in H₂O (6 mL) and MeOH (3 mL) was added NaOH (26.5 mg, 662.36 μmol, 2 eq). The mixture was stirred at 50° C. for 12 hr.

The reaction mixture was concentrated under reduced pressure to remove MeOH. The mixture was adjusted to PH 4 with 1N HCl. The mixture was filtered to give 1-ethyl-4-[[4-(trifluoromethyl) phenyl]methyl]indazole-3-carboxylic acid (90 mg, 258.38 μmol, 78.02% yield) as a white solid, which was used into the next step directly without further purification. LCMS for product (ESI): m/z 349.3 [M+H]⁺.

### Step 4: Preparation of methyl 4-[(1S)-1-[[1-ethyl-4-[[4-(trifluoromethyl) phenyl]methyl]indazole-3-carbonyl]amino]ethyl]benzoate

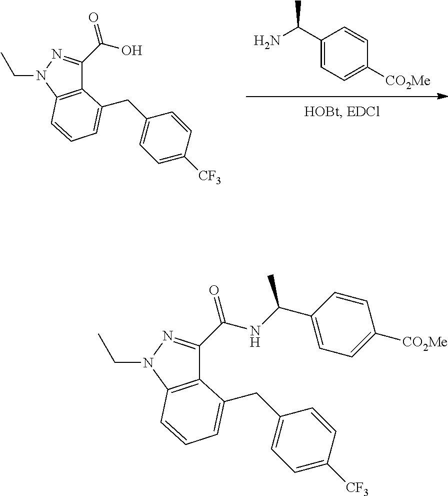 [SMILES: CCn1nc(C(=O)O)c2c(Cc3ccc(C(F)(F)F)cc3)cccc21 ; COC(=O)c1ccc([C@H](C)N)cc1 ; CCn1nc(C(=O)N[C@@H](C)c2ccc(C(=O)OC)cc2)c2c(Cc3ccc(C(F)(F)F)cc3)cccc21]

To a solution of 1-ethyl-4-[[4-(trifluoromethyl) phenyl]methyl]indazole-3-carboxylic acid (45 mg, 129.19 μmol, 1 eq) in DCM (1 mL) was added Et3N (65.36 mg, 645.96 μmol, 89.91 μL, 5 eq), EDCI (49.53 mg, 258.38 μmol, 2 eq) and HOBt (3.49 mg, 25.84 μmol, 0.2 eq) at 0° C. The mixture was stirred at 0° C. for 0.5 hr. Then was added methyl 4-[(1S)-1-aminoethyl]benzoate (46.31 mg, 258.38 μmol, 2 eq), the mixture was stirred at 25° C. for 12 hr.

The residue was diluted with H₂O 5 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO2, PE: EA=2:1). Compound methyl 4-[(1S)-1-[[1-ethyl-4-[[4-(trifluoromethyl) phenyl]methyl]indazole-3-carbonyl]amino]ethyl]benzoate (30 mg, 58.88 μmol, 45.57% yield) was obtained as a white solid, which was used into the next step directly without further purification.

LCMS for product (ESI): m/z 510.4 [M+H]⁺.

### Step 5: Preparation of Compound 9,4-[(1S)-1-[[1-ethyl-4-[[4-(trifluoromethyl) phenyl methyl]indazole-3-carbonyl]amino]ethyl]benzoic acid

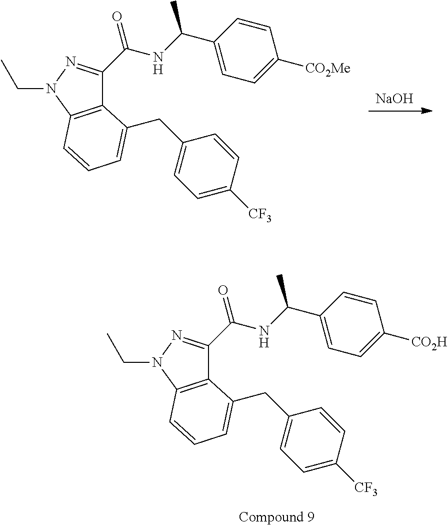 [SMILES: CCn1nc(C(=O)N[C@@H](C)c2ccc(C(=O)OC)cc2)c2c(Cc3ccc(C(F)(F)F)cc3)cccc21 ; "NaOH" ; CCn1nc(C(=O)N[C@@H](C)c2ccc(C(=O)O)cc2)c2c(Cc3ccc(C(F)(F)F)cc3)cccc21]

To a solution of methyl 4-[(1S)-1-[[1-ethyl-4-[[4-(trifluoromethyl) phenyl]methyl]indazole-3-carbonyl]amino]ethyl]benzoate (30 mg, 58.88 μmol, 1 eq) in H₂O (3 mL) and MeOH (1 mL) was added NaOH (7.06 mg, 176.64 μmol, 3 eq). The mixture was stirred at 50° C. for 12 hr.

The reaction mixture was concentrated under reduced pressure to remove MeOH. The mixture was adjusted to PH 4 with 1N HCl. The mixture was filtered to provide Compound 9, 4-[(1S)-1-[[1-ethyl-4-[[4-(trifluoromethyl) phenyl]methyl]indazole-3-carbonyl]amino]ethyl]benzoic acid (15 mg, 29.36 μmol, 49.87% yield, 97% purity) as a white solid. LCMS for product (ESI): m/z 496.2 [M+H]⁺. ¹H NMR (400 MHz, DMSO-d₆) δ=8.89 (d, J=8.4 Hz, 1H), 7.90 (d, J=8.0 Hz, 2H), 7.64 (d, J=8.4 Hz, 1H), 7.54 (d, J=8.4 Hz, 2H), 7.40 (dd, J=7.1, 8.4 Hz, 1H), 7.34 (d, J=8.4 Hz, 2H), 7.19 (d, J=8.0 Hz, 2H), 7.13 (d, J=7.2 Hz, 1H), 5.28-5.21 (m, 1H), 4.72 (d, J=14.4 Hz, 1H), 4.54-4.47 (m, 3H), 1.48-1.40 (m, 6H).

### Example 11—Synthesis of 4-[(1S)-1-[[2-ethyl-4-[[4-(trifluoromethyl) phenyl]methyl]indazole-3-carbonyl]amino]ethyl]benzoic acid (Compound 10)

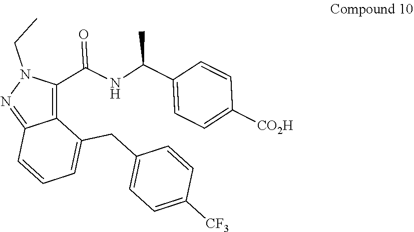 [SMILES: CCn1nc2cccc(Cc3ccc(C(F)(F)F)cc3)c2c1C(=O)N[C@@H](C)c1ccc(C(=O)O)cc1]

### Step 1: Preparation of methyl 2-ethyl-4-[[4-(trifluoromethyl)phenyl]-methyl]indazole-3-carboxylate

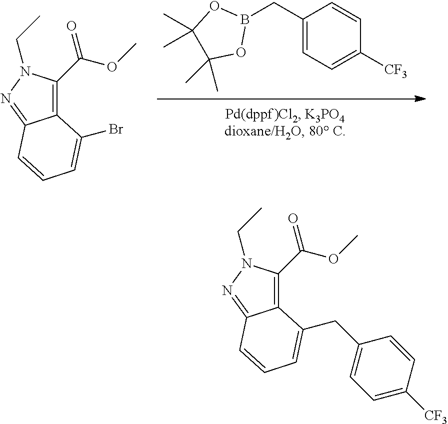

To a solution of methyl 4-bromo-2-ethyl-indazole-3-carboxylate (100.00 mg, 353.21 μmol, 1 eq) and 4,4,5,5-tetramethyl-2-[[4-(trifluoromethyl)phenyl]methyl]-1,3,2-dioxaborolane (101.05 mg, 353.21 μmol, 1 eq) in dioxane (0.4 mL) and H₂O (0.2 mL) was added Pd(dppf)Cl₂ (77.53 mg, 105.96 μmol, 0.3 eq) and K₃PO₄ (74.98 mg, 353.21 μmol, 1 eq). The mixture was stirred at 80° C. for 12 hr.

The reaction mixture was concentrated under reduced pressure to remove dioxane. The residue was diluted with H₂O 10 mL and extracted with DCM 15 mL (5 mL*3). The combined organic layers were washed with brine 10 mL, dried over Na₂SO₄, filtered and concentrated under reduced pressure to give a residue. The residue was purified by prep-TLC (SiO₂, PE: EA=2:1) to provide methyl 2-ethyl-4-[[4-(trifluoromethyl)phenyl]methyl]indazole-3-carboxylate (35 mg, 96.59 μmol, 27.35% yield) as a white solid. HPLC showed the product was clean. LCMS for crude product (ESI): m/z 363.3 [M+H]⁺.

### Step 2: Preparation of 2-ethyl-4-[[4-(trifluoromethyl)phenyl]methyl]indazole-3-carboxylic acid

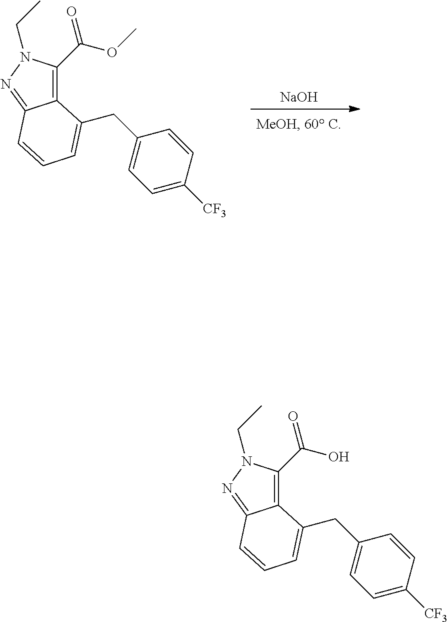 [SMILES: CCn1nc2cccc(Cc3ccc(C(F)(F)F)cc3)c2c1C(=O)OC ; "NaOH" ; CCn1nc2cccc(Cc3ccc(C(F)(F)F)cc3)c2c1C(=O)O]

To a solution of methyl 2-ethyl-4-[[4-(trifluoromethyl)phenyl]methyl]indazole-3-carboxylate (33 mg, 91.07 μmol, 1 eq) in H₂O (0.5 mL) and MeOH (0.5 mL) was added NaOH (10.92 mg, 273.21 μmol, 3 eq). The mixture was stirred at 50° C. for 12 hr.

The reaction mixture was concentrated under reduced pressure to remove MeOH. The mixture was adjusted to pH 4 with 1N HCl. The mixture was filtered and 2-ethyl-4-[[4-(trifluoromethyl)phenyl]methyl]indazole-3-carboxylic acid (20 mg, 57.42 μmol, 63.05% yield) was obtained as a white solid, which was used into the next step directly without further purification.

LCMS for product (ESI): m/z 349.3 [M+H]⁺.

### Step 3: Preparation of methyl 4-[(1S)-1-[[2-ethyl-4-[[4-(trifluoromethyl) phenyl]methyl]indazole-3-carbonyl]amino]ethyl]benzoate

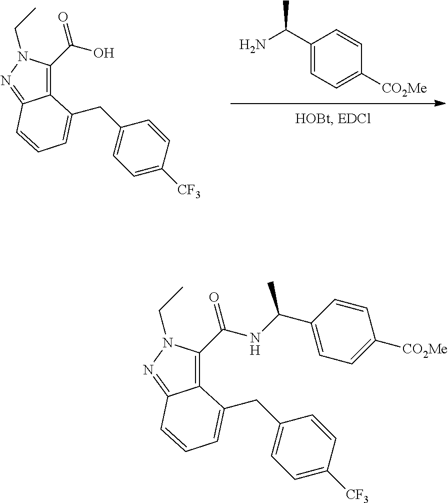 [SMILES: CCn1nc2cccc(Cc3ccc(C(F)(F)F)cc3)c2c1C(=O)O ; COC(=O)c1ccc([C@H](C)N)cc1 ; CCn1nc2cccc(Cc3ccc(C(F)(F)F)cc3)c2c1C(=O)N[C@@H](C)c1ccc(C(=O)OC)cc1]

To a solution of methyl 2-ethyl-4-[[4-(trifluoromethyl)phenyl]methyl]indazole-3-carboxylic acid (10 mg, 28.71 μmol, 1 eq) in DCM (1 mL) was added Et₃N (14.53 mg, 143.55 μmol, 19.98 μL, 5 eq), EDCI (11.01 mg, 57.42 μmol, 2 eq) and HOBt (775.84 ug, 5.74 μmol, 0.2 eq) at 0° C. The mixture was stirred at 0° C. for 0.5 hr. Then was added methyl 4-[(1S)-1-aminoethyl]benzoate (10.29 mg, 57.42 μmol, 2 eq) at 0° C., the mixture was stirred at 25° C. for 12 hr.

The residue was diluted with H₂O 10 mL and extracted with DCM 15 mL (5 mL*3). The combined organic layers were washed with brine 10 mL, dried over Na₂SO₄, filtered and concentrated under reduced pressure to give a residue. The residue was purified by prep-TLC (SiO₂, PE: EA=2:1). Compound methyl 4-[(1S)-1-[[2-ethyl-4-[[4-(trifluoromethyl) phenyl]methyl]indazole-3-carbonyl]amino]ethyl]benzoate (9 mg, 17.66 μmol, 61.53% yield) was obtained as a white solid, which was used into the next step directly without further purification.

LCMS for product (ESI): m/z 510.3 [M+H]⁺.

### Step 4: Preparation of Compound 10,4-[(1S)-1-[[2-ethyl-4-[[4-(trifluoromethyl) phenyl]methyl]indazole-3-carbonyl]amino]ethyl]benzoic acid

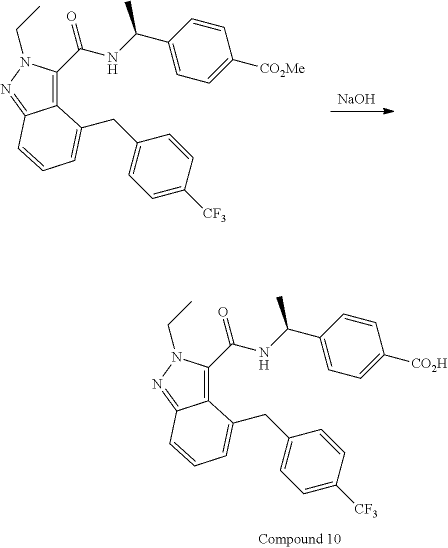 [SMILES: CCn1nc2cccc(Cc3ccc(C(F)(F)F)cc3)c2c1C(=O)N[C@@H](C)c1ccc(C(=O)OC)cc1 ; "NaOH" ; CCn1nc2cccc(Cc3ccc(C(F)(F)F)cc3)c2c1C(=O)N[C@@H](C)c1ccc(C(=O)O)cc1]

To a solution of methyl 4-[(1S)-1-[[2-ethyl-4-[[4-(trifluoromethyl) phenyl]methyl]indazole-3-carbonyl]amino]ethyl]benzoate (9 mg, 17.66 μmol, 1 eq) in H₂O (1 mL) and MeOH (1 mL) was added NaOH (2.12 mg, 52.99 μmol, 3 eq). The mixture was stirred at 50° C. for 12 hr.

The residue was diluted with H₂O 10 mL and extracted with DCM 15 mL (5 mL*3). The combined organic layers were washed with brine 5 mL, dried over Na₂SO₄, filtered and concentrated under reduced pressure to give a residue. The residue was purified by prep-HPLC (HCl condition) and Compound 10, 4-[(1S)-1-[[2-ethyl-4-[[4-(trifluoromethyl) phenyl]methyl]indazole-3-carbonyl]amino]ethyl]benzoic acid (1.5 mg, 2.94 μmol, 16.62% yield, 97% purity), was obtained as a white solid. LCMS for product (ESI): m/z 496.2 [M+H]⁺. ¹H NMR (400 MHz, DMSO-d₆) δ=9.53 (d, J=7.6 Hz, 1H), 7.89 (d, J=8.4 Hz, 2H), 7.55-7.48 (m, 5H), 7.25-7.18 (m, 3H), 6.79 (d, J=6.8 Hz, 1H), 5.21-5.15 (m, 1H), 4.38-4.32 (m, 2H), 4.18 (s, 2H), 1.40-1.31 (m, 6H).

### Example 12—Synthesis of cis-2-[[1-ethyl-4-[[4-(trifluoromethyl) phenyl]methyl]indazole-3-carbonyl]amino]spiro[3.3]heptane-6-carboxylic acid (Compound 11A)

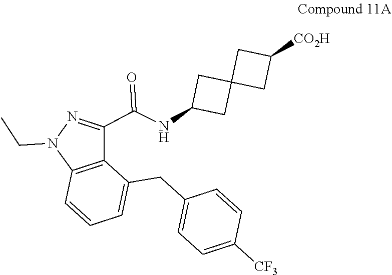 [SMILES: CCn1nc(C(=O)N[C@H]2CC3(C2)C[C@H](C(=O)O)C3)c2c(Cc3ccc(C(F)(F)F)cc3)cccc21]

### Step 1: Preparation of methyl 4-bromo-1-ethyl-indazole-3-carboxylate and methyl 4-bromo-2-ethyl-indazole-3-carboxylate

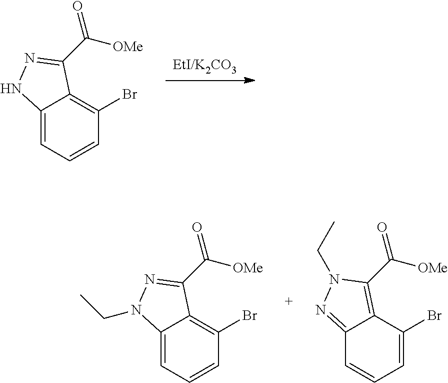 [SMILES: COC(=O)c1n[nH]c2cccc(Br)c12 ; "EtI/K2CO3" ; CCn1nc(C(=O)OC)c2c(Br)cccc21 ; CCn1nc2cccc(Br)c2c1C(=O)OC]

To a solution of methyl 4-bromo-1H-indazole-3-carboxylate (1 g, 3.92 mmol, 1 eq) and iodoethane (3.06 g, 19.60 mmol, 1.57 mL, 5 eq) in DMF (10 mL) was added K₂CO₃ (1.08 g, 7.84 mmol, 2 eq). The mixture was stirred at 25° C. for 12 hr.

The reaction mixture was diluted with H₂O 30 mL and extracted with DCM 60 mL (20 mL×3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=50/1 to 1/1) to provide methyl 4-bromo-1-ethyl-indazole-3-carboxylate (700 mg, 2.47 mmol, 63.06% yield) and methyl 4-bromo-2-ethyl-indazole-3-carboxylate (300 mg, 1.06 mmol, 27.03% yield) as yellow oil.

### Step 2: Preparation of methyl 1-ethyl-4-[[4-(trifluoromethyl) phenyl]methyl]indazole-3-carboxylate

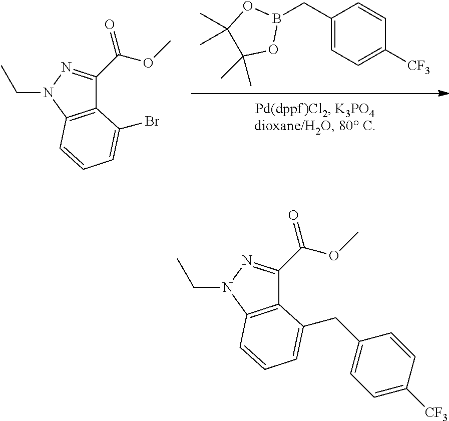

To a solution of methyl 4-bromo-1-ethyl-indazole-3-carboxylate (300 mg, 1.06 mmol, 1 eq) and 4,4,5,5-tetramethyl-2-[[4-(trifluoromethyl) phenyl]methyl]-1,3,2-dioxaborolane (303.15 mg, 1.06 mmol, 1 eq) in dioxane (3 mL) and H₂O (1 mL) was added Pd(dppf)Cl₂ (232 mg, 319 μmol, 0.3 eq) and K₃PO₄ (225 mg, 1.06 mmol, 1 eq). The mixture was stirred at 80° C. for 12 hr.

The reaction mixture was diluted with H₂O 10 mL and extracted with DCM 60 mL (20 mL×3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=50/1 to 1/1). Compound methyl 1-ethyl-4-[[4-(trifluoromethyl) phenyl]methyl]indazole-3-carboxylate (220 mg, 607.16 μmol, 57.30% yield) was obtained as a white solid. LCMS for crude product (ESI): m/z 363.3 [M+H]⁺.

### Step 3: Preparation of 1-ethyl-4-[[4-(trifluoromethyl) phenyl]methyl]indazole-3-carboxylic acid

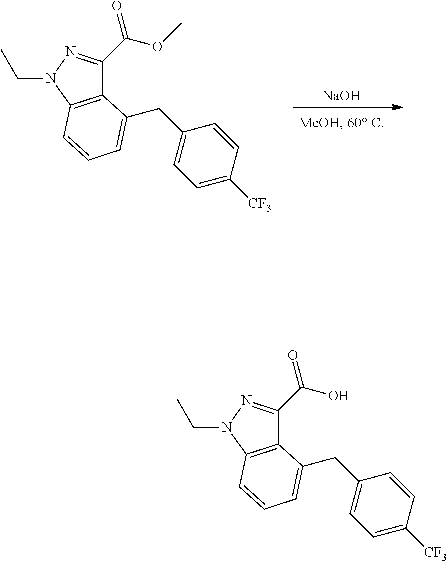 [SMILES: CCn1nc(C(=O)OC)c2c(Cc3ccc(C(F)(F)F)cc3)cccc21 ; "NaOH" ; CCn1nc(C(=O)O)c2c(Cc3ccc(C(F)(F)F)cc3)cccc21]

To a solution of methyl 1-ethyl-4-[[4-(trifluoromethyl) phenyl]methyl]indazole-3-carboxylate (220 mg, 607.16 μmol, 1 eq) in H₂O (5 mL) was added NaOH (24.28 mg, 607.16 μmol, 1 eq) and MeOH (3 mL). The mixture was stirred at 50° C. for 16 hr.

The reaction mixture was concentrated under reduced pressure to remove MeOH. The mixture was adjusted to PH 4 with 1N HCl. The mixture was filtered to give 1-ethyl-4-[[4-(trifluoromethyl) phenyl]methyl]indazole-3-carboxylic acid (170 mg, 488.06 μmol, 80.38% yield) as a white solid. LCMS for product (ESI): m/z 349.3 [M+H]⁺.

### Step 4: Preparation of cis-(S)-1-phenylethyl 6-(1-ethyl-4-(4-(trifluoromethyl)benzyl)-1H-indazole-3-carboxamido)spiro[3.3]heptane-2-carboxylate

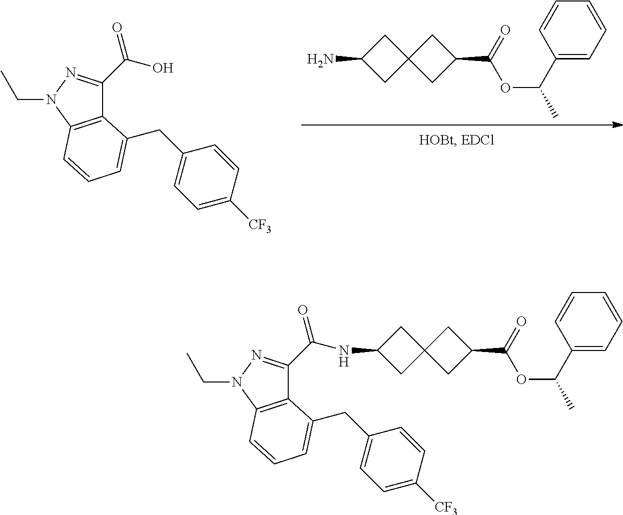 [SMILES: CCn1nc(C(=O)O)c2c(Cc3ccc(C(F)(F)F)cc3)cccc21 ; C[C@H](OC(=O)[C@H]1CC2(C[C@@H](N)C2)C1)c1ccccc1 ; CCn1nc(C(=O)N[C@H]2CC3(C2)C[C@H](C(=O)O[C@@H](C)c2ccccc2)C3)c2c(Cc3ccc(C(F)(F)F)cc3)cccc21]

To a solution of 1-ethyl-4-[[4-(trifluoromethyl) phenyl]methyl]indazole-3-carboxylic acid (50 mg, 143.55 μmol, 1 eq) in DCM (1 mL) was added HOBt (3.88 mg, 28.71 μmol, 0.2 eq), Et3N (72.63 mg, 717.75 μmol, 99.90 μL, 5 eq) and EDCI (55.04 mg, 287.10 μmol, 2 eq) at 0° C. The mixture was stirred at 0° C. for 0.5 hr. Then cis-(S)-1-phenylethyl 2-aminospiro[3.3]heptane-6-carboxylate (40.95 mg, 157.91 μmol, 1.1 eq) was added at 0° C. The mixture was stirred at 25° C. for 16 hr.

The reaction mixture was diluted with H₂O 3 mL and extracted with DCM 15 mL (5 mL×3). The combined organic layers were washed with brine 3 mL, dried over Na₂SO₄, filtered and concentrated under reduced pressure to give a residue. The residue was purified by prep-TLC (SiO₂, PE: EA=2:1). Compound cis-(S)-1-phenylethyl 6-(1-ethyl-4-(4-(trifluoromethyl)benzyl)-1H-indazole-3-carboxamido)spiro[3.3]heptane-2-carboxylate (30 mg, 46.46 μmol, 32.37% yield) was obtained as a white solid, which was used into the next step directly without further purification. LCMS for product (ESI): m/z 590.4 [M+H]⁺.

### Step 5: Preparation of Compound 11A, cis-2-[[1-ethyl-4-[[4-(trifluoromethyl) phenyl]methyl]indazole-3-carbonyl]amino]spiro[3.3]heptane-6-carboxylic acid

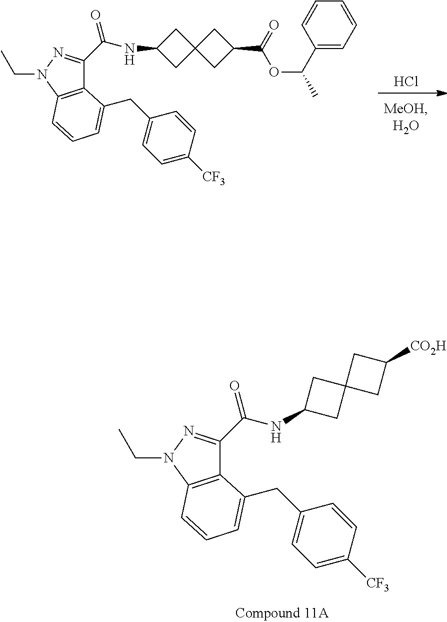

A solution of cis-(S)-1-phenylethyl 6-(1-ethyl-4-(4-(trifluoromethyl)benzyl)-1H-indazole-3-carboxamido)spiro[3.3]heptane-2-carboxylate (25.41 mg, 50.88 μmol, 1 eq) in HCl/MeOH (5 mL, 4M) was stirred at 25° C. for 6 hr.

The reaction mixture was concentrated under reduced pressure to remove MeOH. The mixture was dissolved by NaOH (5 mL, 1M) and filtered. The filtrate was adjusted to PH 4 with 1N HCl. The mixture was filtered to provide Compound 11A, cis-2-[[1-ethyl-4-[[4-(trifluoromethyl) phenyl]methyl]indazole-3-carbonyl]amino]spiro[3.3]heptane-6-carboxylic acid (20 mg, 38.86 μmol, 76.39% yield, 94.34% purity) as a white solid. LCMS for product (ESI): m/z 486.2 [M+H]⁺. ¹H NMR (400 MHz, DMSO-d₆) δ=8.44 (d, J=8.0 Hz, 1H), 7.62 (d, J=8.4 Hz, 1H), 7.54 (d, J=8.0 Hz, 2H), 7.39 (t, J=7.2 Hz, 1H), 7.31 (d, J=8.0 Hz, 2H), 7.10 (d, J=7.2 Hz, 1H), 4.70-4.63 (m, 2H), 4.47 (q, J=7.0 Hz, 2H), 4.32-4.21 (m, 1H), 2.88-2.76 (m, 2H), 2.38-2.35 (m, 2H), 2.25-2.21 (m, 2H), 2.15-2.10 (m, 2H), 2.05-1.95 (m, 2H), 1.41 (t, J=7.2 Hz, 3H)

### Example 13—Synthesis of trans-2-[[1-ethyl-4-[[4-(trifluoromethyl) phenyl]methyl]indazole-3-carbonyl]amino]spiro[3.3]heptane-6-carboxylic acid (Compound 11B)

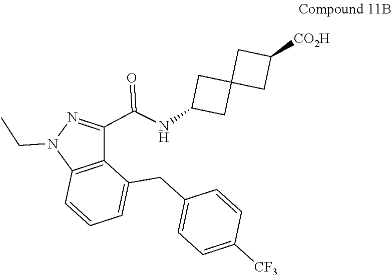 [SMILES: CCn1nc(C(=O)N[C@H]2CC3(C2)C[C@H](C(=O)O)C3)c2c(Cc3ccc(C(F)(F)F)cc3)cccc21]

### Step 1: Preparation of trans-(S)-1-phenylethyl 2-[[1-ethyl-4-[[4-(trifluoromethyl) phenyl]methyl]indazole-3-carbonyl]amino]spiro[3.3]heptane-6-carboxylate

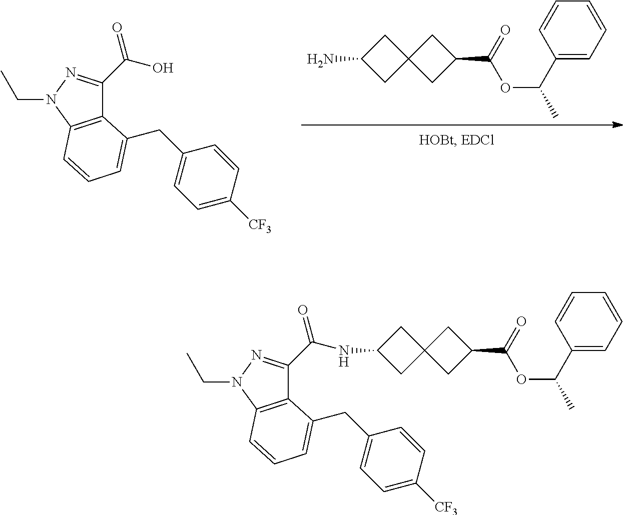 [SMILES: CCn1nc(C(=O)O)c2c(Cc3ccc(C(F)(F)F)cc3)cccc21 ; C[C@H](OC(=O)[C@H]1CC2(C[C@@H](N)C2)C1)c1ccccc1 ; CCn1nc(C(=O)N[C@H]2CC3(C2)C[C@H](C(=O)O[C@@H](C)c2ccccc2)C3)c2c(Cc3ccc(C(F)(F)F)cc3)cccc21]

To a solution of 1-ethyl-4-[[4-(trifluoromethyl) phenyl]methyl]indazole-3-carboxylic acid (50 mg, 143.55 μmol, 1 eq) in DCM (1 mL) was added HOBt (3.88 mg, 28.71 μmol, 0.2 eq), EDCI (55.04 mg, 287.10 μmol, 2 eq) and Et3N (72.63 mg, 717.75 μmol, 99.90 μL, 5 eq). The mixture was stirred at 0° C. for 0.5 hr. Then trans-(S)-1-phenylethyl 2-aminospiro[3.3]heptane-6-carboxylate (40.95 mg, 157.91 μmol, 1.1 eq) was added the mixture. The mixture was stirred at 25° C. for 16 hr.

The reaction mixture was diluted with H₂O 10 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO₂, PE:EA=2:1) to provide trans-(S)-1-phenylethyl 2-[[1-ethyl-4-[[4-(trifluoromethyl) phenyl]methyl]indazole-3-carbonyl]amino]spiro[3.3]heptane-6-carboxylate (30 mg, 61.79 μmol, 43.05% yield) as a white solid, which was used into the next step directly without further purification. LCMS for product (ESI): m/z 590.4 [M+H]⁺.

### Step 2: Preparation of Compound 11B, trans-2-[[1-ethyl-4-[[4-(trifluoromethyl) phenyl]methyl]indazole-3-carbonyl]amino]spiro[3.3]heptane-6-carboxylic acid

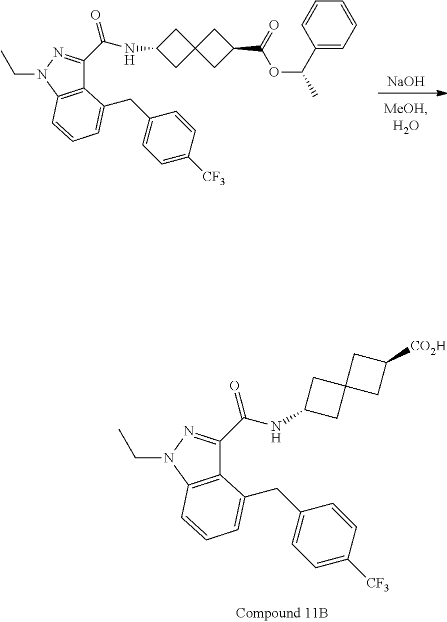

A solution of trans-(S)-1-phenylethyl 2-[[1-ethyl-4-[[4-(trifluoromethyl) phenyl]methyl]indazole-3-carbonyl]amino]spiro[3.3]heptane-6-carboxylate (25.41 mg, 50.88 μmol, 1 eq) in HCl/MeOH (5 mL, 4M) was stirred at 25° C. for 6 hr.

The reaction mixture was concentrated under reduced pressure to remove MeOH. The mixture was dissolved by NaOH (5 mL, 1M) and filtered. The filtrate was adjusted to PH 4 with 1N HCl. The mixture was filtered. Compound 11B, trans-2-[[1-ethyl-4-[[4-(trifluoromethyl) phenyl]methyl]indazole-3-carbonyl]amino]spiro[3.3]heptane-6-carboxylic acid (23 mg, 76% yield, 91.43% purity), was obtained as a white solid. LCMS for product (ESI): m/z 486.2 [M+H]⁺. ¹H NMR (400 MHz, DMSO-d₆) δ=12.00 (br s, 1H), 8.45 (d, J=8.0 Hz, 1H), 7.62 (d, J=8.4 Hz, 1H), 7.54 (d, J=8.0 Hz, 2H), 7.40 (t, J=8.0 Hz, 1H), 7.31 (d, J=8.0 Hz, 2H), 7.10 (d, J=7.2 Hz, 1H), 4.66 (s, 2H), 4.47 (q, J=7.2 Hz, 2H), 4.31-4.23 (m, 1H), 2.95-2.91 (m, 1H), 2.39-2.34 (m, 1H), 2.22-2.17 (m, 3H), 2.12-2.03 (m, 3H), 2.20-1.95 (m, 1H), 1.41 (t, J=7.2 Hz, 3H)

### Example 14—Synthesis of 4-[(1S)-1-[[4-(3-isoquinolylmethyl) pyrazolo[1,5-a]pyridine-3-carbonyl]amino]ethyl]benzoic acid (Compound 12)

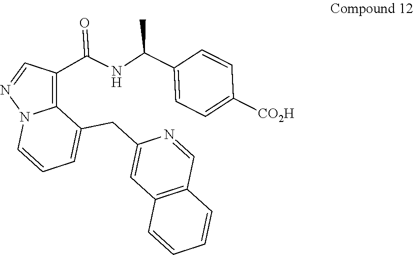 [SMILES: C[C@H](NC(=O)c1cnn2cccc(Cc3cc4ccccc4cn3)c12)c1ccc(C(=O)O)cc1]

### Step 1: Preparation of 3-(bromomethyl) isoquinoline

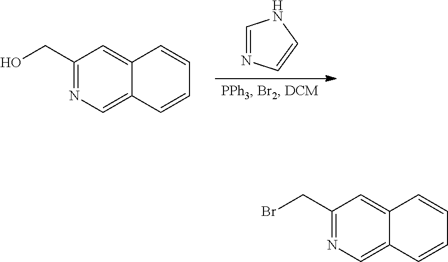

To a solution of 3-isoquinolylmethanol (400 mg, 2.51 mmol, 1 eq) and imidazole (188.18 mg, 2.76 mmol, 1.1 eq) in DCM (4 mL) was added PPh3 (308.12 mg, 2.76 mmol, 1.1 eq) and Br2 (723.12 mg, 1.17 mmol, 1.1 eq) at 0oC. The mixture was stirred at 0° C. for 10 min. LC-MS showed Reactant 1 was consumed completely and one main peak with desired mass. The solvent was quenched by saturation sodium hydrogen sulfite (10 mL) and extracted with DCM 30 (3×10 mL). The organic layer was washed with brine (10 mL), dried by sodium sulfate and concentrated by reduced pressure to give residue. The residue was purified by prep-TLC (SiO2, PE: EA=1:1). Compound 3-(bromomethyl) isoquinoline (400 mg, 1.801 mmol, 71.7% yield) was obtained as a white solid.

### Step 2: Preparation of methyl 4-(4,4,5,5-tetramethyl-1,3,2-dioxaborolan-2-yl)pyrazolo[1,5-a]pyridine-3-carboxylate

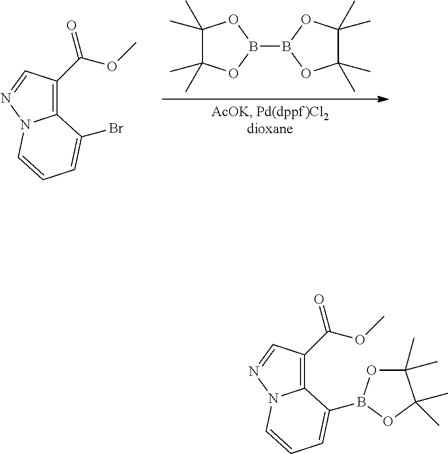

To a solution of methyl 4-bromopyrazolo[1,5-a]pyridine-3-carboxylate (1 g, 3.92 mmol, 1 eq) and 4,4,5,5-tetramethyl-2-(4,4,5,5-tetramethyl-1,3,2-dioxaborolan-2-yl)-1,3,2-dioxaborolane (1.09 g, 1.29 mmol, 1.1 eq) in dioxane (10 mL) was added AcOK (1.15 g, 11.76 mmol, 3 eq) and Pd (dppf) Cl₂ (57.36 mg, 78.4 umol, 0.02 eq) at 25° C. and stirred at 100° C. for 12 hr under nitrogen. LC-MS showed no of Reactant 1 was remained. Several new peaks were shown on LC-MS and 53% of desired compound was detected.

The reaction mixture was concentrated under reduced pressure to remove dioxane. The residue was diluted with H₂O 40 mL and extracted with EA 60 mL (20 mL×3). The combined organic layers were washed with brine 20 mL, dried over Na₂SO₄, filtered and concentrated under reduced pressure to give a residue. The residue was purified by column chromatography (eluted by PE: EA=2:1). Compound methyl 4-(4,4,5,5-tetramethyl-1,3,2-dioxaborolan-2-yl)pyrazolo[1,5-a]pyridine-3-carboxylate (750 mg, 2.48 mmol, 63.3% yield) was obtained as white solid. HPLC showed the product was clean. LCMS for product (ESI): m/z 303.4 [M+H]⁺.

### Step 3: Preparation of methyl 4-(3-isoquinolylmethyl) pyrazolo[1,5-a]pyridine-3-carboxylate

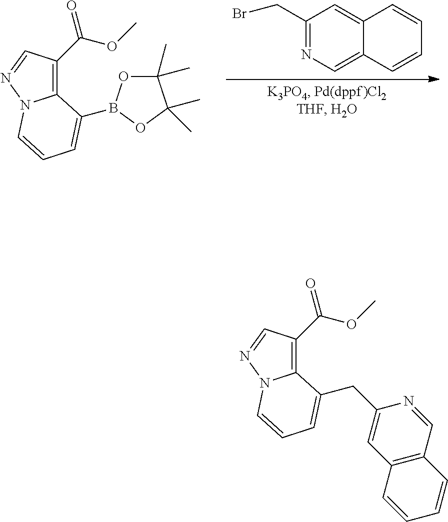

A mixture of methyl 4-(4,4,5,5-tetramethyl-1,3,2-dioxaborolan-2-yl) pyrazolo[1,5-a]pyridine-3-carboxylate (400 mg, 1323.92 umol, 1 eq), 3-(bromomethyl) isoquinoline (394.02 mg, 1323.92 umol, 1 eq), Pd(dppf)Cl₂ (193.74 mg, 264.78 umol, 0.2 eq), K₃PO₄ (281.02 mg, 1.32 mmol, 2 eq) in H₂O (0.5 mL) and THF (1 mL) was degassed and purged with N₂ for 3 times, and then the mixture was stirred at 25° C. for 12 hr under N₂ atmosphere. LC-MS showed Reactant 1 was consumed completely and one main peak with desired MS The reaction mixture was concentrated under reduced pressure to remove THF. The residue was diluted with H₂O 30 mL and extracted with EA 30 mL (10 mL×3). The combined organic layers were washed with brine 10 mL, dried over Na₂SO₄, filtered and concentrated under reduced pressure to give a residue. The residue was purified by prep-TLC (SiO2, PE: EA=1:1). Compound methyl 4-(3-isoquinolylmethyl) pyrazolo[1,5-a]pyridine-3-carboxylate (200 mg, 630.9 umol, 47% yield) was obtained as a white solid, which used directly into next step without further purification. LCMS for product (ESI): m/z 318.1 [M+H]⁺.

### Step 4: Preparation of 4-(3-isoquinolylmethyl) pyrazolo[1,5-a]pyridine-3-carboxylic acid

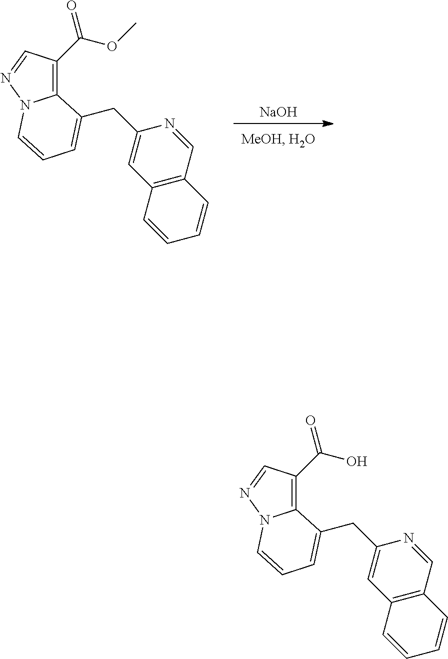 [SMILES: COC(=O)c1cnn2cccc(Cc3cc4ccccc4cn3)c12 ; "NaOH" ; O=C(O)c1cnn2cccc(Cc3cc4ccccc4cn3)c12]

To a solution of methyl 4-(3-isoquinolylmethyl) pyrazolo[1,5-a]pyridine-3-carboxylate (200 mg, 630.22 umol, 1 eq) in H₂O (2 mL) and MeOH (2 mL) was added NaOH (75.62 mg, 1890.75 umol, 3 eq). The mixture was stirred at 50° C. for 12 hr. LC-MS showed Reactant 1 was consumed completely and one main peak with desired Ms. The reaction mixture was concentrated under reduced pressure to remove MeOH. The mixture was adjusted to pH 4 with 1N HCl. The mixture was filtered. Compound 4-(3-isoquinolylmethyl) pyrazolo[1,5-a]pyridine-3-carboxylic acid (160 mg, 528.08 umol, 84% yield) was obtained as a white solid. LCMS for product (ESI): m/z 304.3 [M+H]⁺.

### Step 5: Preparation of 1-phenylethyl 2-[[4-(3-isoquinolylmethyl) pyrazolo[1,5-a]pyridine-3-carbonyl]amino]spiro[3.3]heptane-6-carboxylate

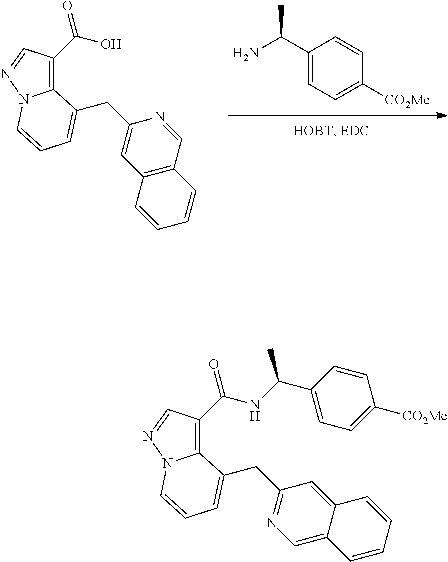 [SMILES: O=C(O)c1cnn2cccc(Cc3cc4ccccc4cn3)c12 ; COC(=O)c1ccc([C@H](C)N)cc1 ; COC(=O)c1ccc([C@H](C)NC(=O)c2cnn3cccc(Cc4cc5ccccc5cn4)c23)cc1]

To a solution of 4-(3-isoquinolylmethyl)pyrazolo[1,5-a]pyridine-3-carboxylic acid (35 mg, 115.39 μmol, 1 eq) in DCM (1 mL) was added HOBt (3.12 mg, 23.08 μmol, 0.2 eq), Et₃N (58.38 mg, 576.96 μmol, 80.31 μL, 5 eq) and EDCI (44.24 mg, 230.78 μmol, 2 eq) at 0° C. for 30 min. Then was added methyl 4-[(1S)-1-aminoethyl]benzoate (41.36 mg, 230.78 μmol, 2 eq) at 0° C. The resulting mixture was stirred at 25° C. for 12 hr.

The residue was diluted with H₂O 5 mL and extracted with DCM 30 mL (10 mL×3). The combined organic layers were washed with brine 5 mL, dried over Na₂SO₄, filtered and concentrated under reduced pressure to give a residue. The residue was purified by prep-TLC (SiO₂, PE: EA=1:1) to provide 1-phenylethyl 2-[[4-(3-isoquinolylmethyl) pyrazolo[1,5-a]pyridine-3-carbonyl]amino]spiro[3.3]heptane-6-carboxylate (30 mg, 55.08 μmol, 41.77% yield) as a white solid, which was used into the next step directly without further purification. LCMS for product (ESI): m/z 465.2 [M+H]⁺.

### Step 6: Preparation of Compound 12,4-[(1S)-1-[[4-(3-isoquinolylmethyl) pyrazolo[1,5-a]pyridine-3-carbonyl]amino]ethyl]benzoic acid

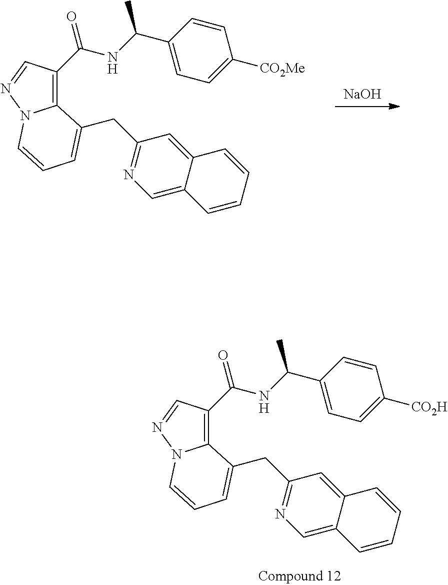 [SMILES: COC(=O)c1ccc([C@H](C)NC(=O)c2cnn3cccc(Cc4cc5ccccc5cn4)c23)cc1 ; "NaOH" ; C[C@H](NC(=O)c1cnn2cccc(Cc3cc4ccccc4cn3)c12)c1ccc(C(=O)O)cc1]

To a solution of methyl 4-[(1S)-1-[[4-(3-isoquinolylmethyl)pyrazolo[1,5-a]pyridine-3-carbonyl]amino]ethyl]benzoate (20 mg, 43.06 μmol, 1 eq) in H₂O (1 mL) and MeOH (1 mL) was added NaOH (5.17 mg, 129.17 μmol, 3 eq). The mixture was stirred at 50° C. for 12 hr.

The reaction mixture was concentrated under reduced pressure to remove MeOH. The mixture was adjusted to pH 4 with 1N HCl. The mixture was filtered. Compound 12, 4-[(1S)-1-[[4-(3-isoquinolylmethyl) pyrazolo[1,5-a]pyridine-3-carbonyl]amino]ethyl]benzoic acid (20 mg, 44.42 μmol, 56.71% yield, 100% purity), was obtained as a white solid. LCMS for product (ESI): m/z 451.2 [M+H]⁺. ¹H NMR (400 MHz, DMSO-d₆) δ=9.13 (s, 1H), 8.71-8.66 (m, 2H), 8.33 (s, 1H), 8.01 (d, J=8.0 Hz, 1H), 7.82 (d, J=8.4 Hz, 2H), 7.70-7.61 (m, 2H), 7.61-7.55 (m, 1H), 7.47-7.42 (m, 3H), 7.27 (d, J=7.2 Hz, 1H), 7.01 (t, J=7.2 Hz, 1H), 5.19 (q, J=7.2 Hz, 1H), 4.79 (s, 2H), 1.33 (d, J=7.2 Hz, 3H)

### Example 15—Synthesis of cis-2-[[4-(3-isoquinolylmethyl) pyrazolo[1,5-a]pyridine-3-carbonyl]amino]spiro[3.3]heptane-6-carboxylic acid (Compound 13A)

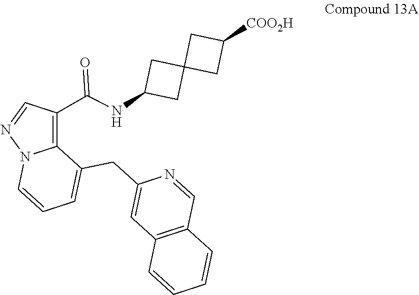

### Step 1: Preparation of cis-(S)-1-phenylethyl 2-[[4-(3-isoquinolylmethyl) pyrazolo[1,5-a]pyridine-3-carbonyl]amino]spiro[3.3]heptane-6-carboxylate

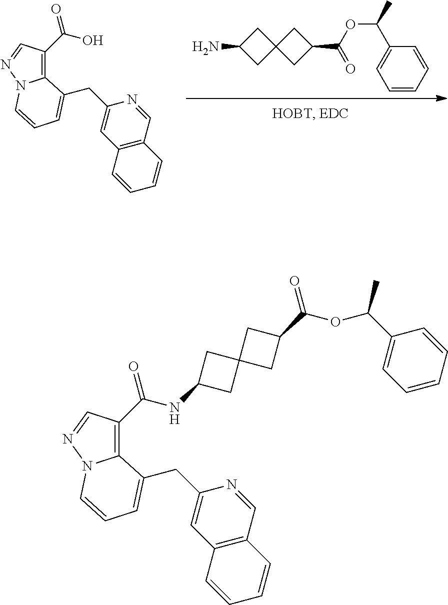 [SMILES: O=C(O)c1cnn2cccc(Cc3cc4ccccc4cn3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3cnn4cccc(Cc5cc6ccccc6cn5)c34)C2)C1)c1ccccc1]

To a solution of 4-(3-isoquinolylmethyl) pyrazolo[1,5-a]pyridine-3-carboxylic acid (40 mg, 131.88 μmol, 1 eq) in DCM (2 mL) was added Et₃N (66.72 mg, 659.40 μmol, 91.78 μL, 5 eq), EDCI (50.56 mg, 263.76 μmol, 2 eq) and HOBt (3.56 mg, 26.38 μmol, 0.2 eq) at 0° C. After addition, the mixture was stirred at this temperature for 0.5 hr, and then cis-(S)-1-phenylethyl 2-aminospiro[3.3]heptane-6-carboxylate (34.20 mg, 131.88 μmol, 1 eq) was added at 0° C. The resulting mixture was stirred at 25° C. for 12 hr.

The residue was diluted with H₂O 10 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO₂, PE: EA=1:1) to provide cis-(S)-1-phenylethyl 2-[[4-(3-isoquinolylmethyl) pyrazolo[1,5-a]pyridine-3-carbonyl]amino]spiro[3.3]heptane-6-carboxylate (30 mg, 55.08 μmol, 41.77% yield) as a white solid, which was used into the next step directly without further purification. LCMS for product (ESI): m/z 545.2 [M+H]⁺.

### Step 2: Preparation of Compound 13A, cis-2-[[4-(3-isoquinolylmethyl) pyrazolo[1,5-a]pyridine-3-carbonyl]amino]spiro[3.3]heptane-6-carboxylic acid

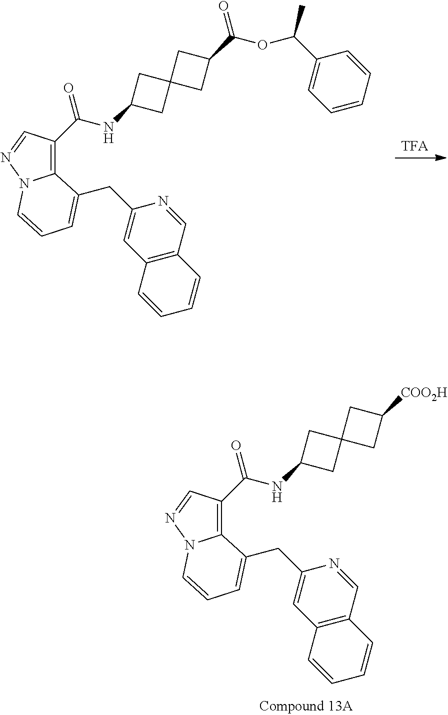

To a solution of cis-(S)-1-phenylethyl 2-[[4-(3-isoquinolylmethyl) pyrazolo[1,5-a]pyridine-3-carbonyl]amino]spiro[3.3]heptane-6-carboxylate (30 mg, 55.08 μmol, 1 eq) in TFA (1 mL) and DCM (3 mL). The mixture was stirred at 25° C. for 5 hr.

The reaction mixture was concentrated under reduced pressure to remove TFA. The residue was dissolved by aqueous NaOH (10 mL, 1N) and filtered. The filtrate was adjusted to pH 4 with 1N HCl. The mixture was filtered. Compound 13A, cis-2-[[4-(3-isoquinolylmethyl) pyrazolo[1,5-a]pyridine-3-carbonyl]amino]spiro[3.3]heptane-6-carboxylic acid (20 mg, 43.81 μmol, 53.66% yield, 93% purity), was obtained as a white solid. LCMS for product (ESI): m/z 441.2 [M+H]⁺. ¹H NMR (400 MHz, DMSO-d₆) δ=12.01 (brs, 1H), 9.16 (s, 1H), 8.64 (d, J=6.8 Hz, 1H), 8.32 (d, J=7.2 Hz, 1H), 8.17 (s, 1H), 8.03 (d, J=8.0 Hz, 1H), 7.81 (J=8.4 Hz, 1H), 7.80-7.73 (m, 1H), 7.61-7.59 (m, 1H), 7.53 (s, 1H), 7.26 (d, J=7.2 Hz, 1H), 6.99 (t, J=6.8 Hz, 1H), 4.79 (s, 2H), 4.23-4.18 (m, 1H), 2.95-2.91 (m, 1H), 2.37-2.21 (m, 2H), 2.18-2.16 (m, 2H), 2.05-2.03 (m, 2H), 1.86-1.82 (m, 2H)

### Example 16—Synthesis of trans-2-[[4-(3-isoquinolylmethyl) pyrazolo[1,5-a]pyridine-3-carbonyl]amino]spiro[3.3]heptane-6-carboxylic acid (Compound 13B)

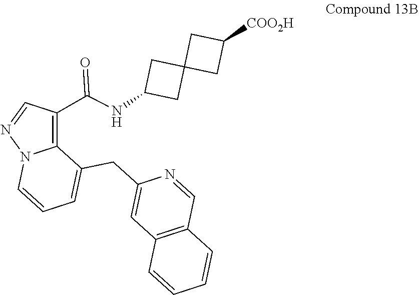

### Step 1: trans-(S)-1-phenylethyl 2-[[4-(3-isoquinolylmethyl) pyrazolo[1,5-a]pyridine-3-carbonyl]amino]spiro[3.3]heptane-6-carboxylate

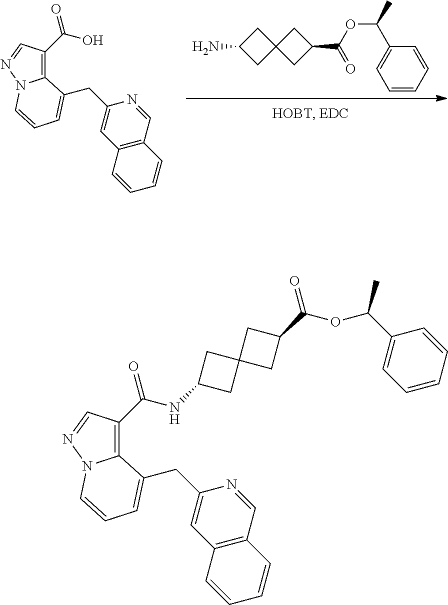 [SMILES: O=C(O)c1cnn2cccc(Cc3cc4ccccc4cn3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3cnn4cccc(Cc5cc6ccccc6cn5)c34)C2)C1)c1ccccc1]

To a solution of 4-(3-isoquinolylmethyl) pyrazolo[1,5-a]pyridine-3-carboxylic acid (40 mg, 131.88 μmol, 1 eq) in DCM (2 mL) was added Et₃N (66.72 mg, 659.40 μmol, 91.78 μL, 5 eq), EDCI (50.56 mg, 263.76 μmol, 2 eq) and HOBt (3.56 mg, 26.38 μmol, 0.2 eq) at 0° C. After addition, the mixture was stirred at this temperature for 0.5 hr, and then trans-(S)-1-phenylethyl 2-aminospiro[3.3]heptane-6-carboxylate (34.20 mg, 131.88 μmol, 1 eq) was added at 0° C. The resulting mixture was stirred at 25° C. for 12 hr.

The residue was diluted with H₂O 5 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO₂, PE: EA=1:1) to provide trans-(S)-1-phenylethyl 2-[[4-(3-isoquinolylmethyl) pyrazolo[1,5-a]pyridine-3-carbonyl]amino]spiro[3.3]heptane-6-carboxylate (30 mg, 55.08 μmol, 41.77% yield) as a white solid, which was used into the next step directly without further purification. LCMS for product (ESI): m/z 545.2 [M+H]⁺.

### Step 2: Preparation of Compound 13B, trans-2-[[4-(3-isoquinolylmethyl) pyrazolo[1,5-a]pyridine-3-carbonyl]amino]spiro[3.3]heptane-6-carboxylic acid

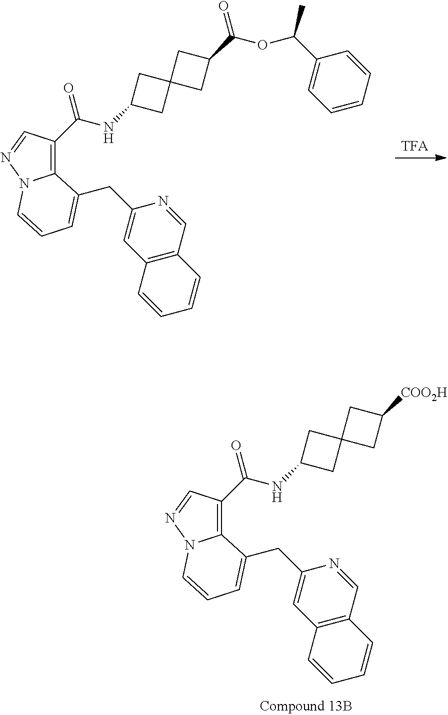

To a solution of trans-(S)-1-phenylethyl 2-[[4-(3-isoquinolylmethyl) pyrazolo[1,5-a]pyridine-3-carbonyl]amino]spiro[3.3]heptane-6-carboxylate (30 mg, 55.08 μmol, 1 eq) in TFA (1 mL) and DCM (3 mL). The mixture was stirred at 25° C. for 5 hr.

The reaction mixture was concentrated under reduced pressure to remove TFA. The residue was dissolved by aqueous NaOH (10 mL, 1N) and filtered. The filtrate was adjusted to pH 4 with 1N HCl. The mixture was filtered. Compound 13B, trans-2-[[4-(3-isoquinolylmethyl) pyrazolo[1,5-a]pyridine-3-carbonyl]amino]spiro[3.3]heptane-6-carboxylic acid (21 mg, 46.00 μmol, 53.66% yield, 93% purity), was obtained as a white solid. LCMS for product (ESI): m/z 441.2 [M+H]⁺. ¹H NMR (400 MHz, DMSO-d₆) δ=12.01 (m, 1H), 9.16 (s, 1H), 8.64 (d, J=6.8 Hz, 1H), 8.32 (d, J=7.2 Hz, 1H), 8.17 (s, 1H), 8.03 (d, J=8.0 Hz, 1H), 7.86-7.78 (m, 1H), 7.75-7.69 (m, 1H), 7.64-7.56 (m, 1H), 7.53 (s, 1H), 7.26 (d, J=7.2 Hz, 1H), 6.99 (t, J=6.8 Hz, 1H), 4.76 (s, 2H), 4.24-4.17 (m, 1H), 2.91 (q, J=8.4 Hz, 1H), 2.36-2.24 (m, 2H), 2.22-2.16 (m, 2H), 2.06-2.03 (m, 2H), 1.86-1.83 (m, 2H).

### Example 17—Synthesis of cis-6-[[4-[[4-(trifluoromethyl) phenyl]methyl]pyrazolo[1,5-a]pyridine-3-carbonyl]amino]spiro[3.3]heptane-2-carboxylic acid (Compound 14A)

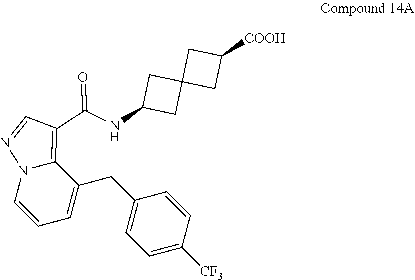 [SMILES: O=C(N[C@H]1CC2(C1)C[C@H](C(=O)O)C2)c1cnn2cccc(Cc3ccc(C(F)(F)F)cc3)c12]

### Step 1: cis-(S)-1-phenylethyl 6-[[4-[[4-(trifluoromethyl) phenyl]methyl]pyrazolo[1,5-a]pyridine-3-carbonyl]amino]spiro[3.3]heptane-2-carboxylate

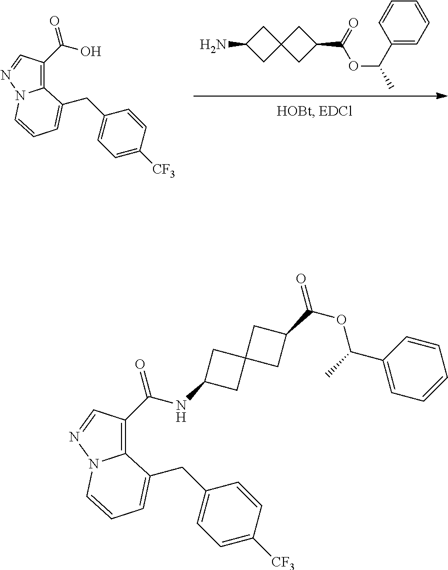 [SMILES: O=C(O)c1cnn2cccc(Cc3ccc(C(F)(F)F)cc3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3cnn4cccc(Cc5ccc(C(F)(F)F)cc5)c34)C2)C1)c1ccccc1]

To a solution of 4-[[4-(trifluoromethyl)phenyl]methyl]pyrazolo[1,5-a]pyridine-3-carboxylic acid (20 mg, 62.45 μmol, 1 eq) in DCM (1 mL) was added HOBt (1.69 mg, 12.49 μmol, 0.2 eq), EDCI (23.95 mg, 124.90 μmol, 2 eq) and Et₃N (31.59 mg, 312.25 μmol, 5 eq) at 0° C. After addition, the mixture was stirred at this temperature for 0.5 hr, and then cis-(S)-1-phenylethyl 6-aminospiro[3.3]heptane-2-carboxylate (24.29 mg, 93.68 μmol, 1.5 eq) was added dropwise at 0° C. The resulting mixture was stirred at 25° C. for 12 hr.

The residue was diluted with H₂O 5 mL and extracted with DCM 15 mL (5 mL*3). The combined organic layers were washed with brine 5 mL, dried over Na₂SO₄, filtered and concentrated under reduced pressure to give a residue. The residue was purified by prep-TLC (SiO₂, PE: EA=1:1) to provide cis-(S)-1-phenylethyl 6-[[4-[[4-(trifluoromethyl) phenyl]methyl]pyrazolo[1,5-a]pyridine-3-carbonyl]amino]spiro [3.3]heptane-2-carboxylate (15 mg, 26.60 μmol, 42.77% yield) as a white solid, which was used into the next step directly without further purification.

### Step 2: Preparation of Compound 14A, cis-6-[[4-[[4-(trifluoromethyl) phenyl]methyl]pyrazolo[1,5-a]pyridine-3-carbonyl]amino]spiro[3.3]heptane-2-carboxylic acid

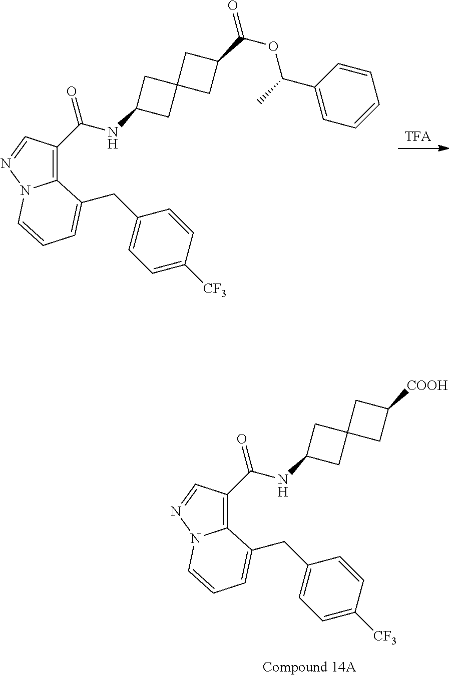

To a solution of cis-(S)-1-phenylethyl 6-[[4-[[4-(trifluoromethyl)phenyl]methyl]pyrazolo[1,5-a]pyridine-3-carbonyl]amino]spiro[3.3]heptane-2-carboxylate (15 mg, 26.60 μmol, 1 eq) in TFA (1 mL) and DCM (3 mL). The mixture was stirred at 25° C. for 5 hr.

The reaction mixture was concentrated under reduced pressure to remove TFA and DCM. The residue was purified by prep-TLC (SiO₂, PE: EA=1:1). Compound 14A, cis-6-[[4-[[4-(trifluoromethyl) phenyl]methyl]pyrazolo[1,5-a]pyridine-3-carbonyl]amino]spiro[3.3]heptane-2-carboxylic acid (3.8 mg, 8.31 μmol, 31.24% yield, 93.3% purity), was obtained as a white solid. LCMS for product (ESI): m/z 458.2 [M+H]⁺. ¹H NMR (400 MHz, DMSO-d₆) δ=8.67 (d, J=6.4 Hz, 1H), 8.31 (d, J=7.2 Hz, 1H), 8.18 (s, 1H), 7.56 (d, J=8.0 Hz, 2H), 7.33-7.26 (m, 3H), 7.02 (t, J=6.8 Hz, 1H), 4.62 (s, 2H), 4.23-4.16 (m, 1H), 2.98-2.89 (m, 1H), 2.42-2.34 (m, 1H), 2.29-2.17 (m, 3H), 2.15-2.05 (m, 2H), 1.95-1.83 (m, 2H)

### Example 18—Synthesis of trans-2-[[4-[[4-(trifluoromethyl) phenyl]methyl]pyrazolo[1,5-al pyridine-3-carbonyl]amino]spiro[3.3]heptane-6-carboxylic acid (Compound 14B)

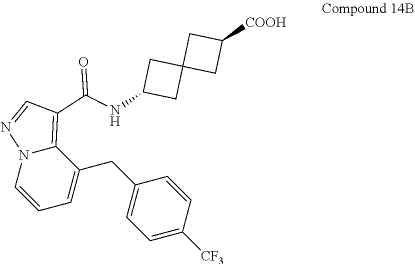 [SMILES: O=C(N[C@H]1CC2(C1)C[C@H](C(=O)O)C2)c1cnn2cccc(Cc3ccc(C(F)(F)F)cc3)c12]

### Step 1: trans-(S)-1-phenylethyl 2-[[4-[[4-(trifluoromethyl) phenyl]methyl]pyrazolo[1,5-a]pyridine-3-carbonyl]amino]spiro[3.3]heptane-6-carboxylate

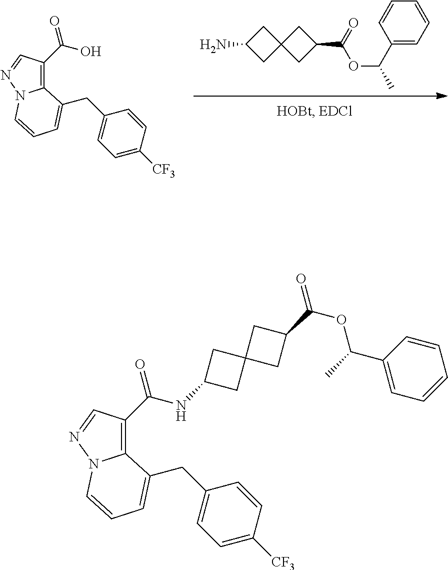 [SMILES: O=C(O)c1cnn2cccc(Cc3ccc(C(F)(F)F)cc3)c12 ; C[C@H](OC(=O)[C@H]1CC2(C[C@@H](N)C2)C1)c1ccccc1 ; C[C@H](OC(=O)[C@H]1CC2(C[C@H](NC(=O)c3cnn4cccc(Cc5ccc(C(F)(F)F)cc5)c34)C2)C1)c1ccccc1]

To a solution of 4-[[4-(trifluoromethyl)phenyl]methyl]pyrazolo[1,5-a]pyridine-3-carboxylic acid (40 mg, 124.90 μmol, 1 eq) in DCM (1 mL) was added HOBt (3.38 mg, 24.98 μmol, 0.2 eq), EDCI (47.89 mg, 249.80 μmol, 2 eq) and Et₃N (63.19 mg, 624.50 μmol, 86.92 μL, 5 eq) at 0° C. After addition, the mixture was stirred at this temperature for 0.5 hr, and then trans-(S)-1-phenylethyl 2-aminospiro[3.3]heptane-6-carboxylate (48.59 mg, 187.35 μmol, 1.5 eq) was added dropwise at 0° C. The resulting mixture was stirred at 25° C. for 12 hr.

The residue was diluted with H₂O 10 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO₂, PE: EA=1:1) to provide trans-(S)-1-phenylethyl 2-[[4-[[4-(trifluoromethyl) phenyl]methyl]pyrazolo[1,5-a]pyridine-3-carbonyl]amino]spiro [3.3]heptane-6-carboxylate (32 mg, 53.42 μmol, 42.77% yield) as a white solid, which was used into the next step directly without further purification. LCMS for product (ESI): m/z 584.1 [M+Na]⁺.

### Step 2: Preparation of Compound 14B, trans-2-[[4-[[4-(trifluoromethyl) phenyl]methyl]pyrazolo[1,5-a]pyridine-3-carbonyl]amino]spiro[3.3]heptane-6-carboxylic acid

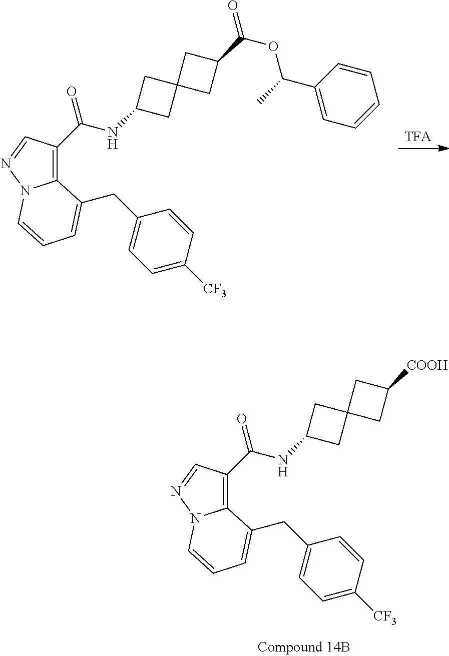

To a solution of trans-(S)-1-phenylethyl 2-[[4-[[4-(trifluoromethyl)phenyl]methyl]pyrazolo[1,5-a]pyridine-3-carbonyl]amino]spiro[3.3]heptane-6-carboxylate (30 mg, 53.42 μmol, 1 eq) in TFA (1 mL) and DCM (3 mL). The mixture was stirred at 25° C. for 5 hr.

The reaction mixture was concentrated under reduced pressure to remove TFA and DCM. The residue was purified by prep-TLC (SiO₂, PE: EA=1:1). Compound 14B, trans-2-[[4-[[4-(trifluoromethyl) phenyl]methyl]pyrazolo[1,5-a]pyridine-3-carbonyl]amino]spiro[3.3]heptane-6-carboxylic acid (21 mg, 45.95 μmol, 53.45% yield, 93.3% purity), was obtained as a white solid. LCMS for product (ESI): m/z 458.2 [M+H]⁺. ¹H NMR (400 MHz, DMSO-d₆) δ=8.67 (d, J=6.8 Hz, 1H), 8.31 (d, J=7.2 Hz, 1H), 8.18 (s, 1H), 7.56 (d, J=8.0 Hz, 2H), 7.33-7.26 (m, 3H), 7.02 (t, J=6.8 Hz, 1H), 4.63 (s, 2H), 4.23-4.16 (m, 1H), 2.98-2.89 (m, 1H), 2.42-2.34 (m, 1H), 2.29-2.17 (m, 3H), 2.15-2.05 (m, 2H), 1.95-1.83 (m, 2H)

### Example 19—Synthesis of 2-[3-[[4-[[4-(trifluoromethyl) phenyl]methyl]pyrazolo[1,5-a]pyridine-3-carbonyl]amino]-1-bicyclo[1.1.1]pentanyl]acetic acid (Compound 15)

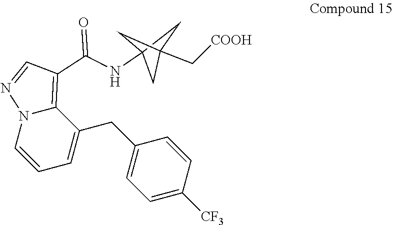 [SMILES: O=C(O)CC12CC(NC(=O)c3cnn4cccc(Cc5ccc(C(F)(F)F)cc5)c34)(C1)C2]

### Step 1: methyl 2-[3-[[4-[[4-(trifluoromethyl) phenyl]methyl]pyrazolo[1,5-a]pyridine-3-carbonylamino]-1-bicyclo[1.1.1]pentanyl]acetate

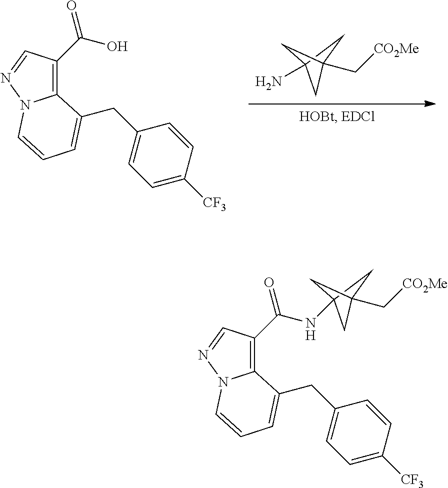

To a solution of 4-[[4-(trifluoromethyl) phenyl]methyl]pyrazolo[1,5-a]pyridine-3-carboxylic acid (60 mg, 187.34 μmol, 1 eq) in DCM (1 mL) was added HOBt (5.06 mg, 37.47 μmol, 0.2 eq), Et3N (94.79 mg, 936.72 μmol, 130.38 μL, 5 eq) and EDCI (71.83 mg, 374.69 μmol, 2 eq) at 0° C. for 0.5 hr. Then was added methyl 2-(3-amino-1-bicyclo[1.1.1]pentanyl) acetate (34.89 mg, 224.81 μmol, 1.2 eq) at 0° C., The mixture was stirred at 25° C. for 12 hr.

The residue was diluted with H₂O 10 mL and extracted with DCM 30 mL (10 mL×3). The combined organic layers were washed with brine 10 mL, dried over Na₂SO₄, filtered and concentrated under reduced pressure to give a residue. The crude product was used into the next step without further purification to provide methyl 2-[3-[[4-[[4-(trifluoromethyl) phenyl]methyl]pyrazolo[1,5-a]pyridine-3-carbonyl]amino]-1-bicyclo[1.1.1]pentanyl]acetate (62 mg, 72% yield) as a white solid, which was used into the next step directly without further purification. LCMS for product (ESI): m/z 458.2 [M+H]⁺.

### Step 2: Preparation of Compound 15,2-[3-[[4-[[4-(trifluoromethyl) phenyl]methyl]pyrazolo[1,5-a]pyridine-3-carbonyl]amino]-1-bicyclo[1.1.1]pentanyl]acetic acid

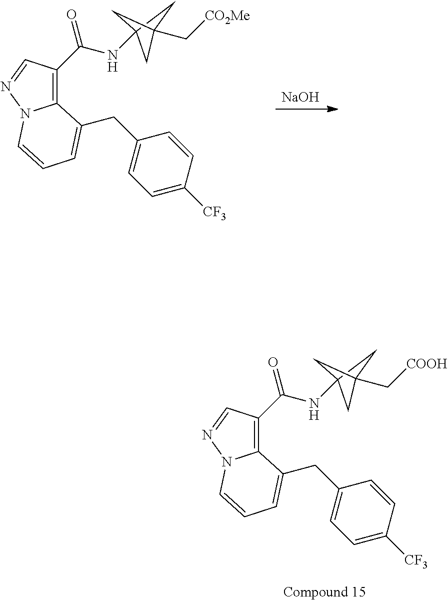 [SMILES: COC(=O)CC12CC(NC(=O)c3cnn4cccc(Cc5ccc(C(F)(F)F)cc5)c34)(C1)C2 ; "NaOH" ; O=C(O)CC12CC(NC(=O)c3cnn4cccc(Cc5ccc(C(F)(F)F)cc5)c34)(C1)C2]

To a solution of methyl 2-[3-[[4-[[4-(trifluoromethyl)phenyl]methyl]pyrazolo[1,5-a]pyridine-3-carbonyl]amino]-1-bicyclo[1.1.1]pentanyl]acetate (62 mg, 135.66 μmol, 1 eq) in H₂O (3 mL) and MeOH (1 mL) was added NaOH (15 mg, 375 μmol, 3 eq). The mixture was stirred at 50° C. for 12 hr.

The reaction mixture was concentrated under reduced pressure to remove MEOH. The residue was diluted with H₂O (10 mL) and filtered. The filtrate was adjusted to pH 4 with 1N HCl. The mixture was filtered. Compound 15, 2-[3-[[4-[[4-(trifluoromethyl) phenyl]methyl]pyrazolo[1,5-a]pyridine-3-carbonyl]amino]-1-bicyclo[1.1.1]pentanyl]acetic acid (50 mg, 110.84 μmol, 83% yield, 98.3% purity), was obtained as a white solid. LCMS for product (ESI): m/z 444.1 [M+H]⁺. ¹H NMR (400 MHz, DMSO-d₆) δ=12.10 (brs, 1H), 8.66 (d, J=8.4 Hz, 2H), 8.16 (s, 1H), 7.55 (d, J=8.0 Hz, 2H), 7.37-7.29 (m, 3H), 7.03 (t, J=6.8 Hz, 1H), 4.65 (s, 2H), 2.50 (s, 2H), 2.00 (s, 6H)

### Example 20—Synthesis of 2-[3-[[4-[[3-(trifluoromethyl) phenyl]methyl]pyrazolo[1,5-a]pyridine-3-carbonyl]amino]-1-bicyclo[1.1.1]pentanyl]acetic acid (Compound 16)

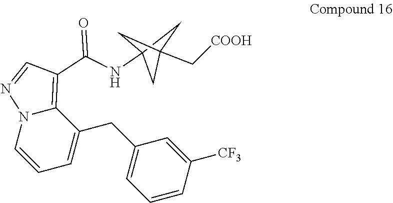 [SMILES: O=C(O)CC12CC(NC(=O)c3cnn4cccc(Cc5cccc(C(F)(F)F)c5)c34)(C1)C2]

### Step 1: Preparation of methyl 4-bromopyrazolo[1,5-a]pyridine-3-carboxylate

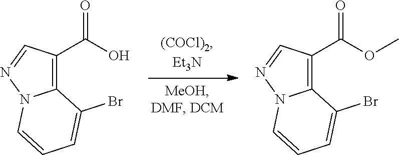

To a solution of 4-bromopyrazolo[1,5-a]pyridine-3-carboxylic acid (200 mg, 829.73 μmol, 1 eq) in DCM (2 mL) and DMF (0.01 mL) was added (COCl)₂ (115.85 mg, 912.71 μmol, 79.89 μL, 1.1 eq) at 25° C. After addition, the mixture was stirred at this temperature for 3 hrs, and then MeOH (265.86 mg, 8.30 mmol, 335.77 μL, 10 eq) and Et₃N (251.88 mg, 2.49 mmol, 346.47 μL, 3 eq) were added at 0° C. The resulting mixture was stirred at 0° C. for 1 hr.

The reaction mixture was concentrated under reduced pressure to remove solvent. The residue was diluted with H₂O 20 mL and extracted with DCM 60 mL (20 mL×3). The combined organic layers were washed with brine 20 mL, dried over Na₂SO₄, filtered and concentrated under reduced pressure to give a residue. The residue was purified by prep-TLC (SiO₂, PE: EA=2:1) to provide methyl 4-bromopyrazolo[1,5-a]pyridine-3-carboxylate (180 mg, 705.69 μmol, 85.05% yield) as white solid.

### Step 2: Preparation of methyl 4-[[3-(trifluoromethyl) phenyl]methyl]pyrazolo[1,5-a]pyridine-3-carboxylate

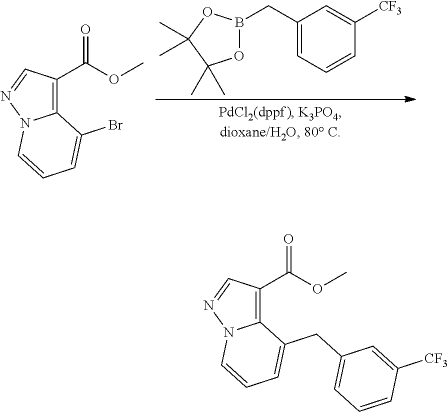

To a solution of methyl 4-bromopyrazolo[1,5-a]pyridine-3-carboxylate (100 mg, 392.05 μmol, 1 eq) and 4,4,5,5-tetramethyl-2-[[3-(trifluoromethyl) phenyl]methyl]-1,3,2-dioxaborolane (112.17 mg, 392.05 μmol, 1 eq) in dioxane (1 mL) and H₂O (0.2 mL) was added Pd(dppf)Cl₂ (86.06 mg, 117.62 μmol, 0.3 eq) and K₃PO₄ (83.22 mg, 392.05 μmol, 1 eq) under nitrogen. The mixture was stirred at 80° C. for 12 hr.

The solvent was removed to afford the crude product. The residue was purified by prep-TLC (SiO₂, PE: EA=1:1). Compound methyl 4-[[3-(trifluoromethyl)phenyl]methyl]pyrazolo[1,5-a]pyridine-3-carboxylate (50 mg, 149.57 μmol, 38.15% yield) was obtained as a white solid.

### Step 3: Preparation of 4-[[3-(trifluoromethyl) phenyl]methyl]pyrazolo[1,5-a]pyridine-3-carboxylic acid

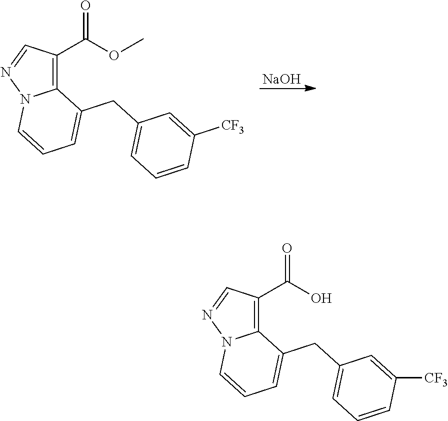 [SMILES: COC(=O)c1cnn2cccc(Cc3cccc(C(F)(F)F)c3)c12 ; "NaOH" ; O=C(O)c1cnn2cccc(Cc3cccc(C(F)(F)F)c3)c12]

To a solution of methyl 4-[[3-(trifluoromethyl) phenyl]methyl]pyrazolo[1,5-a]pyridine-3-carboxylate (50 mg, 149.57 μmol, 1 eq) in H₂O (4 mL) and MeOH (2 mL) was added NaOH (18 mg, 448.71 μmol, 3 eq). The mixture was stirred at 50° C. for 12 hr.

The solvent was removed to afford the crude product. The residue was purified by prep-TLC (SiO2, PE: EA=1:1) to give 4-[[3-(trifluoromethyl) phenyl]methyl]pyrazolo[1,5-a]pyridine-3-carboxylic acid (30 mg, 93.67 μmol, 62.63% yield) as a white solid.

### Step 4: Preparation of methyl 2-[3-[[4-[[3-(trifluoromethyl) phenyl]methyl]pyrazolo[1,5-a]pyridine-3-carbonyl]amino]-1-bicyclo[1.1.1]pentanyl]acetate

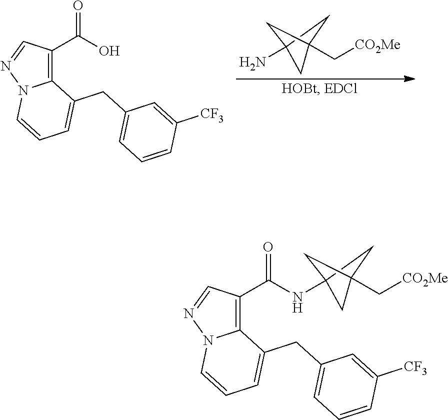

To a solution of 4-[[3-(trifluoromethyl) phenyl]methyl]pyrazolo[1,5-a]pyridine-3-carboxylic acid (30 mg, 93.67 μmol, 1 eq) in DCM (1 mL) was added Et₃N (47.39 mg, 468.36 μmol, 65.19 μL, 5 eq), EDCI (35.91 mg, 187.34 μmol, 2 eq) and HOBt (2.53 mg, 18.73 μmol, 0.2 eq) at 0° C. for 0.5 h. Then was added methyl 2-(3-amino-1-bicyclo[1.1.1]pentanyl) acetate (17.44 mg, 112.41 μmol, 1.2 eq) at 0° C. The mixture was stirred at 25° C. for 12 hr.

The residue was diluted with H₂O 5 mL and extracted with DCM 15 mL (5 mL×3). The combined organic layers were washed with brine 10 mL, dried over Na₂SO₄, filtered and concentrated under reduced pressure to give a residue. The crude product was used into the next step without further purification. Compound methyl 2-[3-[[4-[[3-(trifluoromethyl) phenyl]methyl]pyrazolo[1,5-a]pyridine-3-carbonyl]amino]-1-bicyclo[1.1.1]pentanyl]acetate (30 mg, 65.58 μmol, 70.01% yield) was obtained as a white solid, which was used into the next step directly without further purification. LCMS for product (ESI): m/z 458.3 [M+H]⁺.

### Step 5: Preparation of Compound 16,2-[3-[[4-[[3-(trifluoromethyl) phenyl]methyl]pyrazolo[1,5-a]pyridine-3-carbonyl]amino]-1-bicyclo[1.1.1]pentanyl]acetic acid

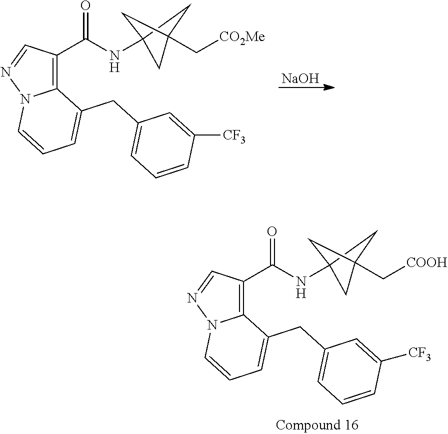 [SMILES: COC(=O)CC12CC(NC(=O)c3cnn4cccc(Cc5cccc(C(F)(F)F)c5)c34)(C1)C2 ; "NaOH" ; O=C(O)CC12CC(NC(=O)c3cnn4cccc(Cc5cccc(C(F)(F)F)c5)c34)(C1)C2]

To a solution of methyl 2-[3-[[4-[[3-(trifluoromethyl) phenyl]methyl]pyrazolo[1,5-a]pyridine-3-carbonyl]amino]-1-bicyclo[1.1.1]pentanyl]acetate (30 mg, 65.58 μmol, 1 eq) in H₂O (3 mL) and MeOH (2 mL) was added NaOH (7.86 mg, 196.74 μmol, 3 eq). The mixture was stirred at 50° C. for 12 hr.

The solvent was removed to afford the crude product. The mixture was adjusted to pH 4 with 1N HCl. The mixture was filtered to provide Compound 16, 2-[3-[[4-[[3-(trifluoromethyl) phenyl]methyl]pyrazolo[1,5-a]pyridine-3-carbonyl]amino]-1-bicyclo[1.1.1]pentanyl]acetic acid (20 mg, 44.47 μmol, 67.81% yield, 98.6% purity) as a white solid. LCMS for product (ESI): m/z 444.1 [M+H]⁺. ¹H NMR (400 MHz, DMSO-d₆) δ=8.73 (s, 1H), 8.66 (d, J=6.8 Hz, 1H), 8.22 (s, 1H), 7.49 (d, J=8.0 Hz, 1H), 7.47-7.44 (m, 3H), 7.39 (t, J=7.2 Hz, 1H), 7.04 (t, J=6.8 Hz, 1H), 4.69 (s, 2H), 2.50 (s, 2H), 2.02 (s, 6H)

### Example 21—Synthesis of 2-[3-[[8-[[4-(trifluoromethyl) phenyl]methyl]imidazo[1,5-a]pyridine-1-carbonyl]amino]-1-bicyclo[1.1.1]pentanyl]acetic acid (Compound 17)

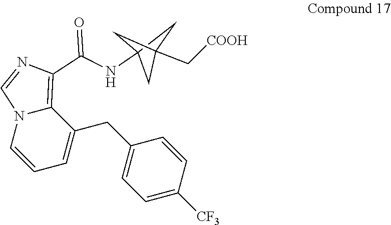 [SMILES: O=C(O)CC12CC(NC(=O)c3ncn4cccc(Cc5ccc(C(F)(F)F)cc5)c34)(C1)C2]

### Step 1: methyl 2-[3-[[8-[[4-(trifluoromethyl) phenyl]methyl]imidazo[1,5-a]pyridine-1-carbonyl]amino]-1-bicyclo[1.1.11pentanyl]acetate

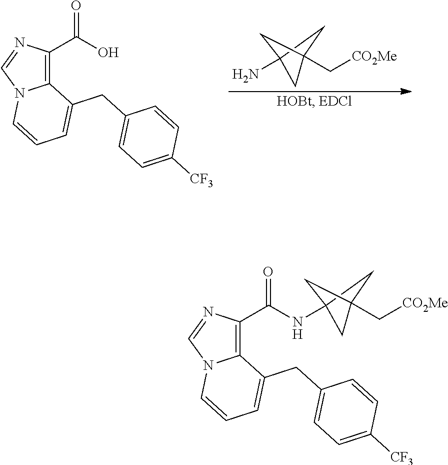

To a solution of 8-[[4-(trifluoromethyl) phenyl]methyl]imidazo[1,5-a]pyridine-1-carboxylic acid (40 mg, 124.89 μmol, 1 eq) in DCM (1 mL) was added EDCI (29.93 mg, 156.12 μmol, 2 eq), Et₃N (39.49 mg, 390.30 μmol, 54.33 μL, 5 eq) and HOBt (2.11 mg, 15.61 μmol, 0.2 eq) at 0° C. The mixture was stirred at 0° C. for 0.5 hr. Then methyl 2-(3-amino-1-bicyclo[1.1.1]pentanyl) acetate (37.5 mg, 241.63 μmol, 1.5 eq) was added at 0° C. The mixture was stirred at 25° C. for 16 hr.

The reaction mixture was diluted with H₂O 5 mL and extracted with DCM 30 mL (10 mL×3). The combined organic layers were washed with brine (10 mL), dried over Na₂SO₄, filtered and concentrated under reduced pressure to give a residue. The crude product was used into the next step without further purification to provide methyl 2-[3-[[8-[[4-(trifluoromethyl) phenyl]methyl]imidazo[1,5-a]pyridine-1-carbonyl]amino]-1-bicyclo[1.1.1]pentanyl]acetate (33 mg, 72.21 μmol, 57.7% yield) as a white solid, which was used into the next step directly without further purification. LCMS for product (ESI): m/z 458.3 [M+H]⁺.

### Step 2: Preparation of Compound 17,2-[3-[[8-[[4-(trifluoromethyl) phenyl]methyl]imidazo[1,5-a]pyridine-1-carbonyl]amino]-1-bicyclo[1.1.1]pentanyl]acetic acid

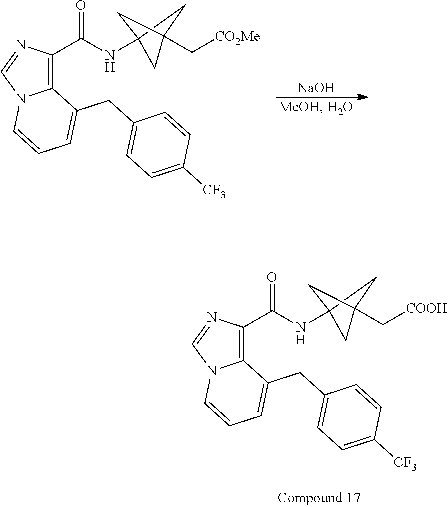

To a solution of methyl 2-[3-[[8-[[4-(trifluoromethyl) phenyl]methyl]imidazo[1,5-a]pyridine-1-carbonyl]amino]-1-bicyclo[1.1.1]pentanyl]acetate (33 mg, 72.21 μmol, 1 eq) in H₂O (2 mL) and MeOH (2 mL) was added NaOH (8.67 mg, 216.63 μmol, 3 eq). The mixture was stirred at 50° C. for 16 hr.

The reaction mixture was concentrated under reduced pressure to remove MeOH. The mixture was adjusted to PH 4 with 1N HCl. The mixture was filtered. Compound 17, 2-[3-[[8-[[4-(trifluoromethyl) phenyl]methyl]imidazo[1,5-a]pyridine-1-carbonyl]amino]-1-bicyclo[1.1.1]pentanyl]acetic acid (24 mg, 54.12 μmol, 75% yield, 95.12% purity), was obtained as a white solid. LCMS for product (ESI): m/z 444.1 [M+H]⁺. ¹H NMR (400 MHz, DMSO-d₆) δ ppm 8.50 (d, J=6.4 Hz, 1H), 8.46 (s, 1H), 8.36 (d, J=6.8 Hz, 1H), 7.58 (br d, J=8.0 Hz, 2H), 7.41 (br d, J=7.6 Hz, 2H), 6.91 (d, J=6.8 Hz, 1H), 6.84-6.80 (m, 1H), 4.80 (s, 2H), 2.30 (s, 2H), 2.02 (s, 6H).

### Example 22—Synthesis of 2-(3-(4-(4-(trifluoromethyl)benzyl)-1H-indazole-3-carboxamido)bicyclo[1.1.1]pentan-1-yl)acetic acid (Compound 18)

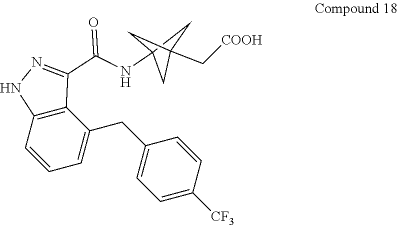 [SMILES: O=C(O)CC12CC(NC(=O)c3n[nH]c4cccc(Cc5ccc(C(F)(F)F)cc5)c34)(C1)C2]

### Step 1: Preparation of methyl2-[3-[[1-tetrahydropyran-2-yl-4-[[4-(trifluoromethyl)phenyl]methyl]indazole-3-carbonyl]amino]-1-bicyclo[1.1.11pentanyl]acetate

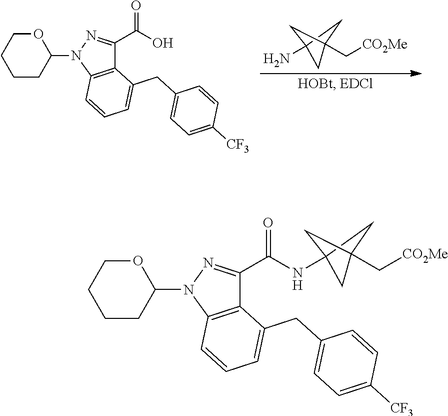

To a solution of 1-tetrahydropyran-2-yl-4-[[4-(trifluoromethyl) phenyl]methyl]indazole-3-carboxylic acid (60 mg, 148.38 μmol, 1 eq) in DCM (2 mL) was added HOBt (4.00 mg, 29.68 μmol, 0.2 eq) EDCI (58.88 mg, 296.74 μmol, 2 eq) and Et3N (75.08 mg, 741.88 μmol, 100 μL, 5 eq). The mixture was stirred at 0° C. for 0.5 hr. Then methyl 2-(3-amino-1-bicyclo[1.1.1]pentanyl) acetate (27.64 mg, 178.04 μmol, 1.2 eq) was added to the mixture. The mixture was stirred at 25° C. for 16 hr.

The reaction mixture was diluted with H₂O 5 mL and extracted with DCM 30 mL (10 mL×3). The combined organic layers were washed with brine 5 mL, dried over Na₂SO₄, filtered and concentrated under reduced pressure to give a residue. The crude product was used into the next step without further purification to provide methyl2-[3-[[1-tetrahydropyran-2-yl-4-[[4-(trifluoromethyl)phenyl]methyl]indazole-3-carbonyl]amino]-1-bicyclo[1.1.1]pentanyl]acetate (50 mg, 92.32 μmol, 62.22% yield) as a white solid, which was used into the next step directly without further purification. LCMS for product (ESI): m/z 542.3 [M+H]⁺.

### Step 2: Preparation of methyl 2-[3-[[4-[[4-(trifluoromethyl) phenyl]methyl]-1H-indazole-3-carbonyl]amino]-1-bicyclo[1.1.11pentanyl]acetate

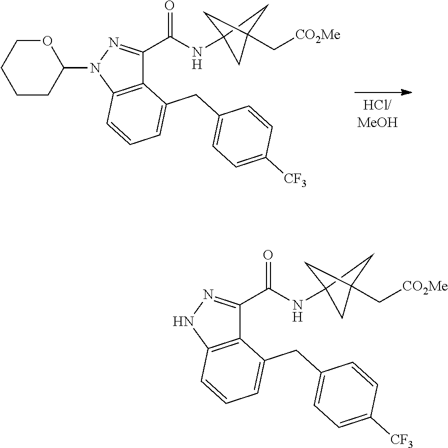 [SMILES: COC(=O)CC12CC(NC(=O)c3nn(C4CCCCO4)c4cccc(Cc5ccc(C(F)(F)F)cc5)c34)(C1)C2 ; COC(=O)CC12CC(NC(=O)c3n[nH]c4cccc(Cc5ccc(C(F)(F)F)cc5)c34)(C1)C2]

To a solution of methyl 2-[3-[[1-tetrahydropyran-2-yl-4-[[4-(trifluoromethyl) phenyl]methyl]indazole-3-carbonyl]amino]-1-bicyclo[1.1.1]pentanyl]acetate (50 mg, 92.32 μmol, 1 eq) in HCl/MeOH (6 mL). The mixture was stirred at 25° C. for 1 hr.

The reaction mixture was diluted with H₂O 10 mL and extracted with DCM 30 mL (10 mL×3). The combined organic layers were washed with brine 10 mL, dried over Na₂SO₄, filtered and concentrated under reduced pressure to give a residue. The crude product was used into the next step without further purification and compound methyl 2-[3-[[4-[[4-(trifluoromethyl) phenyl]methyl]-1H-indazole-3-carbonyl]amino]-1-bicyclo[1.1.1]pentanyl]acetate (40 mg, 87.44 μmol, 94.71% yield) was obtained as a white solid, which was used into the next step directly without further purification. LCMS for product (ESI): m/z 458.1 [M+H]⁺.

### Step 3: Preparation of Compound 18,2-(3-(4-(4-(trifluoromethyl)benzyl)-1H-indazole-3-carboxamido)bicyclo[1.1.1]pentan-1-yl)acetic acid

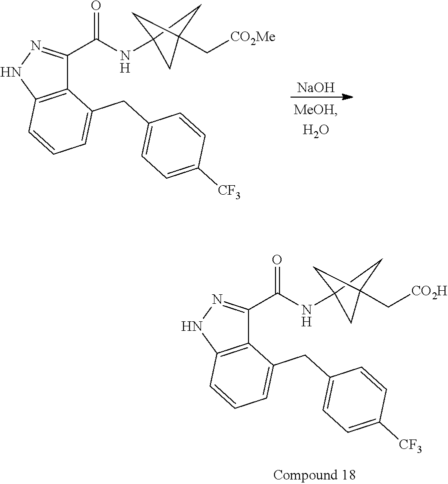 [SMILES: COC(=O)CC12CC(NC(=O)c3n[nH]c4cccc(Cc5ccc(C(F)(F)F)cc5)c34)(C1)C2 ; "NaOH" ; O=C(O)CC12CC(NC(=O)c3n[nH]c4cccc(Cc5ccc(C(F)(F)F)cc5)c34)(C1)C2]

To a solution of methyl 2-[3-[[4-[[4-(trifluoromethyl) phenyl]methyl]-1H-indazole-3-carbonyl]amino]-1-bicyclo[1.1.1]pentanyl]acetate (40 mg, 87.44 μmol, 1 eq) in MeOH (2 mL) and H₂O (2 mL) was added NaOH (10.5 mg, 262.32 μmol, 3 eq). The mixture was stirred at 50° C. for 16 hr.

The reaction mixture was concentrated under reduced pressure to remove MeOH. The mixture was adjusted to pH 4 with 1N HCl. The mixture was filtered and Compound 18, 2-(3-(4-(4-(trifluoromethyl)benzyl)-1H-indazole-3-carboxamido)bicyclo[1.1.1]pentan-1-yl)acetic acid (21 mg, 47.33 μmol, 54.26% yield, 97.44% purity), was obtained as a white solid. LCMS for product (ESI): m/z 444.1 [M+H]⁺. ¹H NMR (400 MHz, DMSO-d₆) δ ppm 13.50 (s, 1H), 12.08 (brs, 1H), 8.81 (s, 1H), 7.53 (d, J=8.0 Hz, 2H), 7.46 (d, J=8.4 Hz, 1H), 7.38-7.32 (m, 3H), 7.08 (d, J=7.2 Hz, 1H), 4.70 (s, 2H), 2.50 (s, 2H), 2.02 (s, 6H).

### Example 23—Synthesis of 2-[3-[[1-ethyl-4-[[4-(trifluoromethyl) phenyl]methyl]indazole-3-carbonyl]amino]-1-bicyclo[1.1.1]pentanyl]acetic acid (Compound 19)

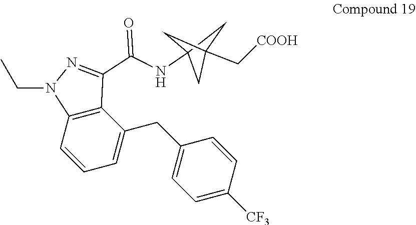 [SMILES: CCn1nc(C(=O)NC23CC(CC(=O)O)(C2)C3)c2c(Cc3ccc(C(F)(F)F)cc3)cccc21]

### Step 1: methyl 2-[3-[[1-ethyl-4-[[4-(trifluoromethyl)phenyl]methyl]indazole-3-carbonyl]amino]-1-bicyclo[1.1.11pentanyl]acetate

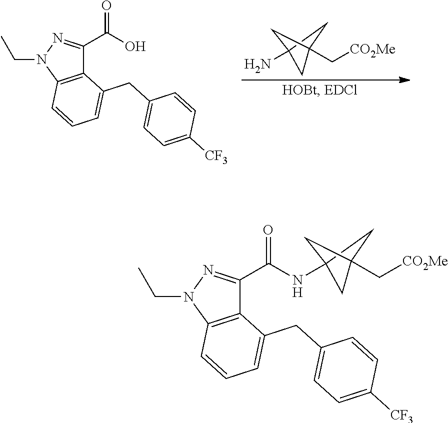

To a solution of 1-ethyl-4-[[4-(trifluoromethyl) phenyl]methyl]indazole-3-carboxylic acid (50 mg, 143.55 μmol, 1 eq) in DCM (1 mL) was added HOBt (3.88 mg, 28.71 μmol, 0.2 eq), Et₃N (72.63 mg, 717.73 μmol, 99.90 μL, 5 eq) and EDCI (55.04 mg, 287.09 μmol, 2 eq). The mixture was stirred at 0° C. for 0.5 hr. Then methyl 2-(3-amino-1-bicyclo[1.1.1]pentanyl) acetate (24.51 mg, 157.90 μmol, 1.1 eq) was added at 0° C. The mixture was stirred at 25° C. for 16 hrs.

The reaction mixture was diluted with H₂O 5 mL and extracted with DCM 9 mL (3 mL×3). The combined organic layers were washed with brine 5 mL, dried over Na₂SO₄, filtered and concentrated under reduced pressure to give a residue. The residue was purified by prep-TLC (SiO₂, PE:EA=2:1) to provide methyl 2-[3-[[1-ethyl-4-[[4-(trifluoromethyl)phenyl]methyl]indazole-3-carbonyl]amino]-1-bicyclo[1.1.1]pentanyl]acetate (40 mg, 82.39 μmol, 57.40% yield) as a white solid, which was used into the next step directly without further purification. LCMS for product (ESI): m/z 486.3 [M+H]⁺.

### Step 2: Preparation of Compound 19,2-[3-[[1-ethyl-4-[[4-(trifluoromethyl) phenyl]methyl]indazole-3-carbonyl]amino]-1-bicyclo[1.1.11pentanyl]acetic acid

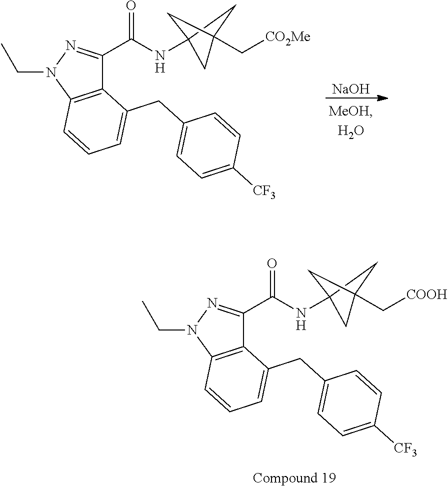 [SMILES: CCn1nc(C(=O)NC23CC(CC(=O)OC)(C2)C3)c2c(Cc3ccc(C(F)(F)F)cc3)cccc21 ; "NaOH" ; CCn1nc(C(=O)NC23CC(CC(=O)O)(C2)C3)c2c(Cc3ccc(C(F)(F)F)cc3)cccc21]

To a solution of methyl 2-[3-[[1-ethyl-4-[[4-(trifluoromethyl) phenyl]methyl]indazole-3-carbonyl]amino]-1-bicyclo[1.1.1]pentanyl]acetate (40 mg, 82.39 μmol, 1 eq) in H₂O (1 mL) and MeOH (1 mL) was added NaOH (9.89 mg, 247.17 μmol, 3 eq). The mixture was stirred at 50° C. for 16 hr.

The reaction mixture was concentrated under reduced pressure to remove MeOH. The mixture was adjusted to PH 4 with 1N HCl. The mixture was filtered. Compound 19, 2-[3-[[1-ethyl-4-[[4-(trifluoromethyl) phenyl]methyl]indazole-3-carbonyl]amino]-1-bicyclo[1.1.1]pentanyl]acetic acid (26 mg, 54.69 μmol, 66.38% yield, 99.17% purity), was obtained as a white solid. LCMS for product (ESI): m/z 472.1 [M+H]⁺. ¹H NMR (400 MHz, CD₃OD) δ=8.67 (s, 1H), 7.52 (d, J=8.4 Hz, 1H), 7.46 (d, J=8.4 Hz, 2H), 7.45-7.41 (m, 1H), 7.27 (d, J=8.0 Hz, 2H), 7.11 (t, J=6.8 Hz, 1H), 4.70 (s, 2H), 4.51-4.44 (m, 2H), 2.58 (s, 2H), 2.12 (s, 6H), 1.48 (t, J=7.2 Hz, 3H)

### Example 24—Synthesis of 4-[(1S)-1-[[1-isopropyl-4-[[4-(trifluoromethyl) phenyl]methyl]indazole-3-carbonyl]amino]ethyl]benzoic acid (Compound 20)

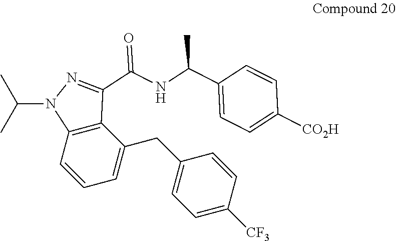 [SMILES: CC(C)n1nc(C(=O)N[C@@H](C)c2ccc(C(=O)O)cc2)c2c(Cc3ccc(C(F)(F)F)cc3)cccc21]

### Step 1: preparation of methyl 4-bromo-1-isopropyl-1H-indazole-3-carboxylate and methyl 4-bromo-2-isopropyl-2H-indazole-3-carboxylate

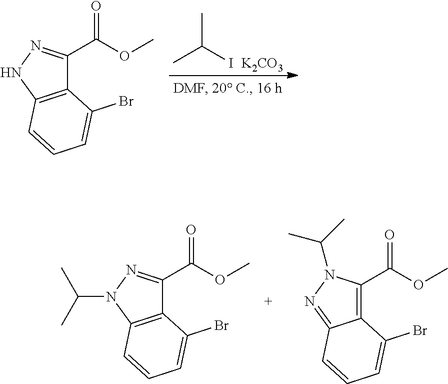

To a solution of methyl 4-bromo-1H-indazole-3-carboxylate (1 g, 3.92 mmol, 1 eq) and 2-iodopropane (3.33 g, 19.60 mmol, 1.96 mL, 5 eq) in DMF (6 mL) was added K₂CO₃ (1.08 g, 7.84 mmol, 2 eq). The mixture was stirred at 25° C. for 5 hr. The reaction mixture was diluted with H₂O 30 mL and extracted with EA 45 mL (15 mL×3). The combined organic layers were washed with brine 30 mL, dried over Na₂SO₄, filtered and concentrated under reduced pressure to give a residue. The residue was purified by column chromatography (SiO₂, Petroleum ether/Ethyl acetate=50/1 to 0/1). Compound methyl 4-bromo-1-isopropyl-indazole-3-carboxylate (620 mg, 51% yield) and methyl 4-bromo-2-isopropyl-2H-indazole-3-carboxylate (410 mg, 35% yield) were obtained as yellow oil.

Methyl 4-bromo-1-isopropyl-indazole-3-carboxylate: ¹H NMR (400 MHz, DMSO-d6) δ=7.90 (d, J=8.4 Hz, 1H), 7.54 (d, J=6.4 Hz, 1H), 7.39-7.35 (m, 1H), 5.15-5.09 (m, 1H), 3.91 (s, 3H), 1.49 (d, J=6.4 Hz, 6H).

methyl 4-bromo-2-isopropyl-2H-indazole-3-carboxylate: ¹H NMR (400 MHz, DMSO-d6) δ=7.79 (d, J=8.0 Hz, 1H), 7.48-7.46 (m, 1H), 7.27-7.23 (m, 1H), 5.11-5.04 (m, 1H), 4.01 (s, 3H), 1.54 (d, J=6.4 Hz, 6H).

### Step 2: methyl 1-isopropyl-4-[[4-(trifluoromethyl) phenyl]methyl]-indazole-3-carboxylate

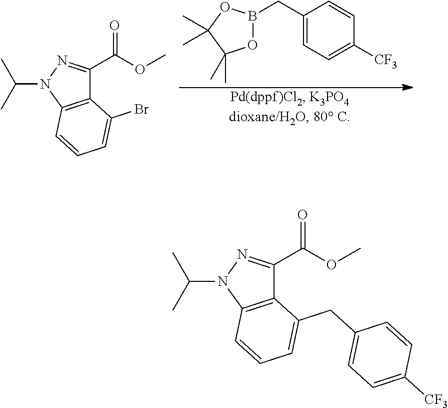

To a solution of 4,4,5,5-tetramethyl-2-[[4-(trifluoromethyl) phenyl]methyl]-1,3,2-dioxaborolane (385.12 mg, 1.35 mmol, 2 eq) and methyl 4-bromo-1-isopropyl-indazole-3-carboxylate (200.00 mg, 673.07 umol, 1 eq) in H₂O (0.5 mL) and dioxane (2 mL) was added K₃PO₄ (142.87 mg, 673.07 umol, 1 eq) and Pd(dppf)Cl₂ (147.75 mg, 201.92 umol, 0.3 eq). The mixture was stirred at 80° C. for 16 hr. Several new peaks were shown on LC-MS and 41.15% of desired compound was detected. The reaction mixture was diluted with H₂O 10 mL and extracted with DCM 30 mL (10 mL×3). The combined organic layers were washed with brine 10 mL, dried over Na₂SO₄, filtered and concentrated under reduced pressure to give a residue. The residue was purifiedm by prep-TLC (SiO₂, PE:EA=3:1). Compound methyl 1-isopropyl-4-[[4-(trifluoromethyl) phenyl]methyl]indazole-3-carboxylate (170 mg, 451.68 umol, 67.11% yield) was obtained as a yellow oil. LCMS for product (ESI): m/z 377.3 [M+H]⁺.

### Step 3: 1-isopropyl-4-[[4-(trifluoromethyl) phenyl]methyl]indazole-3-carboxylic acid

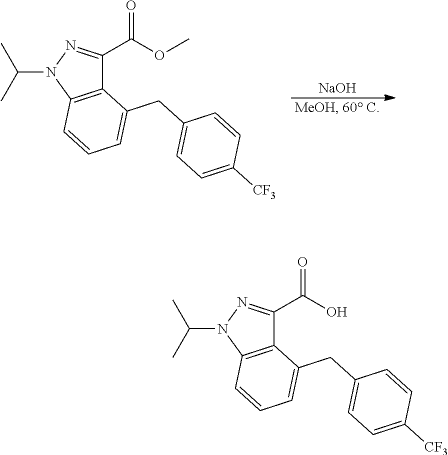 [SMILES: COC(=O)c1nn(C(C)C)c2cccc(Cc3ccc(C(F)(F)F)cc3)c12 ; "MeOH, 60° C." ; "NaOH" ; CC(C)n1nc(C(=O)O)c2c(Cc3ccc(C(F)(F)F)cc3)cccc21]

To a solution of methyl 1-isopropyl-4-[[4-(trifluoromethyl) phenyl]methyl]indazole-3-carboxylate (170 mg, 451.68 umol, 1 eq) in H₂O (1.5 mL) and MeOH (1.5 mL) was added NaOH (54.20 mg, 1.36 mmol, 3 eq). The mixture was stirred at 50° C. for 16 hr. The reaction mixture was concentrated under reduced pressure to remove MeOH. The mixture was adjusted to pH=5 with 1N HCl. The mixture was filtered. Compound 1-isopropyl-4-[[4-(trifluoromethyl) phenyl]methyl]indazole-3-carboxylic acid (130 mg, 358.77 umol, 79.43% yield) was obtained as a white solid.

### Step 4: methyl 4-[(1S)-1-[[1-isopropyl-4-[[4-(trifluoromethyl) phenyl]methyl]-indazole-3-carbonyl]amino ethyl benzoate

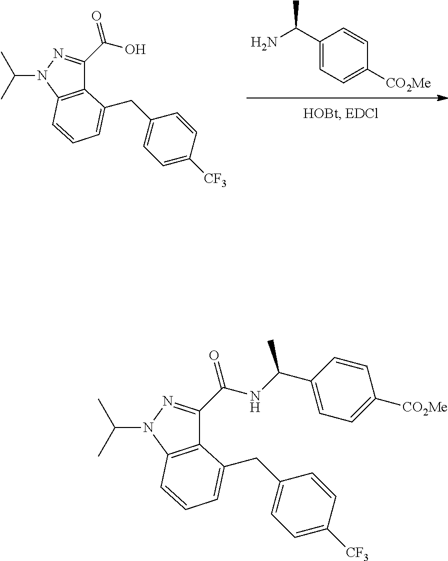 [SMILES: CC(C)n1nc(C(=O)O)c2c(Cc3ccc(C(F)(F)F)cc3)cccc21 ; COC(=O)c1ccc([C@H](C)N)cc1 ; COC(=O)c1ccc([C@H](C)NC(=O)c2nn(C(C)C)c3cccc(Cc4ccc(C(F)(F)F)cc4)c23)cc1]

To a solution of methyl 4-[(1S)-1-aminoethyl]benzoate (19.78 mg, 110.39 umol, 1 eq) in DCM (1 mL) was added HOBt (2.98 mg, 22.08 umol, 0.2 eq), Et₃N (55.85 mg, 551.95 umol, 76.83 uL, 5 eq) and EDCI (42.32 mg, 220.78 umol, 2 eq) at 0° C. The mixture was stirred at 0° C. for 0.5 hr. Then 1-isopropyl-4-[[4-(trifluoromethyl) phenyl]methyl]indazole-3-carboxylic acid (40 mg, 110.39 umol, 1 eq) was added at 0° C. The mixture was stirred at 25° C. for 16 hr. Several new peaks were shown on LC-MS and 51.93% of desired compound was detected. The reaction mixture was diluted with H₂O 2 mL and extracted with DCM 9 mL (3 mL×3). The combined organic layers were washed with brine 5 mL, dried over Na₂SO₄, filtered and concentrated under reduced pressure to give a residue. The residue was purified by prep-TLC (SiO₂, PE:EA=1:1). Compound methyl 4-[(1S)-1-[[1-isopropyl-4-[[4-(trifluoromethyl) phenyl]methyl]indazole-3-carbonyl]amino]ethyl]benzoate (35 mg, 66.85 umol, 60.56% yield) was obtained as a white solid, which was used into the next step directly without further purification. LCMS for product (ESI): m/z 524.3 [M+H]⁺.

### Step 5: 4-[(1S)-1-[[1-isopropyl-4-[[4-(trifluoromethyl)phenyl]methyl]indazole-3-carbonyl]-amino]ethyl]-benzoic acid

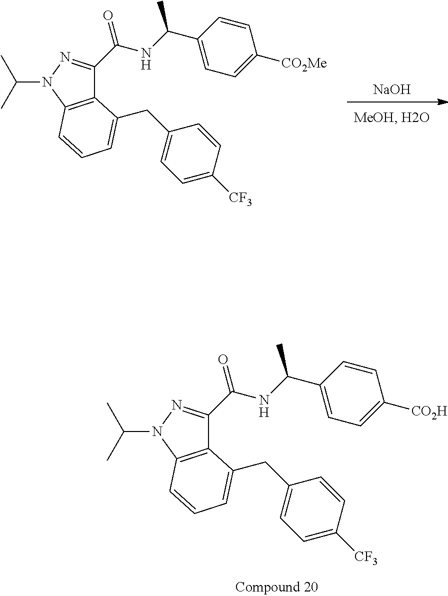 [SMILES: COC(=O)c1ccc([C@H](C)NC(=O)c2nn(C(C)C)c3cccc(Cc4ccc(C(F)(F)F)cc4)c23)cc1 ; "NaOH" ; CC(C)n1nc(C(=O)N[C@@H](C)c2ccc(C(=O)O)cc2)c2c(Cc3ccc(C(F)(F)F)cc3)cccc21]

To a solution of methyl 4-[(1S)-1-[[1-isopropyl-4-[[4-(trifluoromethyl) phenyl]methyl]indazole-3-carbonyl]amino]ethyl]benzoate (35 mg, 66.85 umol, 1 eq) in H₂O (1 mL) and MeOH (1 mL) was added NaOH (8.02 mg, 200.56 umol, 3 eq). The mixture was stirred at 50° C. for 16 hr. The reaction mixture was concentrated under reduced pressure to remove MeOH. The mixture was adjusted to PH=5 with 1N HCl. The mixture was filtered. Compound 4-[(1S)-1-[[1-isopropyl-4-[[4-(trifluoromethyl) phenyl]methyl]indazole-3-carbonyl]amino]ethyl]benzoic acid (30 mg, 55.52 umol, 83.04% yield, 94.29% purity) was obtained as a white solid. LCMS for product (ESI): m/z 510.1 [M+H]⁺. ¹H NMR (400 MHz, DMSO-d₆) δ ppm 12.83 (brs, 1H), 8.79 (d, J=8.4 Hz, 1H), 7.91 (d, J=8.0 Hz, 2H), 7.66 (d, J=8.4 Hz, 1H), 7.55 (d, J=8.4 Hz, 2H), 7.38-7.33 (m, 3H), 7.20 (d, J=8.4 Hz, 2H), 7.12 (d, J=8.0 Hz, 1H), 5.27-5.23 (m, 1H), 5.08-5.02 (m, 1H), 4.71 (d, J=14.4 Hz, 1H), 4.50 (d, J=14.4 Hz, 1H), 1.53 (d, J=6.4, 6H), 1.48 (d, J=7.2 Hz, 3H)

### Example 25—Synthesis of trans-2-[[1-isopropyl-4-[[4-(trifluoromethyl) phenyl]methyl]indazole-3-carbonyl]amino]spiro[3.3]heptane-6-carboxylic acid (Compound

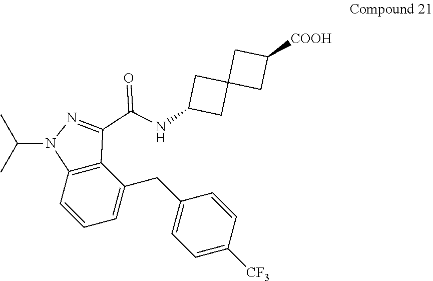 [SMILES: CC(C)n1nc(C(=O)N[C@H]2CC3(C2)C[C@H](C(=O)O)C3)c2c(Cc3ccc(C(F)(F)F)cc3)cccc21]

### Step 1: trans-(S)-1-phenylethyl 2-[[1-isopropyl-4-[[4-(trifluoromethyl) phenyl]methyl]indazole-3-carbonyl]amino]spiro[3.3]heptane-6-carboxylate

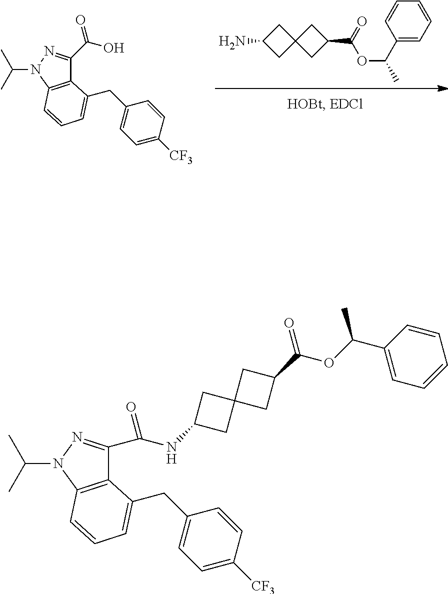 [SMILES: CC(C)n1nc(C(=O)O)c2c(Cc3ccc(C(F)(F)F)cc3)cccc21 ; C[C@H](OC(=O)[C@H]1CC2(C[C@@H](N)C2)C1)c1ccccc1 ; CC(C)n1nc(C(=O)N[C@H]2CC3(C2)C[C@H](C(=O)O[C@@H](C)c2ccccc2)C3)c2c(Cc3ccc(C(F)(F)F)cc3)cccc21]

To a solution of trans-(S)-1-phenylethyl 2-aminospiro[3.3]heptane-6-carboxylate (35.79 mg, 137.99 umol, 1 eq) in DCM (1 mL) was added HOBt (3.73 mg, 27.60 umol, 0.2 eq), Et₃N (69.82 mg, 689.95 umol, 96.03 uL, 5 eq) and EDCI (52.91 mg, 275.98 umol, 2 eq) at 0° C. The mixture was stirred at 0° C. for 0.5 hr. Then 1-isopropyl-4-[[4-(trifluoromethyl) phenyl]methyl]indazole-3-carboxylic acid (50 mg, 137.99 umol, 1 eq) was added at 0° C. The mixture was stirred at 25° C. for 16 hr. Several new peaks were shown on LC-MS and 48.94% of desired compound was detected. The reaction mixture was diluted with H₂O 5 mL and extracted with DCM 15 mL (5 mL×3). The combined organic layers were washed with brine 5 mL, dried over Na₂SO₄, filtered and concentrated under reduced pressure to give a residue.

The residue was purified by prep-TLC (SiO₂, PE:EA=1:1). Compound trans-(S)-1-phenylethyl 2-[[1-isopropyl-4-[[4-(trifluoromethyl) phenyl]methyl]indazole-3-carbonyl]amino]spiro[3.3]heptane-6-carboxylate (45 mg, 74.54 umol, 54.02% yield) was obtained as a white solid, which was used into the next step directly without further purification. LCMS for product (ESI): m/z 604.4 [M+H]⁺.

### Step 2: trans-2-[[1-isopropyl-4-[[4-(trifluoromethyl) phenyl]methyl]indazole-3-carbonyl]amino]spiro[3.3]heptane-6-carboxylic acid

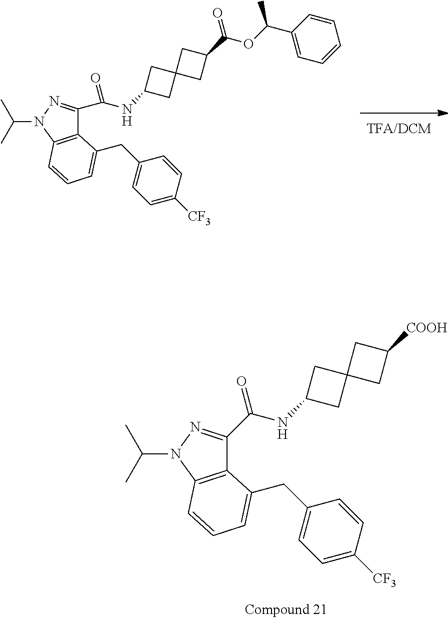 [SMILES: CC(C)n1nc(C(=O)N[C@H]2CC3(C2)C[C@H](C(=O)O[C@@H](C)c2ccccc2)C3)c2c(Cc3ccc(C(F)(F)F)cc3)cccc21 ; CC(C)n1nc(C(=O)N[C@H]2CC3(C2)C[C@H](C(=O)O)C3)c2c(Cc3ccc(C(F)(F)F)cc3)cccc21]

To a solution of trans-(S)-1-phenylethyl 2-[[1-isopropyl-4-[[4-(trifluoromethyl) phenyl]methyl]indazole-3-carbonyl]amino]spiro[3.3]heptane-6-carboxylate (45 mg, 74.54 umol, 1 eq) in TFA (1 mL) and DCM (1 mL). The mixture was stirred at 50° C. for 16 hr. The reaction mixture was concentrated under reduced pressure to remove MeOH. The mixture was adjusted to PH=5 with 1N HCl. The mixture was filtered. Compound trans-2-[[1-isopropyl-4-[[4-(trifluoromethyl) phenyl]methyl]indazole-3-carbonyl]amino]spiro[3.3]heptane-6-carboxylic acid (22 mg, 41.06 umol, 55.08% yield, 93.23% purity) was obtained as a white solid. LCMS for product (ESI): m/z 500.1 [M+H]⁺. ¹H NMR (400 MHz, DMSO-d₆) δ ppm 8.42 (d, J=8.0 Hz, 1H) 7.66 (d, J=8.4 Hz, 1H) 7.56 (d, J=8.4 Hz, 2H) 7.40-7.37 (m, 1H), 7.32 (d, J=8.0 Hz, 2H) 7.11 (d, J=7.2 Hz, 1H) 5.00-5.09 (m, 1H) 4.67 (s, 2H) 4.32-4.25 (m, 1H), 2.97-2.91 (m, 1H), 2.41-2.36 (m, 3H) 2.21-2.12 (m, 2H) 2.11-2.07 (m, 1H) 2.03-2.00 (m, 2H) 1.51 (d, J=6.8 Hz, 6H).

### Example 26—Synthesis of 2-[3-[[1-isopropyl-4-[[4-(trifluoromethyl) phenyl]methyl]indazole-3-carbonyl]amino]-1-bicyclo[1.1.1]pentanyl]acetic acid (Compound 22)

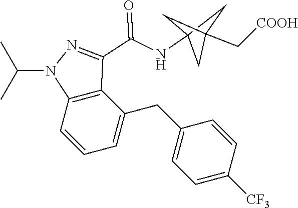 [SMILES: CC(C)n1nc(C(=O)NC23CC(CC(=O)O)(C2)C3)c2c(Cc3ccc(C(F)(F)F)cc3)cccc21]

### Compound 22

### Step 1: methyl 2-[3-[[1-isopropyl-4-[[4-(trifluoromethyl) phenyl]methyl]indazole-3-carbonyl]amino]-1-bicyclo[1.1.11pentanyl]acetate

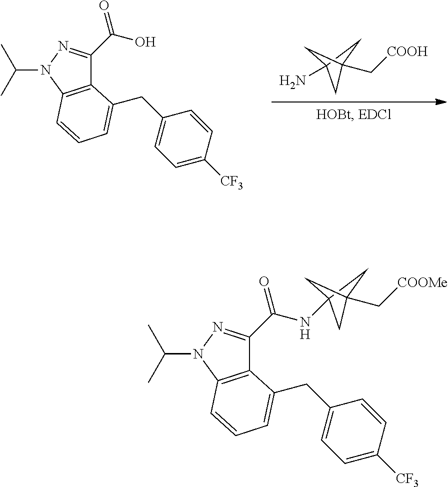

To a solution of methyl 2-(3-amino-1-bicyclo[1.1.1]pentanyl) acetate (18.85 mg, 121.43 umol, 1.1 eq) in DCM (1 mL) was added HOBt (2.98 mg, 22.08 umol, 0.2 eq), Et₃N (55.85 mg, 551.96 umol, 76.83 uL, 5 eq) and EDCI (42.32 mg, 220.78 umol, 2 eq) at 0° C. The mixture was stirred at 0° C. for 0.5 hr. Then 1-isopropyl-4-[[4-(trifluoromethyl) phenyl]methyl]indazole-3-carboxylic acid (40 mg, 110.39 umol, 1 eq) was added at 0° C. The mixture was stirred at 25° C. for 16 hr. Several new peaks were shown on LC-MS and 45.43% of desired compound was detected. The reaction mixture was diluted with H₂O 5 mL and extracted with DCM 15 mL (5 mL×3). The combined organic layers were washed with brine 5 mL, dried over Na₂SO₄, filtered and concentrated under reduced pressure to give a residue. The residue was purified by prep-TLC (SiO₂, PE:EA=1:1). Compound methyl 2-[3-[[1-isopropyl-4-[[4-(trifluoromethyl) phenyl]methyl]indazole-3-carbonyl]amino]-1-bicyclo[1.1.1]pentanyl]acetate (32 mg, 64.06 umol, 58.03% yield) was obtained as a white solid, which was used into the next step directly without further purification. LCMS for product (ESI): m/z 500.3 [M+H]⁺.

### Step 2: 2-[3-[[1-isopropyl-4-[[4-(trifluoromethyl) phenyl]methyl]indazole-3-carbonyl]amino]-1-bicyclo[1.1.11pentanyl]acetic acid

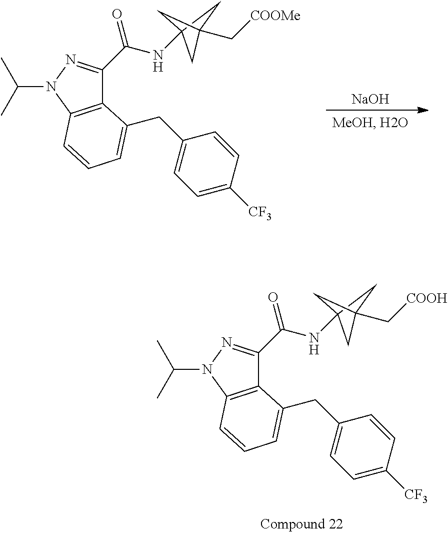 [SMILES: COC(=O)CC12CC(NC(=O)c3nn(C(C)C)c4cccc(Cc5ccc(C(F)(F)F)cc5)c34)(C1)C2 ; "NaOH" ; "MeOH, H2O" ; CC(C)n1nc(C(=O)NC23CC(CC(=O)O)(C2)C3)c2c(Cc3ccc(C(F)(F)F)cc3)cccc21]

To a solution of methyl 2-[3-[[1-isopropyl-4-[[4-(trifluoromethyl) phenyl]methyl]indazole-3-carbonyl]amino]-1-bicyclo[1.1.1]pentanyl]acetate (32 mg, 64.06 umol, 1 eq) in H₂O (1 mL) and MeOH (1 mL) was added NaOH (7.69 mg, 192.18 umol, 3 eq). The mixture was stirred at 50° C. for 16 hr. The reaction mixture was concentrated under reduced pressure to remove MeOH. The mixture was adjusted to pH=5 with 1N HCl. The mixture was filtered. Compound 2-[3-[[1-isopropyl-4-[[4-(trifluoromethyl) phenyl]methyl]indazole-3-carbonyl]amino]-1-bicyclo[1.1.1]pentanyl]acetic acid (18 mg, 35.67 umol, 55.68% yield, 96.20% purity) was obtained as a white solid. LCMS for product (ESI): m/z 486.1 [M+H]⁺. ¹H NMR (400 MHz, DMSO-d₆) δ=12.10 (br s, 1H), 8.74 (s, 1H), 7.65 (d, J=8.4 Hz, 1H), 7.54 (d, J=8.4 Hz, 2H), 7.37 (d, J=8.4 Hz, 1H), 7.37-7.33 (m, 2H), 7.13 (d, J=7.2 Hz, 1H), 5.06-4.99 (m, 1H), 4.68 (s, 2H), 2.50 (s, 2H), 2.05 (s, 6H), 1.49 (d, J=6.4 Hz, 6H).

### Example 27—Synthesis of 4-[(1S)-1-[[1-isobutyl-4-[[4-(trifluoromethyl) phenyl]methyl]indazole-3-carbonyl]amino]ethyl]benzoic acid (Compound 23)

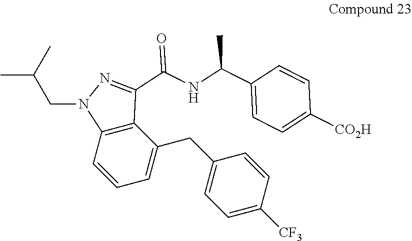 [SMILES: CC(C)Cn1nc(C(=O)N[C@@H](C)c2ccc(C(=O)O)cc2)c2c(Cc3ccc(C(F)(F)F)cc3)cccc21]

### Step 1: methyl 4-bromo-1-isobutyl-indazole-3-carboxylate

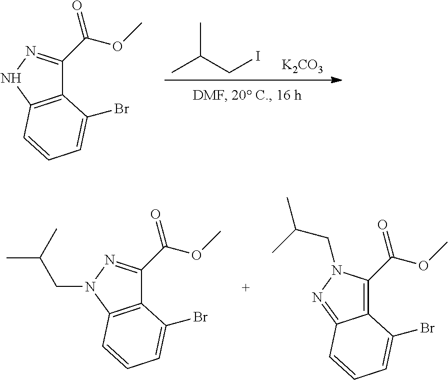

To a solution of methyl 4-bromo-1H-indazole-3-carboxylate (1 g, 3.92 mmol, 1 eq) and 1-iodo-2-methyl-propane (3.61 g, 19.60 mmol, 2.25 mL, 5 eq) in DMF (1 mL) was added K₂CO₃ (1.08 g, 7.84 mmol, 2 eq). The mixture was stirred at 25° C. for 5 hr. TLC indicated Reactant 1 was consumed completely. The reaction mixture was diluted with H₂O 30 mL and extracted with EA 30 mL (10 mL×3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=50/1 to 0/1). Compound methyl 4-bromo-1-isobutyl-indazole-3-carboxylate (750 mg, 61.8% yield) and methyl 4-bromo-2-isobutyl-indazole-3-carboxylate (380 mg, 31.3% yield) was obtained as a yellow oil.

Methyl 4-bromo-1-isobutyl-indazole-3-carboxylate: ¹H NMR (400 MHz, DMSO-d₆) δ=7.89 (d, J=8.0 Hz, 1H), 7.54-7.53 (m, 1H), 7.40-7.36 (m, 1H), 4.32 (d, J=7.2 Hz, 2H), 3.91 (s, 3H), 2.26-2.19 (m, 1H), 0.85 (d, J=6.8 Hz, 6H).

Methyl 4-bromo-2-isobutyl-indazole-3-carboxylate: ¹H NMR (400 MHz, DMSO-d₆) δ=7.78 (d, J=8.4 Hz, 1H), 7.50 (d, J=7.2 Hz, 1H), 7.28-7.23 (m, 1H), 4.39 (d, J=7.2 Hz, 2H), 4.00 (s, 3H), 2.29-2.22 (m, 1H), 0.85 (d, J=6.8 Hz, 6H).

### Step 2: methyl 1-isobutyl-4-[[4-(trifluoromethyl) phenyl]methyl]indazole-3-carboxylate

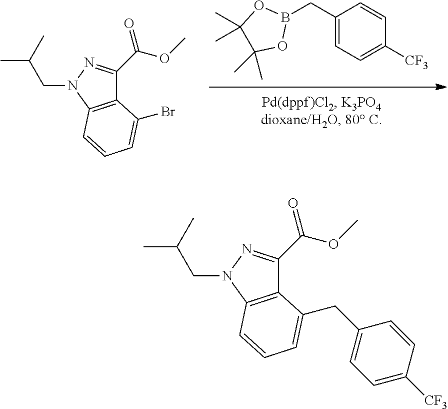

A mixture of methyl 4-bromo-1-isobutyl-indazole-3-carboxylate (200 mg, 642.73 umol, 1 eq), 4,4,5,5-tetramethyl-2-[[4-(trifluoromethyl) phenyl]methyl]-1,3,2-dioxaborolane (367.76 mg, 1.29 mmol, 2 eq), K₃PO₄ (136.43 mg, 642.73 umol, 1 eq), Pd(dppf)Cl₂ (141.09 mg, 192.82 umol, 0.3 eq) in dioxane (1 mL) and H₂O (0.5 mL) was degassed and purged with N₂ for 3 times, and then the mixture was stirred at 80° C. for 12 hr under N₂ atmosphere. The reaction mixture was concentrated under reduced pressure to remove dioxane. The residue was diluted with H₂O 10 mL and extracted with EA 30 mL (10 mL×3). The combined organic layers were washed with brine 10 mL, dried over Na₂SO₄, filtered and concentrated under reduced pressure to give a residue. The residue was purified by prep-TLC (SiO2, PE: EA=3:1). Compound methyl 1-isobutyl-4-[[4-(trifluoromethyl) phenyl]methyl]indazole-3-carboxylate (170 mg, 435.45 umol, 67.75% yield) was obtained as a white solid. HPLC showed the product was clean. LCMS for crude product (ESI): m/z 391.3 [M+H]⁺.

### Step 3: 1-isobutyl-4-[[4-(trifluoromethyl) phenyl]methyl]indazole-3-carboxylic acid

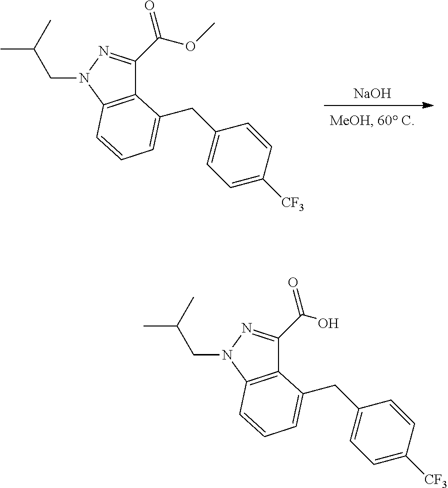 [SMILES: COC(=O)c1nn(CC(C)C)c2cccc(Cc3ccc(C(F)(F)F)cc3)c12 ; "NaOH" ; CC(C)Cn1nc(C(=O)O)c2c(Cc3ccc(C(F)(F)F)cc3)cccc21]

To a solution of methyl 1-isobutyl-4-[[4-(trifluoromethyl) phenyl]methyl]indazole-3-carboxylate (170 mg, 435.45 umol, 1 eq) in H₂O (0.5 mL) and MeOH (2 mL) was added NaOH (52.26 mg, 1.36 mmol, 3 eq). The mixture was stirred at 80° C. for 3 hr. The reaction was clean according to TLC. The reaction mixture was concentrated under reduced pressure to remove MeOH. The mixture was adjusted to pH 4 with 1N HCl. The mixture was filtered. Compound 1-isobutyl-4-[[4-(trifluoromethyl) phenyl]methyl]indazole-3-carboxylic acid (140 mg, 371.97 umol, 85.42% yield) was obtained as a white solid.

### Step 4: methyl 4-[(1S)-1-[[1-isobutyl-4-[[4-(trifluoromethyl) phenyl]methyl]indazole-3-carbonyl]amino]ethyl]benzoate

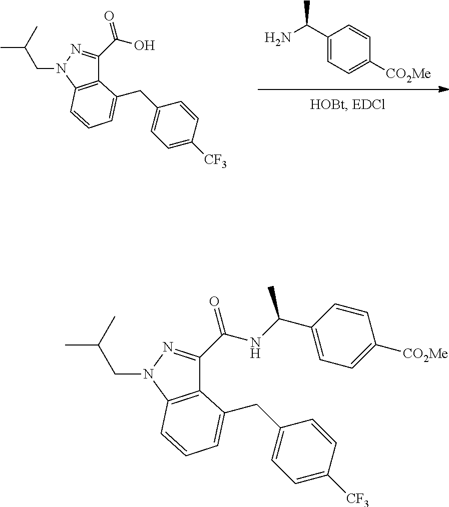 [SMILES: CC(C)Cn1nc(C(=O)O)c2c(Cc3ccc(C(F)(F)F)cc3)cccc21 ; COC(=O)c1ccc([C@H](C)N)cc1 ; COC(=O)c1ccc([C@H](C)NC(=O)c2nn(CC(C)C)c3cccc(Cc4ccc(C(F)(F)F)cc4)c23)cc1]

To a solution of 1-isobutyl-4-[[4-(trifluoromethyl) phenyl]methyl]indazole-3-carboxylic acid (45 mg, 119.56 umol, 1 eq) in DCM (2 mL) was added Et₃N (60.49 mg, 597.81 umol, 83.21 uL, 5 eq), HOBt (3.23 mg, 23.91 umol, 0.2 eq) and EDCI (45.84 mg, 239.13 umol, 2 eq) at 0° C. After addition, the mixture was stirred at this temperature for 0.5 hr, and then methyl 4-[(1S)-1-aminoethyl]benzoate (32.14 mg, 179.34 umol, 1.5 eq) was added at 0° C. The resulting mixture was stirred at 25° C. for 12 hr. The residue was diluted with H₂O 10 mL and extracted with DCM 15 mL (5 mL×3). The combined organic layers were washed with brine 10 mL, dried over Na₂SO₄, filtered and concentrated under reduced pressure to give a residue. The residue was purified by prep-TLC (SiO2, PE: EA=1:1). Compound methyl 4-[(1S)-1-[[1-isobutyl-4-[[4-(trifluoromethyl) phenyl]methyl]indazole-3-carbonyl]amino]ethyl]benzoate (30 mg, 55.81 umol, 46.68% yield) was obtained as a white solid, which was used into the next step directly without further purification.

LCMS for product (ESI): m/z 538.3 [M+H]⁺.

### Step 5: 4-[(1S)-1-[[1-isobutyl-4-[[4-(trifluoromethyl) phenyl]methyl]indazole-3-carbonyl]amino]ethyl]benzoic acid

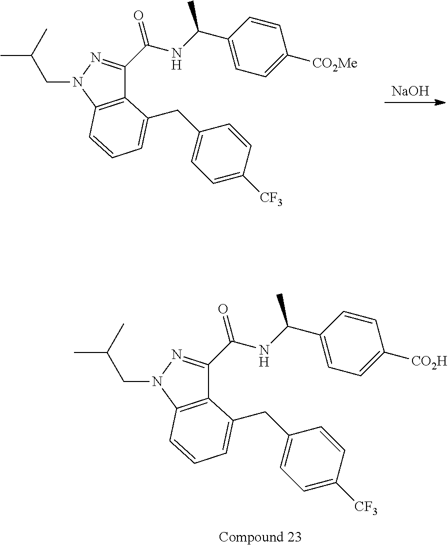 [SMILES: COC(=O)c1ccc([C@H](C)NC(=O)c2nn(CC(C)C)c3cccc(Cc4ccc(C(F)(F)F)cc4)c23)cc1 ; "NaOH" ; CC(C)Cn1nc(C(=O)N[C@@H](C)c2ccc(C(=O)O)cc2)c2c(Cc3ccc(C(F)(F)F)cc3)cccc21]

To a solution of methyl 4-[(1S)-1-[[1-isobutyl-4-[[4-(trifluoromethyl) phenyl]methyl]indazole-3-carbonyl]amino]ethyl]benzoate (30 mg, 55.81 umol, 1 eq) in MeOH (2 mL) and H₂O (2 mL) was added NaOH (6.70 mg, 167.42 umol, 3 eq). The mixture was stirred at 50° C. for 12 hr. The reaction mixture was concentrated under reduced pressure to remove MeOH. The mixture was adjusted to pH=5 with 1N HCl. The mixture was filtered. Compound 4-[(1S)-1-[[1-isobutyl-4-[[4-(trifluoromethyl) phenyl]methyl]indazole-3-carbonyl]amino]ethyl]benzoic acid (10 mg, 17.38 umol, 31.15% yield, 91% purity) was obtained as a white solid. LCMS for product (ESI): m/z 524.1 [M+H]⁺. ¹H NMR (400 MHz, DMSO-d₆) δ=8.75 (d, J=8.0 Hz, 1H), 7.87 (d, J=8.0 Hz, 2H), 7.65 (d, J=8.4 Hz, 1H), 7.46-7.36 (m, 5H), 7.25 (d, J=8.0 Hz, 2H), 7.09 (d, J=7.2 Hz, 1H), 5.24-5.17 (m, 1H), 4.63 (s, 2H), 4.26 (d, J=7.2 Hz, 2H), 2.30-2.23 (m, 1H), 1.44 (d, J=7.2 Hz, 3H), 0.87 (d, J=6.8 Hz, 6H).

### Example 28—Synthesis of trans-2-[[1-isobutyl-4-[[4-(trifluoromethyl) phenyl]methyl]indazole-3-carbonyl]amino]spiro[3.3]heptane-6-carboxylic acid (Compound

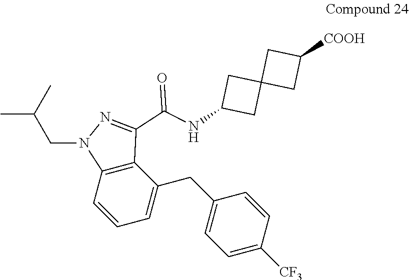 [SMILES: CC(C)Cn1nc(C(=O)N[C@H]2CC3(C2)C[C@H](C(=O)O)C3)c2c(Cc3ccc(C(F)(F)F)cc3)cccc21]

### Step 1: trans-(S)-1-phenylethyl 2-[[1-isobutyl-4-[[4-(trifluoromethyl) phenyl]methyl]indazole-3-carbonyl]amino]spiro[3.3]heptane-6-carboxylate

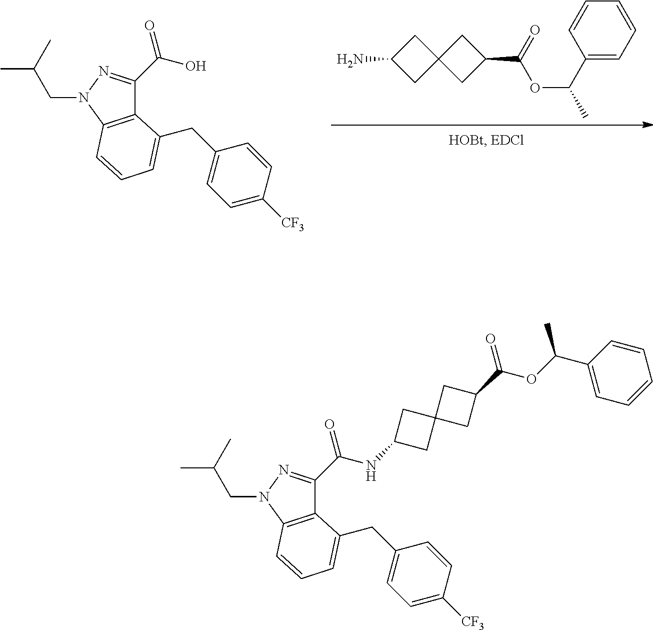 [SMILES: CC(C)Cn1nc(C(=O)O)c2c(Cc3ccc(C(F)(F)F)cc3)cccc21 ; C[C@H](OC(=O)[C@H]1CC2(C[C@@H](N)C2)C1)c1ccccc1 ; CC(C)Cn1nc(C(=O)N[C@H]2CC3(C2)C[C@H](C(=O)O[C@@H](C)c2ccccc2)C3)c2c(Cc3ccc(C(F)(F)F)cc3)cccc21]

To a solution of 1-isobutyl-4-[[4-(trifluoromethyl) phenyl]methyl]indazole-3-carboxylic acid (50 mg, 132.85 umol, 1 eq) in DCM (2 mL) was added Et₃N (67.21 mg, 664.25 umol, 92.45 uL, 5 eq), HOBt (3.59 mg, 26.57 umol, 0.2 eq) and EDCI (50.93 mg, 265.70 umol, 2 eq) at 0° C. After addition, the mixture was stirred at this temperature for 0.5 hr, and then trans-(S)-1-phenylethyl 2-aminospiro[3.3]heptane-6-carboxylate (51.68 mg, 199.28 umol, 1.5 eq) was added dropwise at 0° C. The resulting mixture was stirred at 25° C. for 12 hr. The residue was diluted with H₂O 10 mL and extracted with DCM 30 mL (10 mL×3). The combined organic layers were washed with brine 10 mL, dried over Na₂SO₄, filtered and concentrated under reduced pressure to give a residue. The residue was purified by prep-TLC (SiO2, PE: EA=1:1). Compound trans-(S)-1-phenylethyl 2-[[1-isobutyl-4-[[4-(trifluoromethyl) phenyl]methyl]indazole-3-carbonyl]amino]spiro[3.3]heptane-6-carboxylate (45 mg, 72.85 umol, 54.84% yield) was obtained as a white solid, which was used into the next step directly without further purification. LCMS for product (ESI): m/z 618.4 [M+H]⁺.

### Step 2: trans-2-[[1-isobutyl-4-[[4-(trifluoromethyl) phenyl]methyl]indazole-3-carbonyl]amino]spiro[3.3]heptane-6-carboxylic acid

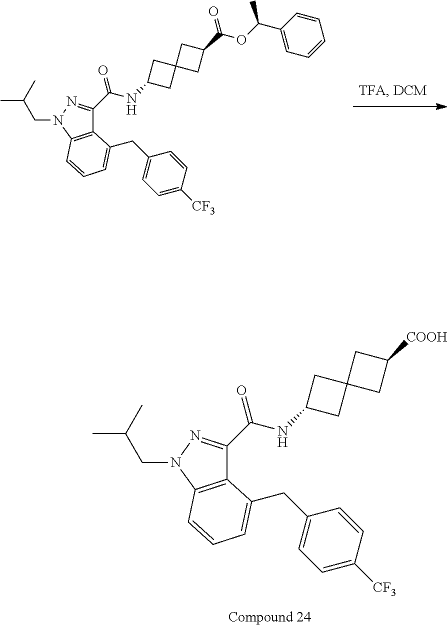 [SMILES: CC(C)Cn1nc(C(=O)N[C@H]2CC3(C2)C[C@H](C(=O)O[C@@H](C)c2ccccc2)C3)c2c(Cc3ccc(C(F)(F)F)cc3)cccc21 ; CC(C)Cn1nc(C(=O)N[C@H]2CC3(C2)C[C@H](C(=O)O)C3)c2c(Cc3ccc(C(F)(F)F)cc3)cccc21]

To a solution of trans-(S)-1-phenylethyl 2-[[1-isobutyl-4-[[4-(trifluoromethyl) phenyl]methyl]indazole-3-carbonyl]amino]spiro[3.3]heptane-6-carboxylate (45 mg, 72.85 umol, 1 eq) in TFA (2 mL) and DCM (2 mL). The mixture was stirred at 25° C. for 12 hr. The reaction mixture was concentrated under reduced pressure to remove solvent. The mixture was dissolved by adding 1M NaOH (10 mL) and extracted with DCM 15 mL (5 mL×3). The solution was filtered and the filtrate was adjusted to pH 4 with 1N HCl. The mixture was filtered. Compound trans-2-[[1-isobutyl-4-[[4-(trifluoromethyl) phenyl]methyl]indazole-3-carbonyl]amino]spiro[3.3]heptane-6-carboxylic acid (31 mg, 55.35 umol, 75.98% yield, 91.7% purity) was obtained as a white solid. LCMS for product (ESI): m/z 514.3 [M+H]⁺. ¹H NMR (400 MHz, DMSO-d₆) δ=8.45 (d, J=8.0 Hz, 1H), 7.64 (d, J=8.4 Hz, 1H), 7.55 (d, J=8.0 Hz, 2H), 7.41-7.36 (m, 1H), 7.30 (d, J=8.0 Hz, 2H), 7.09 (d, J=6.8 Hz, 1H), 4.66 (s, 2H), 4.30-4.21 (m, 3H), 2.94-2.89 (m, 1H), 2.39-2.24 (m, 3H), 2.23-2.13 (m, 4H), 2.04-1.94 (m, 2H), 0.86 (d, J=6.8 Hz, 6H).

### Example 29—Synthesis of 2-[3-[[1-isobutyl-4-[[4-(trifluoromethyl) phenyl]methyl]indazole-3-carbonyl]amino]-1-bicyclo[1.1.1]pentanyl]acetic acid (Compound 25)

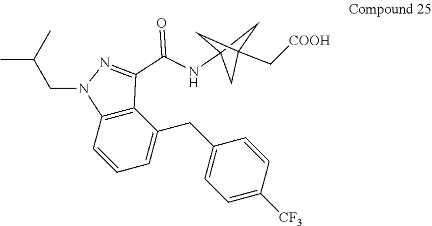 [SMILES: CC(C)Cn1nc(C(=O)NC23CC(CC(=O)O)(C2)C3)c2c(Cc3ccc(C(F)(F)F)cc3)cccc21]

### Step 1: methyl 2-[3-[[1-isobutyl-4-[[4-(trifluoromethyl) phenyl]methyl]indazole-3-carbonyl]amino]-1-bicyclo[1.1.1]pentanyl]acetate

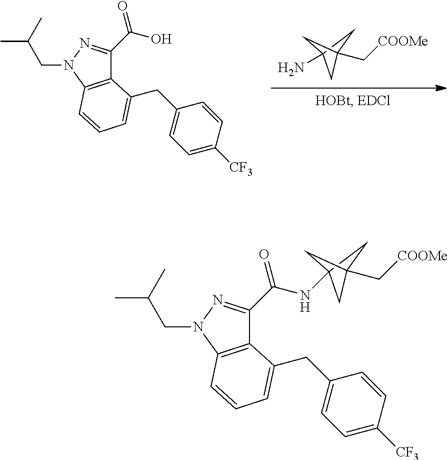

To a solution of 1-isobutyl-4-[[4-(trifluoromethyl) phenyl]methyl]indazole-3-carboxylic acid (45 mg, 119.56 umol, 1 eq) in DCM (2 mL) was added Et3N (60.49 mg, 597.81 umol, 83.21 uL, 5 eq), HOBt (3.23 mg, 23.91 umol, 0.2 eq) and EDCI (45.84 mg, 239.13 umol, 2 eq) at 0° C. After addition, the mixture was stirred at this temperature for 0.5 hr, and then methyl 2-(3-amino-1-bicyclo[1.1.1]pentanyl) acetate (27.83 mg, 179.34 umol, 1.5 eq) was added dropwise at 0° C. The resulting mixture was stirred at 25° C. for 12 hr. The residue was diluted with H₂O 10 mL and extracted with DCM 15 mL (5 mL×3). The combined organic layers were washed with brine 10 mL, dried over Na₂SO₄, filtered and concentrated under reduced pressure to give a residue. The residue was purified by prep-TLC (SiO2, PE: EA=1:1). Compound methyl 2-[3-[[1-isobutyl-4-[[4-(trifluoromethyl) phenyl]methyl]indazole-3-carbonyl]amino]-1-bicyclo[1.1.1]pentanyl]acetate (40 mg, 77.89 umol, 65.15% yield) was obtained as a white solid, which was used into the next step directly without further purification. LCMS for product (ESI): m/z 514.3 [M+H]⁺.

### Step 2: 2-[3-[[1-isobutyl-4-[[4-(trifluoromethyl) phenyl]methyl]indazole-3-carbonyl]amino]-1-bicyclo[1.1.1]pentanyl]acetic acid

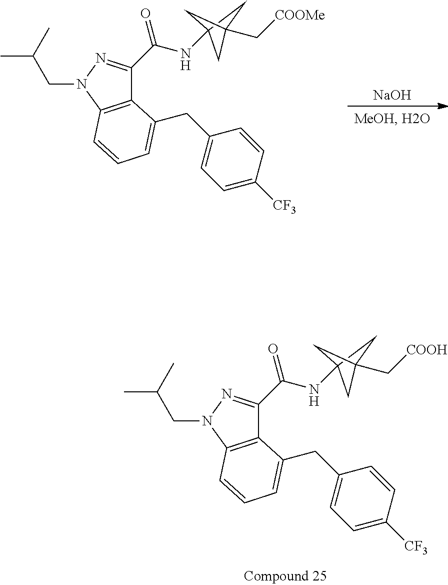 [SMILES: COC(=O)CC12CC(NC(=O)c3nn(CC(C)C)c4cccc(Cc5ccc(C(F)(F)F)cc5)c34)(C1)C2 ; "NaOH" ; "MeOH, H2O" ; CC(C)Cn1nc(C(=O)NC23CC(CC(=O)O)(C2)C3)c2c(Cc3ccc(C(F)(F)F)cc3)cccc21]

To a solution of methyl 2-[3-[[1-isobutyl-4-[[4-(trifluoromethyl) phenyl]methyl]indazole-3-carbonyl]amino]-1-bicyclo[1.1.1]pentanyl]acetate (40 mg, 77.89 umol, 1 eq) in H₂O (2 mL) and MeOH (2 mL) was added NaOH (3.12 mg, 77.89 umol, 1 eq). The mixture was stirred at 50° C. for 12 hr. The reaction mixture was concentrated under reduced pressure to remove solvent. The mixture was adjusted to pH=5 with 1N HCl. The mixture was filtered. Compound 2-[3-[[1-isobutyl-4-[[4-(trifluoromethyl) phenyl]methyl]indazole-3-carbonyl]amino]-1-bicyclo[1.1.1]pentanyl]acetic acid (33 mg, 62.76 umol, 80.57% yield, 95% purity) was obtained as a white solid. LCMS for product (ESI): m/z 500.1 [M+H]⁺. ¹H NMR (400 MHz, DMSO-d₆) δ=8.66 (s, 1H), 7.62 (d, J=8.4 Hz, 1H), 7.53 (d, J=8.0 Hz, 2H), 7.40-7.33 (m, 3H), 7.10 (d, J=7.2 Hz, 1H), 4.67 (s, 2H), 4.20 (d, J=7.2 Hz, 2H), 2.29-2.19 (m, 3H), 1.99 (s, 6H), 0.84 (d, J=6.8 Hz, 6H).

### Example 30—Synthesis of 4-[1-[[4-[[4-(trifluoromethyl)phenyl]methyl]pyrazolo[1,5-a]pyridine-3-carbonyl]amino]cyclopropyl]benzoic acid (Compound 26)

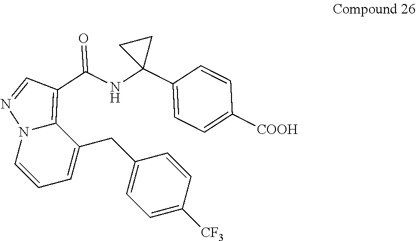 [SMILES: O=C(O)c1ccc(C2(NC(=O)c3cnn4cccc(Cc5ccc(C(F)(F)F)cc5)c34)CC2)cc1]

### Step 1: methyl 4-[1-[[4-[[4-(trifluoromethyl) phenyl]methyl]pyrazolo[1,5-a]pyridine-3-carbonyl]amino]cyclopropyl]benzoate

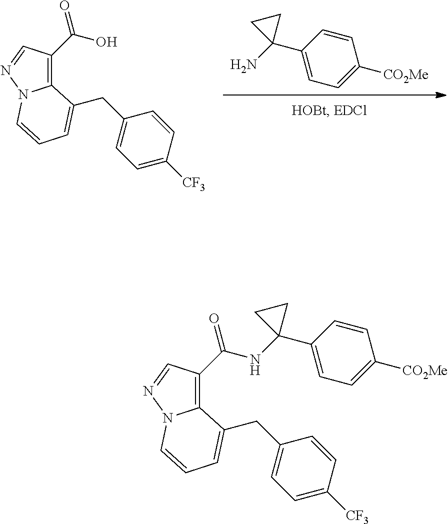 [SMILES: O=C(O)c1cnn2cccc(Cc3ccc(C(F)(F)F)cc3)c12 ; COC(=O)c1ccc(C2(N)CC2)cc1 ; COC(=O)c1ccc(C2(NC(=O)c3cnn4cccc(Cc5ccc(C(F)(F)F)cc5)c34)CC2)cc1]

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added HOBt (1.69 mg, 12.49 umol, 0.2 eq), Et₃N (31.60 mg, 312.24 umol, 43.46 uL, 5 eq) and EDCI (23.94 mg, 124.90 umol, 2 eq) at 0° C. The mixture was stirred at 0° C. for 0.5 hr. Then methyl 4-(1-aminocyclopropyl) benzoate; hydrochloride (14.22 mg, 62.45 umol, 1 eq) was added at 0° C. The mixture was stirred at 25° C. for 16 hr. Several new peaks were shown on LC-MS and 30.44% of desired compound was detected. The reaction mixture was diluted with H₂O 10 mL and extracted with DCM 12 mL (4 mL×3). The combined organic layers were washed with brine 9 mL, dried over Na₂SO₄, filtered and concentrated under reduced pressure to give a residue. The residue was purified by prep-TLC (SiO₂, PE:EA=1:1). Compound methyl 4-[1-[[4-[[4-(trifluoromethyl) phenyl]methyl]pyrazolo[1,5-a]pyridine-3-carbonyl]amino]cyclopropyl]benzoate (20 mg, 40.53 umol, 64.90% yield) was obtained as a white solid, which was used into the next step directly without further purification. LCMS for product (ESI): m/z 494.1 [M+H]⁺.

### Step 2: 4-[1-[[4-[[4-(trifluoromethyl)phenyl]methyl]pyrazolo[1,5-a]pyridine-3-carbonyl]amino]cyclopropyl]benzoic acid

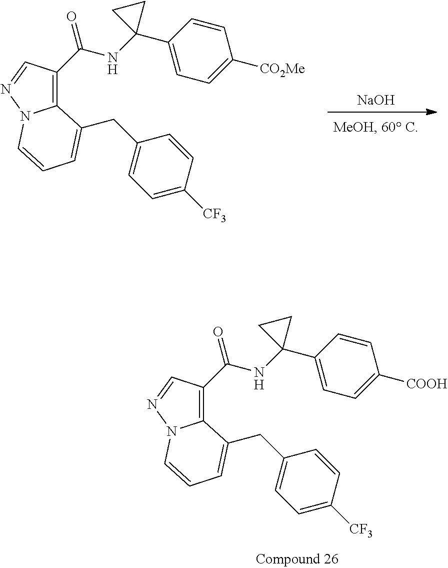 [SMILES: COC(=O)c1ccc(C2(NC(=O)c3cnn4cccc(Cc5ccc(C(F)(F)F)cc5)c34)CC2)cc1 ; "NaOH" ; O=C(O)c1ccc(C2(NC(=O)c3cnn4cccc(Cc5ccc(C(F)(F)F)cc5)c34)CC2)cc1]

To a solution of methyl 4-[1-[[4-[[4-(trifluoromethyl) phenyl]methyl]pyrazolo[1,5-a]pyridine-3-carbonyl]amino]cyclopropyl]benzoate (20 mg, 40.53 umol, 1 eq) in H₂O (1 mL) and MeOH (1 mL) was added NaOH (4.86 mg, 121.59 umol, 3 eq). The mixture was stirred at 50° C. for 16 hr. The reaction mixture was concentrated under reduced pressure to remove MeOH. The mixture was adjusted to PH 4 with 1N HCl. The mixture was filtered. Compound 4-[1-[[4-[[4-(trifluoromethyl)phenyl]methyl]pyrazolo[1,5-a]pyridine-3-carbonyl]amino]cyclopropyl]benzoic acid (7 mg, 14.05 umol, 34.68% yield, 96.26% purity) was obtained as a white solid. LCMS for product (ESI): m/z 480.0 [M+H]⁺¹H NMR (400 MHz, DMSO-d₆) δ ppm 12.78 (br s, 1H) 9.01 (s, 1H) 8.74 (d, J=7.2 Hz, 1H) 8.46 (s, 1H) 7.82 (d, J=8.0 Hz, 2H) 7.53 (d, J=8.4 Hz, 2H) 7.31 (d, J=7.2 Hz, 1H) 7.23-7.29 (m, 4H) 7.07 (t, J=6.8 Hz, 1H) 4.69 (s, 2H) 1.25-1.23 (m, 4H).

### Example 31—Synthesis of 4-[(1R)-1-[[4-[[4-(trifluoromethyl) phenyl]methyl]pyrazolo[1,5-a]pyridine-3-carbonyl]amino]ethyl]benzoic acid (Compound 27)

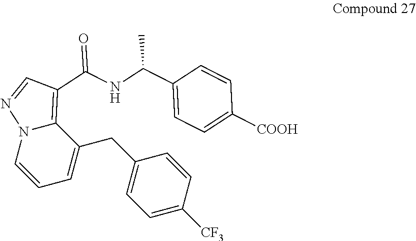 [SMILES: C[C@@H](NC(=O)c1cnn2cccc(Cc3ccc(C(F)(F)F)cc3)c12)c1ccc(C(=O)O)cc1]

### Step 1: methyl 4-[(1R)-1-[[4-[[4-(trifluoromethyl) phenyl]methyl]pyrazolo[1,5-a]pyridine-3-carbonyl]amino]ethyl]benzoate

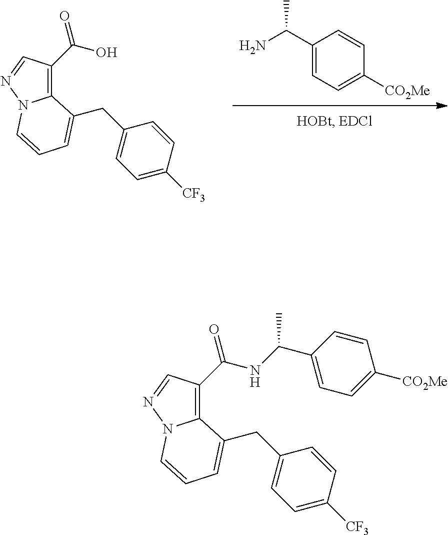 [SMILES: O=C(O)c1cnn2cccc(Cc3ccc(C(F)(F)F)cc3)c12 ; COC(=O)c1ccc([C@@H](C)N)cc1 ; COC(=O)c1ccc([C@@H](C)NC(=O)c2cnn3cccc(Cc4ccc(C(F)(F)F)cc4)c23)cc1]

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added HOBt (1.69 mg, 12.49 umol, 0.2 eq), Et₃N (31.60 mg, 312.24 umol, 43.46 uL, 5 eq) and EDCI (23.94 mg, 124.90 umol, 2 eq) at 0° C. The mixture was stirred at 0° C. for 0.5 hr. Then methyl 4-[(1R)-1-aminoethyl]benzoate (12.31 mg, 68.69 umol, 1.1 eq) was added at 0° C. The mixture was stirred at 25° C. for 16 hr. Several new peaks were shown on LC-MS and 74.29% of desired compound was detected. The reaction mixture was diluted with H₂O 9 mL and extracted with DCM 12 mL (4 mL×3). The combined organic layers were washed with brine 9 mL, dried over Na₂SO₄, filtered and concentrated under reduced pressure to give a residue. The residue was purified by prep-TLC (SiO₂, PE:EA=1:1). Compound methyl 4-[(1R)-1-[[4-[[4-(trifluoromethyl) phenyl]methyl]pyrazolo[1,5-a]pyridine-3-carbonyl]amino]ethyl]benzoate (11 mg, 22.85 umol, 36.59% yield) was obtained as a white solid, which was used into the next step directly without further purification. LCMS for product (ESI): m/z 482.1 [M+H]⁺.

### Step 2: 4-[(1R)-1-[[4-[[4-(trifluoromethyl) phenyl]methyl]pyrazolo[1,5-a]pyridine-3-carbonyl]amino]ethyl]benzoic acid

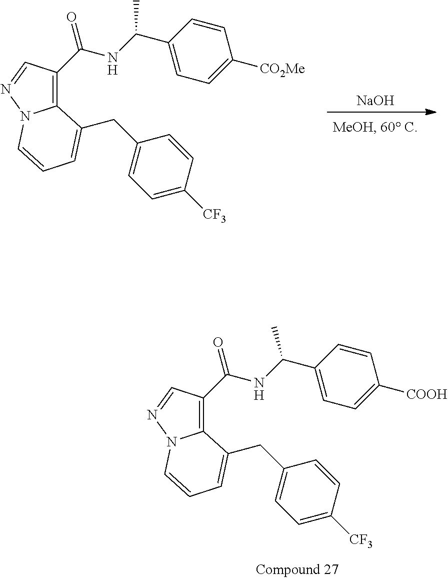 [SMILES: COC(=O)c1ccc([C@@H](C)NC(=O)c2cnn3cccc(Cc4ccc(C(F)(F)F)cc4)c23)cc1 ; "NaOH" ; C[C@@H](NC(=O)c1cnn2cccc(Cc3ccc(C(F)(F)F)cc3)c12)c1ccc(C(=O)O)cc1]

To a solution of methyl 4-[(1R)-1-[[4-[[4-(trifluoromethyl) phenyl]methyl]pyrazolo[1,5-a]pyridine-3-carbonyl]amino]ethyl]benzoate (11 mg, 22.85 umol, 1 eq) in H₂O (1 mL) and MeOH (1 mL) was added NaOH (2.74 mg, 68.54 umol, 3 eq). The mixture was stirred at 50° C. for 16 hr. The reaction mixture was concentrated under reduced pressure to remove MeOH. The mixture was adjusted to pH 4 with 1N HCl. The mixture was filtered. Compound 4-[(1R)-1-[[4-[[4-(trifluoromethyl) phenyl]methyl]pyrazolo[1,5-a]pyridine-3-carbonyl]amino]ethyl]benzoic acid (3 mg, 5.97 umol, 26.14% yield, 93.04% purity) was obtained as a white solid. LCMS for product (ESI): m/z 468.0 [M+H]⁺. ¹H NMR (400 MHz, DMSO-d₆) δ=12.85 (br s, 1H), 8.75-8.69 (m, 2H), 8.35 (s, 1H), 7.92 (d, J=8.4 Hz, 2H), 7.52 (d, J=8.0 Hz, 2H), 7.40-7.34 (m, 3H), 7.20 (d, J=8.0 Hz, 2H), 7.06 (t, J=6.8 Hz, 1H), 5.22-5.15 (m, 1H), 4.71 (d, J=14.8 Hz, 1H), 4.51 (d, J=14.8 Hz, 1H), 1.43 (d, J=7.2 Hz, 3H).

### Example 32—Synthesis of 2-[4-[[4-[[4-(trifluoromethyl)phenyl]methyl]pyrazolo[1,5-a]pyridine-3-carbonyl]amino]phenyl]acetic acid (Compound 28)

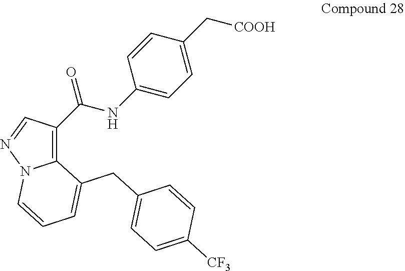 [SMILES: O=C(O)Cc1ccc(NC(=O)c2cnn3cccc(Cc4ccc(C(F)(F)F)cc4)c23)cc1]

### Step 1: ethyl 2-[4-[[4-[[4-(trifluoromethyl)phenyl]methyl]pyrazolo[1,5-a]pyridine-3-carbonyl]amino]phenyl]acetate

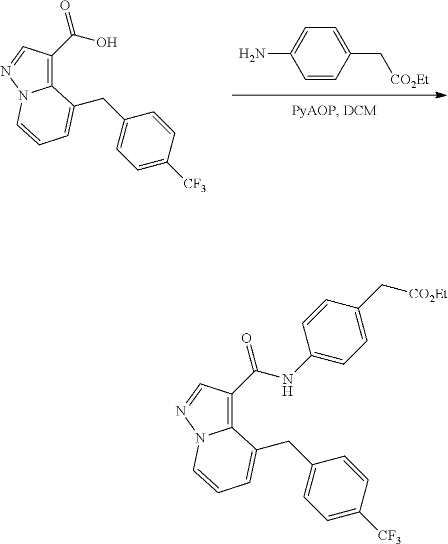 [SMILES: O=C(O)c1cnn2cccc(Cc3ccc(C(F)(F)F)cc3)c12 ; CCOC(=O)Cc1ccc(N)cc1 ; CCOC(=O)Cc1ccc(NC(=O)c2cnn3cccc(Cc4ccc(C(F)(F)F)cc4)c23)cc1]

To a solution of 4-[[4-(trifluoromethyl)phenyl]methyl]pyrazolo[1,5-a]pyridine-3-carboxylic acid (30 mg, 93.67 umol, 1 eq) and ethyl 2-(4-aminophenyl)acetate (16.79 mg, 93.67 umol, 1 eq) in DCM (1 mL) was added PYAOP (53.72 mg, 103.04 umol, 1.1 eq) and DIEA (30.27 mg, 234.18 umol, 40.79 uL, 2.5 eq). The mixture was stirred at 25° C. for 5 hr. LC-MS showed 0% of the benzoic acid was remained. Several new peaks were shown on LC-MS and 76.01% of desired compound was detected. The reaction mixture was diluted with H₂O 10 mL and extracted with DCM 12 mL (4 mL×3). The combined organic layers were washed with brine 9 mL, dried over Na₂SO₄, filtered and concentrated under reduced pressure to give a residue. The residue was purified by prep-TLC (SiO₂, PE:EA=1:1). Compound ethyl 2-[4-[[4-[[4-(trifluoromethyl)phenyl]methyl]pyrazolo[1,5-a]pyridine-3-carbonyl]amino]phenyl]acetate (38 mg, 78.93 umol, 84.26% yield) was obtained as yellow oil, which was used into the next step directly without further purification. LCMS for product (ESI): m/z 482.1 [M+H]⁺.

### Step 2: 2-[4-[[4-[[4-(trifluoromethyl)phenyl]methyl]pyrazolo[1,5-a]pyridine-3-carbonyl]amino]phenyl]acetic acid

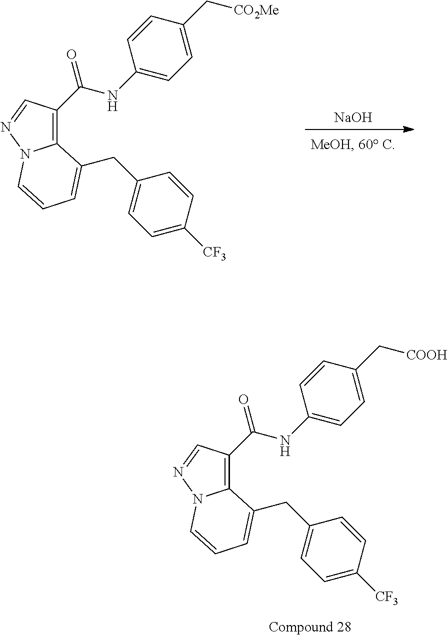 [SMILES: COC(=O)Cc1ccc(NC(=O)c2cnn3cccc(Cc4ccc(C(F)(F)F)cc4)c23)cc1 ; "NaOH" ; O=C(O)Cc1ccc(NC(=O)c2cnn3cccc(Cc4ccc(C(F)(F)F)cc4)c23)cc1]

To a solution of ethyl 2-[4-[[4-[[4-(trifluoromethyl)phenyl]methyl]pyrazolo[1,5-a]pyridine-3-carbonyl]amino]phenyl]acetate (38 mg, 78.93 umol, 1 eq) in H₂O (1 mL) and MeOH (1 mL) was added NaOH (9.47 mg, 236.78 umol, 3 eq). The mixture was stirred at 50° C. for 16 hr. The reaction mixture was concentrated under reduced pressure to remove MeOH. The mixture was adjusted to pH 4 with 1N HCl. The mixture was filtered. Compound 2-[4-[[4-[[4-(trifluoromethyl)phenyl]methyl]pyrazolo[1,5-a]pyridine-3-carbonyl]amino]phenyl]acetic acid (13 mg, 28.49 umol, 36.10% yield, 99.37% purity) was obtained as a white solid. LCMS for product (ESI): m/z 454.0 [M+H]⁺. ¹H NMR (400 MHz, DMSO-d₆) δ ppm 12.31 (br s, 1H) 10.16 (s, 1H) 8.76 (d, J=6.8 Hz, 1H) 8.42 (s, 1H) 7.62 (d, J=8.4 Hz, 2H) 7.52 (d, J=8.0 Hz, 2H) 7.34 (d, J=7.6 Hz, 3H) 7.23 (d, J=8.4 Hz, 2H) 7.10 (t, J=6.94 Hz, 1H) 4.65 (s, 2H) 3.55 (s, 2H).

### Example 33—Synthesis of 4-[(1S)-1-[[2-isopropyl-4-[[4-(trifluoromethyl) phenyl]methyl]indazole-3-carbonyl]amino]ethyl]benzoic acid (Compound 29)

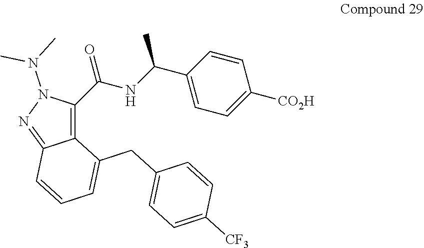 [SMILES: C[C@H](NC(=O)c1c2c(Cc3ccc(C(F)(F)F)cc3)cccc2nn1N(C)C)c1ccc(C(=O)O)cc1]

### Step 1: methyl 2-isopropyl-4-[[4-(trifluoromethyl)phenyl]methyl]indazole-3-carboxylate

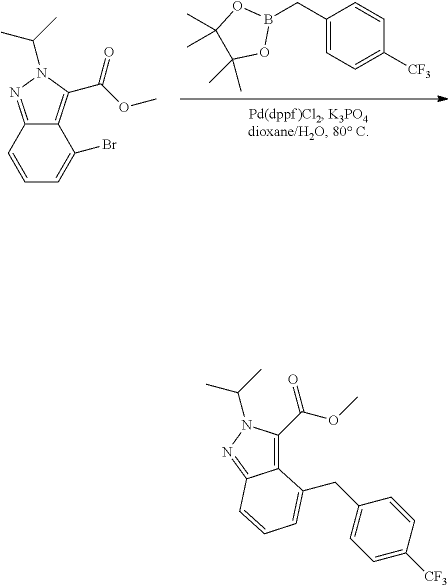 [SMILES: COC(=O)c1c2c(Br)cccc2nn1C(C)C ; CC1(C)OB(Cc2ccc(C(F)(F)F)cc2)OC1(C)C ; COC(=O)c1c2c(Cc3ccc(C(F)(F)F)cc3)cccc2nn1C(C)C]

To a solution of 4,4,5,5-tetramethyl-2-[[4-(trifluoromethyl) phenyl]methyl]-1,3,2-dioxaborolane (770.25 mg, 2.69 mmol, 2 eq) and methyl 4-bromo-2-isopropyl-indazole-3-carboxylate (400.00 mg, 1.35 mmol, 1 eq) in H₂O (2 mL) and dioxane (10 mL) was added K₃PO₄ (285.74 mg, 1.35 mmol, 1 eq) and Pd (dppf) Cl₂ (295.49 mg, 403.84 umol, 0.3 eq). The mixture was stirred at 80° C. for 16 hr. The reaction mixture was diluted with H₂O 20 mL and extracted with DCM 45 mL (15 mL×3). The combined organic layers were washed with brine 30 mL, dried over Na₂SO₄, filtered and concentrated under reduced pressure to give a residue. The residue was purified by prep-TLC (SiO₂, PE:EA=1:1). Compound methyl 2-isopropyl-4-[[4-(trifluoromethyl) phenyl]methyl]indazole-3-carboxylate (320 mg, 850.22 umol, 63.16% yield) was obtained as a white solid. LCMS for crude product (ESI): m/z 377.3 [M+H]⁺.

### Step 2: 2-isopropyl-4-[[4-(trifluoromethyl) phenyl]methyl]indazole-3-carboxylic acid

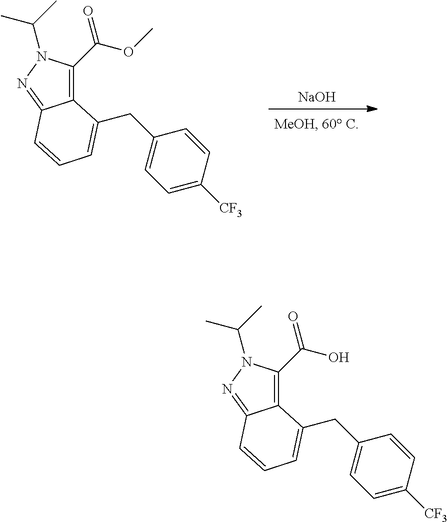 [SMILES: COC(=O)c1c2c(Cc3ccc(C(F)(F)F)cc3)cccc2nn1C(C)C ; "NaOH" ; CC(C)n1nc2cccc(Cc3ccc(C(F)(F)F)cc3)c2c1C(=O)O]

To a solution of methyl 2-isopropyl-4-[[4-(trifluoromethyl) phenyl]methyl]indazole-3-carboxylate (300 mg, 797.08 umol, 1 eq) in H₂O (5 mL) and MeOH (3 mL) was added NaOH (95.65 mg, 2.39 mmol, 3 eq). The mixture was stirred at 50° C. for 16 hr. The reaction mixture was concentrated under reduced pressure to remove MeOH. The mixture was adjusted to PH 4 with 1N HCl. The mixture was filtered and the filter cake was dried to give compound 2-isopropyl-4-[[4-(trifluoromethyl) phenyl]methyl]indazole-3-carboxylic acid (200 mg, 551.96 umol, 69.25% yield) was obtained as a white solid.

### Step 3: methyl 4-[(1S)-1-[[2-isopropyl-4-[[4-(trifluoromethyl)phenyl]methyl]indazole-3-carbonyl]amino]ethyl]benzoate

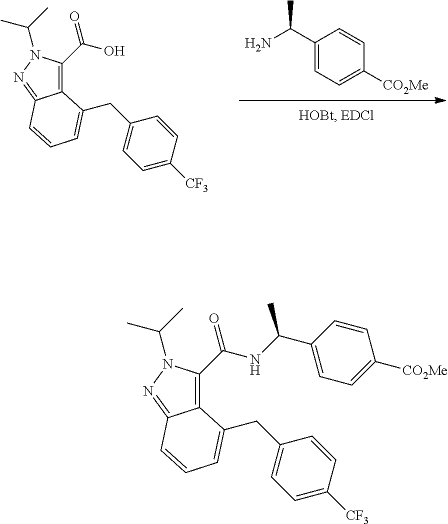 [SMILES: CC(C)n1nc2cccc(Cc3ccc(C(F)(F)F)cc3)c2c1C(=O)O ; COC(=O)c1ccc([C@H](C)N)cc1 ; COC(=O)c1ccc([C@H](C)NC(=O)c2c3c(Cc4ccc(C(F)(F)F)cc4)cccc3nn2C(C)C)cc1]

To a solution of methyl 4-[(1S)-1-aminoethyl]benzoate (27.20 mg, 151.79 umol, 1.1 eq) in DCM (2 mL) was added HOBt (3.73 mg, 27.60 umol, 0.2 eq), Et₃N (69.82 mg, 689.95 umol, 96.03 uL, 5 eq) and EDCI (52.91 mg, 275.98 umol, 2 eq). The mixture was stirred at 0° C. for 0.5 hr. Then 2-isopropyl-4-[[4-(trifluoromethyl) phenyl]methyl]indazole-3-carboxylic acid (50.00 mg, 137.99 umol, 1 eq) was added at 0° C. The mixture was stirred at 25° C. for 16 hr. The reaction mixture was diluted with H₂O 5 mL and extracted with DCM 15 mL (5 mL×3). The combined organic layers were washed with brine 10 mL, dried over Na₂SO₄, filtered and concentrated under reduced pressure to give a residue. The residue was purified by prep-TLC (SiO₂, PE:EA=1:1). Compound methyl 4-[(1S)-1-[[2-isopropyl-4-[[4-(trifluoromethyl)phenyl]methyl]indazole-3-carbonyl]amino]ethyl]benzoate (40 mg, 76.40 umol, 55.37% yield) was obtained as a white solid, which was used into the next step directly without further purification. LCMS for product (ESI): m/z 524.3 [M+H]⁺.

### Step 4: 4-[(1S)-1-[[2-isopropyl-4-[[4-(trifluoromethyl) phenyl]methyl]indazole-3-carbonyl]amino]ethyl]benzoic acid

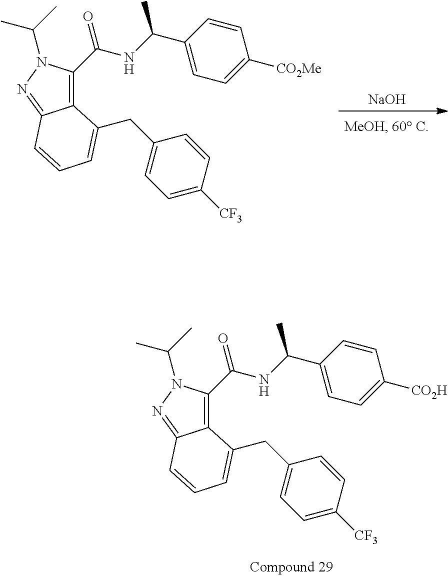 [SMILES: COC(=O)c1ccc([C@H](C)NC(=O)c2c3c(Cc4ccc(C(F)(F)F)cc4)cccc3nn2C(C)C)cc1 ; "NaOH" ; CC(C)n1nc2cccc(Cc3ccc(C(F)(F)F)cc3)c2c1C(=O)N[C@@H](C)c1ccc(C(=O)O)cc1]

To a solution of methyl 4-[(1S)-1-[[2-isopropyl-4-[[4-(trifluoromethyl)phenyl]methyl]indazole-3-carbonyl]amino]ethyl]benzoate (40 mg, 76.40 umol, 1 eq) in H₂O (2 mL) and MeOH (2 mL) was added NaOH (9.17 mg, 229.21 umol, 3 eq). The mixture was stirred at 50° C. for 16 hr. The reaction mixture was concentrated under reduced pressure to remove MeOH. The mixture was adjusted to PH 4 with 1N HCl. The mixture was filtered and the filter cake was dried to give compound 4-[(1S)-1-[[2-isopropyl-4-[[4-(trifluoromethyl) phenyl]methyl]indazole-3-carbonyl]amino]ethyl]benzoic acid (35 mg, 67.52 umol, 88.38% yield, 98.30% purity) was obtained as a white solid. LCMS for product (ESI): m/z 510.1 [M+H]⁺. ¹H NMR (400 MHz, DMSO-d₆) δ ppm 12.89 (br s, 1H), 9.60 (d, J=7.6 Hz, 1H), 7.90 (d, J=8.4 Hz, 2H), 7.59-7.56 (m, 3H), 7.51 (d, J=7.2 Hz, 2H), 7.26 (d, J=8.0 Hz, 2H), 7.22 (d, J=8.0 Hz, 1H), 6.74 (d, J=6.8 Hz, 1H), 5.23 (t, J=7.2 Hz, 1H), 4.76-4.69 (m, 1H), 4.17 (s, 2H), 1.52 (d, J=6.8 Hz, 3H), 1.47 (d, J=6.8 Hz, 3H), 1.42 (d, J=7.2 Hz, 3H).

### Example 34—Synthesis of 4-[(1S)-1-[[1-isobutyl-4-[[4-(trifluoromethyl) phenyl]methyl]indazole-3-carbonyl]amino]ethyl]benzoic acid (Compound 30)

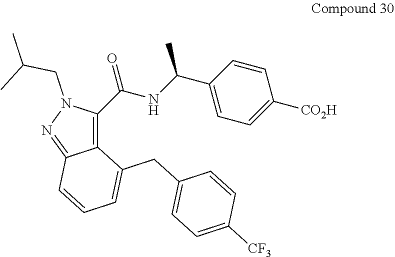 [SMILES: CC(C)Cn1nc2cccc(Cc3ccc(C(F)(F)F)cc3)c2c1C(=O)N[C@@H](C)c1ccc(C(=O)O)cc1]

### Step 1: methyl 2-isobutyl-4-[[4-(trifluoromethyl) phenyl]methyl]indazole-3-carboxylate

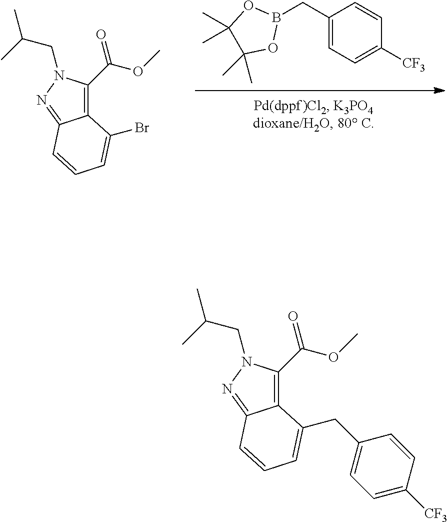 [SMILES: COC(=O)c1c2c(Br)cccc2nn1CC(C)C ; CC1(C)OB(Cc2ccc(C(F)(F)F)cc2)OC1(C)C ; COC(=O)c1c2c(Cc3ccc(C(F)(F)F)cc3)cccc2nn1CC(C)C]

A mixture of methyl 4-bromo-2-isobutyl-indazole-3-carboxylate (200.00 mg, 642.73 umol, 1 eq), 4,4,5,5-tetramethyl-2-[[4-(trifluoromethyl) phenyl]methyl]-1,3,2-dioxaborolane (367.76 mg, 1.29 mmol, 2 eq), K₃PO₄ (136.43 mg, 642.73 umol, 1 eq), Pd(dppf)Cl₂ (141.09 mg, 192.82 umol, 0.3 eq) in dioxane (1 mL) and H₂O (0.5 mL) was degassed and purged with N₂ for 3 times, and then the mixture was stirred at 80° C. for 12 hrunder N₂ atmosphere. Several new peaks were shown on LC-MS and 75% of desired compound was detected. The reaction mixture was concentrated under reduced pressure to remove dioxane. The residue was diluted with H₂O 30 mL and extracted with EA 30 mL (10 mL×3). The combined organic layers were washed with brine 15 mL, dried over Na₂SO₄, filtered and concentrated under reduced pressure to give a residue. The residue was purified by prep-TLC (SiO₂, PE: EA=3:1). Compound methyl 2-isobutyl-4-[[4-(trifluoromethyl) phenyl]methyl]indazole-3-carboxylate (170 mg, 435.45 umol, 67.75% yield) was obtained as a white solid. LCMS for product (ESI): m/z 391.3 [M+H]⁺.

### Step 2: 2-isobutyl-4-[[4-(trifluoromethyl) phenyl]methyl]indazole-3-carboxylic acid

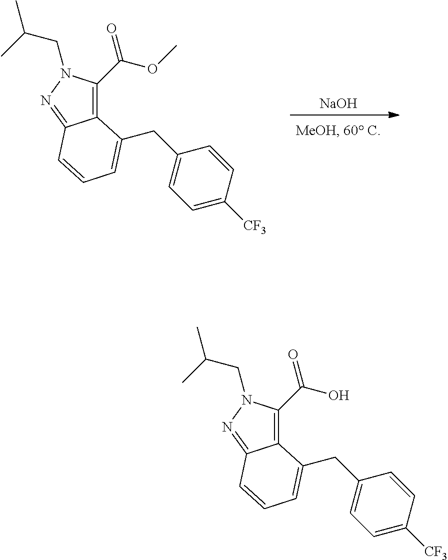 [SMILES: COC(=O)c1c2c(Cc3ccc(C(F)(F)F)cc3)cccc2nn1CC(C)C ; "NaOH" ; CC(C)Cn1nc2cccc(Cc3ccc(C(F)(F)F)cc3)c2c1C(=O)O]

To a solution of methyl 2-isobutyl-4-[[4-(trifluoromethyl) phenyl]methyl]indazole-3-carboxylate (170.00 mg, 435.45 umol, 1 eq) in H₂O (0.5 mL) and MeOH (3 mL) was added NaOH (52.26 mg, 1.3 mmol, 3 eq). The mixture was stirred at 80° C. for 3 hr. The reaction was clean according to TLC. The reaction mixture was concentrated under reduced pressure to remove MeOH. The mixture was adjusted to pH 4 with 1N HCl. The mixture was filtered. Compound 2-isobutyl-4-[[4-(trifluoromethyl) phenyl]methyl]indazole-3-carboxylic acid (100 mg, 265.69 umol, 61.02% yield) was obtained as a white solid.

### Step 3: methyl 4-[(1S)-1-[[2-isobutyl-4-[[4-(trifluoromethyl) phenyl]methyl]indazole-3-carbonyl]amino]ethyl]benzoate

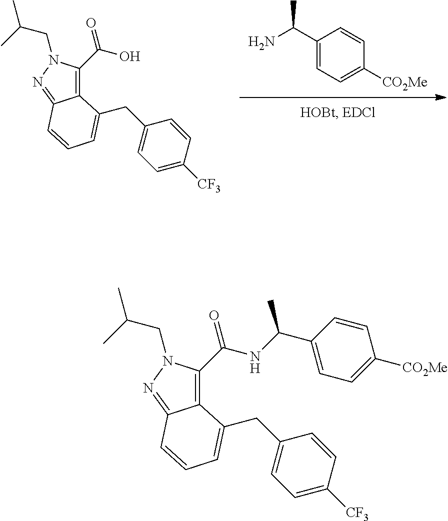 [SMILES: CC(C)Cn1nc2cccc(Cc3ccc(C(F)(F)F)cc3)c2c1C(=O)O ; COC(=O)c1ccc([C@H](C)N)cc1 ; COC(=O)c1ccc([C@H](C)NC(=O)c2c3c(Cc4ccc(C(F)(F)F)cc4)cccc3nn2CC(C)C)cc1]

To a solution of 2-isobutyl-4-[[4-(trifluoromethyl) phenyl]methyl]indazole-3-carboxylic acid (30.00 mg, 79.71 umol, 1 eq) in DCM (2 mL) was added dropwise Et₃N (40.33 mg, 398.54 umol, 55.47 uL, 5 eq), HOBt (2.15 mg, 15.94 umol, 0.2 eq) and EDCI (30.56 mg, 159.42 umol, 2 eq) at 0° C. After addition, the mixture was stirred at this temperature for 0.5 hr, and then methyl 4-[(1S)-1-aminoethyl]benzoate (21.43 mg, 119.56 umol, 1.5 eq) was added dropwise at 0° C. The resulting mixture was stirred at 25° C. for 12 hr. The residue was diluted with H₂O 5 mL and extracted with DCM 15 mL (5 mL×3). The combined organic layers were washed with brine 5 mL, dried over Na₂SO₄, filtered and concentrated under reduced pressure to give a residue. The residue was purified by prep-TLC (SiO2, PE:EA=1:1). Compound methyl 4-[(1S)-1-[[2-isobutyl-4-[[4-(trifluoromethyl) phenyl]methyl]indazole-3-carbonyl]amino]ethyl]benzoate (20 mg, 37.20 umol, 46.68% yield) was obtained as a white solid, which was used into the next step directly without further purification. LCMS for product (ESI): m/z 538.2 [M+H]⁺.

### Step 4: 4-[(1S)-1-[[2-isobutyl-4-[[4-(trifluoromethyl) phenyl]methyl]indazole-3-carbonyl]amino]ethyl]benzoic acid

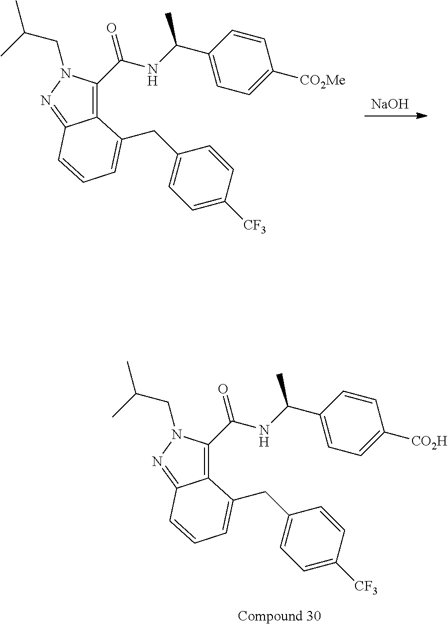 [SMILES: COC(=O)c1ccc([C@H](C)NC(=O)c2c3c(Cc4ccc(C(F)(F)F)cc4)cccc3nn2CC(C)C)cc1 ; "NaOH" ; CC(C)Cn1nc2cccc(Cc3ccc(C(F)(F)F)cc3)c2c1C(=O)N[C@@H](C)c1ccc(C(=O)O)cc1]

To a solution of methyl 4-[(1S)-1-[[2-isobutyl-4-[[4-(trifluoromethyl) phenyl]methyl]indazole-3-carbonyl]amino]ethyl]benzoate (20 mg, 37.20 umol, 1 eq) in H₂O (2 mL) and MeOH (2 mL) was added NaOH (1.49 mg, 37.20 umol, 1 eq). The mixture was stirred at 50° C. for 12 hr. The reaction mixture was concentrated under reduced pressure to remove solvent. The mixture was adjusted to pH=5 with 1N HCl. The mixture was filtered. Compound 4-[(1S)-1-[[2-isobutyl-4-[[4-(trifluoromethyl) phenyl]methyl]indazole-3-carbonyl]amino]ethyl]benzoic acid (8 mg, 15.28 umol, 41.07% yield, 100% purity) was obtained as a white solid. LCMS for product (ESI): m/z 524.1 [M+H]⁺. ¹H NMR (400 MHz, DMSO-d₆) δ=9.41 (d, J=8.0 Hz, 1H), 7.87 (d, J=8.0 Hz, 2H), 7.55 (t, J=8.0 Hz, 3H), 7.40 (d, J=7.6 Hz, 2H), 7.27-7.20 (m, 3H), 6.85 (d, J=6.8 Hz, 1H), 5.17-5.09 (m, 1H), 4.32 (d, J=15.6 Hz, 1H), 4.22 (d, J=15.6 Hz, 1H), 4.14-4.07 (m, 2H), 2.04-1.97 (m, 1H), 1.34 (d, J=7.2 Hz, 3H), 0.68-0.66 (d, J=6.8 Hz, 3H), 0.62-0.60 (d, J=6.4 Hz, 3H).

### Example 35—Synthesis of 1-[4-[[4-[[4-(trifluoromethyl)phenyl]methyl]pyrazolo[1,5-a]pyridine-3-carbonyl]amino]phenyl]cyclopropanecarboxylic acid (Compound 31)

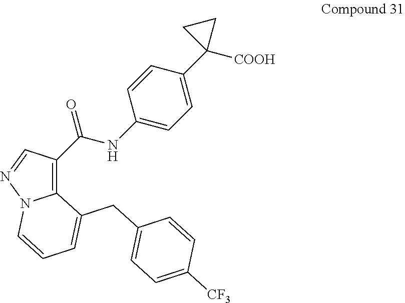 [SMILES: O=C(Nc1ccc(C2(C(=O)O)CC2)cc1)c1cnn2cccc(Cc3ccc(C(F)(F)F)cc3)c12]

### Step 1: methyl 1-[4-[[4-[[4-(trifluoromethyl) phenyl]methyl]pyrazolo[1,5-a]pyridine-3-carbonyl]amino]phenyl]cyclopropanecarboxylate

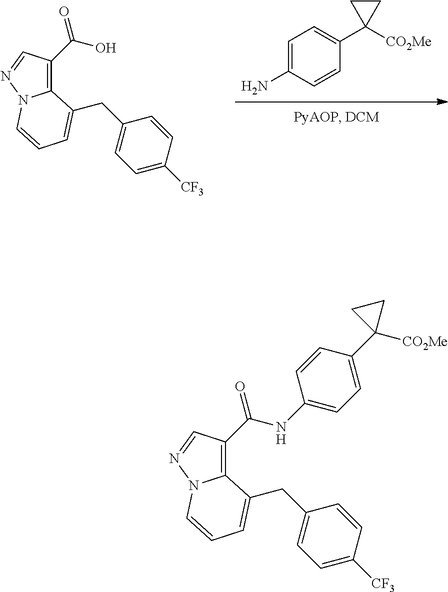 [SMILES: O=C(O)c1cnn2cccc(Cc3ccc(C(F)(F)F)cc3)c12 ; COC(=O)C1(c2ccc(N)cc2)CC1 ; COC(=O)C1(c2ccc(NC(=O)c3cnn4cccc(Cc5ccc(C(F)(F)F)cc5)c34)cc2)CC1]

To a solution of 4-[[4-(trifluoromethyl) phenyl]methyl]pyrazolo[1,5-a]pyridine-3-carboxylic acid (30 mg, 93.67 umol, 1 eq) and methyl 1-(4-aminophenyl) cyclopropanecarboxylate (19.70 mg, 103.04 umol, 1.1 eq) in DCM (1 mL) was added PYAOP (53.72 mg, 103.04 umol, 1.1 eq) and DIEA (30.27 mg, 234.18 umol, 40.79 uL, 2.5 eq). The mixture was stirred at 25° C. for 5 hr. Several new peaks were shown on LC-MS and 75.50% of desired compound was detected. The reaction mixture was diluted with H₂O 10 mL and extracted with DCM 9 mL (3 mL×3). The combined organic layers were washed with brine 5 mL, dried over Na₂SO₄, filtered and concentrated under reduced pressure to give a residue. The residue was purified by prep-TLC (SiO₂, PE:EA=1:1). Compound methyl 1-[4-[[4-[[4-(trifluoromethyl) phenyl]methyl]pyrazolo[1,5-a]pyridine-3-carbonyl]amino]phenyl]cyclopropanecarboxylate (40 mg, 81.06 umol, 86.53% yield) was obtained as yellow oil, which was used into the next step directly without further purification. LCMS for product (ESI): m/z 494.1 [M+H]⁺.

### Step 2: 1-[4-[[4-[[4-(trifluoromethyl)phenyl]methyl]pyrazolo[1,5-a]pyridine-3-carbonyl]amino]phenyl]cyclopropanecarboxylic acid

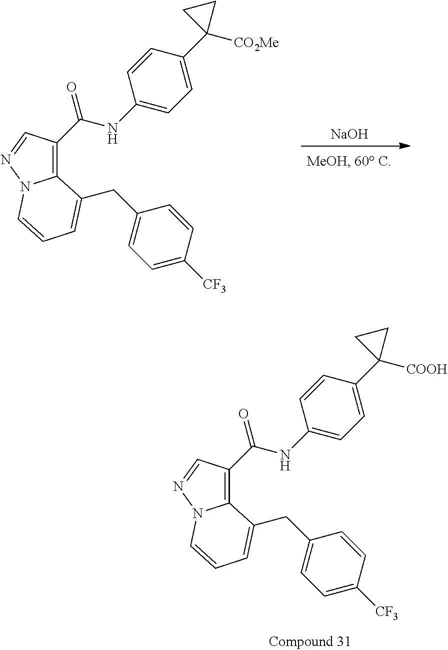 [SMILES: COC(=O)C1(c2ccc(NC(=O)c3cnn4cccc(Cc5ccc(C(F)(F)F)cc5)c34)cc2)CC1 ; "NaOH" ; O=C(Nc1ccc(C2(C(=O)O)CC2)cc1)c1cnn2cccc(Cc3ccc(C(F)(F)F)cc3)c12]

To a solution of methyl 1-[4-[[4-[[4-(trifluoromethyl) phenyl]methyl]pyrazolo[1,5-a]pyridine-3-carbonyl]amino]phenyl]cyclopropanecarboxylate (40 mg, 81.06 umol, 1 eq) in H₂O (1 mL) and MeOH (1 mL) was added NaOH (9.73 mg, 243.17 umol, 3 eq). The mixture was stirred at 50° C. for 16 hr. HPLC showed 0% of S1-031-A was remained. The reaction mixture was concentrated under reduced pressure to remove MeOH. The mixture was adjusted to pH 4 with 1N HCl. The mixture was filtered. Compound 1-[4-[[4-[[4-(trifluoromethyl)phenyl]methyl]pyrazolo[1,5-a]pyridine-3-carbonyl]amino]phenyl]cyclopropanecarboxylic acid (30 mg, 62.57 umol, 77.19% yield) was obtained as a white solid. LCMS for product (ESI): m/z 480.0 [M+H]⁺. ¹H NMR (400 MHz, DMSO-d₆) δ ppm 10.16 (s, 1H) 8.76 (d, J=6.8 Hz, 1H) 8.42 (s, 1H) 7.59 (d, J=8.4 Hz, 2H) 7.52 (d, J=8.0 Hz, 2H) 7.34 (br d, J=8.4 Hz, 3H) 7.29 (d, J=8.4 Hz, 2H) 7.10 (t, J=6.8 Hz, 1H) 4.65 (s, 2H) 1.42-1.47 (m, 2H) 1.10-1.14 (m, 2H).

### Example 36—Synthesis of (1s,4s)-4-[[4-[[4-(trifluoromethyl)phenyl]methyl]pyrazolo[1,5-a]pyridine-3-carbonyl]amino]cyclohexanecarboxylic acid (Compound 32)

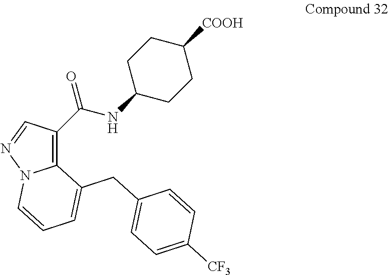 [SMILES: O=C(N[C@H]1CC[C@@H](C(=O)O)CC1)c1cnn2cccc(Cc3ccc(C(F)(F)F)cc3)c12]

### Step 1: (2,5-dioxopyrrolidin-1-yl) 4-[[4-(trifluoromethyl)phenyl]methyl]pyrazolo[1,5-a]pyridine-3-carboxylate

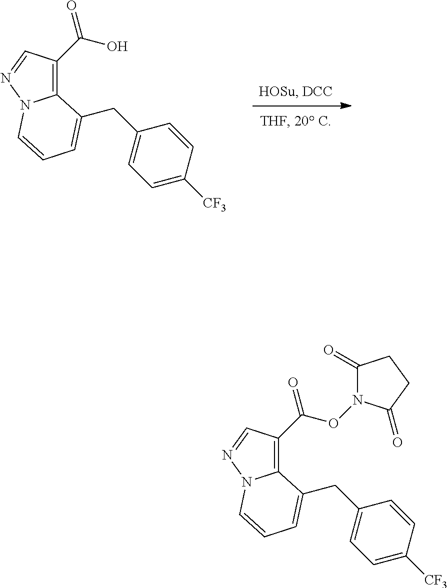 [SMILES: O=C(O)c1cnn2cccc(Cc3ccc(C(F)(F)F)cc3)c12 ; "HOSu, DCC" ; O=C(ON1C(=O)CCC1=O)c1cnn2cccc(Cc3ccc(C(F)(F)F)cc3)c12]

To a solution of 4-[[4-(trifluoromethyl)phenyl]methyl]pyrazolo[1,5-a]pyridine-3-carboxylic acid (100 mg, 312.24 umol, 1 eq) and 1-hydroxypyrrolidine-2,5-dione (43.12 mg, 374.69 umol, 1.2 eq) in THF (1 mL) was added DCC (83.75 mg, 405.91 umol, 82.11 uL, 1.3 eq). The mixture was stirred at 25° C. for 16 hr. Several new peaks were shown on LC-MS and 50.43% of desired compound was detected. The reaction mixture was diluted with H₂O 10 mL and extracted with DCM 12 mL (4 mL×3). The combined organic layers were washed with brine 5 mL, dried over Na₂SO₄, filtered and concentrated under reduced pressure to give a residue. The crude product was used into the next step without further purification. Compound (2,5-dioxopyrrolidin-1-yl) 4-[[4-(trifluoromethyl)phenyl]methyl]pyrazolo[1,5-a]pyridine-3-carboxylate (100 mg, 239.61 umol, 76.74% yield) was obtained as yellow oil, which used to next step directly without further purification. LCMS for product (ESI): m/z 418.2 [M+H]⁺.

### Step 2: (1s,4s)-4-[[4-[[4-(trifluoromethyl)phenyl]methyl]pyrazolo[1,5-a]pyridine-3-carbonyl]amino]cyclohexanecarboxylic acid

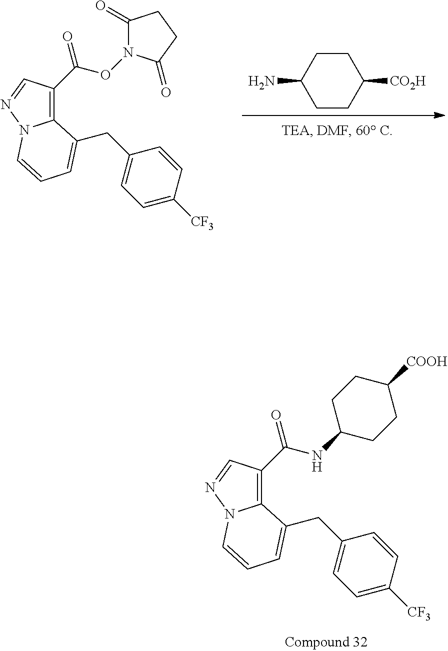 [SMILES: O=C(ON1C(=O)CCC1=O)c1cnn2cccc(Cc3ccc(C(F)(F)F)cc3)c12 ; N[C@H]1CC[C@@H](C(=O)O)CC1 ; O=C(N[C@H]1CC[C@@H](C(=O)O)CC1)c1cnn2cccc(Cc3ccc(C(F)(F)F)cc3)c12]

To a solution of (2,5-dioxopyrrolidin-1-yl) 4-[[4-(trifluoromethyl)phenyl]methyl]pyrazolo[1,5-a]pyridine-3-carboxylate (35 mg, 83.86 umol, 1 eq) and trans-4-aminocyclohexanecarboxylic acid (60.04 mg, 419.32 umol, 5 eq) in DMF (1 mL) was added Et₃N (152.75 mg, 1.51 mmol, 210.11 uL, 18 eq). The mixture was stirred at 60° C. for 16 hr. Several new peaks were shown on LC-MS and 2.81% of desired compound was detected. The reaction mixture was concentrated under reduced pressure to remove MeOH. The mixture was adjusted to pH 4 with 1N HCl. The mixture was filtered. Compound (1s,4s)-4-[[4-[[4-(trifluoromethyl)phenyl]methyl]pyrazolo[1,5-a]pyridine-3-carbonyl]amino]cyclohexanecarboxylic acid (10 mg, 22.19 umol, 26.45% yield, 98.82% purity) was obtained as a white solid. LCMS for product (ESI): m/z 446.0 [M+H]⁺. ¹H NMR (400 MHz, DMSO-d₆) δ ppm 12.10 (br s, 1H) 8.69 (d, J=6.8 Hz, 1H) 8.20 (s, 1H) 8.02 (d, J=7.6 Hz, 1H) 7.56 (d, J=8.0 Hz, 2H) 7.34-7.29 (m, 3H) 7.04 (t, J=6.8 Hz, 1H) 4.65 (s, 2H) 3.79-3.73 (m, 1H) 2.51-2.42 (m, 1H) 1.93-1.85 (m, 2H) 1.59-1.45 (m, 6H).

### Example 37—Synthesis of (1r,4r)-4-[[4-[[4-(trifluoromethyl) phenyl]methyl]pyrazolo[1,5-a]pyridine-3-carbonyl]amino]cyclohexanecarboxylic acid (Compound 33)

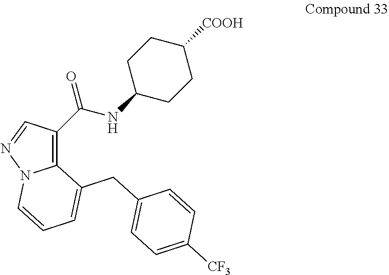 [SMILES: O=C(N[C@H]1CC[C@H](C(=O)O)CC1)c1cnn2cccc(Cc3ccc(C(F)(F)F)cc3)c12]

### Step 1: ethyl (1r,4r)-4-[[4-[[4-(trifluoromethyl) phenyl]methyl]pyrazolo[1,5-a]pyridine-3-carbonyl]amino]cyclohexanecarboxylate

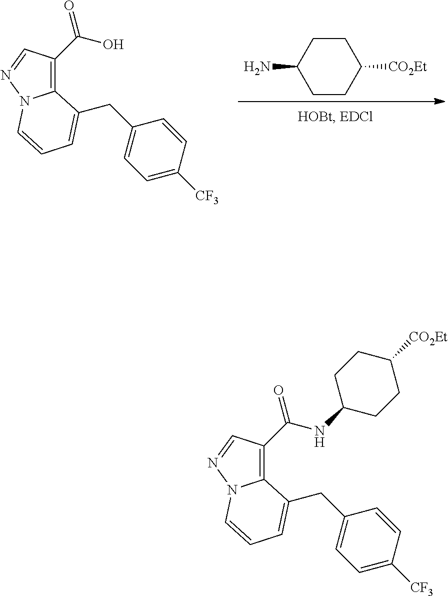 [SMILES: O=C(O)c1cnn2cccc(Cc3ccc(C(F)(F)F)cc3)c12 ; CCOC(=O)[C@H]1CC[C@H](N)CC1 ; CCOC(=O)[C@H]1CC[C@H](NC(=O)c2cnn3cccc(Cc4ccc(C(F)(F)F)cc4)c23)CC1]

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added EDCI (23.94 mg, 124.90 umol, 2 eq), HOBt (1.69 mg, 12.49 umol, 0.2 eq) and Et₃N (31.60 mg, 312.24 umol, 43.46 uL, 5 eq) at 0° C. After addition, the mixture was stirred at this temperature for 30 min, and then ethyl trans-4-aminocyclohexanecarboxylate; hydrochloride (19.46 mg, 93.67 umol, 1.5 eq) was added at 0° C. The resulting mixture was stirred at 25° C. for 12 hr. The residue was diluted with H₂O 10 mL and extracted with DCM 15 mL (5 mL×3). The combined organic layers were washed with brine 5 mL, dried over Na₂SO₄, filtered and concentrated under reduced pressure to give a residue. The crude product was purified by Pre-TLC Compound ethyl (1r,4r)-4-[[4-[[4-(trifluoromethyl) phenyl]methyl]pyrazolo[1,5-a]pyridine-3-carbonyl]amino]cyclohexanecarboxylate (15 mg, 31.68 umol, 50.73% yield) was obtained as a brown solid, which was used into the next step directly without further purification. LCMS for product (ESI): m/z 460.2 [M+H]⁺.

### Step 2: (1r,4r)-4-[[4-[[4-(trifluoromethyl) phenyl]methyl]pyrazolo[1,5-a]pyridine-3-carbonyl]amino]cyclohexanecarboxylic acid

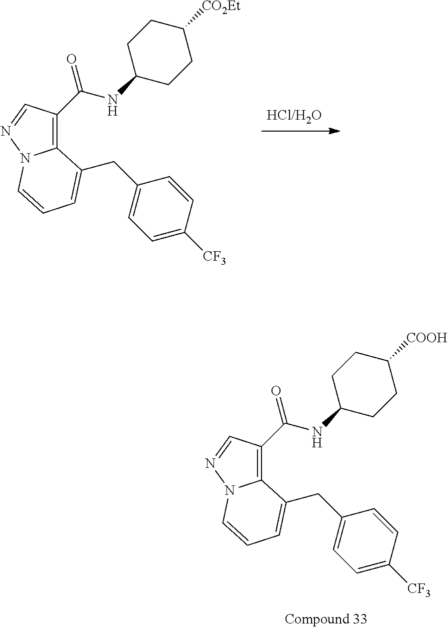 [SMILES: CCOC(=O)[C@H]1CC[C@H](NC(=O)c2cnn3cccc(Cc4ccc(C(F)(F)F)cc4)c23)CC1 ; O=C(N[C@H]1CC[C@H](C(=O)O)CC1)c1cnn2cccc(Cc3ccc(C(F)(F)F)cc3)c12]

To a solution of ethyl (1r,4r)-4-[[4-[[4-(trifluoromethyl) phenyl]methyl]pyrazolo[1,5-a]pyridine-3-carbonyl]amino]cyclohexanecarboxylate (15 mg, 31.68 umol, 1 eq) in H₂O (1 mL) was added HCl (12 M, 13.20 uL, 5 eq). The mixture was stirred at 80° C. for 12 hr. The resulting product was filtered to collect the insoluble. The residue was purified by prep-HPLC (HCl condition). Compound (1r,4r)-4-[[4-[[4-(trifluoromethyl) phenyl]methyl]pyrazolo[1,5-a]pyridine-3-carbonyl]amino]cyclohexanecarboxylic acid (10 mg, 22.02 umol, 69.52% yield, 98.1% purity) was obtained as a white solid. LCMS for product (ESI): m/z 446.0 [M+H]⁺. ¹H NMR (400 MHz, DMSO-d₆) δ=12.06 (br s, 1H), 8.67 (d, J=7.2 Hz, 1H), 8.16 (s, 1H), 8.01 (d, J=8.0 Hz, 1H), 7.56 (d, J=8.0 Hz, 2H), 7.29 (d, J=8.0 Hz, 3H), 7.02 (t, J=7.2 Hz, 1H), 4.64 (s, 2H), 3.65-3.53 (m, 1H), 2.17-2.06 (m, 1H), 1.90 (d, J=11.6 Hz, 2H), 1.82-1.72 (m, 2H), 1.45-1.31 (m, 2H), 1.26-1.11 (m, 2H).

### Example 38—Synthesis of 2-[4-[4-[4-(trifluoromethyl) phenyl]methyl]pyrazolo[1,5-a]pyridine-3-carbonyl]amino]cyclohexyl]acetic acid (Compound 34)

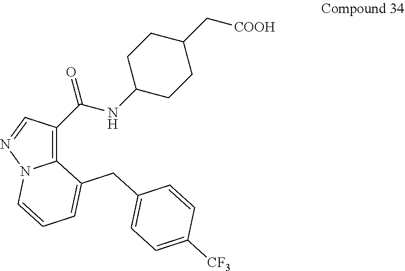 [SMILES: O=C(O)CC1CCC(NC(=O)c2cnn3cccc(Cc4ccc(C(F)(F)F)cc4)c23)CC1]

### Step 1: methyl 2-[4-[[4-[[4-(trifluoromethyl) phenyl]methyl]pyrazolo[1,5-a]pyridine-3-carbonyl]amino]cyclohexyl]acetate

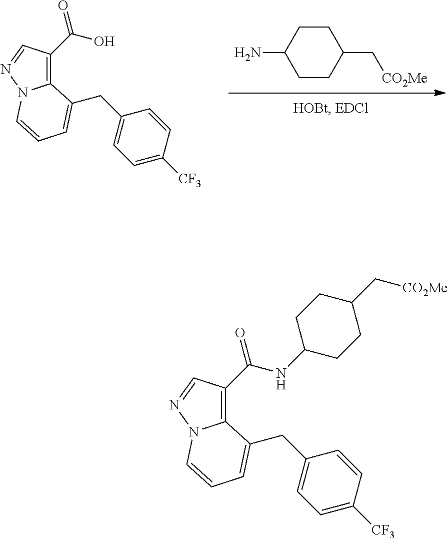 [SMILES: O=C(O)c1cnn2cccc(Cc3ccc(C(F)(F)F)cc3)c12 ; COC(=O)CC1CCC(N)CC1 ; COC(=O)CC1CCC(NC(=O)c2cnn3cccc(Cc4ccc(C(F)(F)F)cc4)c23)CC1]

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added dropwise EDCI (23.94 mg, 124.90 umol, 2 eq), HOBt (1.69 mg, 12.49 umol, 0.2 eq) and Et₃N (31.60 mg, 312.25 umol, 43.46 uL, 5 eq) at 0° C. After addition, the mixture was stirred at this temperature for 0.5 hr, and then methyl 2-(4-aminocyclohexyl) acetate (16.04 mg, 93.68 umol, 1.5 eq) was added at 0° C. The resulting mixture was stirred at 25° C. for 12 hr. The residue was diluted with H₂O 10 mL and extracted with DCM 15 mL (5 mL×3). The combined organic layers were washed with brine 5 mL, dried over Na₂SO₄, filtered and concentrated under reduced pressure to give a residue. The residue was purified by prep-TLC (SiO2, PE: EA=1:1). Compound methyl 2-[4-[[4-[[4-(trifluoromethyl) phenyl]methyl]pyrazolo[1,5-a]pyridine-3-carbonyl]amino]cyclohexyl]acetate (17 mg, 35.90 umol, 57.49% yield) was obtained as a white solid, which was used into the next step directly without further purification. LCMS for product (ESI): m/z 474.2 [M+H]⁺.

### Step 2: 2-[4-[[4-[[4-(trifluoromethyl) phenyl]methyl]pyrazolo[1,5-a]pyridine-3-carbonyl]amino]cyclohexyl]acetic acid

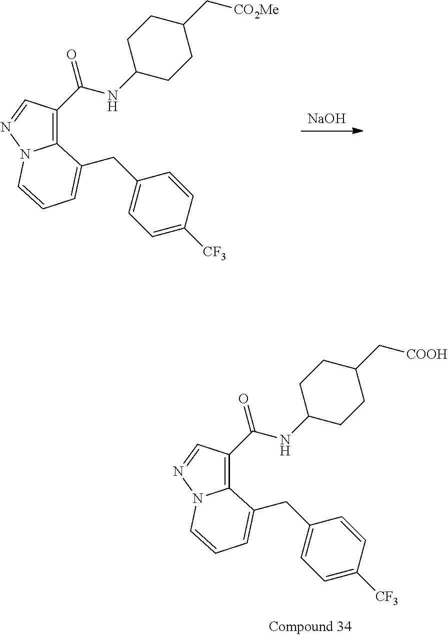 [SMILES: COC(=O)CC1CCC(NC(=O)c2cnn3cccc(Cc4ccc(C(F)(F)F)cc4)c23)CC1 ; "NaOH" ; O=C(O)CC1CCC(NC(=O)c2cnn3cccc(Cc4ccc(C(F)(F)F)cc4)c23)CC1]

To a solution of methyl 2-[4-[[4-[[4-(trifluoromethyl) phenyl]methyl]pyrazolo[1,5-a]pyridine-3-carbonyl]amino]cyclohexyl]acetate (17 mg, 35.90 umol, 1 eq) in H₂O (1 mL) and MeOH (1 mL) was added NaOH (2.88 mg, 71 umol, 2 eq). The mixture was stirred at 50° C. for 12 hr. The reaction mixture was concentrated under reduced pressure to remove solvent. The residue was purified by prep-HPLC (HCl condition). Compound 2-[4-[[4-[[4-(trifluoromethyl) phenyl]methyl]pyrazolo[1,5-a]pyridine-3-carbonyl]amino]cyclohexyl]acetic acid (3 mg, 6.41 umol, 17.84% yield, 98.1% purity) was obtained as a white solid. LCMS for product (ESI): m/z 460.0 [M+H]⁺. ¹H NMR (400 MHz, DMSO-d₆) δ=12.03 (br s, 1H), 8.63 (t, J=6.8 Hz, 1H), 8.17 (d, J=6.8 Hz, 1H), 8.04-7.85 (m, 1H), 7.55 (t, J=8.0 Hz, 2H), 7.33-7.25 (m, 3H), 7.04-7.02 (m, 1H), 4.67-4.61 (m, 2H), 3.87-3.85 (m, 0.5H), 3.86-3.55 (m, 0.5H), 2.16-2.09 (m, 2H), 1.85-1.67 (m, 3H), 1.61-1.42 (m, 2H), 1.40-1.30 (m, 1H), 1.25-0.99 (m, 3H).

### Example 39—Synthesis of 2-(4-(4-(4-(trifluoromethyl)benzyl)pyrazolo[1,5-a]pyridine-3-carboxamido)bicyclo[2.2.2]octan-1-yl)acetic acid (Compound 35)

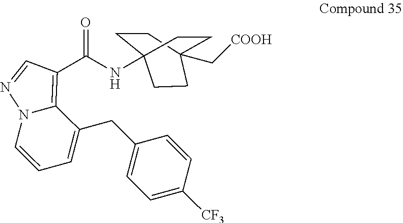 [SMILES: O=C(O)CC12CCC(NC(=O)c3cnn4cccc(Cc5ccc(C(F)(F)F)cc5)c34)(CC1)CC2]

### Step 1: methyl 2-(4-(4-(4-(trifluoromethyl)benzyl)pyrazolo[1,5-a]pyridine-3-carboxamido)bicyclo[2.2.2]octan-1-yl)acetate

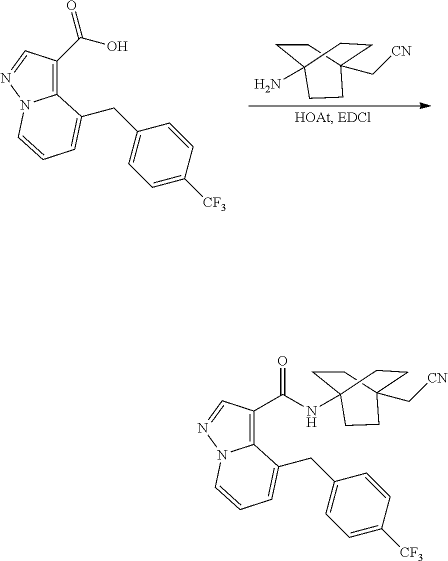

To a solution of 4-[[4-(trifluoromethyl) phenyl]methyl]pyrazolo[1,5-a]pyridine-3-carboxylic acid (90 mg, 281.02 umol, 1 eq) and 2-(4-aminobicyclo[2.2.2]octan-1-yl)acetic cyanic anhydride (83.1 mg, 421.53 umol, 1.5 eq) in DCM (8 mL) was added HOAt (7.65 mg, 56.20 umol, 7.86 uL, 0.2 eq), Et₃N (142.18 mg, 1.41 mmol, 195.57 uL, 5 eq) and EDCI (109.06 mg, 702.54 umol, 2.5 eq) at 25° C. and stirred at 25° C. for 16 hr. LCMS showed all the carboxylic acid was consumed completely and desired product was detected. The solvent was removed to afford the crude product. Crude product was purified by Pre-TLC. Compound N-[1-(cyanomethyl)-4-bicyclo[2.2.2]octanyl]-4-[[4-(trifluoromethyl)phenyl]methyl]pyrazolo[1,5-a]pyridine-3-carboxamide (80 mg, 57% yield) was obtained as a white solid. HPLC showed the product was clean. LCMS for product (ESI): m/z 467.3 [M+H]⁺.

### Step 2: 2-(4-(4-(4-(trifluoromethyl)benzyl)pyrazolo[1,5-a]pyridine-3-carboxamido)bicyclo[2.2.2]octan-1-yl)acetic acid

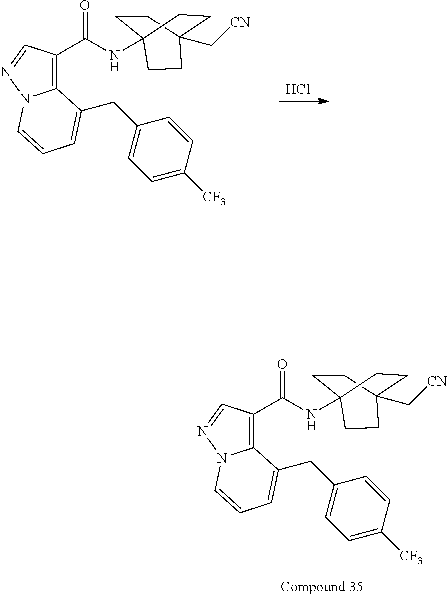 [SMILES: N#CCC12CCC(NC(=O)c3cnn4cccc(Cc5ccc(C(F)(F)F)cc5)c34)(CC1)CC2 ; Cl ; N#CCC12CCC(NC(=O)c3cnn4cccc(Cc5ccc(C(F)(F)F)cc5)c34)(CC1)CC2]

To a solution of N-[1-(cyanomethyl)-4-bicyclo[2.2.2]octanyl]-4-[[4-(trifluoromethyl)phenyl]methyl]pyrazolo[1,5-a]pyridine-3-carboxamide (35 mg, 75.03 umol, 1 eq) in H₂O (0.5 mL) was added HCl (12 M, 6.25 uL, 1 eq). The mixture was stirred at 90° C. for 24 hr. The reaction mixture was concentrated to remove the solvent. The residue was purified by prep-HPLC (HCl condition). 2-(4-(4-(4-(trifluoromethyl)benzyl)pyrazolo[1,5-a]pyridine-3-carboxamido)bicyclo[2.2.2]octan-1-yl)acetic acid (10 mg, 26% yield, 97% purity) was obtained as a white solid. LCMS for product (ESI): m/z 486.1 [M+H]⁺. ¹H NMR (400 MHz, DMSO-d₆) δ=11.95 (s, 1H), 8.65 (d, J=7.2 Hz, 1H), 8.14 (s, 1H), 7.58-7.55 (m, 3H), 7.33-7.26 (m, 3H), 7.00 (t, J=7.2 Hz, 1H), 4.61 (s, 2H), 1.99 (s, 2H), 1.85-1.81 (m, 6H), 1.57-1.52 (m, 6H).

### Example 40—Synthesis of 4-[[4-[[4-(trifluoromethyl) phenyl]methyl]pyrazolo[1,5-a]pyridine-3-carbonyl]amino]bicyclo[2.2.2]octane-1-carboxylic acid (Compound 36)

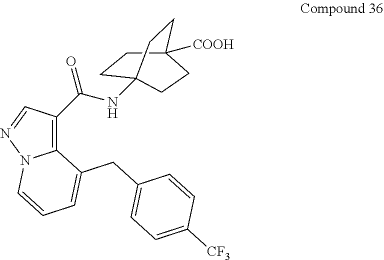 [SMILES: O=C(NC12CCC(C(=O)O)(CC1)CC2)c1cnn2cccc(Cc3ccc(C(F)(F)F)cc3)c12]

### Step 1: methyl 4-[[4-[[4-(trifluoromethyl) phenyl]methyl]pyrazolo[1,5-a]pyridine-3-carbonyl]amino]bicyclo[2.2.2]octane-1-carboxylate

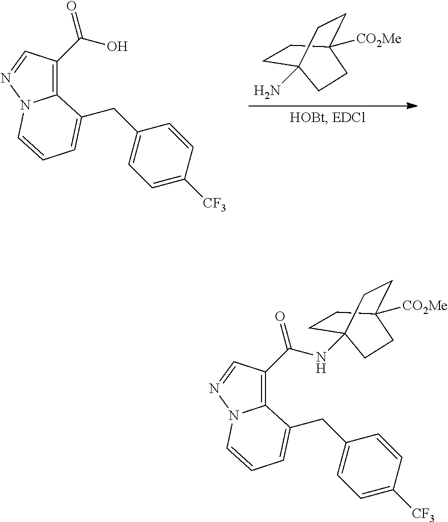

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added EDCI (23.94 mg, 124.90 umol, 2 eq), HOBt (1.69 mg, 12.49 umol, 0.2 eq) and Et₃N (31.60 mg, 312.25 umol, 43.46 uL, 5 eq) at 0° C. After addition, the mixture was stirred at this temperature for 0.5 hr, and then methyl 4-aminobicyclo[2.2.2]octane-1-carboxylate (17.17 mg, 93.68 umol, 1.5 eq) was added at 0° C. The resulting mixture was stirred at 25° C. for 12 hr. LC-MS showed the carboxylic acid was consumed completely and one main peak with desired mass was detected. The residue was diluted with H₂O 10 mL and extracted with DCM 15 mL (5 mL×3). The combined organic layers were washed with brine 10 mL, dried over Na₂SO₄, filtered and concentrated under reduced pressure to give a residue. The crude product was used into the next step without further purification. Compound methyl 4-[[4-[[4-(trifluoromethyl) phenyl]methyl]pyrazolo[1,5-a]pyridine-3-carbonyl]amino]bicyclo[2.2.2]octane-1-carboxylate (15 mg, 30.90 umol, 49.47% yield) was obtained as a white solid, which was used directly to next step without further purification. LCMS for product (ESI): m/z 486.4 [M+H]⁺.

### Step 2: 4-[[4-[[4-(trifluoromethyl) phenyl]methyl]pyrazolo[1,5-a]pyridine-3-carbonyl]amino]bicyclo[2.2.2]octane-1-carboxylic acid

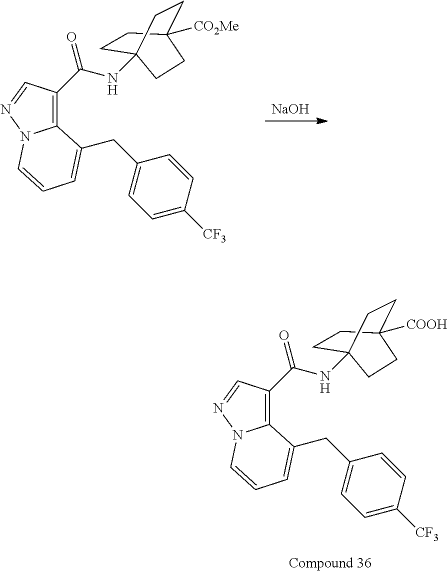 [SMILES: COC(=O)C12CCC(NC(=O)c3cnn4cccc(Cc5ccc(C(F)(F)F)cc5)c34)(CC1)CC2 ; "NaOH" ; O=C(NC12CCC(C(=O)O)(CC1)CC2)c1cnn2cccc(Cc3ccc(C(F)(F)F)cc3)c12]

To a solution of methyl 4-[[4-[[4-(trifluoromethyl) phenyl]methyl]pyrazolo[1,5-a]pyridine-3-carbonyl]amino]bicyclo[2.2.2]octane-1-carboxylate (15 mg, 30.90 umol, 1 eq) in H₂O (1 mL) and MeOH (1 mL) was added NaOH (1.24 mg, 30.90 umol, 1 eq). The mixture was stirred at 50° C. for 12 hr. The reaction mixture was concentrated under reduced pressure to remove solvent. The mixture was adjusted to pH 4 with 1N HCl. The mixture was filtered. Compound 4-[[4-[[4-(trifluoromethyl) phenyl]methyl]pyrazolo[1,5-a]pyridine-3-carbonyl]amino]bicyclo[2.2.2]octane-1-carboxylic acid (6 mg, 12.57 umol, 40.70% yield, 98.8% purity) was obtained as a white solid. LCMS for product (ESI): m/z 472.0 [M+H]⁺. ¹H NMR (400 MHz, DMSO-d₆) δ=8.65 (d, J=6.8 Hz, 1H), 8.15 (s, 1H), 7.63 (s, 1H), 7.58 (d, J=8.0 Hz, 2H), 7.33 (d, J=8.0 Hz, 2H), 7.27 (d, J=7.2 Hz, 1H), 7.00 (t, J=6.8 Hz, 1H), 4.61 (s, 2H), 1.90-1.82 (m, 6H), 1.78-1.71 (m, 6H).

### Example 41—Synthesis of 2-[3-[[4-[[4-(trifluoromethyl) phenyl]methyl]pyrazolo[1,5-a]pyridine-3-carbonyl]amino]cyclopentyl]acetic acid (Compound 37)

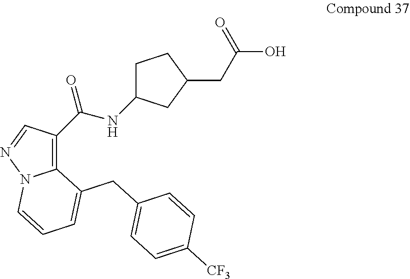 [SMILES: O=C(O)CC1CCC(NC(=O)c2cnn3cccc(Cc4ccc(C(F)(F)F)cc4)c23)C1]

### Step 1: ethyl (2E)-2-[3-(tert-butoxycarbonylamino) cyclopentylidene]acetate

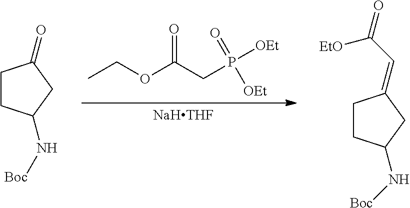

To a solution of ethyl 2-diethoxyphosphorylacetate (1 g, 4.46 mmol, 884.96 uL, 1.5 eq) in THF (8 mL) was added dropwise NaH (178.40 mg, 4.46 mmol, 60% purity, 1.5 eq) at 0° C. over 10 min. After addition, the mixture was stirred at 25° C. for 1 hr, and then tert-butyl N— (3-oxocyclopentyl) carbamate (592.49 mg, 2.97 mmol, 1 eq) was added dropwise at 0° C. The resulting mixture was stirred at 25° C. for 1 hr. LC-MS showed ketone was consumed completely and one main peak with desired mass was detected. The reaction mixture was quenched by addition water 10 mL at 0° C., and then diluted with water 10 mL and extracted with EA 20 (20 mL×2). The combined organic layers were washed with brine 50 mL, dried over Na₂SO₄, filtered and concentrated under reduced pressure to give a residue. The residue was purified by prep-TLC (SiO₂, PE:EA=7:1). Compound ethyl (2E)-2-[3-(tert-butoxycarbonylamino) cyclopentylidene]acetate (760 mg, 2.82 mmol, 94.89% yield) was obtained as colorless oil.

### Step 2: ethyl 2-[3-(tert-butoxycarbonylamino) cyclopentyl]acetate

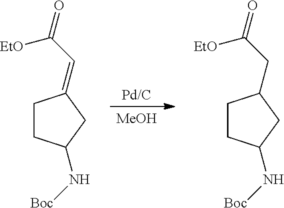 [SMILES: "MeOH" ; CCOC(=O)/C=C1\CCC(NC(=O)OC(C)(C)C)C1 ; "Pd/C" ; CCOC(=O)CC1CCC(NC(=O)OC(C)(C)C)C1]

To a solution of ethyl (2E)-2-[3-(tert-butoxycarbonylamino) cyclopentylidene]acetate (300 mg, 1.11 mmol, 1 eq) in MeOH (2 mL) was added Pd/C (10%, 300 mg) under N₂ atmosphere. The suspension was degassed and purged with H₂ for 5 times. The mixture was stirred under H₂ (15 Psi) at 25° C. for 2 hr. TLC indicated no double bond compound was remained, and one major new spot with larger polarity was detected. The resulting mixture was filtered by celite and then concentrated under reduced pressure to remove MeOH. The crude product was used in next step without purification. Compound ethyl 2-[3-(tert-butoxycarbonylamino) cyclopentyl]acetate (280 mg, 1.03 mmol, 92.64% yield) was obtained as a colorless oil.

### Step 3: ethyl 2-(3-aminocyclopentyl) acetate

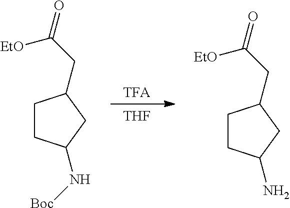 [SMILES: CCOC(=O)CC1CCC(NC(=O)OC(C)(C)C)C1 ; CCOC(=O)CC1CCC(N)C1 ; "TFA" ; "THF"]

To a solution of ethyl 2-[3-(tert-butoxycarbonylamino) cyclopentyl]acetate (150 mg, 552.79 umol, 1 eq) in THE (1.5 mL) was added TFA (1.54 g, 13.51 mmol, 1 mL, 24.43 eq). The mixture was stirred at 25° C. for 2 hr. TLC indicated 0% of Boc protected amine was remained, and one major new spot with larger polarity was detected. The reaction mixture was oncentrated under reduced pressure to give a residue. The crude product was used in next step without purification. Compound ethyl 2-(3-aminocyclopentyl) acetate (90 mg, 525.59 umol, 95.08% yield) was obtained as a colorless oil.

### Step 4: ethyl 2-[3-[[4-[[4-(trifluoromethyl) phenyl]methyl]pyrazolo[1,5-a]pyridine-3-carbonyl]amino]cyclopentyl]acetate

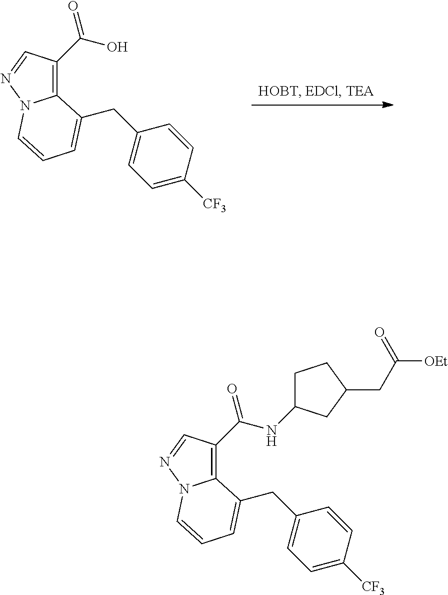 [SMILES: O=C(O)c1cnn2cccc(Cc3ccc(C(F)(F)F)cc3)c12 ; "HOBT, EDCl, TEA" ; CCOC(=O)CC1CCC(NC(=O)c2cnn3cccc(Cc4ccc(C(F)(F)F)cc4)c23)C1]

To a solution of 4-[[4-(trifluoromethyl) phenyl]methyl]pyrazolo[1,5-a]pyridine-3-carboxylic acid (20 mg, 62.45 umol, 1 eq) ethyl 2-(3-aminocyclopentyl) acetate (12.83 mg, 74.94 umol, 1.2 eq) in DCM (0.6 mL) was added dropwise EDCI (23.94 mg, 124.90 umol, 2.0 eq), HOBt (1.69 mg, 12.49 umol, 0.2 eq) at 25° C. After addition, the mixture was stirred at this temperature for 30 min, and then TEA (28.44 mg, 281.02 umol, 39.11 uL, 4.5 eq) was added dropwise at 0° C. The resulting mixture was stirred at 25° C. for 16 hr. TLC indicated no carboxylic acid was remained, and one major new spot with lower polarity was detected. The reaction mixture was concentrated under reduced pressure to remove DCM to give a residue. The residue was purified by prep-TLC (SiO₂, PE: EA=1:1). Compound ethyl 2-[3-[[4-[[4-(trifluoromethyl) phenyl]methyl]pyrazolo[1,5-a]pyridine-3-carbonyl]amino]cyclopentyl]acetate (28 mg, 59.14 umol, 94.70% yield) was obtained as a yellow oil, which was used into the next step directly without further purification.

### Step 5: 2-[3-[[4-[[4-(trifluoromethyl) phenyl]methyl]pyrazolo[1,5-a]pyridine-3-carbonyl]amino]cyclopentyl]acetic acid

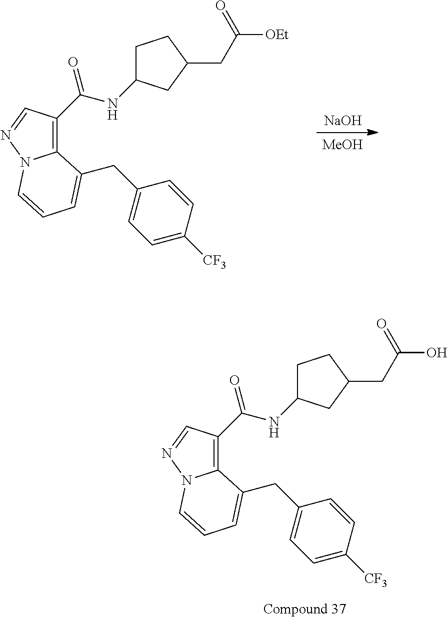 [SMILES: "MeOH" ; CCOC(=O)CC1CCC(NC(=O)c2cnn3cccc(Cc4ccc(C(F)(F)F)cc4)c23)C1 ; "NaOH" ; O=C(O)CC1CCC(NC(=O)c2cnn3cccc(Cc4ccc(C(F)(F)F)cc4)c23)C1]

To a solution of ethyl 2-[3-[[4-[[4-(trifluoromethyl) phenyl]methyl]pyrazolo[1,5-a]pyridine-3-carbonyl]amino]cyclopentyl]acetate (28 mg, 59.14 umol, 1 eq) in EtOH (3 mL) and H₂O (0.5 mL) was added NaOH (23.65 mg, 591.36 umol, 10 eq). The mixture was stirred at 35° C. for 2 hr. TLC indicated ˜0% of the ester was remained, and one major new spot with larger polarity was detected. The reaction mixture was quenched by addition 1 M HCl 1 mL at 0° C., and then concentrated under reduced pressure to give a residue. The residue was purified by prep-TLC (SiO₂, DCM: MeOH=5:1). Compound 2-[3-[[4-[[4-(trifluoromethyl) phenyl]methyl]pyrazolo[1,5-a]pyridine-3-carbonyl]amino]cyclopentyl]acetic acid (5 mg, 11.02 umol, 18.64% yield, 98.21% purity) was obtained as a white solid. LCMS for product (ESI): m/z 446.2 [M+H]⁺. ¹H NMR (400 MHz, DMSO-d₆) δ=12.02 (br s, 1H), 8.69 (d, J=6.4 Hz, 1H), 8.28-8.06 (m, 2H), 7.64-7.48 (m, 2H), 7.39-7.26 (m, 3H), 7.04 (t, J=6.8 Hz, 1H), 4.66 (s, 2H), 4.33-4.05 (m, 2H), 2.30-2.21 (m, 2H), 2.21-2.10 (m, 1H), 2.01-1.63 (m, 2H), 1.57-1.38 (m, 1H), 1.35-1.22 (m, 1H), 1.21-1.01 (m, 1H).

### Example 42—Synthesis of 2-((1r,4r)-4-(4-(4-(trifluoromethyl)benzyl)pyrazolo[1,5-a]pyridine-3-carboxamido)cyclohexyl)acetic acid (Compound 38)

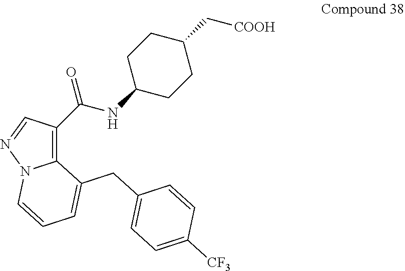 [SMILES: O=C(O)C[C@H]1CC[C@H](NC(=O)c2cnn3cccc(Cc4ccc(C(F)(F)F)cc4)c23)CC1]

### Step 1: 2-((1r,4r)-4-(4-(4-(trifluoromethyl)benzyl)pyrazolo[1,5-a]pyridine-3-carboxamido)cyclohexyl)acetic acid ethyl ester

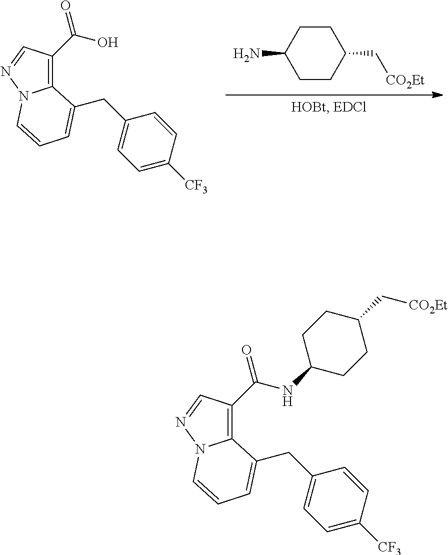

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added dropwise EDCI (23.94 mg, 124.90 umol, 2 eq), HOBt (1.69 mg, 12.49 umol, 0.2 eq) and Et₃N (31.60 mg, 312.25 umol, 43.46 uL, 5 eq) at 0° C. After addition, the mixture was stirred at this temperature for 0.5 hr, and then ethyl trans 2-(4-aminocyclohexyl) acetate (17.35 mg, 93.68 umol, 1.5 eq) was added at 0° C. The resulting mixture was stirred at 25° C. for 12 hr. The residue was diluted with H₂O 10 mL and extracted with DCM 15 mL (5 mL×3). The combined organic layers were washed with brine 10 mL, dried over Na₂SO₄, filtered and concentrated under reduced pressure to give a residue. The crude product was used into the next step without further purification. Compound 2-((1r,4r)-4-(4-(4-(trifluoromethyl)benzyl)pyrazolo[1,5-a]pyridine-3-carboxamido)cyclohexyl)acetic acid ethyl ester (20 mg, 41.02 umol, 65.69% yield) was obtained as a white solid. LCMS for product (ESI): m/z 488.3 [M+H]⁺.

### Step 2: 2-((1r,4r)-4-(4-(4-(trifluoromethyl)benzyl)pyrazolo[1,5-a]pyridine-3-carboxamido)cyclohexyl)acetic acid

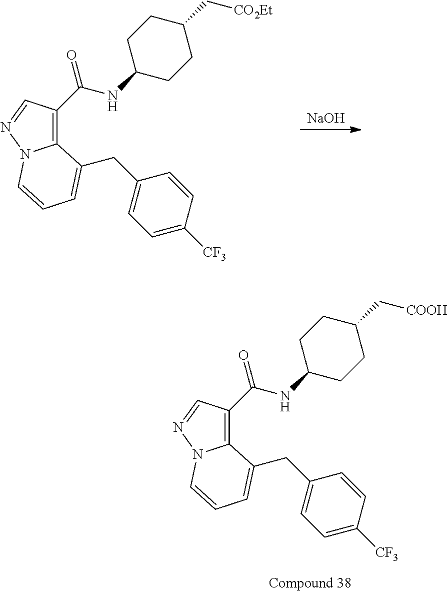 [SMILES: CCOC(=O)C[C@H]1CC[C@H](NC(=O)c2cnn3cccc(Cc4ccc(C(F)(F)F)cc4)c23)CC1 ; "NaOH" ; O=C(O)C[C@H]1CC[C@H](NC(=O)c2cnn3cccc(Cc4ccc(C(F)(F)F)cc4)c23)CC1]

To a solution of 2-((1r,4r)-4-(4-(4-(trifluoromethyl)benzyl)pyrazolo[1,5-a]pyridine-3-carboxamido)cyclohexyl)acetic acid ethyl ester (20 mg, 41.02 umol, 1 eq) in H₂O (1 mL) and MeOH (1 mL) was added NaOH (1.64 mg, 41.02 umol, 1 eq). The mixture was stirred at 50° C. for 12 hr. The reaction mixture was concentrated under reduced pressure to remove solvent. The mixture was adjusted to pH 4 with 1N HCl. The mixture was filtered. Compound 2-((1r,4r)-4-(4-(4-(trifluoromethyl)benzyl)pyrazolo[1,5-a]pyridine-3-carboxamido)cyclohexyl)acetic acid (15 mg, 31.83 umol, 77.59% yield, 97.5% purity) was obtained as a white solid. LCMS for product (ESI): m/z 460.0 [M+H]⁺. ¹H NMR (400 MHz, DMSO-d₆) δ=8.67 (d, J=6.8 Hz, 1H), 8.17 (s, 1H), 7.98 (d, J=8.0 Hz, 1H), 7.56 (d, J=8.0 Hz, 2H), 7.30 (d, J=8.4 Hz, 3H), 7.02 (t, J=6.8 Hz, 1H), 4.64 (s, 2H), 3.65-3.54 (m, 1H), 2.11 (d, J=7.2 Hz, 2H), 1.73 (d, J=10.0 Hz, 4H), 1.64-1.52 (m, 1H), 1.20-1.14 (m, 2H), 1.08-1.02 (m, 2H).

### Example 43—Synthesis of (1R,3S)-3-[[4-[[4-(trifluoromethyl) phenyl]methyl]pyrazolo[1,5-a]pyridine-3-carbonyl]amino]cyclopentanecarboxylic acid (Compound 39)

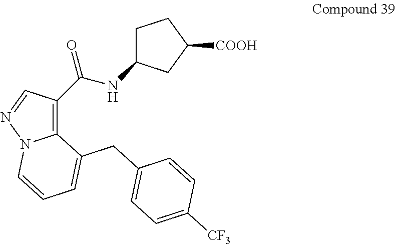 [SMILES: O=C(N[C@H]1CC[C@@H](C(=O)O)C1)c1cnn2cccc(Cc3ccc(C(F)(F)F)cc3)c12]

### Step 1: (1R,3S)-3-[[4-[[4-(trifluoromethyl) phenyl]methyl]pyrazolo[1,5-a]pyridine-3-carbonyl]amino]cyclopentanecarboxylic acid

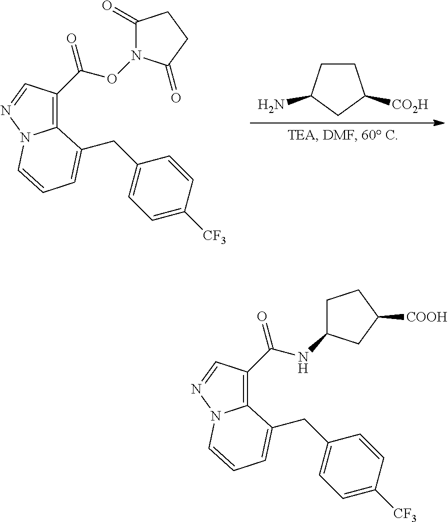

To a solution of (2,5-dioxopyrrolidin-1-yl) 4-[[4-(trifluoromethyl) phenyl]methyl]pyrazolo[1,5-a]pyridine-3-carboxylate (50 mg, 119.81 umol, 1 eq) and (1R,3S)-3-aminocyclopentanecarboxylic acid (77.37 mg, 599.04 umol, 5 eq) in DMF (2 mL) was added Et₃N (218.22 mg, 2.16 mmol, 300.16 uL, 18 eq). The mixture was stirred at 60° C. for 12 hr. Several new peaks were shown on LC-MS and 64% of desired compound was detected. The reaction mixture was concentrated under reduced pressure to remove DMF. The residue was diluted with H₂O 10 mL and extracted with EA 15 mL (5 mL×3). The combined organic layers were washed with brine 5 mL, dried over Na₂SO₄, filtered and concentrated under reduced pressure to give a residue. The residue was purified by prep-HPLC (HCl condition). Compound (1R,3S)-3-[[4-[[4-(trifluoromethyl) phenyl]methyl]pyrazolo[1,5-a]pyridine-3-carbonyl]amino]cyclopentanecarboxylic acid (2.5 mg, 5.67 umol, 4.73% yield, 97.8% purity) was obtained as a white solid. LCMS for product (ESI): m/z 432.0 [M+H]⁺. ¹H NMR (400 MHz, DMSO-d₆) δ=12.12 (br s, 1H), 8.67 (d, J=6.8 Hz, 1H), 8.20-8.15 (m, 2H), 7.56 (d, J=8.0 Hz, 2H), 7.32 (d, J=7.2 Hz, 3H), 7.03 (t, J=7.2 Hz, 1H), 4.65 (s, 2H), 4.21-4.13 (m, 1H), 2.78-2.69 (m, 1H), 2.17-2.14 (m, 1H), 1.89-1.79 (m, 3H), 1.67-1.63 (m, 1H), 1.51-1.43 (m, 1H).

### Example 44—Synthesis of (1r,4r)-4-[[[4-[[4-(trifluoromethyl) phenyl]methyl]pyrazolo[1,5-a]pyridine-3-carbonyl]amino]methyl]cyclohexanecarboxylic acid (Compound 40)

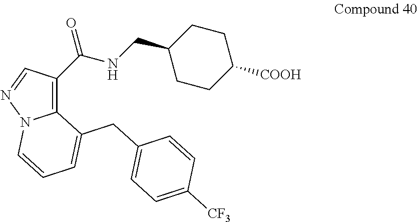 [SMILES: O=C(NC[C@H]1CC[C@H](C(=O)O)CC1)c1cnn2cccc(Cc3ccc(C(F)(F)F)cc3)c12]

### Step 1: methyl (1r,4r)-4-[[[4-[[4-(trifluoromethyl) phenyl]methyl]pyrazolo[1,5-a]pyridine-3-carbonyl]amino]methyl]cyclohexanecarboxylate

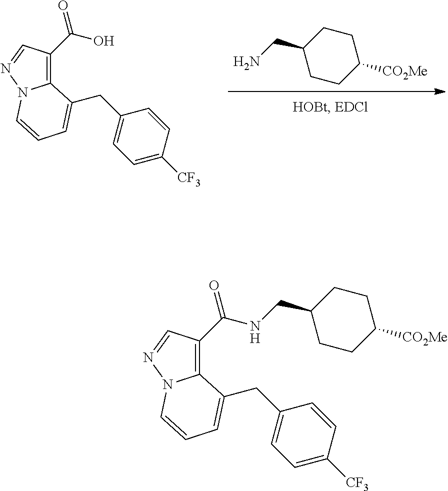 [SMILES: O=C(O)c1cnn2cccc(Cc3ccc(C(F)(F)F)cc3)c12 ; COC(=O)[C@H]1CC[C@H](CN)CC1 ; COC(=O)[C@H]1CC[C@H](CNC(=O)c2cnn3cccc(Cc4ccc(C(F)(F)F)cc4)c23)CC1]

To a solution of 4-[[4-(trifluoromethyl) phenyl]methyl]pyrazolo[1,5-a]pyridine-3-carboxylic acid (20 mg, 62.45 umol, 1 eq) in DCM (1 mL) was added EDCI (23.94 mg, 124.90 umol, 2 eq), HOBt (1.69 mg, 12.49 umol, 0.2 eq) and Et₃N (31.60 mg, 312.25 umol, 43.46 uL, 5 eq) at 0° C. After addition, the mixture was stirred at this temperature for 0.5 hr, and then methyl trans-4-(aminomethyl) cyclohexanecarboxylate (16.04 mg, 93.68 umol, 1.5 eq) was added at 0° C. The resulting mixture was stirred at 25° C. for 12 hr. The residue was diluted with H₂O 10 mL and extracted with DCM 15 mL (5 mL×3). The combined organic layers were washed with brine 5 mL, dried over Na₂SO₄, filtered and concentrated under reduced pressure to give a residue. The crude product was used into the next step without further purification. Compound methyl (1r,4r)-4-[[[4-[[4-(trifluoromethyl) phenyl]methyl]pyrazolo[1,5-a]pyridine-3-carbonyl]amino]methyl]cyclohexanecarboxylate (20 mg, 42.24 umol, 67.64% yield) was obtained as a white solid, which was used into the next step directly without further purification. LCMS for product (ESI): m/z 474.3 [M+H]⁺.

### Step 2: (1r,4r)-4-[[[4-[[4-(trifluoromethyl) phenyl]methyl]pyrazolo[1,5-a]pyridine-3-carbonyl]amino]methyl]cyclohexanecarboxylic acid

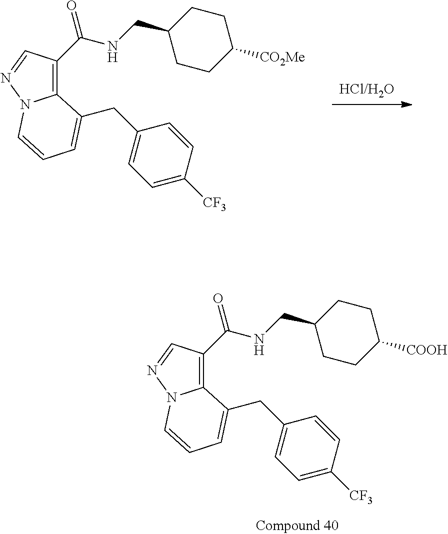 [SMILES: COC(=O)[C@H]1CC[C@H](CNC(=O)c2cnn3cccc(Cc4ccc(C(F)(F)F)cc4)c23)CC1 ; O=C(NC[C@H]1CC[C@H](C(=O)O)CC1)c1cnn2cccc(Cc3ccc(C(F)(F)F)cc3)c12]

To a solution of methyl (1r,4r)-4-[[[4-[[4-(trifluoromethyl) phenyl]methyl]pyrazolo[1,5-a]pyridine-3-carbonyl]amino]methyl]cyclohexanecarboxylate (20 mg, 42.24 umol, 1 eq) in H₂O (1 mL) was added HCl (12 M, 3.52 uL, 1 eq). The mixture was stirred at 80° C. for 12 hr. The resulting product was filtered to collect the insoluble. The residue was purified by prep-HPLC (HCl condition). Compound (1r,4r)-4-[[[4-[[4-(trifluoromethyl) phenyl]methyl]pyrazolo[1,5-a]pyridine-3-carbonyl]amino]methyl]cyclohexanecarboxylic acid (15 mg, 32.39 umol, 76.67% yield, 99.2% purity) was obtained as a white solid. LCMS for product (ESI): m/z 460.0 [M+H]⁺. ¹H NMR (400 MHz, DMSO-d₆) δ=11.97 (s, 1H), 8.68 (d, J=6.8 Hz, 1H), 8.23 (s, 1H), 8.17 (t, J=5.6 Hz, 1H), 7.55 (d, J=8.0 Hz, 2H), 7.29 (d, J=8.8 Hz, 3H), 7.02 (t, J=6.8 Hz, 1H), 4.68 (s, 2H), 3.04 (t, J=6.4 Hz, 2H), 2.15-2.06 (m, 1H), 1.86 (d, J=10.4 Hz, 2H), 1.70 (d, J=10.4 Hz, 2H), 1.46-1.41 (m, 1H), 1.24-1.20 (m, 2H), 0.92-0.89 (m, 2H).

### Example 45—Synthesis of trans-2-[[1-ethyl-4-[[4-(trifluoromethyl)phenyl]methyl]indole-3-carbonyl]amino]spiro[3.31]heptane-6-carboxylic acid (Compound 41)

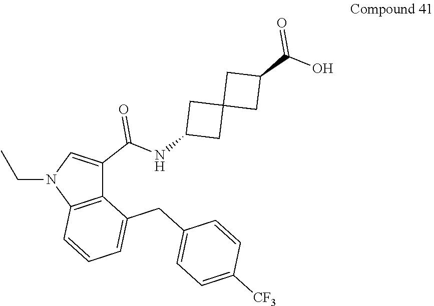 [SMILES: CCn1cc(C(=O)N[C@H]2CC3(C2)C[C@H](C(=O)O)C3)c2c(Cc3ccc(C(F)(F)F)cc3)cccc21]

### Step 1: trans-(S)-1-phenylethyl 2-[[1-ethyl-4-[[4-(trifluoromethyl)phenyl]methyl]indole-3-carbonyl]amino]spiro[3.3]heptane-6-carboxylate

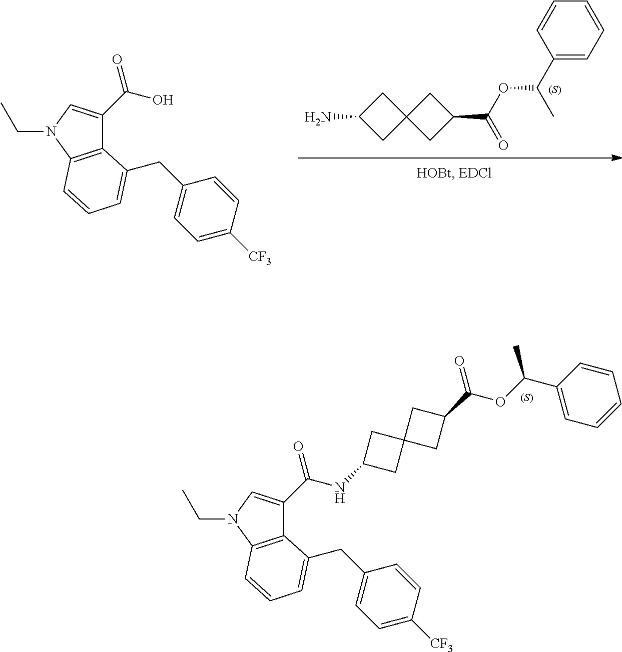

To a solution of 1-ethyl-4-[[4-(trifluoromethyl)phenyl]methyl]indole-3-carboxylic acid (50 mg, 143.96 umol, 1 eq) in DCM (2 mL) was added HOBt (3.89 mg, 28.79 umol, 0.2 eq), EDCI (55.19 mg, 287.91 umol, 2 eq) and Et₃N (72.83 mg, 719.78 umol, 100.18 uL, 5 eq) at 0° C. The mixture was stirred at 0° C. for 0.5 hr. Then trans-(S)-1-phenylethyl 2-aminospiro[3.3]heptane-6-carboxylate (37.33 mg, 143.96 umol, 1 eq) was added the mixture. The mixture was stirred at 25° C. for 16 hr. The reaction mixture was diluted with H₂O 3 mL and extracted with DCM 9 mL (3 mL×3). The combined organic layers were washed with brine 5 mL, dried over Na₂SO₄, filtered and concentrated under reduced pressure to give a residue. The crude product was used into the next step without further purification. Compound trans-(S)-1-phenylethyl 2-[[1-ethyl-4-[[4-(trifluoromethyl)phenyl]methyl]indole-3-carbonyl]amino]spiro[3.3]heptane-6-carboxylate (30 mg, 50.96 umol, 35.40% yield) was obtained as a white solid, which was used into the next step directly without further purification. LCMS for product (ESI): m/z 589.4 [M+H]⁺.

### Step 2: trans-2-[[1-ethyl-4-[[4-(trifluoromethyl)phenyl]methyl]indole-3-carbonyl]amino]spiro[3.3]heptane-6-carboxylic acid

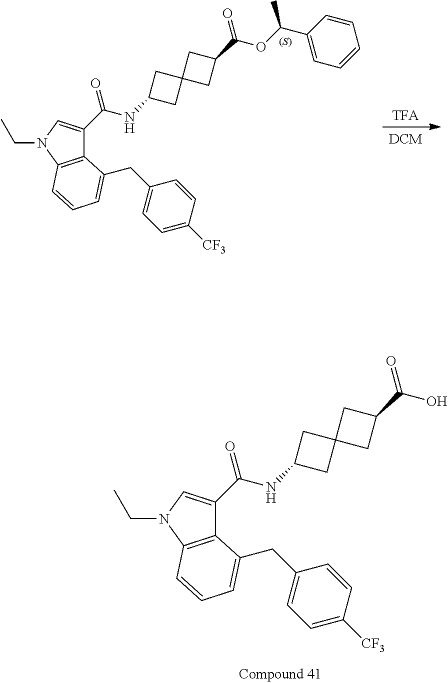

To a solution of trans-(S)-1-phenylethyl 2-[[1-ethyl-4-[[4-(trifluoromethyl)phenyl]methyl]indole-3-carbonyl]amino]spiro[3.3]heptane-6-carboxylate (30 mg, 50.96 umol, 1 eq) in DCM (1.5 mL) was added TFA (5.81 mg, 50.96 umol, 3.77 uL, 1 eq). The mixture was stirred at 25° C. for 16 hr. The reaction mixture was concentrated under reduced pressure to remove MeOH. The mixture was adjusted to pH 4 with 1N HCl. The mixture was filtered. Compound trans-2-[[1-ethyl-4-[[4-(trifluoromethyl)phenyl]methyl]indole-3-carbonyl]amino]spiro[3.3]heptane-6-carboxylic acid (6 mg, 12.14 umol, 23.82% yield, 98.02% purity) was obtained as a white solid. LCMS for product (ESI): m/z 485.1 [M+H]⁺. ¹H NMR (400 MHz, DMSO-d₆) δ ppm 12.05 (br s, 1H) 8.13 (d, J=7.6 Hz, 1H) 7.71 (s, 1H) 7.52 (d, J=8.4 Hz, 2H) 7.42 (d, J=8.4 Hz, 1H) 7.28 (d, J=8.00 Hz, 2H) 7.19 (t, J=7.6 Hz, 1H) 7.03 (d, J=7.2 Hz, 1H) 4.62 (s, 2H) 4.25-4.18 (m, 3H) 2.95 (t, J=8.4 Hz, 1H) 2.44-2.29 (m, 2H) 2.25-2.22 (m, 2H) 2.15-2.05 (m, 2H) 1.96-1.85 (m, 2H) 1.38 (t, J=7.13 Hz, 3H).

### Example 46—Synthesis of 4-[(1S)-1-[[1-ethyl-4-[[4-(trifluoromethyl)phenyl]methyl]indole-3-carbonyl]amino]ethyl]benzoic acid (Compound 42)

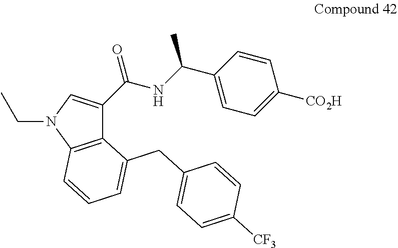 [SMILES: CCn1cc(C(=O)N[C@@H](C)c2ccc(C(=O)O)cc2)c2c(Cc3ccc(C(F)(F)F)cc3)cccc21]

### Step 1: methyl 4-bromo-1H-indole-3-carboxylate

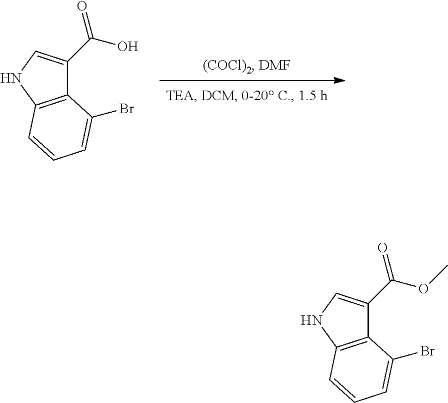

To a solution of 4-bromo-1H-indole-3-carboxylic acid (5 g, 20.83 mmol, 1 eq) and DMF (304.49 mg, 4.17 mmol, 320.52 uL, 0.2 eq) in DCM (60 mL) was added (COCl)₂ (2.91 g, 22.91 mmol, 2.01 mL, 1.1 eq). The mixture was stirred at 25° C. for 2 h, then TEA (6.32 g, 62.49 mmol, 8.70 mL, 3 eq) and MeOH (6.67 g, 208.29 mmol, 8.43 mL, 10 eq) was added into the reaction at 0° C., the reaction was stirred at 25° C. for 2 h. LC-MS showed 0% of the carboxylic acid was remained. Several new peaks were shown on LC-MS and 96.89% of desired compound was detected. The reaction mixture was diluted with H₂O 20 mL and the organic layer was separated. The aqueous was extracted with EA 90 mL (30 mL×3). The combined organic layers were combined and washed with brine (20 mL), dried over Na₂SO₄, filtered and concentrated under reduced pressure to give a residue. The crude product was used into the next step without further purification. Compound methyl 4-bromo-1H-indole-3-carboxylate (5.1 g, 20.07 mmol, 96.37% yield) was obtained as yellow oil.

### Step 2: methyl 4-bromo-1-ethyl-indole-3-carboxylate

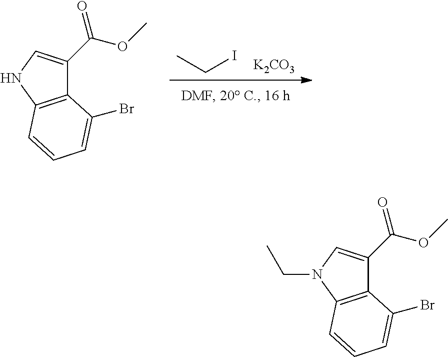

To a solution of methyl 4-bromo-1H-indole-3-carboxylate (1 g, 3.94 mmol, 1 eq) and iodoethane (3.07 g, 19.68 mmol, 1.57 mL, 5 eq) in DMF (2 mL) was added K₂CO₃ (1.09 g, 7.87 mmol, 2 eq). The mixture was stirred at 25° C. for 5 hr. TLC indicated the starting material was consumed completely and one new spot generated. The reaction mixture was diluted with H₂O 10 mL and extracted with MTBE 30 mL (10 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=50/1 to 0/1). Compound methyl 4-bromo-1-ethyl-indole-3-carboxylate (1 g, 3.54 mmol, 90.06% yield) was obtained as yellow oil, which was used into the next step directly without further purification. LCMS for product (ESI): m/z 281.9 [M+H]⁺.

### Step 3: methyl 1-ethyl-4-[[4-(trifluoromethyl)phenyl]methyl]indole-3-carboxylate

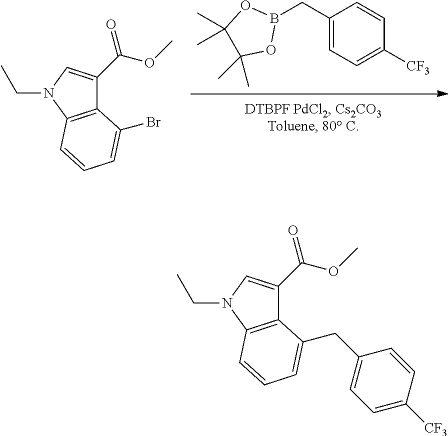

To a solution of 4,4,5,5-tetramethyl-2-[[4-(trifluoromethyl)phenyl]methyl]-1,3,2-dioxaborolane (454.89 mg, 1.59 mmol, 1.5 eq) and methyl 4-bromo-1-ethyl-indole-3-carboxylate (300 mg, 1.06 mmol, 1 eq) in toluene (4 mL) was added Cs₂CO₃ (690.74 mg, 2.12 mmol, 2 eq) and ditert-butyl(cyclopentyl)phosphane;dichloropalladium;iron (69.09 mg, 106.00 umol, 0.1 eq). The mixture was stirred at 80° C. for 16 hr. LC-MS showed 0% of the bromide was remained. Several new peaks were shown on LC-MS and 41.65% of desired compound was detected. The reaction mixture was diluted with H₂O 5 mL and extracted with DCM 15 mL (5 mL×3). The combined organic layers were washed with brine (10 mL), dried over Na₂SO₄, filtered and concentrated under reduced pressure to give a residue. The residue was purified by prep-TLC (SiO2, PE: EA=2:1). Compound methyl 1-ethyl-4-[[4-(trifluoromethyl)phenyl]methyl]indole-3-carboxylate (150 mg, 415.10 umol, 39.16% yield) was obtained as a white solid. LCMS for product (ESI): m/z 362.3 [M+H]⁺.

### Step 4: Compound 1-ethyl-4-[[4-(trifluoromethyl)phenyl]methyl]indole-3-carboxylic acid

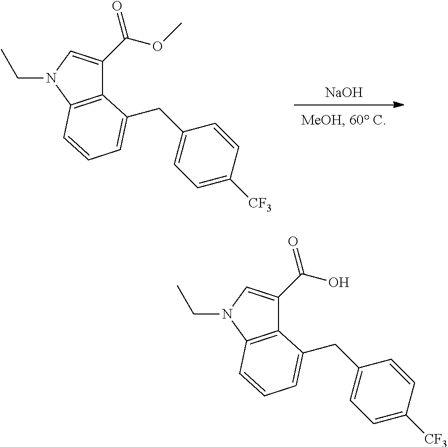 [SMILES: CCn1cc(C(=O)OC)c2c(Cc3ccc(C(F)(F)F)cc3)cccc21 ; "NaOH" ; CCn1cc(C(=O)O)c2c(Cc3ccc(C(F)(F)F)cc3)cccc21]

To a solution of methyl 1-ethyl-4-[[4-(trifluoromethyl)phenyl]methyl]indole-3-carboxylate (150 mg, 415.10 umol, 1 eq) in MeOH (2 mL) and H₂O (1 mL) was added NaOH (16.60 mg, 415.10 umol, 1 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 1-ethyl-4-[[4-(trifluoromethyl)phenyl]methyl]indole-3-carboxylic acid (130 mg, 374.28 umol, 90.17% yield) was obtained as a white solid. LCMS for product (ESI): m/z 348.1 [M+H]⁺.

### Step 5: methyl 4-[(1S)-1-[[1-ethyl-4-[[4-(trifluoromethyl)phenyl]methyl]indole-3-carbonyl]amino]ethyl]benzoate

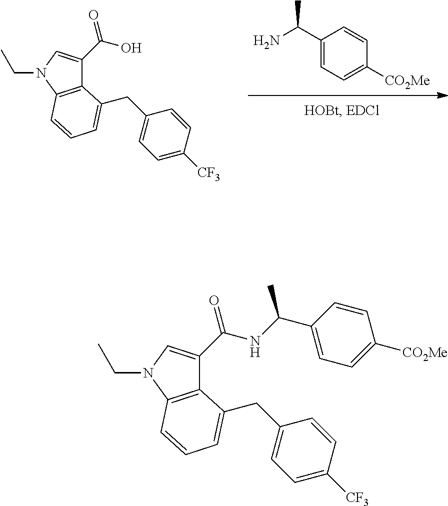 [SMILES: CCn1cc(C(=O)O)c2c(Cc3ccc(C(F)(F)F)cc3)cccc21 ; COC(=O)c1ccc([C@H](C)N)cc1 ; CCn1cc(C(=O)N[C@@H](C)c2ccc(C(=O)OC)cc2)c2c(Cc3ccc(C(F)(F)F)cc3)cccc21]

To a solution of 1-ethyl-4-[[4-(trifluoromethyl)phenyl]methyl]indole-3-carboxylic acid (70 mg, 201.54 umol, 1 eq) in DCM (1 mL) was added HOBt (5.45 mg, 40.31 umol, 0.2 eq), Et₃N (101.97 mg, 1.01 mmol, 140.26 uL, 5 eq) and EDCI (77.27 mg, 403.07 umol, 2 eq). The mixture was stirred at 0° C. for 0.5 hr. Then methyl 4-[(1S)-1-aminoethyl]benzoate (36.12 mg, 201.54 umol, 1 eq) was added the mixture. The mixture was stirred at 25° C. for 16 hr. LC-MS showed 0% of the carboxylic acid was remained. Several new peaks were shown on LC-MS and 44.39% of desired compound was detected. The reaction mixture was diluted with H₂O 5 mL and extracted with DCM 15 mL (5 mL×3). The combined organic layers were washed with brine (5 mL), dried over Na₂SO₄, filtered and concentrated under reduced pressure to give a residue. The residue was purified by prep-TLC (SiO2, PE:EA=1:1). Compound methyl 4-[(1S)-1-[[1-ethyl-4-[[4-(trifluoromethyl)phenyl]methyl]indole-3-carbonyl]amino]ethyl]benzoate (40 mg, 78.66 umol, 39.03% yield) was obtained as a white solid, which was used into the next step directly without further purification. LCMS for product (ESI): m/z 509.3 [M+H]⁺.

### Step 6: 4-[(1S)-1-[[1-ethyl-4-[[4-(trifluoromethyl)phenyl]methyl]indole-3-carbonyl]amino]ethyl]benzoic acid

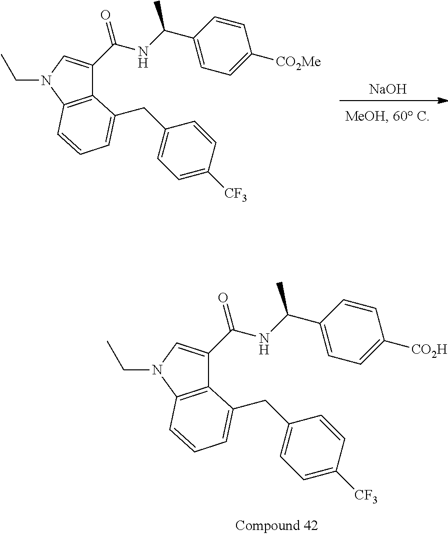 [SMILES: CCn1cc(C(=O)N[C@@H](C)c2ccc(C(=O)OC)cc2)c2c(Cc3ccc(C(F)(F)F)cc3)cccc21 ; "NaOH" ; CCn1cc(C(=O)N[C@@H](C)c2ccc(C(=O)O)cc2)c2c(Cc3ccc(C(F)(F)F)cc3)cccc21]

To a solution of methyl 4-[(1S)-1-[[1-ethyl-4-[[4-(trifluoromethyl)phenyl]methyl]indole-3-carbonyl]amino]ethyl]benzoate (40 mg, 78.66 umol, 1 eq) in H₂O (1 mL) and MeOH (1 mL) was added NaOH (9.44 mg, 235.97 umol, 3 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 4-[(1S)-1-[[1-ethyl-4-[[4-(trifluoromethyl)phenyl]methyl]indole-3-carbonyl]amino]ethyl]benzoic acid (30 mg, 60.31 umol, 76.68% yield, 99.42% purity) was obtained as a white solid. LCMS for product (ESI): m/z 495.2 [M+H]⁺. ¹H NMR (400 MHz, DMSO-d₆) δ ppm 12.84 (br s, 1H) 8.51 (d, J=8.0 Hz, 1H) 7.92 (d, J=8.0 Hz, 2H) 7.93 (s, 1H) 7.53 (d, J=8.0 Hz, 2H) 7.44 (d, J=8.4 Hz, 1H) 7.33 (d, J=8.0 Hz, 2H) 7.16-7.22 (m, 3H) 7.04 (d, J=7.2 Hz, 1H) 5.19 (t, J=7.6 Hz, 1H) 4.69 (d, J=14.4 Hz, 1H) 4.50 (d, J=14.4 Hz, 1H) 4.25 (q, J=7.2 Hz, 2H) 1.43-1.38 (m, 6H).

### Example 47—Synthesis of 4-[(1S)-1-[[1-(2-fluoroethyl)-4-[[4-(trifluoromethyl)phenyl]methyl]indole-3-carbonyl]amino]ethyl]benzoic acid (Compound 43)

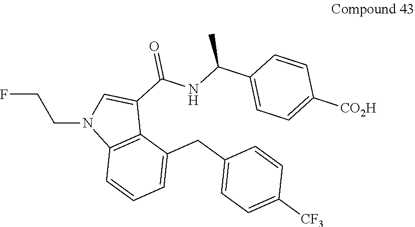 [SMILES: C[C@H](NC(=O)c1cn(CCF)c2cccc(Cc3ccc(C(F)(F)F)cc3)c12)c1ccc(C(=O)O)cc1]

### Step 1: methyl 4-bromo-1-(2-fluoroethyl)indole-3-carboxylate

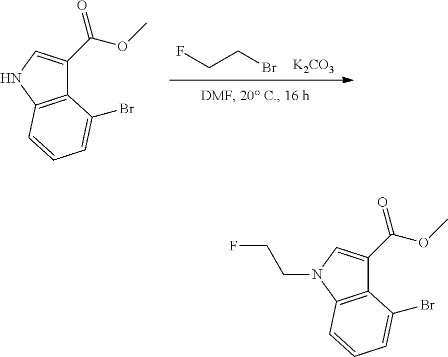

To a solution of methyl 4-bromo-1H-indole-3-carboxylate (1 g, 3.94 mmol, 1 eq) and 1-bromo-2-fluoro-ethane (2.50 g, 19.68 mmol, 5 eq) in DMF (2 mL) was added K₂CO₃ (1.09 g, 7.87 mmol, 2 eq). The mixture was stirred at 25° C. for 5 hr. TLC indicated the starting material was consumed completely. The reaction mixture was diluted with H₂O 20 mL and extracted with EA 30 mL (10 mL×3). The combined organic layers were washed with brine (15 mL), dried over Na₂SO₄, filtered and concentrated under reduced pressure to give a residue. The residue was purified by column chromatography (SiO₂, Petroleum ether/Ethyl acetate=50/1 to 0/1). Compound methyl 4-bromo-1-(2-fluoroethyl)indole-3-carboxylate (1.05 g, 3.33 mmol, 84.66% yield) was obtained as yellow oil, which was used into the next step directly without further purification.

### Step 2: methyl 1-(2-fluoroethyl)-4-[[4-(trifluoromethyl)phenyl]methyl]indole-3-carboxylate

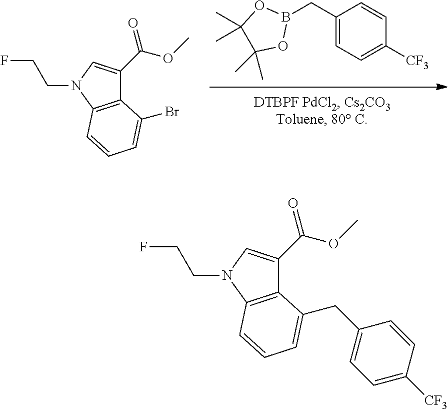

To a solution of 4,4,5,5-tetramethyl-2-[[4-(trifluoromethyl)phenyl]methyl]-1,3,2-dioxaborolane (428.97 mg, 1.50 mmol, 1.5 eq) and methyl 4-bromo-1-(2-fluoroethyl)indole-3-carboxylate (300 mg, 999.59 umol, 1 eq) in toluene (2 mL) was added Cs₂CO₃ (651.37 mg, 2.00 mmol, 2 eq) and ditert-butyl(cyclopentyl)phosphane;dichloropalladium;iron (65.15 mg, 99.96 umol, 0.1 eq). The mixture was stirred at 80° C. for 16 hr. LC-MS showed 0% of the bromide was remained. Several new peaks were shown on LC-MS and 45.96% of desired compound was detected. The reaction mixture was diluted with H₂O 5 mL and extracted with DCM 15 mL (5 mL×3). The combined organic layers were washed with brine (5 mL), dried over Na₂SO₄, filtered and concentrated under reduced pressure to give a residue. The residue was purified by prep-TLC (SiO₂, PE: EA=2:1). Compound methyl 1-(2-fluoroethyl)-4-[[4-(trifluoromethyl)phenyl]methyl]indole-3-carboxylate (160 mg, 421.78 umol, 42.19% yield) was obtained as yellow oil. LCMS for product (ESI): m/z 380.1 [M+H]⁺.

### Step 3: 1-ethyl-4-[[4-(trifluoromethyl)phenyl]methyl]indole-3-carboxylic acid

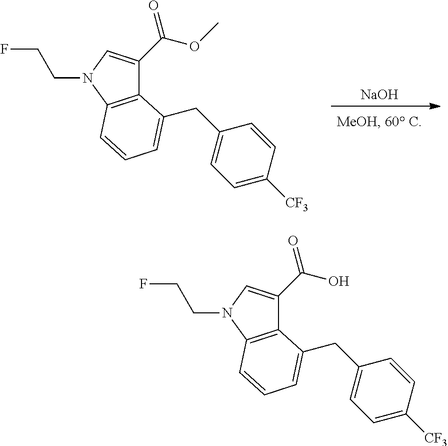

To a solution of methyl 1-(2-fluoroethyl)-4-[[4-(trifluoromethyl)phenyl]methyl]indole-3-carboxylate (160 mg, 421.78 umol, 1 eq) in MeOH (2 mL) and H₂O (1 mL) was added NaOH (16.87 mg, 421.78 umol, 1 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 1-ethyl-4-[[4-(trifluoromethyl)phenyl]methyl]indole-3-carboxylic acid (130 mg, 374.28 umol, 90.17% yield) was obtained as a white solid.

### Step 4: methyl 4-[(1S)-1-[[1-(2-fluoroethyl)-4-[[4-(trifluoromethyl)phenyl]methyl]indole-3-carbonyl]amino]ethyl]benzoate

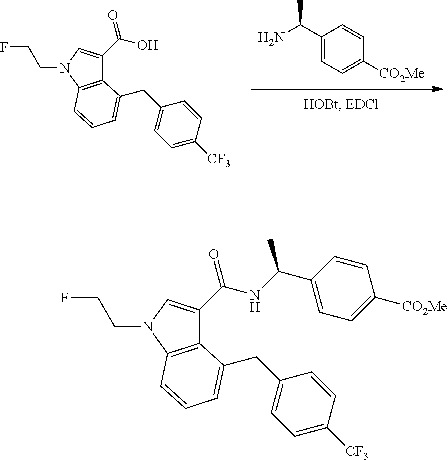 [SMILES: O=C(O)c1cn(CCF)c2cccc(Cc3ccc(C(F)(F)F)cc3)c12 ; COC(=O)c1ccc([C@H](C)N)cc1 ; COC(=O)c1ccc([C@H](C)NC(=O)c2cn(CCF)c3cccc(Cc4ccc(C(F)(F)F)cc4)c23)cc1]

To a solution of methyl 4-[(1S)-1-aminoethyl]benzoate (24.53 mg, 136.87 umol, 1 eq) in DCM (2 mL) was added HOBt (3.70 mg, 27.37 umol, 0.2 eq), Et₃N (69.25 mg, 684.33 umol, 95.25 uL, 5 eq) and EDCI (52.47 mg, 273.73 umol, 2 eq). The mixture was stirred at 0° C. for 0.5 hr. Then 1-(2-fluoroethyl)-4-[[4-(trifluoromethyl)phenyl]methyl]indole-3-carboxylic acid (50 mg, 136.87 umol, 1 eq) was added the mixture. The mixture was stirred at 25° C. for 16 hr. HPLC showed 0% of the carboxylic acid was remained. The reaction mixture was diluted with H₂O 5 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound methyl 4-[(1S)-1-[[1-(2-fluoroethyl)-4-[[4-(trifluoromethyl)phenyl]methyl]indole-3-carbonyl]amino]ethyl]benzoate (25 mg, 47.48 umol, 34.69% yield) was obtained as a white solid, which was used into the next step directly without further purification. LCMS for product (ESI): m/z 527.3 [M+H]⁺.

### Step 5: 4-[(1S)-1-[[1-(2-fluoroethyl)-4-[[4-(trifluoromethyl)phenyl]methyl]indole-3-carbonyl]amino]ethyl]benzoic acid

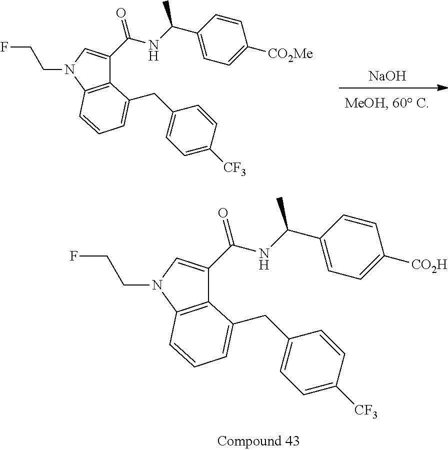 [SMILES: COC(=O)c1ccc([C@H](C)NC(=O)c2cn(CCF)c3cccc(Cc4ccc(C(F)(F)F)cc4)c23)cc1 ; "NaOH" ; C[C@H](NC(=O)c1cn(CCF)c2cccc(Cc3ccc(C(F)(F)F)cc3)c12)c1ccc(C(=O)O)cc1]

To a solution of methyl 4-[(1S)-1-[[1-(2-fluoroethyl)-4-[[4-(trifluoromethyl)phenyl]methyl]indole-3-carbonyl]amino]ethyl]benzoate (25 mg, 47.48 umol, 1 eq), in H₂O (1 mL) and MeOH (1 mL) was added NaOH (5.70 mg, 142.44 umol, 3 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 4-[(1S)-1-[[1-(2-fluoroethyl)-4-[[4-(trifluoromethyl)phenyl]methyl]indole-3-carbonyl]amino]ethyl]benzoic acid C₂₈H₂₄N₂O₃F₄ (5 mg, 9.76 umol, 20.55% yield, 100% purity) was obtained as a white solid. LCMS for product (ESI): m/z 513.0 [M+H]⁺. ¹H NMR (400 MHz, DMSO-d₆) δ ppm 8.57 (d, J=8.0 Hz, 1H) 7.90 (d, J=8.4 Hz, 2H) 7.82 (s, 1H) 7.53-7.46 (m, 3H) 7.34 (d, J=8.0 Hz, 2H) 7.17-7.23 (m, 3H) 7.06 (d, J=7.2 Hz, 1H) 5.19 (t, J=7.2 Hz, 1H) 4.83 (t, J=4.4 Hz, 1H) 4.73-4.64 (m, 2H) 4.59 (t, J=4.4 Hz, 1H) 4.54-4.48 (m, 2H) 1.41 (d, J=7.2 Hz, 3H).

### Example 48—Synthesis of trans-2-[[1-(2-fluoroethyl)-4-[[4-(trifluoromethyl)phenyl]methyl]-indole-3-carbonyl]amino]spiro[3.3]heptane-6-carboxylic acid (Compound 44)

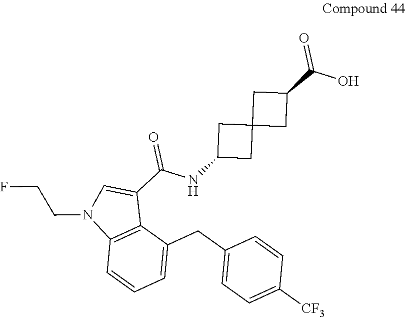 [SMILES: O=C(N[C@H]1CC2(C1)C[C@H](C(=O)O)C2)c1cn(CCF)c2cccc(Cc3ccc(C(F)(F)F)cc3)c12]

### Step 1: trans-(S)-1-phenylethyl 2-[[1-(2-fluoroethyl)-4-[[4-(trifluoromethyl)phenyl]methyl]indole-3-carbonyl]amino]spiro[3.3]heptane-6-carboxylate

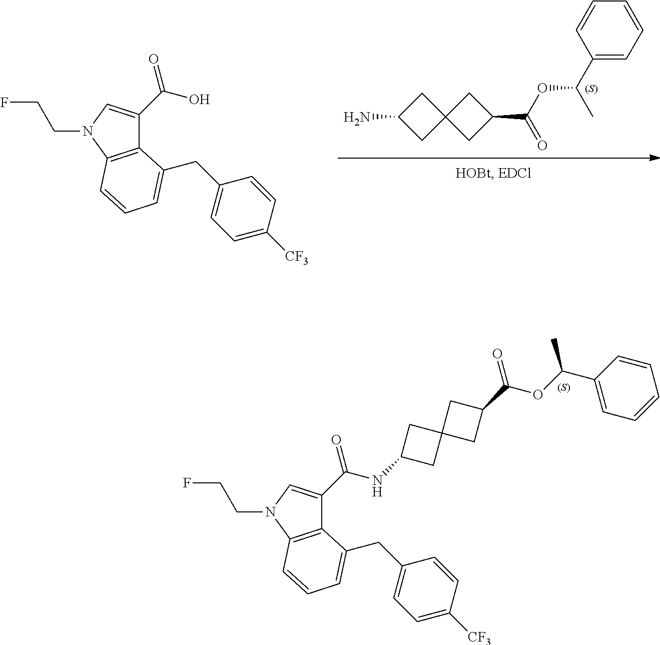

To a solution of 1-(2-fluoroethyl)-4-[[4-(trifluoromethyl)phenyl]methyl]indole-3-carboxylic acid (75 mg, 205.30 umol, 1 eq) in DCM (1 mL) was added HOBt (5.55 mg, 41.06 umol, 0.2 eq), Et₃N (103.87 mg, 1.03 mmol, 142.88 uL, 5 eq) and EDCI (78.71 mg, 410.60 umol, 2 eq). The mixture was stirred at 0° C. for 0.5 hr. Then trans-(S)-1-phenylethyl 2-aminospiro[3.3]heptane-6-carboxylate (53.24 mg, 205.30 umol, 1 eq) was added the mixture. The mixture was stirred at 25° C. for 16 hr. HPLC showed 0% of the carboxylic acid was remained. The reaction mixture was diluted with H₂O 5 mL and extracted with DCM (5 mL×3). The combined organic layers were washed with brine 5 mL, dried over Na₂SO₄, filtered and concentrated under reduced pressure to give a residue. The crude product was used into the next step without further purification. Compound trans-(S)-1-phenylethyl 2-[[1-(2-fluoroethyl)-4-[[4-(trifluoromethyl)phenyl]methyl]indole-3-carbonyl]amino]spiro[3.3]heptane-6-carboxylate (63 mg, 103.85 umol, 50.58% yield) was obtained as a white solid, which was used into the next step directly without further purification. LCMS for product (ESI): m/z 607.4 [M+H]⁺.

### Step 2: trans-2-[[1-(2-fluoroethyl)-4-[[4-(trifluoromethyl)phenyl]methyl]indole-3-carbonyl]amino]spiro[3.3]heptane-6-carboxylic acid

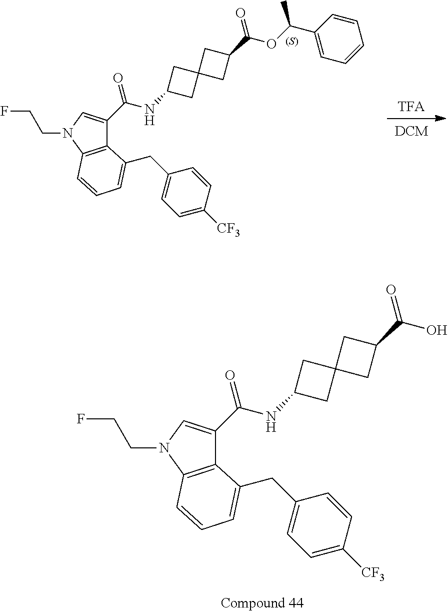

To a solution of trans-(S)-1-phenylethyl 2-[[1-(2-fluoroethyl)-4-[[4-(trifluoromethyl)phenyl]methyl]indole-3-carbonyl]amino]spiro[3.3]heptane-6-carboxylate (63 mg, 103.85 umol, 1 eq) in DCM (2 mL) was added TFA (11.84 mg, 103.85 umol, 7.69 uL, 1 eq). The mixture was stirred at 25° C. for 16 hr. HPLC showed 0% of the ester was remained. The reaction mixture was concentrated under reduced pressure to remove MeOH. The mixture was adjusted to pH 4 with 1N HCl. The mixture was filtered. Compound trans-2-[[1-(2-fluoroethyl)-4-[[4-(trifluoromethyl)phenyl]methyl]indole-3-carbonyl]amino]spiro[3.3]heptane-6-carboxylic acid (45 mg, 85.03 umol, 81.88% yield, 94.95% purity) was obtained as a white solid. LCMS for product (ESI): m/z 504.2 [M+H]⁺. ¹H NMR (400 MHz, DMSO-d₆) δ ppm 8.17 (d, J=8.0 Hz, 1H) 7.69 (s, 1H) 7.51 (d, J=8.0 Hz, 2H) 7.45 (d, J=8.0 Hz, 1H) 7.27 (d, J=8.0 Hz, 2H) 7.19 (t, J=8.0 Hz, 1H) 7.04 (d, J=7.2 Hz, 1H) 4.80 (t, J=4.4 Hz, 1H) 4.68 (t, J=4.4 Hz, 1H) 4.61 (s, 2H) 4.55 (t, J=4.4 Hz, 1H) 4.48 (t, J=4.4 Hz, 1H) 4.24-4.18 (m, 1H) 2.93 (d, J=8.4 Hz, 1H) 2.42-2.34 (m, 2H) 2.25-2.20 (m, 2H) 2.13-2.04 (m, 2H) 1.95-1.83 (m, 2H).

### Example 49—Synthesis of trans-2-[[1-ethyl-4-[[4-(trifluoromethyl)phenyl]methyl]-pyrrolo[2,3-b]pyridine-3-carbonyl]amino]spiro[3.3]heptane-6-carboxylic acid (Compound 45)

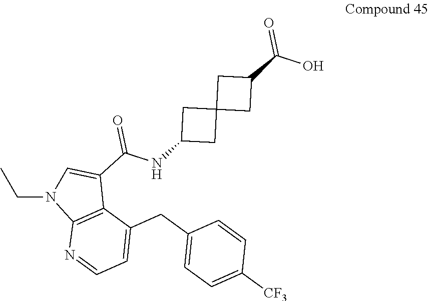

### Step 1: trans-(S)-1-phenylethyl 2-[[1-ethyl-4-[[4-(trifluoromethyl)phenyl]methyl]-pyrrolo[2,3-b]pyridine-3-carbonyl]amino]spiro[3.3]heptane-6-carboxylate

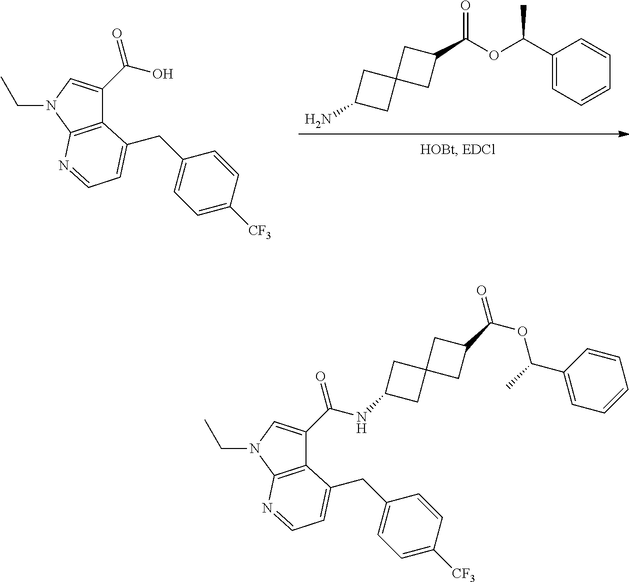

To a solution of 1-ethyl-4-[[4-(trifluoromethyl)phenyl]methyl]pyrrolo[2,3-b]pyridine-3-carboxylic acid (60 mg, 172.26 umol, 1 eq) in DCM (1 mL) was added Et₃N (87.15 mg, 861.28 umol, 119.88 uL, 5 eq), HOBt (4.66 mg, 34.45 umol, 0.2 eq) and EDCI (66.04 mg, 344.51 umol, 2 eq) at 0° C. After addition, the mixture was stirred at this temperature for 0.5 hr, and then trans-(S)-1-phenylethyl 2-aminospiro[3.3]heptane-6-carboxylate (67.01 mg, 258.38 umol, 1.5 eq) was added at 0° C. The resulting mixture was stirred at 25° C. for 12 hr. LCMS showed the carboxylic acid was consumed completely. The residue was diluted with H₂O 3 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound trans-(S)-1-phenylethyl 2-[[1-ethyl-4-[[4-(trifluoromethyl)phenyl]methyl]pyrrolo[2,3-b]pyridine-3-carbonyl]amino]spiro[3.3]heptane-6-carboxylate (80 mg, crude) was obtained as a white solid, which was used into the next step directly without further purification. LCMS for product (ESI): m/z 590.3 [M+H]⁺.

### Step 2: trans-2-[[1-ethyl-4-[[4-(trifluoromethyl)phenyl]methyl]pyrrolo[2,3-b]pyridine-3-carbonyl]amino]spiro[3.3]heptane-6-carboxylic acid

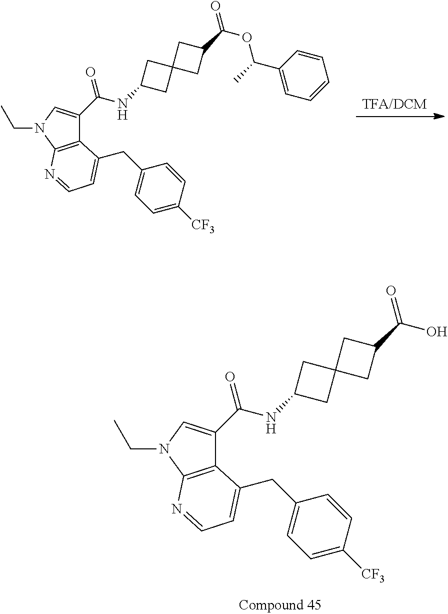

To a solution of trans-(S)-1-phenylethyl 2-[[1-ethyl-4-[[4-(trifluoromethyl)phenyl]methyl]pyrrolo[2,3-b]pyridine-3-carbonyl]amino]spiro[3.3]heptane-6-carboxylate (80 mg, 135.68 umol, 1 eq) in DCM (1 mL) and TFA (1 mL). The mixture was stirred at 25° C. for 1 hr. The reaction mixture was concentrated under reduced pressure to remove solvent. The mixture was added 1M NaOH. Then was adjusted to pH 4 with 1N HCl. The mixture was filtered. Compound trans-2-[[1-ethyl-4-[[4-(trifluoromethyl)phenyl]methyl]pyrrolo[2,3-b]pyridine-3-carbonyl]amino]spiro[3.3]heptane-6-carboxylic acid (59 mg, 121.2 umol, 89.6% yield, 97% purity) was obtained as a white solid. LCMS for product (ESI): m/z 486.1 [M+H]⁺. ¹H NMR (400 MHz, DMSO-d₆) δ=12.01 (br s, 1H), 8.29-8.23 (m, 2H), 8.02 (s, 1H), 7.56-7.51 (m, 2H), 7.40-7.27 (m, 2H), 7.07 (s, 1H), 4.68 (s, 2H), 4.34-4.15 (m, 3H), 2.96-2.82 (m, 1H), 2.35-2.19 (m, 4H), 2.16-2.05 (m, 2H), 2.00-1.86 (m, 2H), 1.45-1.32 (m, 3H).

### Example 50—Synthesis of 4-[(1S)-1-[[1-ethyl-4-[[4-(trifluoromethyl)phenyl]methyl]-pyrrolo[2,3-b]pyridine-3-carbonyl]amino]ethyl]benzoic acid (Compound 46)

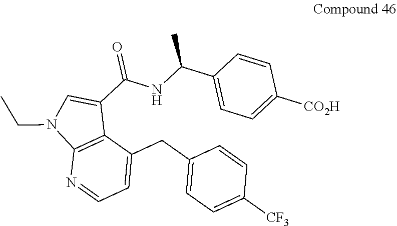 [SMILES: CCn1cc(C(=O)N[C@@H](C)c2ccc(C(=O)O)cc2)c2c(Cc3ccc(C(F)(F)F)cc3)ccnc21]

### Step 1: methyl 4-bromo-1-ethyl-pyrrolo[2,3-b]pyridine-3-carboxylate

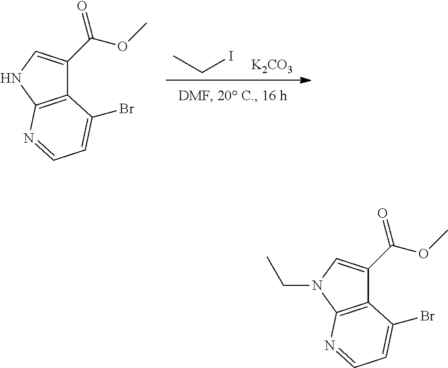

To a solution of methyl 4-bromo-1H-pyrrolo[2,3-b]pyridine-3-carboxylate (1 g, 3.92 mmol, 1 eq) in DMF (10 mL) was added K₂CO₃ (1.08 g, 7.84 mmol, 2 eq) and iodoethane (3.06 g, 19.60 mmol, 1.57 mL, 5 eq). The mixture was stirred at 25° C. for 2 hr. LC-MS showed no starting material was remained. Several new peaks were shown on LC-MS and 80% of desired compound was detected. The reaction mixture was concentrated under reduced pressure to remove dioxane. The residue was diluted with H₂O 5 mL and extracted with EA 30 mL (10 mL×3). The combined organic layers were washed with brine 5 mL, dried over Na₂SO₄, filtered and concentrated under reduced pressure to give a residue. The residue was purified by column chromatography (SiO₂, Petroleum ether/Ethyl acetate=10/1 to 0/1). Compound methyl 4-bromo-1-ethyl-pyrrolo[2,3-b]pyridine-3-carboxylate (850 mg, 3.00 mmol, 76.58% yield) was obtained as a yellow oil. HPLC showed the product was clean. LCMS for product (ESI): m/z 282.9 [M+H]⁺.

### Step 2: methyl 1-ethyl-4-[[4-(trifluoromethyl)phenyl]methyl]pyrrolo[2,3-b]pyridine-3-carboxylate

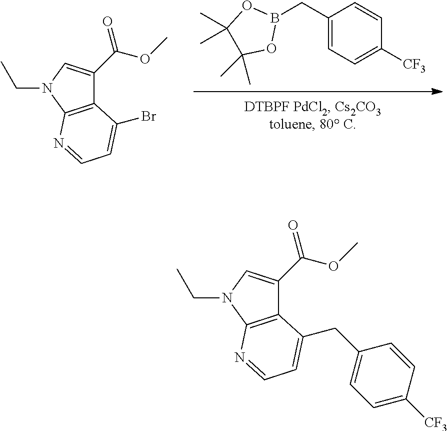

To a solution of methyl 4-bromo-1-ethyl-pyrrolo[2,3-b]pyridine-3-carboxylate (300 mg, 1.06 mmol, 1 eq) and 4,4,5,5-tetramethyl-2-[[4-(trifluoromethyl)phenyl]methyl]-1,3,2-dioxaborolane (454.73 mg, 1.59 mmol, 1.5 eq) in toluene (1 mL) was added ditert-butyl(cyclopentyl)phosphane;dichloropalladium;iron (69.06 mg, 105.96 umol, 0.1 eq) and Cs₂CO₃ (690.49 mg, 2.12 mmol, 2 eq). The mixture was stirred at 80° C. for 12 hr. LC-MS showed 0% of the bromide was remained. Several new peaks were shown on LC-MS and 24% of desired compound was detected. The reaction mixture was concentrated under reduced pressure to remove Toluene. The residue was diluted with H₂O 5 mL and extracted with EA 15 mL (5 mL×3). The combined organic layers were washed with brine (5 mL), dried over Na₂SO₄, filtered and concentrated under reduced pressure to give a residue. The residue was purified by prep-TLC (SiO₂, PE:EA=1:1). Compound methyl 1-ethyl-4-[[4-(trifluoromethyl)phenyl]methyl]pyrrolo[2,3-b]pyridine-3-carboxylate (180 mg, 496.76 umol, 46.88% yield) was obtained as a white solid.

LCMS for product (ESI): m/z 363.1 [M+H]⁺.

### Step 3: 1-ethyl-4-[[4-(trifluoromethyl)phenyl]methyl]pyrrolo[2,3-b]pyridine-3-carboxylic acid

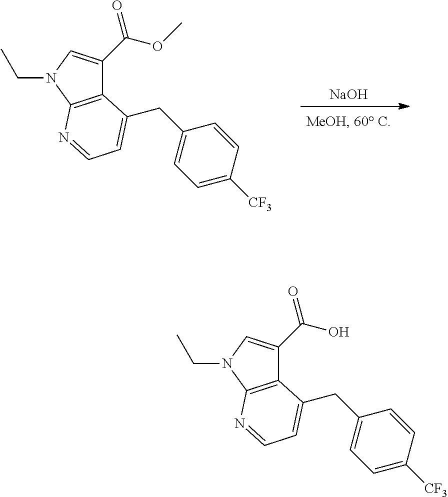 [SMILES: CCn1cc(C(=O)OC)c2c(Cc3ccc(C(F)(F)F)cc3)ccnc21 ; "NaOH" ; CCn1cc(C(=O)O)c2c(Cc3ccc(C(F)(F)F)cc3)ccnc21]

To a solution of methyl 1-ethyl-4-[[4-(trifluoromethyl)phenyl]methyl]pyrrolo[2,3-b]pyridine-3-carboxylate (180 mg, 496.76 umol, 1 eq) in H₂O (1 mL) and MeOH (1 mL) was added NaOH (19.87 mg, 496.76 umol, 1 eq). The mixture was stirred at 80° C. for 2 hr. HPLC showed the methyl ester was consumed completely. The reaction mixture was concentrated under reduced pressure to remove MeOH. The mixture was adjusted to Ph 4 with 1N HCl. The mixture was filtered. Compound 1-ethyl-4-[[4-(trifluoromethyl)phenyl]methyl]pyrrolo[2,3-b]pyridine-3-carboxylic acid (120 mg, 344.51 umol, 69.35% yield) was obtained as a white solid.

### Step 4: methyl 4-[(1S)-1-[[1-ethyl-4-[[4-(trifluoromethyl)phenyl]methyl]pyrrolo[2,3-b]pyridine-3-carbonyl]amino]ethyl]benzoate

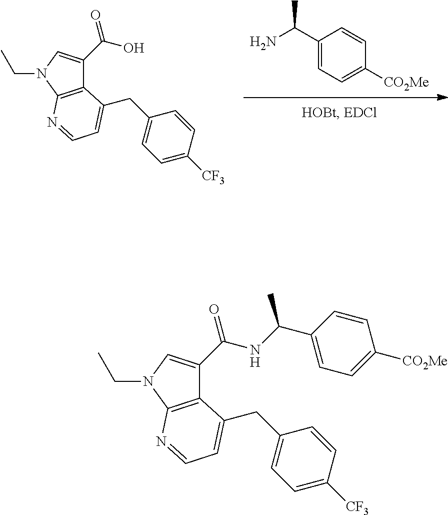 [SMILES: CCn1cc(C(=O)O)c2c(Cc3ccc(C(F)(F)F)cc3)ccnc21 ; COC(=O)c1ccc([C@H](C)N)cc1 ; CCn1cc(C(=O)N[C@@H](C)c2ccc(C(=O)OC)cc2)c2c(Cc3ccc(C(F)(F)F)cc3)ccnc21]

To a solution of 1-ethyl-4-[[4-(trifluoromethyl)phenyl]methyl]pyrrolo[2,3-b]pyridine-3-carboxylic acid (60 mg, 172.26 umol, 1 eq) in DCM (1 mL) was added dropwise Et₃N (87.15 mg, 861.28 umol, 119.88 uL, 5 eq), HOBt (4.66 mg, 34.45 umol, 0.2 eq) and EDCI (66.04 mg, 344.51 umol, 2 eq) at 0° C. After addition, the mixture was stirred at this temperature for 0.5 hr, and then methyl 4-[(1S)-1-aminoethyl]benzoate (46.31 mg, 258.38 umol, 1.5 eq) was added dropwise at 0° C. The resulting mixture was stirred at 25° C. for 12 hr. LCMS showed the carboxylic acid was consumed completely. The residue was diluted with H₂O 3 mL and extracted with DCM 15 mL (5 mL×3). The combined organic layers were washed with brine (3 mL), dried over Na₂SO₄, filtered and concentrated under reduced pressure to give a residue. The crude product was used into the next step without further purification. Compound methyl 4-[(1S)-1-[[1-ethyl-4-[[4-(trifluoromethyl)phenyl]methyl]pyrrolo[2,3-b]pyridine-3-carbonyl]amino]ethyl]benzoate (60 mg, crude) was obtained as a white solid, which was used into the next step directly without further purification. LCMS for product (ESI): m/z 510.3 [M+H]⁺.

### Step 5: 4-[(1S)-1-[[1-ethyl-4-[[4-(trifluoromethyl)phenyl]methyl]pyrrolo[2,3-b]pyridine-3-carbonyl]amino]ethyl]benzoic acid

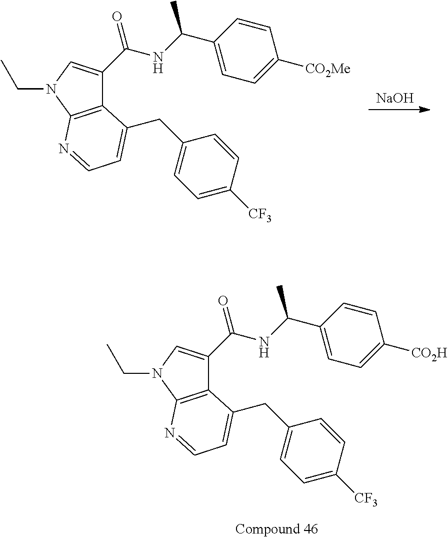 [SMILES: CCn1cc(C(=O)N[C@@H](C)c2ccc(C(=O)OC)cc2)c2c(Cc3ccc(C(F)(F)F)cc3)ccnc21 ; "NaOH" ; CCn1cc(C(=O)N[C@@H](C)c2ccc(C(=O)O)cc2)c2c(Cc3ccc(C(F)(F)F)cc3)ccnc21]

To a solution of methyl 4-[(1S)-1-[[1-ethyl-4-[[4-(trifluoromethyl)phenyl]methyl]pyrrolo[2,3-b]pyridine-3-carbonyl]amino]ethyl]benzoate (60 mg, 117.76 umol, 1 eq) in H₂O (5 mL) and MeOH (5 mL) was added NaOH (14.13 mg, 353.27 umol, 3 eq). The mixture was stirred at 80° C. for 2 hr. HPLC showed the methyl ester was consumed completely. The reaction mixture was concentrated under reduced pressure to remove solvent. The mixture was adjusted to pH=5 with 1N HCl. The mixture was filtered. Compound 4-[(1S)-1-[[1-ethyl-4-[[4-(trifluoromethyl)phenyl]methyl]pyrrolo[2,3-b]pyridine-3-carbonyl]amino]ethyl]benzoic acid (55 mg, 111.11 umol, 94.6% yield, 100% purity) was obtained as a white solid. LCMS for product (ESI): m/z 496.0 [M+H]⁺. ¹H NMR (400 MHz, DMSO-d₆) δ=8.63 (d, J=8.0 Hz, 1H), 8.26-8.22 (m, 1H), 8.11 (s, 1H), 7.91 (d, J=8.0 Hz, 2H), 7.51 (d, J=8.0 Hz, 2H), 7.38 (d, J=8.4 Hz, 2H), 7.24 (d, J=8.4 Hz, 2H), 7.10 (d, J=4.8 Hz, 1H), 5.25-5.16 (m, 1H), 4.75 (d, J=13.6 Hz, 1H), 4.54 (d, J=13.6 Hz, 1H), 4.36-4.28 (m, 2H), 1.47-1.38 (m, 6H).

### Example 51—Synthesis of 4-[(1S)-1-[[1-(2-fluoroethyl)-4-[[4-(trifluoromethyl)phenyl]methyl]pyrrolo[2,3-b]pyridine-3-carbonyl]amino]ethyl]benzoic acid (Compound 47)

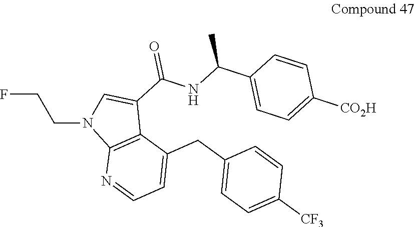 [SMILES: C[C@H](NC(=O)c1cn(CCF)c2nccc(Cc3ccc(C(F)(F)F)cc3)c12)c1ccc(C(=O)O)cc1]

### Step 1: methyl 4-[(1S)-1-[[1-(2-fluoroethyl)-4-[[4-(trifluoromethyl)phenyl]methyl]pyrrolo[2,3-b]pyridine-3-carbonyl]amino]ethyl]benzoate

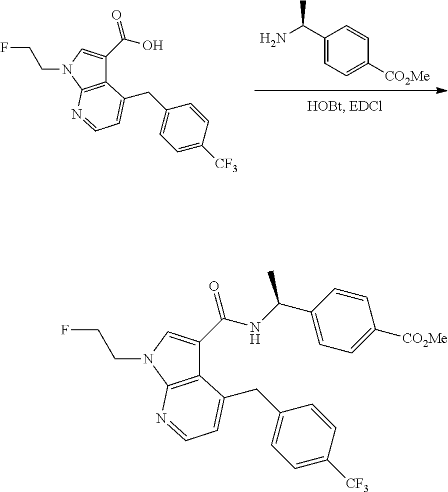 [SMILES: O=C(O)c1cn(CCF)c2nccc(Cc3ccc(C(F)(F)F)cc3)c12 ; COC(=O)c1ccc([C@H](C)N)cc1 ; COC(=O)c1ccc([C@H](C)NC(=O)c2cn(CCF)c3nccc(Cc4ccc(C(F)(F)F)cc4)c23)cc1]

To a solution of 1-(2-fluoroethyl)-4-[[4-(trifluoromethyl)phenyl]methyl]pyrrolo[2,3-b]pyridine-3-carboxylic acid (60 mg, 163.80 umol, 1 eq) in DCM (1 mL) was added dropwise Et₃N (82.87 mg, 818.98 umol, 113.99 uL, 5 eq), HOBt (4.43 mg, 32.76 umol, 0.2 eq) and EDCI (62.80 mg, 327.59 umol, 2 eq) at 0° C. After addition, the mixture was stirred at this temperature for 0.5 hr, and then methyl 4-[(1S)-1-aminoethyl]benzoate (44.03 mg, 245.69 umol, 1.5 eq) was added at 0° C. The resulting mixture was stirred at 25° C. for 12 hr. LCMS showed the carboxylic acid was consumed completely. The residue was diluted with H₂O 5 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound methyl 4-[(1S)-1-[[1-(2-fluoroethyl)-4-[[4-(trifluoromethyl)phenyl]methyl]pyrrolo[2,3-b]pyridine-3-carbonyl]amino]ethyl]benzoate (60 mg, crude) was obtained as a white solid, which was used into the next step directly without further purification. LCMS for product (ESI): m/z 528.3 [M+H]⁺.

### Step 2: 4-[(1S)-1-[[1-(2-fluoroethyl)-4-[[4-(trifluoromethyl)phenyl]methyl]pyrrolo[2,3-b]pyridine-3-carbonyl]amino]ethyl]benzoic acid

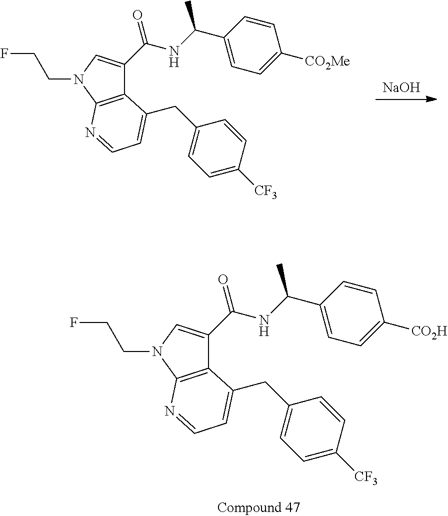 [SMILES: COC(=O)c1ccc([C@H](C)NC(=O)c2cn(CCF)c3nccc(Cc4ccc(C(F)(F)F)cc4)c23)cc1 ; "NaOH" ; C[C@H](NC(=O)c1cn(CCF)c2nccc(Cc3ccc(C(F)(F)F)cc3)c12)c1ccc(C(=O)O)cc1]

To a solution of methyl 4-[(1S)-1-[[1-(2-fluoroethyl)-4-[[4-(trifluoromethyl)phenyl]methyl]pyrrolo[2,3-b]pyridine-3-carbonyl]amino]ethyl]benzoate (60 mg, 113.74 umol, 1 eq) in H₂O (1 mL) and MeOH (1 mL) was added NaOH (4.55 mg, 113.74 umol, 1 eq). The mixture was stirred at 80° C. for 1 hr. HPLC showed the methyl ester was consumed completely. The reaction mixture was concentrated under reduced pressure to remove MeOH. The mixture was adjusted to pH=5 with 1N HCl. The mixture was filtered. Compound 4-[(1S)-1-[[1-(2-fluoroethyl)-4-[[4-(trifluoromethyl)phenyl]methyl]pyrrolo[2,3-b]pyridine-3-carbonyl]amino]ethyl]benzoic acid (46 mg, 89.66 umol, 79.35% yield, 98.4% purity) was obtained as a white solid. LCMS for product (ESI): m/z 514.0 [M+H]⁺. ¹H NMR (400 MHz, DMSO-d₆) δ=8.69 (d, J=8.0 Hz, 1H), 8.25 (d, J=4.8 Hz, 1H), 8.10 (s, 1H), 7.90 (d, J=8.0 Hz, 2H), 7.50 (d, J=8.0 Hz, 2H), 7.39 (d, J=8.0 Hz, 2H), 7.24 (d, J=8.0 Hz, 2H), 7.13 (d, J=4.8 Hz, 1H), 5.25-5.15 (m, 1H), 4.89 (t, J=4.4 Hz, 1H), 4.79-4.70 (m, 2H), 4.65 (t, J=4.8 Hz, 1H), 4.60-4.52 (m, 2H), 1.44 (d, J=7.2 Hz, 3H).

### Example 52—Synthesis of trans-2-[[1-(2-fluoroethyl)-4-[[4-(trifluoromethyl)phenyl]methyl]-pyrrolo[2,3-b]pyridine-3-carbonyl]amino]spiro[3.3]heptane-6-carboxylic acid (Compound 48)

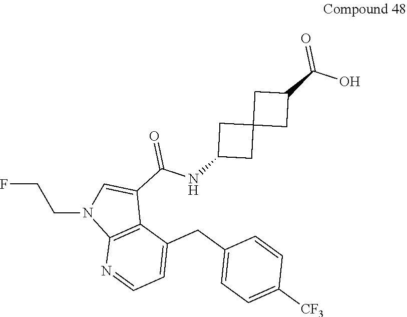

### Step 1: methyl 4-bromo-1-(2-fluoroethyl)pyrrolo[2,3-b]pyridine-3-carboxylate

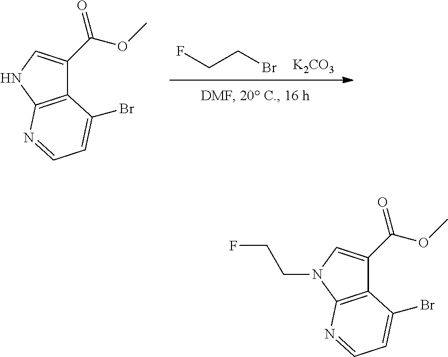

To a solution of methyl 4-bromo-1H-pyrrolo[2,3-b]pyridine-3-carboxylate (1 g, 3.92 mmol, 1 eq) in DMF (1 mL) was added K₂CO₃ (1.08 g, 7.84 mmol, 2 eq) and 1-bromo-2-fluoro-ethane (2.49 g, 19.60 mmol, 5 eq). The mixture was stirred at 25° C. for 16 hr. LC-MS showed no starting material was remained. Several new peaks were shown on LC-MS and 80% of desired compound was detected. The reaction mixture was concentrated under reduced pressure to remove dioxane. The residue was diluted with H₂O 5 mL and extracted with EA 15 mL (5 mL×3). The combined organic layers were washed with brine 5 mL, dried over Na₂SO₄, filtered and concentrated under reduced pressure to give a residue. The residue was purified by column chromatography (SiO₂, Petroleum ether/Ethyl acetate=10/1 to 0/1). Compound methyl 4-bromo-1-(2-fluoroethyl)pyrrolo[2,3-b]pyridine-3-carboxylate (900 mg, 2.99 mmol, 76.24% yield) was obtained as a white solid. HPLC showed the product was clean. LCMS for product (ESI): m/z 300.9 [M+H]⁺.

### Step 2: methyl 1-(2-fluoroethyl)-4-[[4-(trifluoromethyl)phenyl]methyl]pyrrolo[2,3-b]pyridine-3-carboxylate

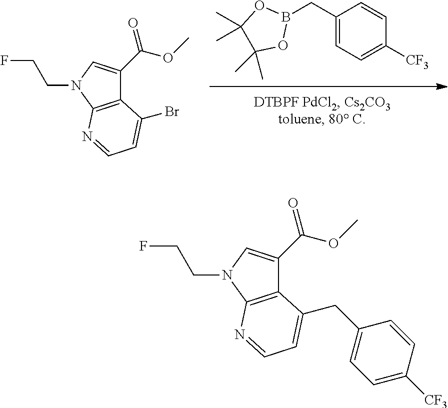

To a solution of methyl 4-bromo-1-(2-fluoroethyl)pyrrolo[2,3-b]pyridine-3-carboxylate (406.51 mg, 1.35 mmol, 1 eq) and 4,4,5,5-tetramethyl-2-[[4-(trifluoromethyl)phenyl]methyl]-1,3,2-dioxaborolane (579.37 mg, 2.03 mmol, 1.5 eq) in toluene (3 mL) was added Cs₂CO₃ (879.74 mg, 2.70 mmol, 2 eq) and ditert-butyl(cyclopentyl)phosphane;dichloropalladium;iron (87.99 mg, 135.00 umol, 0.1 eq). The mixture was stirred at 80° C. for 12 hr. LC-MS showed 0% of the bromide was remained. Several new peaks were shown on LC-MS and 24% of desired compound was detected. The reaction mixture was concentrated under reduced pressure to remove TOLUENE. The residue was diluted with H₂O 5 mL and extracted with EA 15 mL (5 mL×3). The combined organic layers were washed with brine (5 mL), dried over Na₂SO₄, filtered and concentrated under reduced pressure to give a residue. The residue was purified by prep-TLC (SiO₂, PE:EA=1:1). Compound methyl 1-(2-fluoroethyl)-4-[[4-(trifluoromethyl)phenyl]methyl]pyrrolo[2,3-b]pyridine-3-carboxylate (150 mg, 394.39 umol, 29.21% yield) was obtained as a white solid. HPLC showed the product was clean. LCMS for product (ESI): m/z 381.1 [M+H]⁺.

### Step 3: 1-(2-fluoroethyl)-4-[[4-(trifluoromethyl)phenyl]methyl]pyrrolo[2,3-b]pyridine-3-carboxylic acid

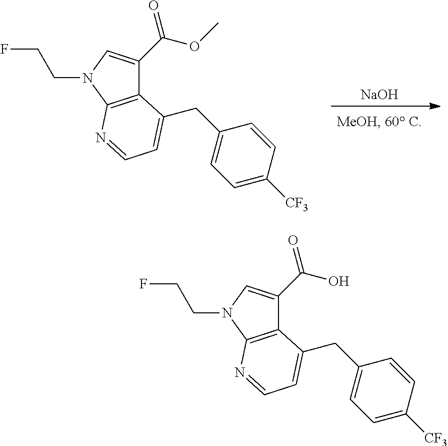

To a solution of methyl 1-(2-fluoroethyl)-4-[[4-(trifluoromethyl)phenyl]methyl]pyrrolo[2,3-b]pyridine-3-carboxylate (150 mg, 394.39 umol, 1 eq) in H₂O (1 mL) and MeOH (1 mL) was added NaOH (15.77 mg, 394.39 umol, 1 eq). The mixture was stirred at 80° C. for 3 hr. HPLC showed the methyl ester was consumed completely. The reaction mixture was concentrated under reduced pressure to remove MeOH. The mixture was adjusted to Ph 4 with 1N HCl. The mixture was filtered. Compound 1-(2-fluoroethyl)-4-[[4-(trifluoromethyl)phenyl]methyl]pyrrolo[2,3-b]pyridine-3-carboxylic acid (120 mg, 327.59 umol, 83.06% yield) was obtained as a white solid.

### Step 4: trans-(S)-1-phenylethyl 2-[[1-(2-fluoroethyl)-4-[[4-(trifluoromethyl)-phenyl]methyl]-pyrrolo[2,3-b]pyridine-3-carbonyl]amino]-spiro[3.3]heptane-6-carboxylate

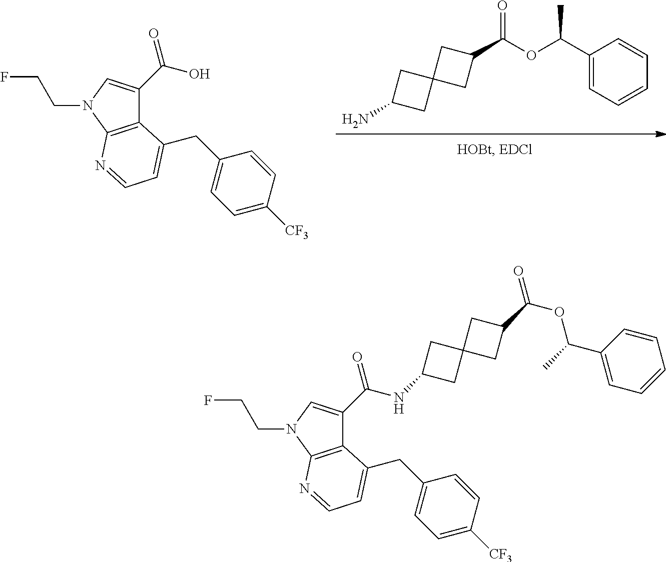

To a solution of 1-(2-fluoroethyl)-4-[[4-(trifluoromethyl)phenyl]methyl]pyrrolo[2,3-b]pyridine-3-carboxylic acid (30 mg, 81.90 umol, 1 eq) in DCM (1 mL) was added dropwise Et₃N (41.44 mg, 409.50 umol, 57.00 uL, 5 eq), HOBt (2.21 mg, 16.38 umol, 0.2 eq) and EDCI (31.40 mg, 163.80 umol, 2 eq) at 0° C. After addition, the mixture was stirred at this temperature for 0.5 hr, and then trans-(S)-1-phenylethyl 2-aminospiro[3.3]heptane-6-carboxylate (31.86 mg, 122.85 umol, 1.5 eq) was added dropwise at 0° C. The resulting mixture was stirred at 25° C. for 12 hr. LCMS showed the carboxylic acid was consumed completely. The residue was diluted with H₂O 3 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound trans-(S)-1-phenylethyl 2-[[1-(2-fluoroethyl)-4-[[4-(trifluoromethyl)phenyl]methyl]pyrrolo[2,3-b]pyridine-3-carbonyl]amino]spiro[3.3]heptane-6-carboxylate (15 mg, 24.69 umol, 30.14% yield) was obtained as a white solid, which was used into the next step without further purification.

LCMS for product (ESI): m/z 608.3 [M+H]⁺.

### Step 5: trans-2-[[1-(2-fluoroethyl)-4-[[4-(trifluoromethyl)phenyl]methyl]-pyrrolo[2,3-b]pyridine-3-carbonyl]amino]spiro[3.3]heptane-6-carboxylic acid

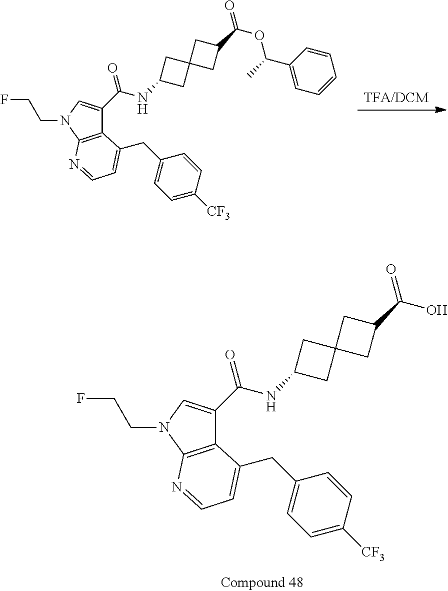

To a solution of trans-(S)-1-phenylethyl 2-[[1-(2-fluoroethyl)-4-[[4-(trifluoromethyl)phenyl]methyl]pyrrolo[2,3-b]pyridine-3-carbonyl]amino]spiro[3.3]heptane-6-carboxylate (15 mg, 24.69 umol, 1 eq) in DCM (1 mL) and TFA (1 mL). The mixture was stirred at 25° C. for 0.5 hr. HPLC showed the ester was consumed completely. The reaction mixture was concentrated under reduced pressure to remove solvent. The residue was added 1M NaOH(aq) and diluted with H₂O 3 mL and extracted with DCM 15 mL (5 mL×3). The combined organic layers were washed with brine (5 mL), dried over Na₂SO₄, filtered and concentrated under reduced pressure to give a residue. The mixture was adjusted to pH=5 with 1N HCl. The mixture was filtered. Compound trans-2-[[1-(2-fluoroethyl)-4-[[4-(trifluoromethyl)phenyl]methyl]pyrrolo[2,3-b]pyridine-3-carbonyl]amino]spiro[3.3]heptane-6-carboxylic acid (4.5 mg, 8.54 umol, 34.61% yield, 95.6% purity) was obtained as a white solid. LCMS for product (ESI): m/z 504.0 [M+H]⁺. ¹H NMR (400 MHz, DMSO-d₆) δ=8.32 (d, J=7.6 Hz, 1H), 8.24 (d, J=4.8 Hz, 1H), 7.97 (s, 1H), 7.55 (d, J=8.0 Hz, 2H), 7.34 (d, J=8.0 Hz, 2H), 7.10 (d, J=4.8 Hz, 1H), 4.87 (t, J=4.4 Hz, 1H), 4.75 (t, J=4.8 Hz, 1H), 4.68 (s, 2H), 4.61 (t, J=4.4 Hz, 1H), 4.55 (t, J=4.4 Hz, 1H), 4.28-4.19 (m, 1H), 2.94-2.87 (m, 1H), 2.43-2.40 (m, 1H), 2.25-2.20 (m, 3H), 2.14-2.05 (m, 2H), 1.99-1.88 (m, 2H).

### Example 53—Synthesis of 2-[4-[[4-[[4-(trifluoromethyl)phenyl]methyl]pyrazolo[1,5-a]pyridine-3-carbonyl]amino]phenyl]propanoic acid (Compound 49)

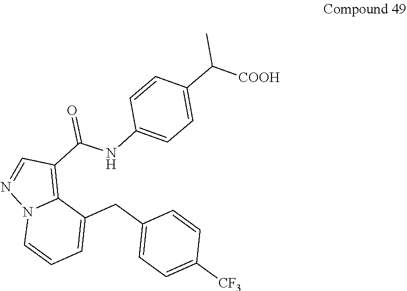 [SMILES: CC(C(=O)O)c1ccc(NC(=O)c2cnn3cccc(Cc4ccc(C(F)(F)F)cc4)c23)cc1]

### Step 1: methyl 2-[4-[[4-[[4-(trifluoromethyl)phenyl]methyl]pyrazolo[1,5-a]pyridine-3-carbonyl]amino]phenyl]propanoate

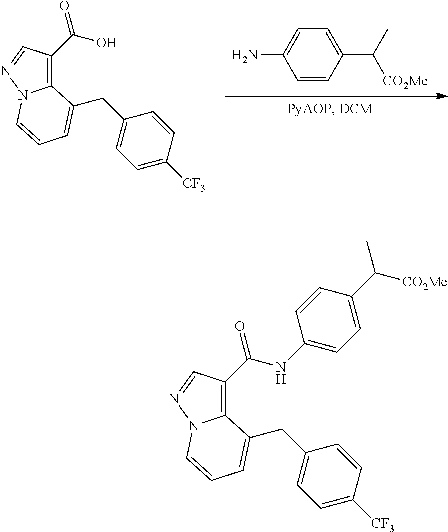

To a solution of 4-[[4-(trifluoromethyl)phenyl]methyl]pyrazolo[1,5-a]pyridine-3-carboxylic acid (20 mg, 62.45 umol, 1 eq) and methyl 2-(4-aminophenyl)propanoate (13.43 mg, 74.94 umol, 1.2 eq) in DCM (1 mL) was added PYAOP (35.82 mg, 68.69 umol, 1.1 eq) and DIEA (20.18 mg, 156.12 umol, 27.19 uL, 2.5 eq). The mixture was stirred at 25° C. for 5 hr. LC-MS showed 0% of the carboxylic acid was remained. Several new peaks were shown on LC-MS and 61.38% of desired compound was detected. The reaction mixture was diluted with H₂O 2 mL and extracted with DCM 9 mL (3 mL×3). The combined organic layers were washed with brine (3 mL), dried over Na₂SO₄, filtered and concentrated under reduced pressure to give a residue. The residue was purified by prep-TLC (SiO₂, PE:EA=1:1). Compound methyl 2-[4-[[4-[[4-(trifluoromethyl)phenyl]methyl]pyrazolo[1,5-a]pyridine-3-carbonyl]amino]phenyl]propanoate (20 mg, 41.54 umol, 66.52% yield) was obtained as a white solid, which was used into the next step directly without further purification. LCMS for product (ESI): m/z 482.2 [M+H]⁺.

### Step 2: 2-[4-[[4-[[4-(trifluoromethyl)phenyl]methyl]pyrazolo[1,5-a]pyridine-3-carbonyl]amino]phenyl]propanoic acid

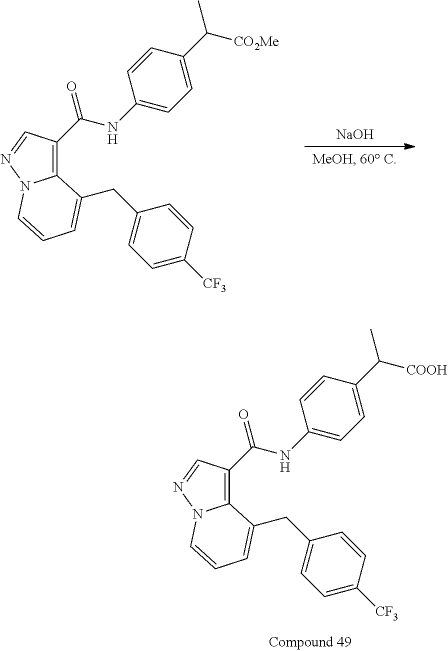 [SMILES: COC(=O)C(C)c1ccc(NC(=O)c2cnn3cccc(Cc4ccc(C(F)(F)F)cc4)c23)cc1 ; "NaOH" ; "MeOH, 60° C." ; CC(C(=O)O)c1ccc(NC(=O)c2cnn3cccc(Cc4ccc(C(F)(F)F)cc4)c23)cc1]

To a solution of methyl 2-[4-[[4-[[4-(trifluoromethyl)phenyl]methyl]pyrazolo[1,5-a]pyridine-3-carbonyl]amino]phenyl]propanoate (20 mg, 41.54 umol, 1 eq) in H₂O (1 mL) and MeOH (1 mL) was added NaOH (4.98 mg, 124.62 umol, 3 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 2-[4-[[4-[[4-(trifluoromethyl)phenyl]methyl]pyrazolo[1,5-a]pyridine-3-carbonyl]amino]phenyl]propanoic acid (15 mg, 31.79 umol, 76.54% yield, 99.08% purity) was obtained as a white solid. LCMS for product (ESI): m/z 468.2 [M+H]⁺. ¹H NMR (400 MHz, DMSO-d₆) δ ppm 12.28 (br s, 1H) 10.16 (s, 1H) 8.76 (d, J=6.8 Hz, 1H) 8.42 (s, 1H) 7.63 (d, J=8.4 Hz, 2H) 7.52 (d, J=8.0 Hz, 2H) 7.35-7.31 (m, 3H) 7.26 (d, J=8.8 Hz, 2H) 7.10 (t, J=7.2 Hz, 1H) 4.66 (s, 2H) 3.66 (q, J=7.2 Hz, 1H) 1.38 (d, J=7.2 Hz, 3H).

### Example 54—Synthesis of 4-[[[4-[[4-(trifluoromethyl) phenyl]methyl]pyrazolo[1,5-a]pyridine-3-carbonyl]amino]methyl]norbornane-1-carboxylic acid (Compound 50)

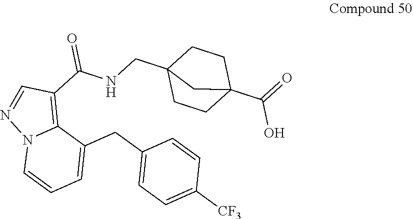 [SMILES: O=C(NCC12CCC(C(=O)O)(CC1)C2)c1cnn2cccc(Cc3ccc(C(F)(F)F)cc3)c12]

### Step 1: (2,5-dioxopyrrolidin-1-yl) 4-[[4-(trifluoromethyl) phenyl]methyl]pyrazolo[1,5-a]pyridine-3-carboxylate

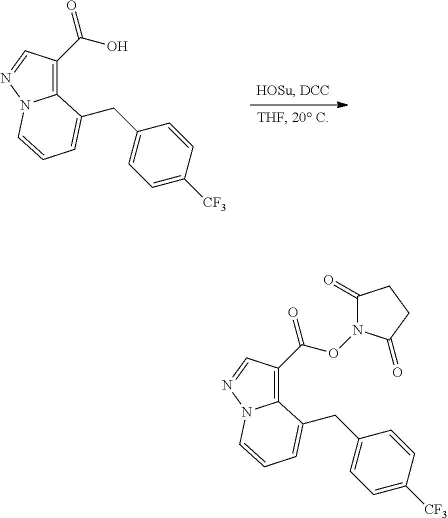 [SMILES: O=C(O)c1cnn2cccc(Cc3ccc(C(F)(F)F)cc3)c12 ; "HOSu, DCC" ; "THF, 20° C." ; O=C(ON1C(=O)CCC1=O)c1cnn2cccc(Cc3ccc(C(F)(F)F)cc3)c12]

To a solution of 4-[[4-(trifluoromethyl) phenyl]methyl]pyrazolo[1,5-a]pyridine-3-carboxylic acid (50 mg, 156.12 umol, 1 eq) and 1-hydroxypyrrolidine-2,5-dione (21.56 mg, 187.34 umol, 1.2 eq) in THF (1 mL) was added DCC (41.88 mg, 202.96 umol, 41.05 uL, 1.3 eq). The mixture was stirred at 25° C. for 16 hr. LC-MS showed 0% of the carboxylic acid was remained. Several new peaks were shown on LC-MS and 50.69% of desired compound was detected. The reaction mixture was concentrated under reduced pressure to remove THF. The residue was diluted with H₂O 3 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound (2,5-dioxopyrrolidin-1-yl) 4-[[4-(trifluoromethyl) phenyl]methyl]pyrazolo[1,5-a]pyridine-3-carboxylate (48 mg, 115.01 umol, 73.67% yield) was obtained as black oil.

### Step 2: 4-[[[4-[[4-(trifluoromethyl) phenyl]methyl]pyrazolo[1,5-a]pyridine-3-carbonyl]amino]methyl]norbornane-1-carboxylic acid

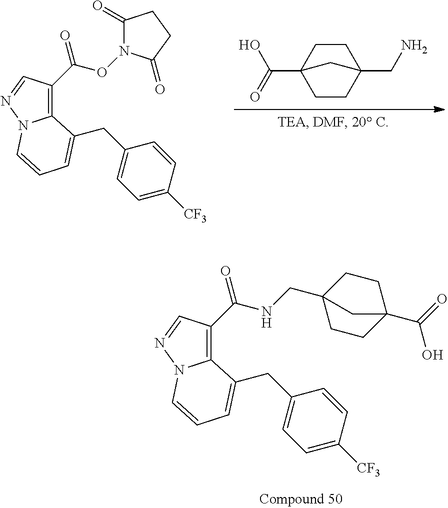

To a solution of (2,5-dioxopyrrolidin-1-yl) 4-[[4-(trifluoromethyl) phenyl]methyl]pyrazolo[1,5-a]pyridine-3-carboxylate (40 mg, 95.85 umol, 1 eq) and 4-(aminomethyl) norbornane-1-carboxylic acid (32.44 mg, 191.69 umol, 2 eq) in DMF (1 mL) was added Et₃N (174.58 mg, 1.73 mmol, 240.13 uL, 18 eq). The mixture was stirred at 60° C. for 16 hr. LC-MS showed 0% of the ester was remained. Several new peaks were shown on LC-MS and 30.14% of desired compound was detected. The reaction mixture was concentrated under reduced pressure to remove DMF. The residue was diluted with H₂O 3 mL and extracted with EA 15 mL (5 mL×3). The combined organic layers were washed with brine (5 mL), dried over Na₂SO₄, filtered and concentrated under reduced pressure to give a residue. The residue was purified by prep-HPLC (HCl condition). Compound 4-[[[4-[[4-(trifluoromethyl) phenyl]methyl]pyrazolo[1,5-a]pyridine-3-carbonyl]amino]methyl]norbornane-1-carboxylic acid (6 mg, 12.53 umol, 13.08% yield, 98.49% purity) was obtained as a white solid. LCMS for product (ESI): m/z 472.0 [M+H]⁺. ¹H NMR (400 MHz, DMSO-d₆) δ=12.01 (s, 1H), 8.71 (d, J=6.8 Hz, 1H), 8.30 (s, 1H), 8.20 (t, J=6.0 Hz, 1H), 7.58 (d, J=8.0 Hz, 2H), 7.32-7.28 (m, 3H), 7.04 (t, J=6.8 Hz, 1H), 4.70 (s, 2H), 2.69 (s, 2H), 1.88-1.78 (m, 2H), 1.59-1.50 (m, 4H), 1.46 (s, 2H), 1.30-1.24 (m, 2H).

### Example 55—Synthesis of 2-[3-[[1-ethyl-4-[[4-(trifluoromethyl) phenyl]methyl]indole-3-carbonyl]amino]-1-bicyclo[1.1.1]pentanyl]acetic acid (Compound 51)

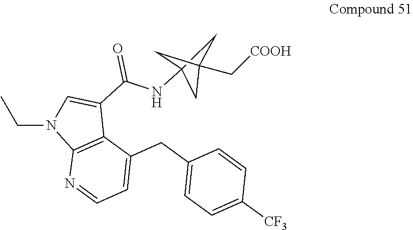 [SMILES: CCn1cc(C(=O)NC23CC(CC(=O)O)(C2)C3)c2c(Cc3ccc(C(F)(F)F)cc3)ccnc21]

### Step 1: methyl 2-[3-[[1-ethyl-4-[[4-(trifluoromethyl) phenyl]methyl]indole-3-carbonyl]amino]-1-bicyclo[1.1.11pentanyl]acetate

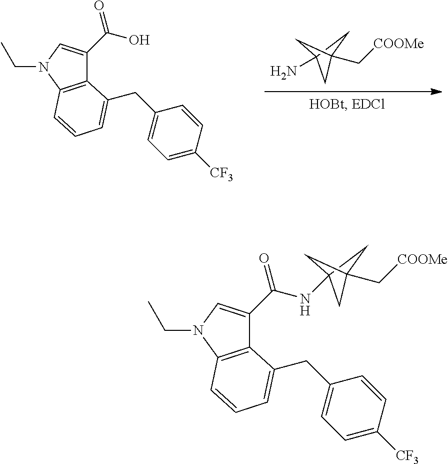

To a solution of 1-ethyl-4-[[4-(trifluoromethyl) phenyl]methyl]indole-3-carboxylic acid (20 mg, 57.58 umol, 1 eq) in DCM (2 mL) was added HOBt (1.56 mg, 11.52 umol, 0.2 eq), EDCI (22.08 mg, 115.16 umol, 2 eq) and Et₃N (29.13 mg, 287.91 umol, 40.07 uL, 5 eq) at 0° C. The mixture was stirred at 0° C. for 0.5 hr. Then methyl 2-(3-amino-1-bicyclo[1.1.1]pentanyl) acetate (13.40 mg, 86.37 umol, 1.5 eq) was added the mixture. The mixture was stirred at 25° C. for 16 hr. LC-MS showed 0% of the carboxylic acid was remained. Several new peaks were shown on LC-MS and 62.99% of desired compound was detected. The reaction mixture was diluted with H₂O 3 mL and extracted with DCM 9 mL (3 mL×3). The combined organic layers were washed with brine 5 mL, dried over Na₂SO₄, filtered and concentrated under reduced pressure to give a residue. The crude product was used into the next step without further purification. Compound methyl 2-[3-[[1-ethyl-4-[[4-(trifluoromethyl) phenyl]methyl]indole-3-carbonyl]amino]-1-bicyclo[1.1.1]pentanyl]acetate (10 mg, 20.64 umol, 35.84% yield) was obtained as yellow oil, which was used into next step without further purification. LCMS for product (ESI): m/z 485.3 [M+H]⁺.

### Step 2: 2-[3-[[1-ethyl-4-[[4-(trifluoromethyl) phenyl]methyl]indole-3-carbonyl]amino]-1-bicyclo[1.1.1]pentanyl]acetic acid

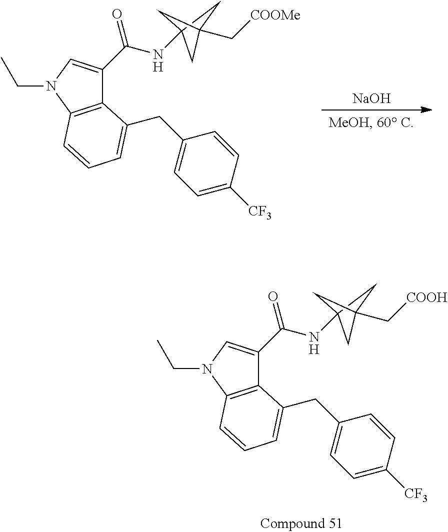 [SMILES: CCn1cc(C(=O)NC23CC(CC(=O)OC)(C2)C3)c2c(Cc3ccc(C(F)(F)F)cc3)cccc21 ; "NaOH" ; "MeOH, 60° C." ; CCn1cc(C(=O)NC23CC(CC(=O)O)(C2)C3)c2c(Cc3ccc(C(F)(F)F)cc3)cccc21]

To a solution of methyl 2-[3-[[1-ethyl-4-[[4-(trifluoromethyl) phenyl]methyl]indole-3-carbonyl]amino]-1-bicyclo[1.1.1]pentanyl]acetate (10 mg, 20.64 umol, 1 eq) in H₂O (1 mL) and MeOH (1 mL) was added NaOH (825.52 ug, 20.64 umol, 1 eq). The mixture was stirred at 50° C. for 16 hr. HPLC showed 0% of the methyl ester was remained. The reaction mixture was concentrated under reduced pressure to remove MeOH. The residue was purified by prep-HPLC (HCl condition). Compound 2-[3-[[1-ethyl-4-[[4-(trifluoromethyl) phenyl]methyl]indole-3-carbonyl]amino]-1-bicyclo[1.1.1]pentanyl]acetic acid (6 mg, 12.68 umol, 61.42% yield, 99.41% purity) was obtained as a white solid. LCMS for product (ESI): m/z 471.2 [M+H]⁺. ¹H NMR (400 MHz, DMSO-d₆) δ ppm 12.05 (br s, 1H) 8.41 (s, 1H) 7.68 (s, 1H) 7.48 (d, J=7.6 Hz, 2H) 7.39 (d, J=8.4 Hz, 1H) 7.29 (d, J=8.0 Hz, 2H) 7.19-7.13 (m, 1H) 7.04-7.00 (m, 1H) 4.61 (s, 2H) 4.20-4.13 (m, 2H) 2.5 (s, 2H), 1.97 (s, 6H) 1.33 (t, J=6.8 Hz, 3H).

### Example 56—Synthesis of 2-[3-[[1-(2-fluoroethyl)-4-[[4-(trifluoromethyl)phenyl]methyl]-indole-3-carbonyl]amino]-1-bicyclo[1.1.1.1]pentanyl]acetic acid (Compound 52)

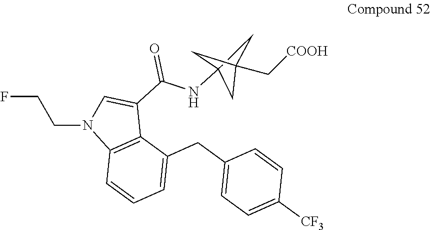 [SMILES: O=C(O)CC12CC(NC(=O)c3cn(CCF)c4cccc(Cc5ccc(C(F)(F)F)cc5)c34)(C1)C2]

### Step 1: methyl 1-(2-fluoroethyl)-4-[[4-(trifluoromethyl)phenyl]methyl]indole-3-carboxylate

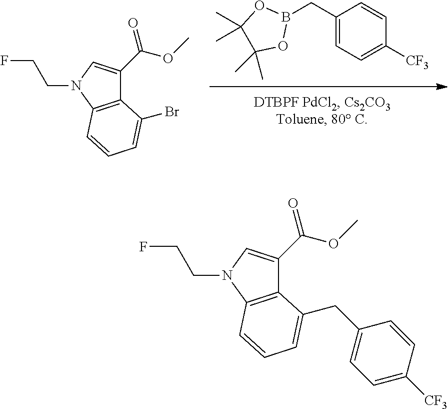

To a solution of methyl 4-bromo-1-(2-fluoroethyl)indole-3-carboxylate (300 mg, 999.59 umol, 1 eq) in toluene (3 mL) and 4,4,5,5-tetramethyl-2-[[4-(trifluoromethyl)phenyl]methyl]-1,3,2-dioxaborolane (428.97 mg, 1.50 mmol, 1.5 eq) was added Cs₂CO₃ (651.37 mg, 2.00 mmol, 2 eq) and ditert-butyl(cyclopentyl)phosphane;dichloropalladium;iron (65.15 mg, 99.96 umol, 0.1 eq). The mixture was stirred at 80° C. for 16 hr. LC-MS showed 0% of the bromide was remained. Several new peaks were shown on LC-MS and 22.2% of desired compound was detected. The reaction mixture was diluted with H₂O 3 mL and extracted with DCM 15 mL (5 mL×3). The combined organic layers were washed with brine 3 mL, dried over Na₂SO₄, filtered and concentrated under reduced pressure to give a residue. The residue was purified by prep-TLC (SiO₂,PE:EA=2:1). Compound methyl 1-(2-fluoroethyl)-4-[[4-(trifluoromethyl)phenyl]methyl]indole-3-carboxylate (130 mg, 342.69 umol, 34.28% yield) was obtained as yellow oil.

### Step 2: 1-(2-fluoroethyl)-4-[[4-(trifluoromethyl)phenyl]methyl]indole-3-carboxylic acid

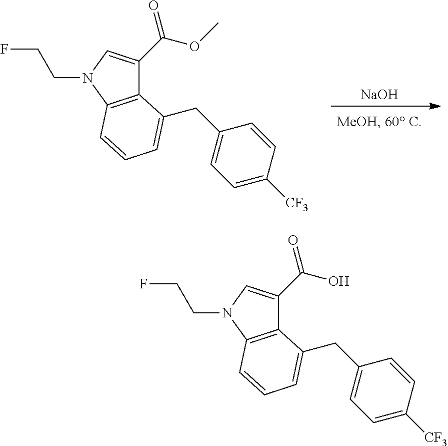

To a solution of methyl 1-(2-fluoroethyl)-4-[[4-(trifluoromethyl)phenyl]methyl]indole-3-carboxylate (130 mg, 342.69 umol, 1 eq) in MeOH (2 mL) and H₂O (1 mL) was added NaOH (13.71 mg, 342.69 umol, 1 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 1-(2-fluoroethyl)-4-[[4-(trifluoromethyl)phenyl]methyl]indole-3-carboxylic acid (110 mg, 301.11 umol, 87.86% yield) was obtained as a white solid.

### Step 3: methyl 2-[3-[[1-(2-fluoroethyl)-4-[[4-(trifluoromethyl)phenyl]methyl]indole-3-carbonyl]amino]-1-bicyclo[1.1.11pentanyl]acetate

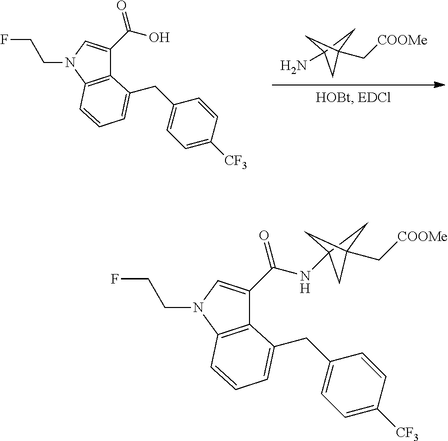

To a solution of 1-(2-fluoroethyl)-4-[[4-(trifluoromethyl)phenyl]methyl]indole-3-carboxylic acid (40 mg, 109.49 umol, 1 eq) in DCM (2 mL) was added HOBt (2.96 mg, 21.90 umol, 0.2 eq), EDCI (41.98 mg, 218.99 umol, 2 eq) and Et₃N (55.40 mg, 547.46 umol, 76.20 uL, 5 eq) at 0° C. The mixture was stirred at 0° C. for 0.5 hr. Then methyl 2-(3-amino-1 bicyclo[1.1.1]pentanyl)acetate (25.49 mg, 164.24 umol, 1.5 eq) was added the mixture. The mixture was stirred at 25° C. for 16 hr. LC-MS showed 0% of the carboxylic acid was remained. Several new peaks were shown on LC-MS and 53.32% of desired compound was detected. The reaction mixture was diluted with H₂O 3 mL and extracted with DCM 15 mL (5 mL×3). The combined organic layers were washed with brine 5 mL, dried over Na₂SO₄, filtered and concentrated under reduced pressure to give a residue. The crude product was used into the next step without further purification. Compound methyl 2-[3-[[1-(2-fluoroethyl)-4-[[4-(trifluoromethyl)phenyl]methyl]indole-3-carbonyl]amino]-1-bicyclo[1.1.1]pentanyl]acetate (30 mg, 59.70 umol, 54.53% yield) was obtained as yellow oil, which was used into the next step without further purification. LCMS for product (ESI): m/z 503.3 [M+H]⁺.

### Step 4: 2-[3-[[1-(2-fluoroethyl)-4-[[4-(trifluoromethyl)phenyl]methyl]indole-3-carbonyl]amino]-1-bicyclo[1.1.1]pentanyl]acetic acid

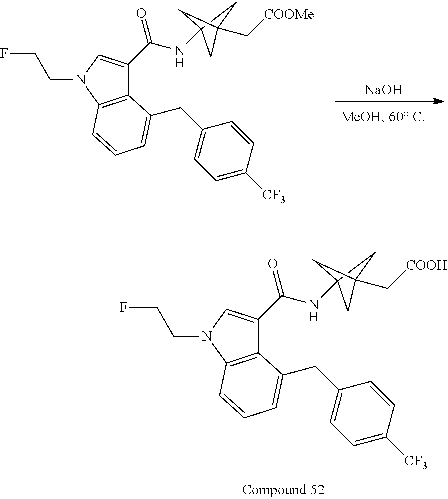 [SMILES: COC(=O)CC12CC(NC(=O)c3cn(CCF)c4cccc(Cc5ccc(C(F)(F)F)cc5)c34)(C1)C2 ; "NaOH" ; O=C(O)CC12CC(NC(=O)c3cn(CCF)c4cccc(Cc5ccc(C(F)(F)F)cc5)c34)(C1)C2]

To a solution of methyl 1-(2-fluoroethyl)-4-[[4-(trifluoromethyl)phenyl]methyl]indole-3-carboxylate (30 mg, 342.69 umol, 1 eq) in MeOH (2 mL) and H₂O (1 mL) was added NaOH (13.71 mg, 342.69 umol, 1 eq). The mixture was stirred at 50° C. for 16 hr. HPLC showed 0% of the methyl ester was remained. The reaction mixture was concentrated under reduced pressure to remove MeOH. The mixture was adjusted to pH 4 with 1N HCl. The mixture was filtered. Compound 2-[3-[[1-(2-fluoroethyl)-4-[[4-(trifluoromethyl)phenyl]methyl]indole-3-carbonyl]amino]-1-bicyclo[1.1.1]pentanyl]acetic acid (11 mg, 22.52 umol, 37.72% yield, 100% purity) was obtained as a white solid. LCMS for product (ESI): m/z 489.1 [M+H]⁺, Rt: 2.576 min. ¹H NMR (400 MHz, DMSO-d₆) δ ppm 8.53 (s, 1H) 7.70 (s, 1H) 7.52 (d, J=8.00 Hz, 2H) 7.46 (d, J=8.4 Hz, 1H) 7.32 (d, J=8.0 Hz, 2H) 7.21 (t, J=7.6 Hz, 1H) 7.08 (d, J=7.2 Hz, 1H) 4.80 (t, J=4.4 Hz, 1H) 4.68 (m, 1H) 4.64 (s, 2H) 4.52-4.56 (m, 1H) 4.48 (d, J=4.13 Hz, 1H) 3.15-3.31 (m, 2H), 2.50 (s, 2H), 2.01 (s, 6H).

### Example 57—Synthesis of 2-[3-[[1-ethyl-4-[[4-(trifluoromethyl)phenyl]methyl]indole-3-carbonyl]amino]-1-bicyclo[1.1.1]pentanyl]acetic acid (Compound 53)

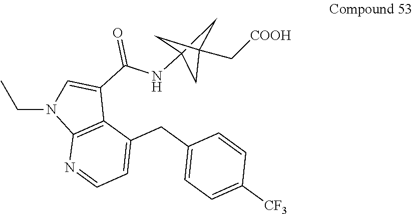 [SMILES: CCn1cc(C(=O)NC23CC(CC(=O)O)(C2)C3)c2c(Cc3ccc(C(F)(F)F)cc3)ccnc21]

### Step 1: methyl 2-[3-[[1-ethyl-4-[[4-(trifluoromethyl)phenyl]methyl]indole-3-carbonyl]amino]-1-bicyclo[1.1.11pentanyl]acetate

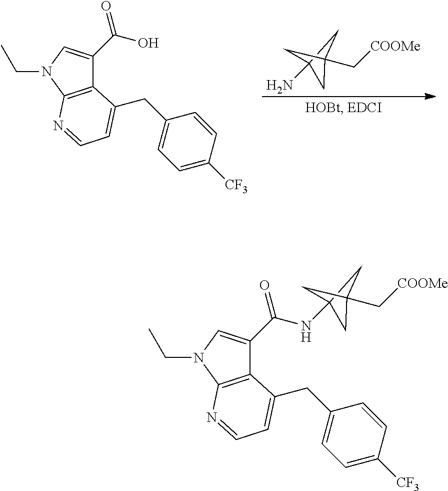 [SMILES: CCn1cc(C(=O)O)c2c(Cc3ccc(C(F)(F)F)cc3)ccnc21 ; COC(=O)CC12CC(N)(C1)C2 ; CCn1cc(C(=O)NC23CC(CC(=O)OC)(C2)C3)c2c(Cc3ccc(C(F)(F)F)cc3)ccnc21]

To a solution of 1-ethyl-4-[[4-(trifluoromethyl)phenyl]methyl]pyrrolo[2,3-b]pyridine-3-carboxylic acid (40 mg, 114.84 umol, 1 eq) in DCM (1 mL) was added dropwise HOBt (3.10 mg, 22.97 umol, 0.2 eq), Et₃N (58.10 mg, 574.19 umol, 79.92 uL, 5 eq) and EDCI (44.03 mg, 229.67 umol, 2 eq) at 0° C. After addition, the mixture was stirred at this temperature for 30 min, and then methyl 2-(3-amino-1-bicyclo[1.1.1]pentanyl)acetate (21.39 mg, 137.80 umol, 1.2 eq) was added at 0° C. The resulting mixture was stirred at 25° C. for 12 hr. LCMS showed the carboxylic acid was consumed completely. The residue was diluted with H₂O 3 mL and extracted with DCM 15 mL (5 mL×3). The combined organic layers were washed with brine (5 mL), dried over Na₂SO₄, filtered and concentrated under reduced pressure to give a residue. The crude product was used into the next step without further purification. Compound methyl 2-[3-[[1-ethyl-4-[[4-(trifluoromethyl)phenyl]methyl]indole-3-carbonyl]amino]-1-bicyclo[1.1.1]pentanyl]acetate (25 mg, 51.60 umol, 44.93% yield) was obtained as a white solid, which used into the next step without further purification. LCMS for product (ESI): m/z 486.1 [M+H]⁺.

### Step 2: 2-[3-[[1-ethyl-4-[[4-(trifluoromethyl)phenyl]methyl]indole-3-carbonyl]amino]-1-bicyclo[1.1.11pentanyl]acetic acid

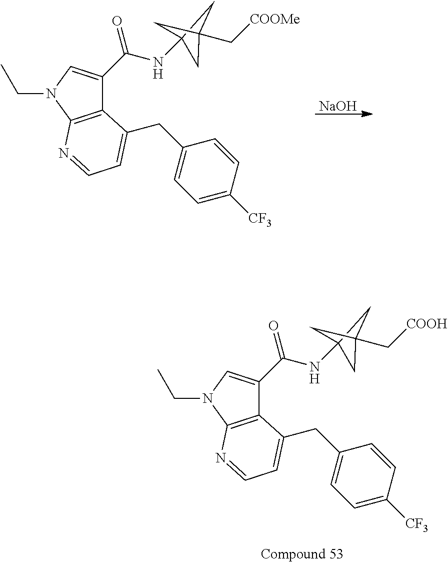 [SMILES: CCn1cc(C(=O)NC23CC(CC(=O)OC)(C2)C3)c2c(Cc3ccc(C(F)(F)F)cc3)ccnc21 ; "NaOH" ; CCn1cc(C(=O)NC23CC(CC(=O)O)(C2)C3)c2c(Cc3ccc(C(F)(F)F)cc3)ccnc21]

To a solution of methyl 2-[3-[[1-ethyl-4-[[4-(trifluoromethyl)phenyl]methyl]indole-3-carbonyl]amino]-1-bicyclo[1.1.1]pentanyl]acetate (25 mg, 51.60 umol, 1 eq) in H₂O (1 mL) and MeOH (1 mL) was added NaOH (2.06 mg, 51.60 umol, 1 eq). The mixture was stirred at 80° C. for 1 hr. HPLC showed the methyl ester was consumed completely. The reaction mixture was concentrated under reduced pressure to remove solvent. The residue was purified by prep-HPLC (HCl condition). Compound 2-[3-[[1-ethyl-4-[[4-(trifluoromethyl)phenyl]methyl]indole-3-carbonyl]amino]-1-bicyclo[1.1.1]pentanyl]acetic acid (8 mg, 18.88 umol, 36.59% yield, 98.7% purity) was obtained as a white solid. LCMS for product (ESI): m/z 472.0 [M+H]⁺, Rt: 2.448 min. ¹H NMR (400 MHz, DMSO-d₆) δ=8.63 (br s, 1H), 8.25 (d, J=4.8 Hz, 1H), 7.98 (s, 1H), 7.55 (d, J=8.0 Hz, 2H), 7.37 (d, J=8.0 Hz, 2H), 7.14-7.09 (m, 1H), 4.70 (s, 2H), 4.29-4.24 (m, 2H), 2.50 (s, 2H), 2.01 (s, 6H), 1.37 (t, J=7.2 Hz, 3H).

### Example 58—Synthesis of 2-[3-[[1-(2-fluoroethyl)-4-[[4-(trifluoromethyl)phenyl]methyl]-pyrrolo[2,3-b]pyridine-3-carbonyl]amino]-1-bicyclo[1.1.1]pentanyl]acetic acid (Compound

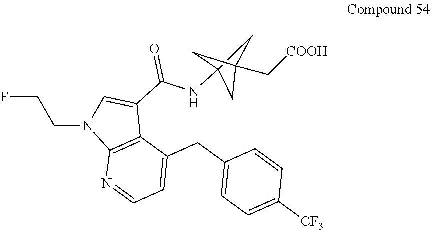 [SMILES: O=C(O)CC12CC(NC(=O)c3cn(CCF)c4nccc(Cc5ccc(C(F)(F)F)cc5)c34)(C1)C2]

### Step 1: methyl 2-[3-[[1-(2-fluoroethyl)-4-[[4-(trifluoromethyl)phenyl]methyl]pyrrolo[2,3-b]pyridine-3-carbonyl]amino]-1-bicyclo[1.1.1]pentanyl]acetate

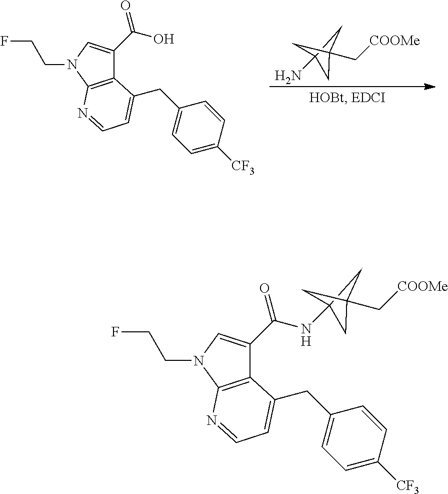 [SMILES: O=C(O)c1cn(CCF)c2nccc(Cc3ccc(C(F)(F)F)cc3)c12 ; COC(=O)CC12CC(N)(C1)C2 ; COC(=O)CC12CC(NC(=O)c3cn(CCF)c4nccc(Cc5ccc(C(F)(F)F)cc5)c34)(C1)C2]

To a solution of 1-(2-fluoroethyl)-4-[[4-(trifluoromethyl)phenyl]methyl]pyrrolo[2,3-b]pyridine-3-carboxylic acid (60 mg, 163.80 umol, 1 eq) in DCM (1 mL) was added dropwise Et₃N (82.87 mg, 819.00 umol, 113.99 uL, 5 eq), HOBt (4.43 mg, 32.76 umol, 0.2 eq) and EDCI (62.80 mg, 327.60 umol, 2 eq) at 0° C. After addition, the mixture was stirred at this temperature for 0.5 hr, and then methyl 2-(3-amino-1-bicyclo[1.1.1]pentanyl)acetate (38.13 mg, 245.70 umol, 1.5 eq) was added dropwise at 0° C. The resulting mixture was stirred at 25° C. for 12 hr. LC-MS showed no carboxylic acid was remained. Several new peaks were shown on LC-MS and 80% of desired compound was detected. The reaction mixture was concentrated under reduced pressure to remove dioxane. The residue was diluted with H₂O 3 mL and extracted with EA 15 mL (5 mL×3). The combined organic layers were washed with brine 5 mL, dried over Na₂SO₄, filtered and concentrated under reduced pressure to give a residue. The crude product was used into the next step without further purification. Compound methyl 2-[3-[[1-(2-fluoroethyl)-4-[[4-(trifluoromethyl)phenyl]methyl]pyrrolo[2,3-b]pyridine-3-carbonyl]amino]-1-bicyclo[1.1.1]pentanyl]acetate (60 mg, 119.17 umol, 72.75% yield) was obtained as a white solid, which was used into the next step without further purification. LCMS for product (ESI): m/z 504.2 [M+H]⁺.

### Step 2: 2-[3-[[1-(2-fluoroethyl)-4-[[4-(trifluoromethyl)phenyl]methyl]pyrrolo[2,3-b]pyridine-3-carbonyl]amino]-1-bicyclo[1.1.11pentanyl]acetic acid

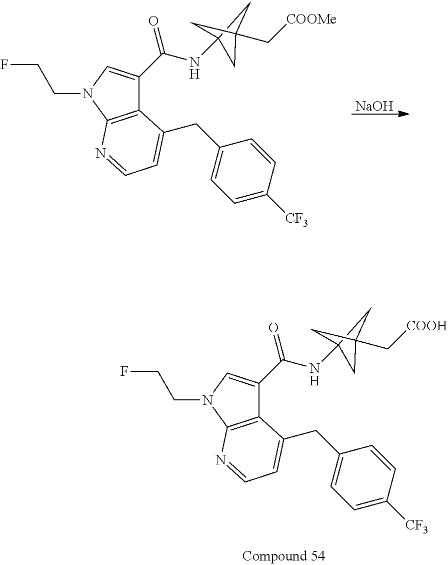 [SMILES: COC(=O)CC12CC(NC(=O)c3cn(CCF)c4nccc(Cc5ccc(C(F)(F)F)cc5)c34)(C1)C2 ; "NaOH" ; O=C(O)CC12CC(NC(=O)c3cn(CCF)c4nccc(Cc5ccc(C(F)(F)F)cc5)c34)(C1)C2]

To a solution of methyl 2-[3-[[1-(2-fluoroethyl)-4-[[4-(trifluoromethyl)phenyl]methyl]pyrrolo[2,3-b]pyridine-3-carbonyl]amino]-1-bicyclo[1.1.1]pentanyl]acetate (60 mg, 119.17 umol, 1 eq) in H₂O (1 mL) and MeOH (1 mL) was added NaOH (14.30 mg, 357.51 umol, 3 eq). The mixture was stirred at 80° C. for 2 hr. HPLC showed the methyl ester was consumed completely. The reaction mixture was concentrated under reduced pressure to remove solvent. The residue was purified by prep-HPLC (HCl condition).

Compound 2-[3-[[1-(2-fluoroethyl)-4-[[4-(trifluoromethyl)phenyl]methyl]pyrrolo[2,3-b]pyridine-3-carbonyl]amino]-1-bicyclo[1.1.1]pentanyl]acetic acid (30 mg, 59.70 umol, 50.10% yield, 97.4% purity) was obtained as a white solid. LCMS for product (ESI): m/z 490.2 [M+H]⁺. ¹H NMR (400 MHz, DMSO-d6) δ=8.70 (s, 1H), 8.27 (d, J=4.8 Hz, 1H), 7.97 (s, 1H), 7.57 (d, J=8.0 Hz, 2H), 7.39 (d, J=8.0 Hz, 2H), 7.15 (d, J=4.8 Hz, 1H), 4.87 (t, J=4.8 Hz, 1H), 4.76-4.70 (m, 3H), 4.62 (t, J=4.8 Hz, 1H), 4.55 (t, J=4.8 Hz, 1H), 2.50 (s, 2H), 2.03 (s, 6H).

### Example 59—Synthesis of (1r,4r)-4-[[[1-ethyl-4-[[4-(trifluoromethyl) phenyl]methyl]indole-3-carbonyl]amino]methyl]cyclohexanecarboxylic acid (Compound 55)

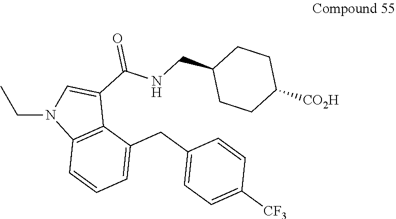 [SMILES: CCn1cc(C(=O)NC[C@H]2CC[C@H](C(=O)O)CC2)c2c(Cc3ccc(C(F)(F)F)cc3)cccc21]

### Step 1: methyl (1r,4r)-4-[[[1-ethyl-4-[[4-(trifluoromethyl) phenyl]methyl]indole-3-carbonyl]amino]methyl]cyclohexanecarboxylate

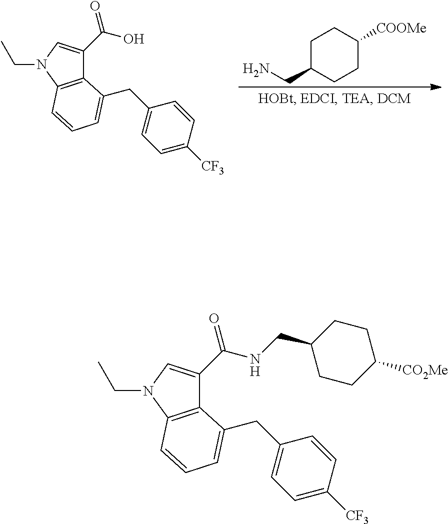 [SMILES: CCn1cc(C(=O)O)c2c(Cc3ccc(C(F)(F)F)cc3)cccc21 ; COC(=O)[C@H]1CC[C@H](CN)CC1 ; CCn1cc(C(=O)NC[C@H]2CC[C@H](C(=O)OC)CC2)c2c(Cc3ccc(C(F)(F)F)cc3)cccc21]

To a solution of 1-ethyl-4-[[4-(trifluoromethyl) phenyl]methyl]indole-3-carboxylic acid (20 mg, 57.58 umol, 1 eq) in DCM (2 mL) was added HOBt (1.56 mg, 11.52 umol, 0.2 eq), EDCI (22.08 mg, 115.16 umol, 2 eq) and Et3N (29.13 mg, 287.91 umol, 40.07 uL, 5 eq) at 0° C. The mixture was stirred at 0° C. for 0.5 hr. Then methyl 4-(aminomethyl) cyclohexanecarboxylate; hydrochloride (17.94 mg, 86.37 umol, 1.5 eq) was added the mixture. The mixture was stirred at 25° C. for 16 hr. LC-MS showed 0% of the carboxylic acid was remained. Several new peaks were shown on LC-MS and 68.75% of desired compound was detected. The reaction mixture was diluted with H₂O 3 mL and extracted with DCM 9 mL (3 mL×3). The combined organic layers were washed with brine 3 mL, dried over Na₂SO₄, filtered and concentrated under reduced pressure to give a residue. The crude product was used into the next step without further purification. Compound methyl (1r,4r)-4-[[[1-ethyl-4-[[4-(trifluoromethyl) phenyl]methyl]indole-3-carbonyl]amino]methyl]cyclohexanecarboxylate (13 mg, 25.97 umol, 45.10% yield) was obtained as yellow oil. The crude product was used into the next step without further purification. LCMS for product (ESI): m/z 501.3 [M+H]⁺.

### Step 2: (1r,4r)-4-[[[1-ethyl-4-[[4-(trifluoromethyl) phenyl]methyl]indole-3-carbonyl]amino]methyl]cyclohexanecarboxylic acid

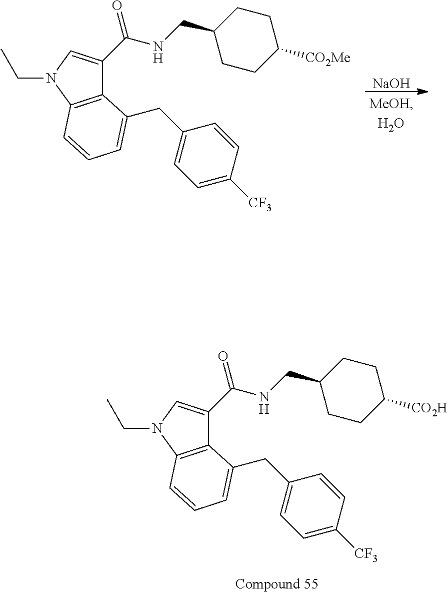 [SMILES: CCn1cc(C(=O)NC[C@H]2CC[C@H](C(=O)OC)CC2)c2c(Cc3ccc(C(F)(F)F)cc3)cccc21 ; "NaOH" ; CCn1cc(C(=O)NC[C@H]2CC[C@H](C(=O)O)CC2)c2c(Cc3ccc(C(F)(F)F)cc3)cccc21]

To a solution of methyl (1r,4r)-4-[[[1-ethyl-4-[[4-(trifluoromethyl) phenyl]methyl]indole-3-carbonyl]amino]methyl]cyclohexanecarboxylate (13 mg, 25.97 umol, 1 eq) in H₂O (1 mL) and MeOH (1 mL) was added NaOH (1.04 mg, 25.97 umol, 1 eq). The mixture was stirred at 50° C. for 16 hr. HPLC showed 0% of the methyl ester was remained. The reaction mixture was concentrated under reduced pressure to remove MeOH. The residue was purified by prep-HPLC (HCl condition). Compound (1r,4r)-4-[[[1-ethyl-4-[[4-(trifluoromethyl) phenyl]methyl]indole-3-carbonyl]amino]methyl]cyclohexanecarboxylic acid (5 mg, 10.28 umol, 39.57% yield, 100% purity) was obtained as a white solid. LCMS for product (ESI): m/z 487.1 [M+H]⁺. ¹H NMR (400 MHz, DMSO-d₆) 12.02 (br s, 1H) 7.97 (t, J=5.6 Hz, 1H) 7.75 (s, 1H) 7.51 (d, J=8.0 Hz, 2H) 7.43 (d, J=8.0 Hz, 1H) 7.29 (d, J=8.0 Hz, 2H) 7.19 (t, J=7.8 Hz, 1H) 7.00 (d, J=7.2 Hz, 1H) 4.66 (s, 2H) 4.23 (t, J=6.8 Hz, 2H) 3.04 (t, J=6.0 Hz, 2H) 2.10-2.16 (m, 1H) 1.88 (d, J=6.8 Hz, 2H) 1.73 (d, J=6.8 Hz, 2H) 1.35-1.46 (m, 4H) 1.28-1.22 (m, 2H) 0.98-0.90 (m, 2H).

### Example 60—Synthesis of 2-[(1r,4r)-4-[[1-ethyl-4-[[4-(trifluoromethyl) phenyl]methyl]indole-3-carbonyl]amino]cyclohexyl]acetic acid (Compound 56)

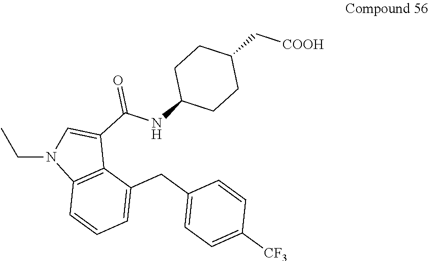 [SMILES: CCn1cc(C(=O)N[C@H]2CC[C@H](CC(=O)O)CC2)c2c(Cc3ccc(C(F)(F)F)cc3)cccc21]

### Step 1: ethyl 2-[(1r,4r)-4-[[1-ethyl-4-[[4-(trifluoromethyl) phenyl]methyl]indole-3-carbonyl]amino]cyclohexyl]acetate

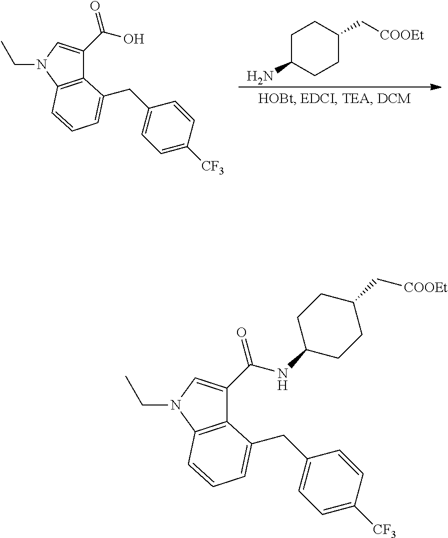 [SMILES: CCn1cc(C(=O)O)c2c(Cc3ccc(C(F)(F)F)cc3)cccc21 ; CCOC(=O)C[C@H]1CC[C@H](N)CC1 ; CCOC(=O)C[C@H]1CC[C@H](NC(=O)c2cn(CC)c3cccc(Cc4ccc(C(F)(F)F)cc4)c23)CC1]

To a solution of 1-ethyl-4-[[4-(trifluoromethyl) phenyl]methyl]indole-3-carboxylic acid (20 mg, 57.58 umol, 1 eq) in DCM (2 mL) was added HOBt (1.56 mg, 11.52 umol, 0.2 eq), EDCI (22.08 mg, 115.16 umol, 2 eq) and Et₃N (29.13 mg, 287.91 umol, 40.07 uL, 5 eq) at 0° C. The mixture was stirred at 0° C. for 0.5 hr. Then ethyl trans-2-(4-aminocyclohexyl) acetate; hydrochloride (19.15 mg, 86.37 umol, 1.5 eq) was added the mixture. The mixture was stirred at 25° C. for 16 hr. LC-MS showed 0% of the carboxylic acid was remained. Several new peaks were shown on LC-MS and 65.83% of desired compound was detected. The reaction mixture was diluted with H₂O 3 mL and extracted with DCM 9 mL (3 mL×3). The combined organic layers were washed with brine 3 mL, dried over Na₂SO₄, filtered and concentrated under reduced pressure to give a residue. The crude product was used into the next step without further purification. Compound C₂₉H₃₃N₂O₃F₃ (17 mg, 33.04 umol, 57.37% yield) was obtained as yellow oil, which was used into the next step without further purification. LCMS for product (ESI): m/z 515.3 [M+H]⁺.

### Step 2: 2-[(1r,4r)-4-[[1-ethyl-4-[[4-(trifluoromethyl) phenyl]methyl]indole-3-carbonyl]amino]cyclohexyl]acetic acid

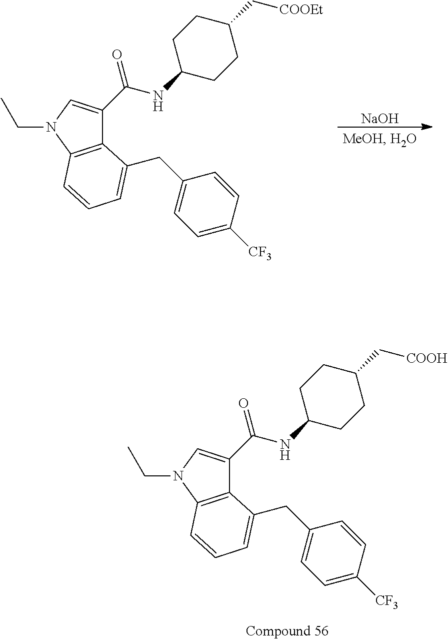 [SMILES: CCOC(=O)C[C@H]1CC[C@H](NC(=O)c2cn(CC)c3cccc(Cc4ccc(C(F)(F)F)cc4)c23)CC1 ; "NaOH" ; CCn1cc(C(=O)N[C@H]2CC[C@H](CC(=O)O)CC2)c2c(Cc3ccc(C(F)(F)F)cc3)cccc21]

To a solution of ethyl 2-[(1r,4r)-4-[[1-ethyl-4-[[4-(trifluoromethyl) phenyl]methyl]indole-3-carbonyl]amino]cyclohexyl]acetate (17 mg, 33.04 umol, 1 eq) in H₂O (1 mL) and MeOH (1 mL) was added NaOH (1.32 mg, 33.04 umol, 1 eq). The mixture was stirred at 50° C. for 16 hr. HPLC showed 0% of the ethyl ester was remained. The reaction mixture was concentrated under reduced pressure to remove MeOH. The residue was purified by prep-HPLC (HCl condition). Compound 2-[(1r,4r)-4-[[1-ethyl-4-[[4-(trifluoromethyl) phenyl]methyl]indole-3-carbonyl]amino]cyclohexyl]acetic acid (6 mg, 12.33 umol, 37.33% yield, 100% purity) was obtained as a white solid. LCMS for product (ESI): m/z 487.1 [M+H]⁺. ¹H NMR (400 MHz, DMSO-d₆) 12.06 (br s, 1H) 7.78 (d, J=7.8 Hz, 1H) 7.68 (s, 1H) 7.52 (d, J=8.0 Hz, 2H) 7.43 (d, J=8.0 Hz, 1H) 7.29 (d, J=8.00 Hz, 2H) 7.18 (t, J=8.0 Hz, 1H) 7.01 (d, J=7.2 Hz, 1H) 4.64 (s, 2H) 4.20-4.25 (m, 2H) 3.56-3.65 (m, 1H) 2.13 (d, J=6.8 Hz, 2H) 1.76-1.72 (m, 4H) 1.64-1.57 (m, 1H) 1.41-1.36 (m, 3H) 1.26-1.18 (m, 2H) 1.12-1.10 (m, 2H).

### Example 61—Synthesis of 2-[(1r,4r)-4-[[1-(2-fluoroethyl)-4-[[4-(trifluoromethyl)-phenyl]methyl]pyrrolo[2,3-b]pyridine-3-carbonyl]amino]cyclohexyl]acetic acid (Compound 57)

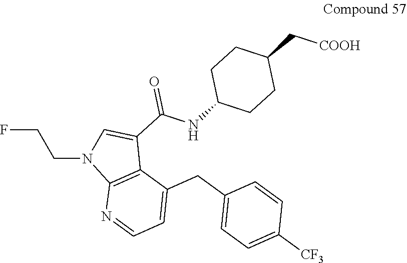 [SMILES: O=C(O)C[C@H]1CC[C@H](NC(=O)c2cn(CCF)c3nccc(Cc4ccc(C(F)(F)F)cc4)c23)CC1]

### Step 1: ethyl 2-[(1r,4r)-4-[[1-(2-fluoroethyl)-4-[[4-(trifluoromethyl)-phenyl]methyl]pyrrolo[2,3-b]pyridine-3-carbonyl]amino]cyclohexyl]acetate

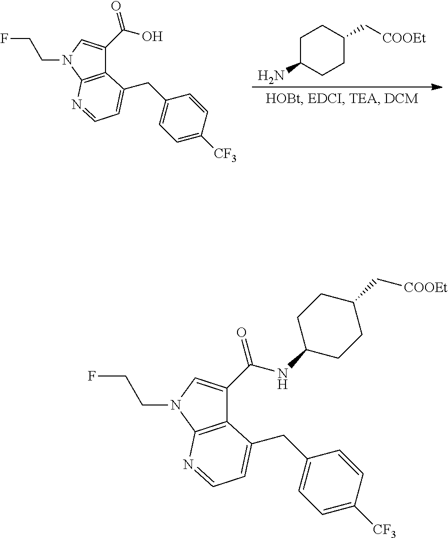 [SMILES: O=C(O)c1cn(CCF)c2nccc(Cc3ccc(C(F)(F)F)cc3)c12 ; CCOC(=O)C[C@H]1CC[C@H](N)CC1 ; CCOC(=O)C[C@H]1CC[C@H](NC(=O)c2cn(CCF)c3nccc(Cc4ccc(C(F)(F)F)cc4)c23)CC1]

To a solution of 1-(2-fluoroethyl)-4-[[4-(trifluoromethyl)phenyl]methyl]pyrrolo[2,3-b]pyridine-3-carboxylic acid (40 mg, 109.20 umol, 1 eq) in DCM (1 mL) was added dropwise Et₃N (11.05 mg, 109.20 umol, 15.20 uL, 1 eq), HOBt (14.75 mg, 109.20 umol, 1 eq) and EDCI (41.87 mg, 218.40 umol, 2 eq) at 0° C. After addition, the mixture was stirred at this temperature for 0.5 hr, and then ethyl trans-2-(4-aminocyclohexyl)acetate;hydrochloride (29.05 mg, 131.04 umol, 1.2 eq) was added dropwise at 0° C. The resulting mixture was stirred at 25° C. for 12 hr. Several new peaks were shown on LC-MS and 80% of desired compound was detected. The reaction mixture was concentrated under reduced pressure to remove dioxane. The residue was diluted with H₂O 3 mL and extracted with EA 15 mL (5 mL×3). The combined organic layers were washed with brine 5 mL, dried over Na₂SO₄, filtered and concentrated under reduced pressure to give a residue. The crude product was used into the next step without further purification. Compound ethyl 2-[(1r,4r)-4-[[1-(2-fluoroethyl)-4-[[4-(trifluoromethyl)phenyl]methyl]pyrrolo[2,3-b]pyridine-3-carbonyl]amino]cyclohexyl]acetate (40 mg, crude) was obtained as a white solid, which was used into the next step without further purification. LCMS for product (ESI): m/z 534.2 [M+H]⁺.

### Step 2: 2-[(1r,4r)-4-[[1-(2-fluoroethyl)-4-[[4-(trifluoromethyl)phenyl]methyl]-pyrrolo[2,3-b]pyridine-3-carbonyl]amino]cyclohexyl]acetic acid

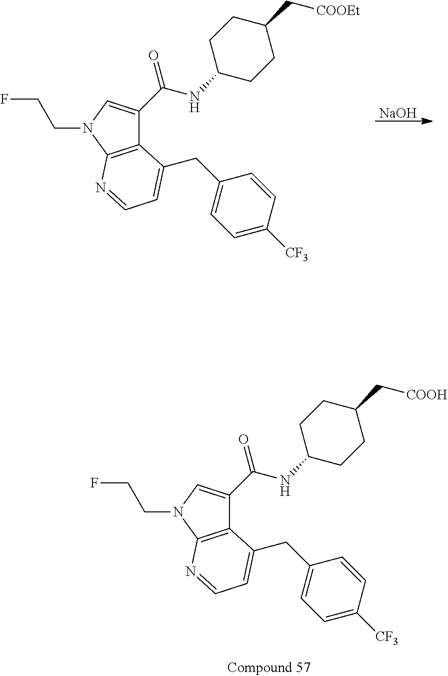 [SMILES: CCOC(=O)C[C@H]1CC[C@H](NC(=O)c2cn(CCF)c3nccc(Cc4ccc(C(F)(F)F)cc4)c23)CC1 ; "NaOH" ; O=C(O)C[C@H]1CC[C@H](NC(=O)c2cn(CCF)c3nccc(Cc4ccc(C(F)(F)F)cc4)c23)CC1]

To a solution of ethyl 2-[(1r,4r)-4-[[1-(2-fluoroethyl)-4-[[4-(trifluoromethyl)phenyl]methyl]pyrrolo[2,3-b]pyridine-3-carbonyl]amino]cyclohexyl]acetate (40 mg, 74.97 umol, 1 eq) in H₂O (1 mL) and MeOH (1 mL) was added NaOH (3.00 mg, 74.97 umol, 1 eq). The mixture was stirred at 80° C. for 2 hr. HPLC showed the ethyl ester was consumed completely. The reaction mixture was concentrated under reduced pressure to remove solvent. The residue was purified by prep-HPLC (HCl condition). Compound 2-[(1r,4r)-4-[[1-(2-fluoroethyl)-4-[[4-(trifluoromethyl)phenyl]methyl]pyrrolo[2,3-b]pyridine-3-carbonyl]amino]cyclohexyl]acetic acid (13 mg, 25.72 umol, 34.30% yield, 100% purity) was obtained as a white solid. LCMS for product (ESI): m/z 506.1 [M+H]⁺. ¹H NMR (400 MHz, DMSO-d₆) δ=8.24 (d, J=4.4 Hz, 1H), 8.00 (d, J=7.6 Hz, 1H), 7.94 (s, 1H), 7.55 (d, J=8.0 Hz, 2H), 7.35 (d, J=8.0 Hz, 2H), 7.09 (d, J=4.8 Hz, 1H), 4.86 (t, J=4.4 Hz, 1H), 4.74 (t, J=4.0 Hz, 1H), 4.69 (s, 2H), 4.62 (d, J=4.4 Hz, 1H), 4.55 (d, J=4.4 Hz, 1H), 3.63-3.60 (s, 1H), 2.11 (d, J=6.8 Hz, 2H), 1.82-1.70 (m, 4H), 1.59 (br s, 1H), 1.26-1.17 (m, 2H), 1.07-0.94 (m, 2H).

### Example 62—Synthesis of (1r,4r)-4-[[[1-(2-fluoroethyl)-4-[[4-(trifluoromethyl)-phenyl]methyl]-pyrrolo[2,3-b]pyridine-3-carbonyl]amino]methyl]cyclohexanecarboxylic acid (Compound 58)

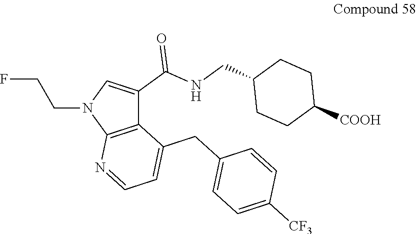 [SMILES: O=C(NC[C@H]1CC[C@H](C(=O)O)CC1)c1cn(CCF)c2nccc(Cc3ccc(C(F)(F)F)cc3)c12]

### Step 1: methyl (1r,4r)-4-[[[1-(2-fluoroethyl)-4-[[4-(trifluoromethyl)-phenyl]methyl]pyrrolo[2,3-b]pyridine-3-carbonyl]amino]methyl]cyclohexanecarboxylate

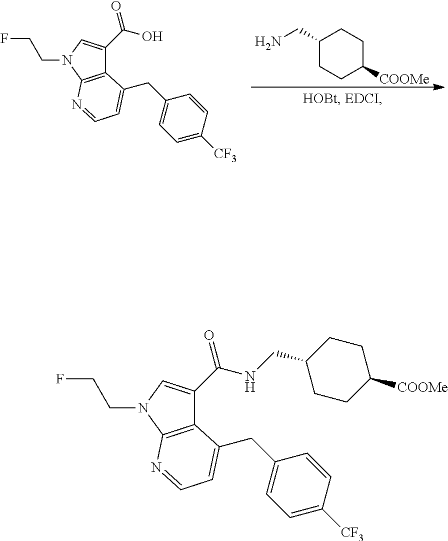 [SMILES: O=C(O)c1cn(CCF)c2nccc(Cc3ccc(C(F)(F)F)cc3)c12 ; COC(=O)[C@H]1CC[C@H](CN)CC1 ; COC(=O)[C@H]1CC[C@H](CNC(=O)c2cn(CCF)c3nccc(Cc4ccc(C(F)(F)F)cc4)c23)CC1]

To a solution of 1-(2-fluoroethyl)-4-[[4-(trifluoromethyl)phenyl]methyl]pyrrolo[2,3-b]pyridine-3-carboxylic acid (40 mg, 109.20 umol, 1 eq) in DCM (1 mL) was added dropwise Et₃N (55.25 mg, 545.99 umol, 76.00 uL, 5 eq), HOBt (2.95 mg, 21.84 umol, 0.2 eq) and EDCI (41.87 mg, 218.39 umol, 2 eq) at 0° C. After addition, the mixture was stirred at this temperature for 0.5 hr, and then methyl trans-4-(aminomethyl)cyclohexanecarboxylate;hydrochloride (27.22 mg, 131.04 umol, 1.2 eq) was added dropwise at 0° C. The resulting mixture was stirred at 25° C. for 12 hr. LC-MS showed no carboxylic acid was remained. Several new peaks were shown on LC-MS and 80% of desired compound was detected. The reaction mixture was concentrated under reduced pressure to remove dioxane. The residue was diluted with H₂O 3 mL and extracted with EA 15 mL (5 mL×3). The combined organic layers were washed with brine 5 mL, dried over Na₂SO₄, filtered and concentrated under reduced pressure to give a residue. The crude product was used into the next step without further purification. Compound methyl (1r,4r)-4-[[[1-(2-fluoroethyl)-4-[[4-(trifluoromethyl)phenyl]methyl]pyrrolo[2,3-b]pyridine-3-carbonyl]amino]methyl]cyclohexanecarboxylate (56 mg, crude) was obtained as a white solid, which was used into the next step without further purification. LCMS for product (ESI): m/z 520.2 [M+H]⁺.

### Step 2: (1r,4r)-4-[[[1-(2-fluoroethyl)-4-[[4-(trifluoromethyl)phenyl]methyl]-pyrrolo[2,3-b]pyridine-3-carbonyl]amino]methyl]cyclohexanecarboxylic acid

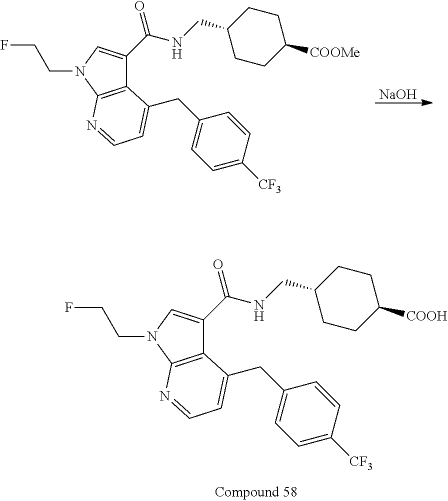 [SMILES: COC(=O)[C@H]1CC[C@H](CNC(=O)c2cn(CCF)c3nccc(Cc4ccc(C(F)(F)F)cc4)c23)CC1 ; "NaOH" ; O=C(NC[C@H]1CC[C@H](C(=O)O)CC1)c1cn(CCF)c2nccc(Cc3ccc(C(F)(F)F)cc3)c12]

To a solution of methyl (1r,4r)-4-[[[1-(2-fluoroethyl)-4-[[4-(trifluoromethyl)phenyl]methyl]pyrrolo[2,3-b]pyridine-3-carbonyl]amino]methyl]cyclohexanecarboxylate (56 mg, 107.79 umol, 1 eq) in MeOH (1 mL) and H₂O (1 mL) was added NaOH (4.31 mg, 107.79 umol, 1 eq). The mixture was stirred at 80° C. for 2 hr. HPLC showed the methyl ester was consumed completely. The reaction mixture was concentrated under reduced pressure to remove solvent. The residue was purified by prep-HPLC (HCl condition). Compound (1r,4r)-4-[[[1-(2-fluoroethyl)-4-[[4-(trifluoromethyl)phenyl]methyl]pyrrolo[2,3-b]pyridine-3-carbonyl]amino]methyl]cyclohexanecarboxylic acid (40 mg, 76.99 umol, 71.43% yield, 97.3% purity) was obtained as a white solid. LCMS for product (ESI): m/z 506.1 [M+H]⁺. ¹H NMR (400 MHz, DMSO-d₆) δ=11.98 (br s, 1H), 8.25 (d, J=4.8 Hz, 1H), 8.17 (t, J=5.6 Hz, 1H), 8.00 (s, 1H), 7.56 (d, J=8.0 Hz, 2H), 7.36 (d, J=8.0 Hz, 2H), 7.10 (d, J=4.8 Hz, 1H), 4.88 (t, J=4.8 Hz, 1H), 4.78-4.73 (m, 3H), 4.64 (t, J=4.4 Hz, 1H), 4.59-4.56 (m, 1H), 3.07 (t, J=6.0 Hz, 2H), 2.18-2.07 (m, 1H), 1.92-1.87 (m, 2H), 1.79-1.74 (m, 2H), 1.45 (br s, 1H), 1.27-1.22 (m, 2H), 0.99-0.92 (m, 2H).

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Biological Examples

### Example B-1: Prostanoid EP4 Binding Assay

HEK293 cells transfected with human Prostanoid EP4 Receptor (EP4) are maintained in DMEM supplemented with L-Glutamine, 10% FBS and 300 μg/ml G418. Cells are harvested and membranes prepared by differential centrifugation, following lysis of the cells in the presence of protease inhibitors, for use in the receptor binding assay. EP4 binding assay is performed in 50 mM HEPES pH7.4, 5 mM MgCl2, 1 mM CaCl2) and 0.2% BSA. Testing compounds are diluted with DMSO to make 8-point 4-fold serial dilution. Cell membrane homogenates are incubated at 24° C. with ³H-prostaglandin E2 in the absence or presence of the testing compound in assay buffer. Nonspecific binding (Low control: LC) and total binding (High control: HC) are determined in the presence or absence of saturated cold ligand Prostaglandin E2 (PGE2). Specific binding is calculated by subtracting nonspecific binding from total binding. When binding assays are completed, the reaction mixture is filtered rapidly under vacuum through glass fiber filters (GF/C, Packard) presoaked with 0.5% BSA for at least 0.5 hour at room temperature and then washed for 4 times with cold wash buffer (50 mM HEPES pH7.4, 500 mM NaCl, 0.1% BSA) using a 96-sample Perkin Elmer Filtermate Harvester (Unifilter, Packard). The filters are dried for 1 hour at 50 degrees then counted for radioactivity in a scintillation counter using Perkin Elmer Microscint 20 cocktail. ³H trapped on filter is counted using Perkin Elmer MicroBeta2 Reader. Inhibition is calculated using following equation: % Inhibition=(1−(Assay well−Average_LC)/(Average_HC−Average_LC))×100%. The results are expressed as a percent inhibition of the control radioligand specific binding. Data are analyzed and IC50 is calculated using GraphPad Prism 5 and the model “log(inhibitor) vs. response—Variable slope”. The binding affinity of the compounds is determined by using the Cheng and Prusoff equation Ki=IC50/(1+[radioligand]/Kd).

Illustrative binding affinities for representative compounds are described in Table 1. The potencies are divided into three criteria: +means that Kᵢ is greater than 500 nM; ++means Kᵢ is between 100 nM and 499 nM; +++means Kᵢ is below 100 nM. Inactive compounds are designated NA.

TABLE 1

| Compound No. | EP4 Binding Ki (nM) |
|---|---|
| Compound 1 | +++ |
| Compound 2 | +++ |
| Compound 3 | +++ |
| Compound 4 | +++ |
| Compound 5 | ++ |
| Compound 6A | + |
| Compound 6B | +++ |
| Compound 7 | +++ |
| Compound 8 | +++ |
| Compound 9 | +++ |
| Compound 10 | ++ |
| Compound 11A | ++ |
| Compound 11B | +++ |
| Compound 12 | +++ |
| Compound 13A | + |
| Compound 13B | +++ |
| Compound 14A | ++ |
| Compound 14B | +++ |
| Compound 15 | +++ |
| Compound 16 | + |
| Compound 17 | ++ |
| Compound 18 | ++ |
| Compound 19 | ++ |
| Compound 20 | +++ |
| Compound 21 | +++ |
| Compound 22 | + |
| Compound 23 | +++ |
| Compound 24 | +++ |
| Compound 25 | +++ |
| Compound 26 | +++ |
| Compound 27 | +++ |
| Compound 28 | ++ |
| Compound 29 | +++ |
| Compound 30 | ++ |
| Compound 31 | + |
| Compound 32 | + |
| Compound 33 | ++ |
| Compound 34 | +++ |
| Compound 35 | ++ |
| Compound 36 | ++ |
| Compound 37 | +++ |
| Compound 38 | +++ |
| Compound 39 | +++ |
| Compound 40 | +++ |
| Compound 41 | +++ |
| Compound 42 | +++ |
| Compound 43 | +++ |
| Compound 44 | +++ |
| Compound 45 | +++ |
| Compound 46 | +++ |
| Compound 47 | +++ |
| Compound 48 | +++ |
| Compound 49 | ++ |
| Compound 50 | + |
| Compound 51 | +++ |
| Compound 52 | +++ |
| Compound 53 | +++ |
| Compound 54 | +++ |
| Compound 55 | +++ |
| Compound 56 | +++ |
| Compound 57 | +++ |
| Compound 58 | +++ |

### Example B-2: Prostanoid EP4 and EP2 FLIPR Assay

HEK293 cells transfected with human Prostanoid EP4 Receptor (EP4) or CHO cells transfected with human Prostanoid EP2 Receptor (EP2) are maintained in DMEM supplemented with L-Glutamine, 10% FBS and 300 μg/ml G418. Cells are plated at 20,000 cells/well in black 384-well Poly-D-Lysine protein coating plate and incubated at 37° C., 5% CO₂ for 24 h. Prior to the assay, calcium-sensitive dye Fluo-4 Direct™ (Molecular Devices) is reconstituted according to manufacturer instructions. The reconstituted dye was diluted in assay buffer (1× HBSS, 20 mM HEPES, and 2.5 mM Probenicid, pH 7.4). Growth media is removed and 20 μl of this diluted dye is added to each well at a final concentration of 2 μM. Plates are incubated for 50 min at 37° C. 5% C₀₂ and 10 min at RT after dye addition. Testing compounds are diluted with DMSO to make 10-point 4-fold serial dilution. After the pre-treatment incubation, fluorescence intensity is measured on a FlexStation II fluorometric imaging plate reader (Molecular Devices). Relative fluorescence units (RFU) are measured before (20 readings) and after (40 readings) the PGE2 addition for a total 60 sec read time (Excitation=485 nm, Emission=525 nm). Data are analyzed and IC50 is calculated using GraphPad Prism 5 and the model “log(inhibitor) vs. response—Variable slope”.

Illustrative data for representative compounds are described in Table 2. The potencies are divided into four criteria: +means that Kᵢ is greater than 1,000 nM; ++means that Kᵢ is between 500 nM and 1,000 nM; +++means Kᵢ is between 100 nM and 499 nM; ++++means Kᵢ is below 100 nM.

TABLE 2

| Compound No. | EP4 FLIPR (nM) | EP2 FLIPR (μM) |
|---|---|---|
| Compound 1 | ++++ | + |
| Compound 2 | ++++ | + |
| Compound 3 | ++++ | + |
| Compound 4 | ++++ | + |
| Compound 7 | ++++ | + |
| Compound 8 | ++++ | + |
| Compound 9 | ++++ | + |
| Compound 10 | ++++ | + |
| Compound 12 | ++++ | + |
| Compound 15 | ++++ | + |
| Compound 14A | +++ | + |
| Compound 14B | ++++ | + |
| Compound 13A | +++ | |
| Compound 16 | +++ | |
| Compound 18 | ++++ | |

The examples and embodiments described herein are for illustrative purposes only and various modifications or changes suggested to persons skilled in the art are to be included within the spirit and purview of this application and scope of the appended claims.

What is claimed is:

1. A compound of Formula (I), or a pharmaceutically acceptable salt or solvate thereof: 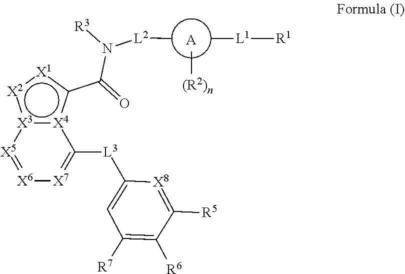
wherein,
R¹ is —CO₂H, —CO₂(C₁-C₆alkyl), —C(═O)NHSO₂R¹², —C(═O)N(R¹³)₂, tetrazolyl, or a carboxylic acid bioisostere;
L¹ is absent, C₁-C₄alkylene, or C₃-C₆cycloalkylene;
ring A is a phenyl, naphthyl, C₃-C₁₂cycloalkyl, C₂-C₁₀heterocycloalkyl, or heteroaryl;
L² is absent, C₁-C₄alkylene, or C₃-C₆cycloalkylene;
each R² is independently selected from H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R³ is H or C₁-C₆alkyl;
or R³ and L² are taken together with the N-atom to which they are attached to form a N-containing C₂-C₆heterocycloalkyl that is unsubstituted or substituted with 1, 2, 3 or 4 R²;
or R³ and ring A are taken together with the intervening atoms to which they are attached to form a substituted or unsubstituted N-containing heterocycloalkyl or a substituted or unsubstituted N-containing heteroaryl, wherein if the ring is substituted then the ring is substituted with 1-4 R²;
or R³ and one R² on ring A are taken together with the intervening atoms to which they are attached to form a form a substituted or unsubstituted fused ring with ring A that is a substituted or unsubstituted fused N-containing heterocycloalkyl or a substituted or unsubstituted fused 5-membered or 6-membered heteroaryl, wherein if the fused ring is substituted then the fused ring is substituted with 1-4 R²;
X¹ is N, C—R^{a}, or N—R^{c};
X² is N, C—R^{b}, or N—R^{c};
R^{a} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
R^{b} is H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, or C₁-C₆heteroalkyl;
R^{c} is H, C₁-C₆alkyl, C₁-C₆fluoroalkyl, C₁-C₆heteroalkyl, —C₁-C₄alkylene-(substituted or unsubstituted aryl), —C₁-C₄alkylene-(substituted or unsubstituted heteroaryl), —C(═O)R¹², —S(═O)₂R¹², —S(═O)₂N(R¹³)₂, —OC(═O)R¹², —CO₂R¹³, or —C(═O)N(R¹³)₂;
X³ is C or N;
X⁴ is C or N; provided that both X³ and X⁴ are not N at the same time;
L³ is C₁-C₄alkylene, —O—C₁-C₄alkylene-, —NR d-C₁-C₄alkylene-, or —NR^{d}—;
R^{d} is H, C₁-C₆alkyl, C₁-C₆fluoroalkyl, or C₁-C₆heteroalkyl;
X⁵ is C—R⁸ or N;
X⁶ is C—R⁹ or N;
X⁷ is C—R¹⁰ or N;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
or R⁵ and R⁶ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
or R⁶ and R⁷ are taken together with the intervening atoms to which they are attached to form a substituted or unsubstituted fused ring that is a substituted or unsubstituted fused phenyl or a substituted or unsubstituted fused 5-membered or 6-membered heteroaryl, wherein if the fused ring is substituted then the fused ring is substituted with 1-4 R¹¹;
each R⁸, R⁹, R¹⁰, and R¹¹ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, 2, 3, or 4.

2. The compound of claim 1, or a pharmaceutically acceptable salt or solvate thereof, wherein:
X¹ is C—R^{a}; X² is N or C—R^{b}; X³ is N; and X⁴ is C; or
X¹ is C—R^{a}; X² is N—R^{c}; X³ is C; and X⁴ is C or N; or
X¹ is N; X² is C—R^{b}; X³ is N; and X⁴ is C; or
X¹ is N or N—R^{c}; X² is C—R^{b} or N; X³ is C; and X⁴ is C or N.

3. The compound of claim 1, or a pharmaceutically acceptable salt or solvate thereof, wherein:
X¹ is C—R^{a}; X² is N or C—R^{b}; X³ is C or N; and X⁴ is C or N; or
X¹ is N; X² is N—R^{c}, or C—R^{b}; X³ is C or N; and X⁴ is C or N.

4. The compound of claim 1, or a pharmaceutically acceptable salt or solvate thereof, wherein:
X¹ is C—R^{a}; X² is N; X³ is N; and X⁴ is C; or
X¹ is N; X² is C—R^{b}; X³ is N; and X⁴ is C; or
X¹ is N; X² is C—R^{b}; X³ is C; and X⁴ is N; or
X¹ is N; X² is N—R; X³ is C; and X⁴ is C; or
X¹ is C—R^{a}; X² is N—R^{c}; X³ is C; and X⁴ is C; or
X¹ is C—R^{a}; X² is N—R^{c}; X³ is C; and X⁴ is N; or
X¹ is C—R^{a}; X² is C—R^{b}; X³ is N; and X⁴ is C; or
X¹ is N—R^{c}; X² is N; X³ is C; and X⁴ is C.

5. The compound of any one of claims 1-4, or a pharmaceutically acceptable salt or solvate thereof, wherein:
X⁵ is N; X⁶ is C—R⁹; and X⁷ is C—R¹⁰; or
X⁵ is C—R⁸; X⁶ is N; and X⁷ is C—R¹⁰; or
X⁵ is C—R⁸; X⁶ is C—R⁹; and X⁷ is N; or
X⁵ is N; X⁶ is C—R⁹; and X⁷ is N.

6. The compound of claim 1, or a pharmaceutically acceptable salt or solvate thereof, wherein:

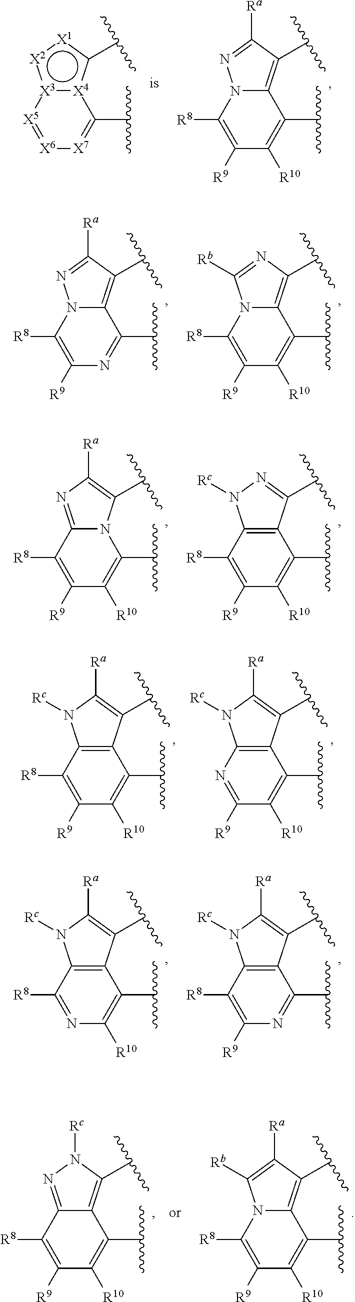

7. The compound of any one of claims 1-6, or a pharmaceutically acceptable salt or solvate thereof, wherein:
R¹ is —CO₂H or —CO₂(C₁-C₆alkyl).

8. The compound of any one of claims 1-6, or a pharmaceutically acceptable salt or solvate thereof, wherein:
R¹ is —CO₂H.

9. The compound of claim 1, or a pharmaceutically acceptable salt or solvate thereof, wherein the compound of Formula (I) has the structure of Formula (II), Formula (III), Formula (IV), or a pharmaceutically acceptable salt or solvate thereof:

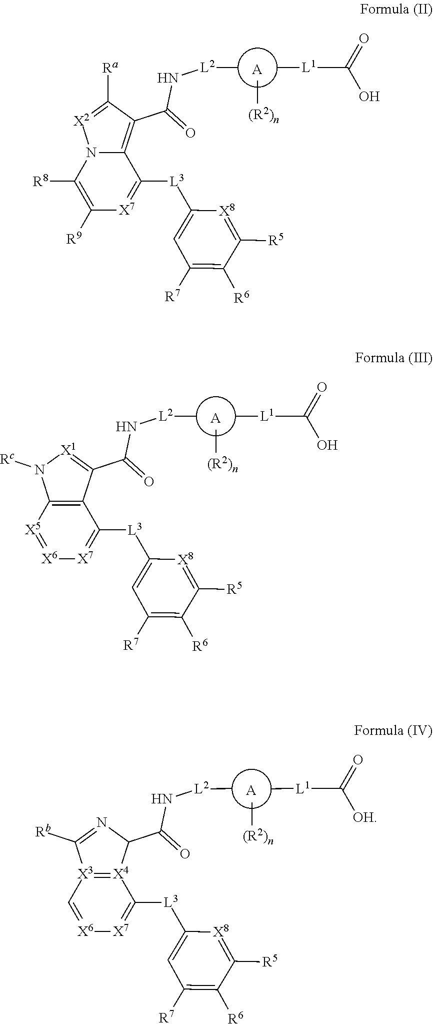

10. The compound of claim 1, or a pharmaceutically acceptable salt or solvate thereof, wherein the compound of Formula (I) has the structure of Formula (V), Formula (VI), Formula (VII), Formula (VIII), or a pharmaceutically acceptable salt or solvate thereof:

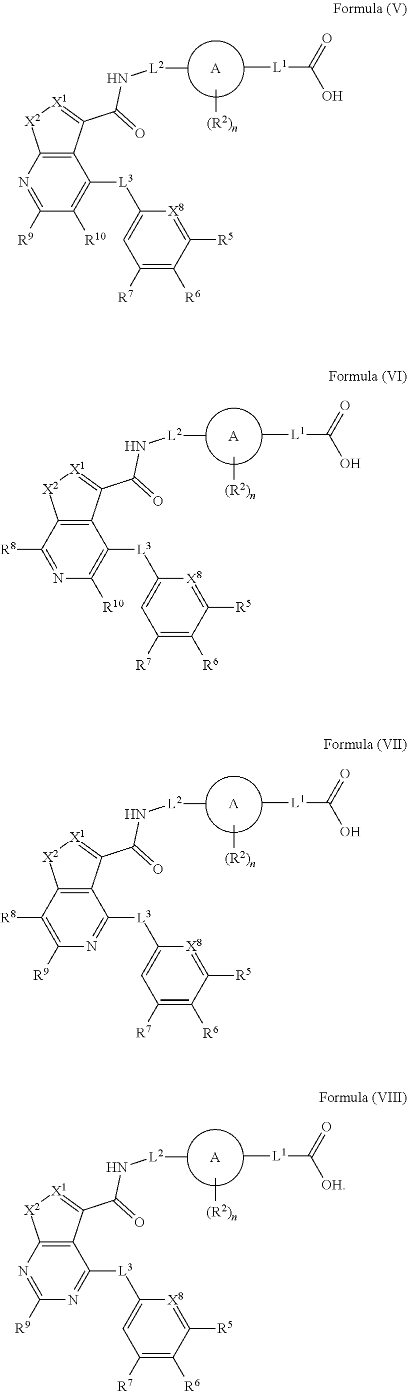

11. The compound of any one of claims 1-10, or a pharmaceutically acceptable salt or solvate thereof, wherein:
L³ is —CH₂—, —CH₂CH₂—, —CH₂CH₂CH₂—, —CH(CH₃)—, —C(CH₃)₂—, —OCH₂—, —OCH₂CH₂—, —OCH₂CH₂CH₂—, —OCH(CH₃)—, —OC(CH₃)₂—, —NH—, —NHCH₂—, —NHCH₂CH₂—, —NHCH₂CH₂CH₂—, —NHCH(CH₃)—, or —NHC(CH₃)₂—.

12. The compound of any one of claims 1-10, or a pharmaceutically acceptable salt or solvate thereof, wherein:
L³ is —CH₂—, —CH₂CH₂—, —CH(CH₃)—, —OCH₂—, —OCH₂CH₂—, —OCH(CH₃)—, —NH—, —NHCH₂—, —NHCH₂CH₂—, or —NHCH(CH₃)—.

13. The compound of any one of claims 1-10, or a pharmaceutically acceptable salt or solvate thereof, wherein:
L³ is —CH₂—.

14. The compound of claim 1, or a pharmaceutically acceptable salt or solvate thereof, wherein the compound of Formula (I) has the structure of Formula (IX), or a pharmaceutically acceptable salt or solvate thereof:

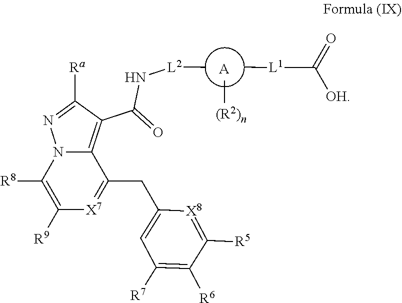

15. The compound of claim 1, or a pharmaceutically acceptable salt or solvate thereof, wherein the compound of Formula (I) has the structure of Formula (X), or a pharmaceutically acceptable salt or solvate thereof:

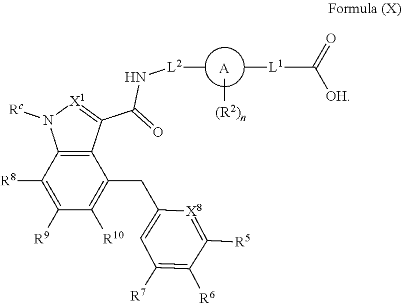

16. The compound of claim 1, or a pharmaceutically acceptable salt or solvate thereof, wherein the compound of Formula (I) has the structure of Formula (XI), or a pharmaceutically acceptable salt or solvate thereof:

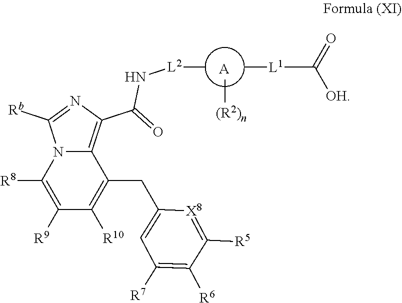

17. The compound of any one of claims 1-16, or a pharmaceutically acceptable salt or solvate thereof, wherein:
L¹ is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl;
L² is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl.

18. The compound of any one of claims 1-16, or a pharmaceutically acceptable salt or solvate thereof, wherein:
L¹ is absent, —CH₂—, —CH₂CH₂—, —CH₂CH₂CH₂—, —CH(CH₃)—, —C(CH₃)₂—, —CH(CH₂CH₃)—, —C(CH₂CH₃)₂—, cyclopropyl-1,1-diyl, cyclobutyl-1,1-diyl, cyclopentyl-1,1-diyl or cyclohexyl-1,1-diyl;
L² is absent, —CH₂—, —CH₂CH₂—, —CH₂CH₂CH₂—, or —CH(CH₃)—.

19. The compound of any one of claims 1-16, or a pharmaceutically acceptable salt or solvate thereof, wherein:
L¹ is absent, —CH₂—, —CH(CH₃)—, —C(CH₃)₂—, or cyclopropyl-1,1-diyl; and
L² is absent, —CH₂—, —CH(CH₃)—, —C(CH₃)₂—, or cyclopropyl-1,1-diyl.

20. The compound of any one of claims 1-19, or a pharmaceutically acceptable salt or solvate thereof, wherein:
L¹ is absent, —CH₂— 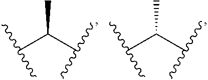
or cyclopropyl-1,1-diyl;
L² is absent, —CH₂—, 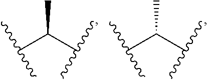
or cyclopropyl-1,1-diyl.

21. The compound of any one of claims 1-19, or a pharmaceutically acceptable salt or solvate thereof, wherein:

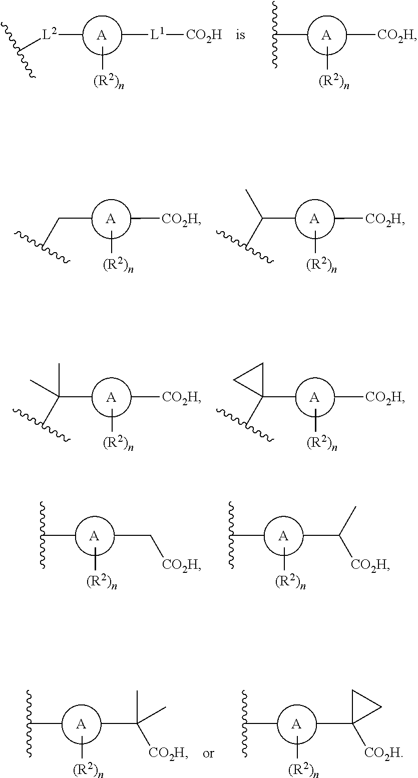

22. The compound of any one of claims 1-21, or a pharmaceutically acceptable salt or solvate thereof, wherein:
ring A is a phenyl.

23. The compound of any one of claims 1-22, or a pharmaceutically acceptable salt or solvate thereof, wherein:

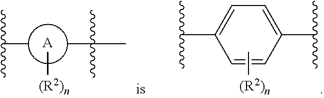

24. The compound of any one of claims 1-21, or a pharmaceutically acceptable salt or solvate thereof, wherein:
ring A is a monocyclic C₃-C₈cycloalkyl, or bicyclic C₇-C₁₂cycloalkyl.

25. The compound of any one of claims 1-21, or a pharmaceutically acceptable salt or solvate thereof, wherein:
ring A is a monocyclic C₃-C₈cycloalkyl; or
ring A is a bicyclic C₇-C₁₂cycloalkyl that is a fused bicyclic C₇-C₁₂cycloalkyl, bridged bicyclic C₇-C₁₂cycloalkyl, or spiro bicyclic C₇-C₁₂cycloalkyl.

26. The compound of any one of claims 1-21, or a pharmaceutically acceptable salt or solvate thereof, wherein:
ring A is cyclobutyl, cyclopentyl, or cyclohexyl; or
ring A is a bicyclic C₇-C₁₂cycloalkyl that is a spiro[2.2]pentanyl, spiro[3.3]heptanyl, spiro[4.3]octanyl, spiro[3.4]octanyl, spiro[3.5]nonanyl, spiro[4.4]nonanyl, spiro[4.5]decanyl, spiro[5.4]decanyl, spiro[5.5]undecanyl, bicyclo[1.1.1]pentanyl, bicyclo[2.2.2]octanyl, bicyclo[2.2.1]heptanyl, adamantyl, or decalinyl.

27. The compound of any one of claims 1-21, or a pharmaceutically acceptable salt or solvate thereof, wherein:
ring A is cyclobutyl, cyclopentyl, or cyclohexyl; or
ring A is spiro[3.3]heptanyl, bicyclo[1.1.1]pentanyl, or bicyclo[2.2.2]octanyl.

28. The compound of any one of claims 1-21, or a pharmaceutically acceptable salt or solvate thereof, wherein:
ring A is cyclohexyl; or
ring A is spiro[3.3]heptanyl, bicyclo[1.1.1]pentanyl, or bicyclo[2.2.2]octanyl.

29. The compound of any one of claims 1-21, or a pharmaceutically acceptable salt or solvate thereof, wherein:

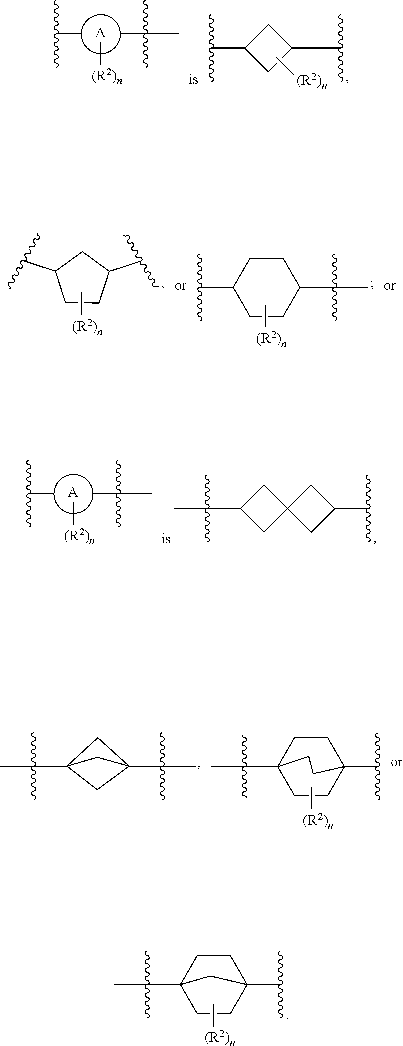

30. The compound of any one of claims 1-21, or a pharmaceutically acceptable salt or solvate thereof, wherein:

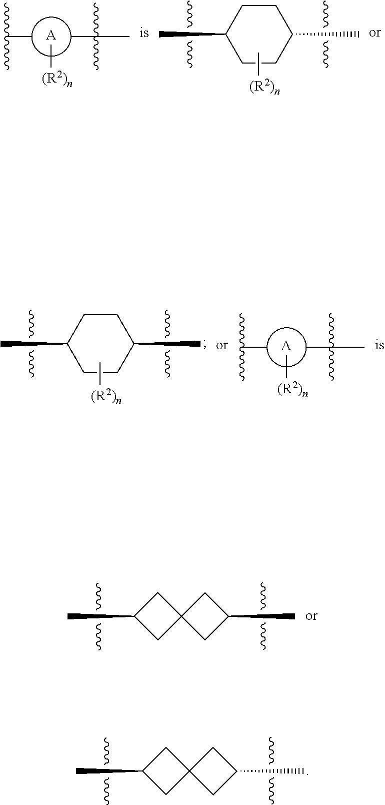

31. The compound of any one of claims 1-21, or a pharmaceutically acceptable salt or solvate thereof, wherein:
ring A is a monocyclic C₂-C₆heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

32. The compound of any one of claims 1-21, or a pharmaceutically acceptable salt or solvate thereof, wherein:
ring A is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

33. The compound of any one of claims 1-21, or a pharmaceutically acceptable salt or solvate thereof, wherein:
ring A is a monocyclic heteroaryl.

34. The compound of any one of claims 1-21, or a pharmaceutically acceptable salt or solvate thereof, wherein:
ring A is a monocyclic heteroaryl selected from furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, and triazinyl.

35. The compound of any one of claims 1-21, or a pharmaceutically acceptable salt or solvate thereof, wherein:

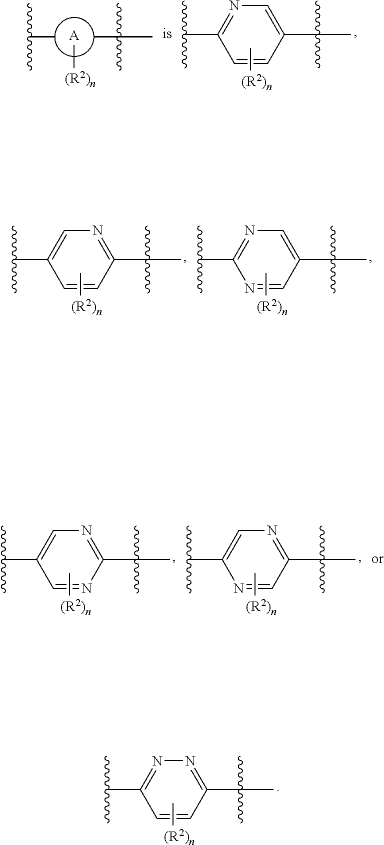

36. The compound of any one of claims 1-21, or a pharmaceutically acceptable salt or solvate thereof, wherein:
ring A is a bicyclic heteroaryl selected from indolyl, benzofuranyl, benzothienyl, benzoxazolyl, benzisoxazolyl, benzimidazolyl, imidazopyrdinyl, imidazopyridazinyl, purinyl, quinolinyl, quinazolinyl, and pyridopyrimidinyl.

37. The compound of any one of claims 1-36, or a pharmaceutically acceptable salt or solvate thereof, wherein:
each R² is independently selected from H, F, Cl, Br, —OH, —CN, —NH₂, —NH(CH₃), —N(CH₃)₂, —CH₃, —OCH₃, —CF₃, and —OCF₃;
n is 0, 1, or 2.

38. The compound of any one of claims 1-16, or a pharmaceutically acceptable salt or solvate thereof, wherein:

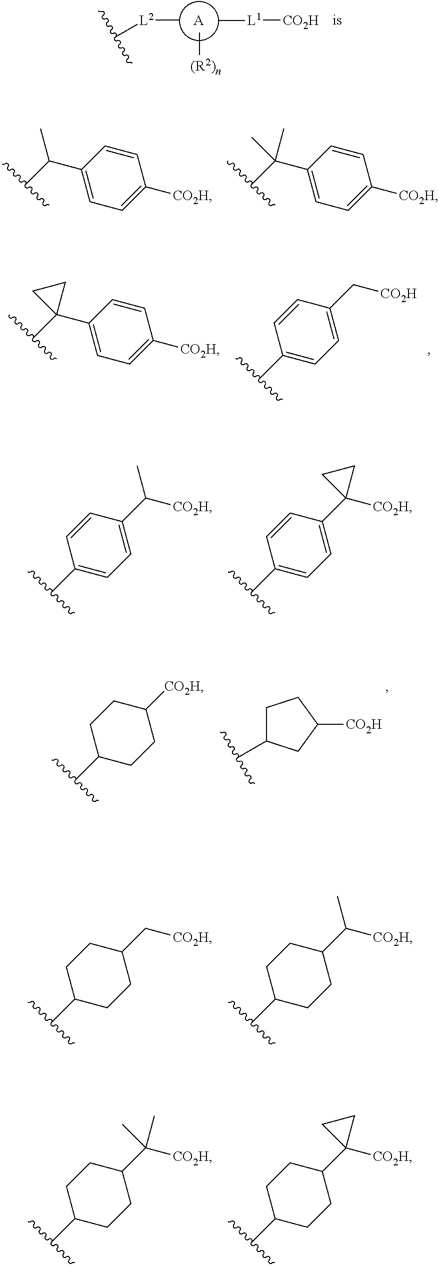

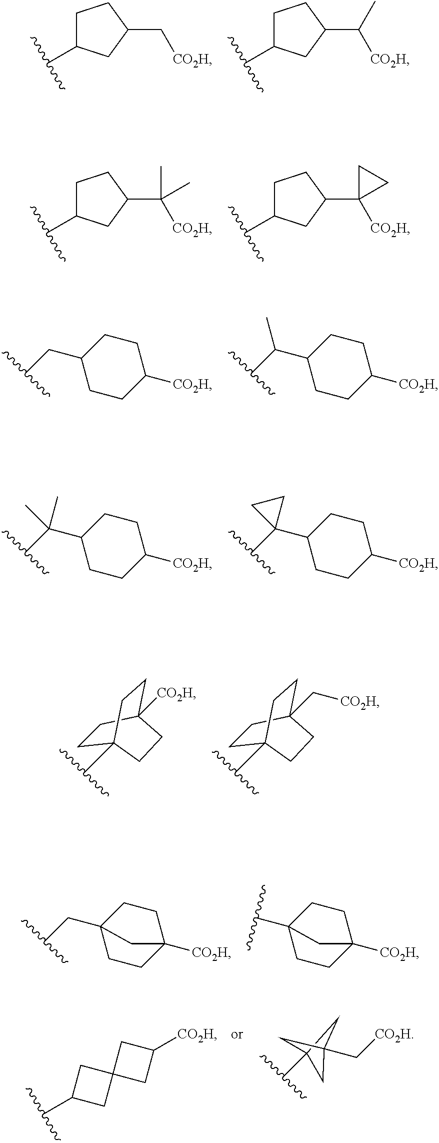

39. The compound of any one of claims 1-38, or a pharmaceutically acceptable salt or solvate thereof, wherein:
X⁸ is C—R⁴ or N;
R⁴ is selected from H, F, Cl, Br, —OH, —CN, —NH₂, —NH(CH₃), —N(CH₃)₂, —CH₃, —OCH₃, —CF₃, and —OCF₃;
R⁵ is selected from H, F, Cl, Br, —OH, —CN, —NH₂, —NH(CH₃), —N(CH₃)₂, —CH₃, —OCH₃, —CF₃, and —OCF₃;
R⁶ is selected from H, F, Cl, Br, —OH, —CN, —NH₂, —NH(CH₃), —N(CH₃)₂, —CH₃, —OCH₃, —CF₃, and —OCF₃;
or R⁵ and R⁶ are taken together with the intervening atoms to which they are attached to form a substituted or unsubstituted fused phenyl, wherein if the fused phenyl is substituted then the fused phenyl is substituted with 1-4 R¹¹;
R⁷ is selected from H, F, Cl, Br, —OH, —CN, —NH₂, —NH(CH₃), —N(CH₃)₂, —CH₃, —OCH₃, —CF₃, and —OCF₃;
or R⁶ and R⁷ are taken together with the intervening atoms to which they are attached to form a substituted or unsubstituted fused phenyl, wherein if the fused phenyl is substituted then the fused ring is substituted with 1-4 R¹¹.

40. The compound of any one of claims 1-38, or a pharmaceutically acceptable salt or solvate thereof, wherein:
X⁸ is C—R⁴;
R⁴ is H;
R⁵ is selected from H, F, Cl, Br, —OH, —CN, —CH₃, —OCH₃, —CF₃, and —OCF₃;
R⁶ is selected from H, F, Cl, Br, —OH, —CN, —CH₃, —OCH₃, —CF₃, and —OCF₃;
R⁷ is H.

41. The compound of any one of claims 1-38, or a pharmaceutically acceptable salt or solvate thereof, wherein:
X⁸ is N;
R⁵ and R⁶ are taken together with the intervening atoms to which they are attached to form a substituted or unsubstituted fused phenyl, wherein if the fused phenyl is substituted then the fused phenyl is substituted with 1-4 R¹¹;
R⁷ is H.

42. The compound of any one of claims 1-38, or a pharmaceutically acceptable salt or solvate thereof, wherein:
X⁸ is N;
R⁵ is H;
R⁶ and R⁷ are taken together with the intervening atoms to which they are attached to form a substituted or unsubstituted fused phenyl, wherein if the fused phenyl is substituted then the fused phenyl is substituted with 1-4 R¹¹.

43. The compound of any one of claims 1-42, or a pharmaceutically acceptable salt or solvate thereof, wherein:
each R⁸, R⁹ and R¹⁰ is independently selected from H, F, Cl, Br, —OH, —CN, —NH₂, —NH(CH₃), —N(CH₃)₂, —CH₃, —OCH₃, —CF₃, and —OCF₃.

44. The compound of claim 1, or a pharmaceutically acceptable salt or solvate thereof, wherein the compound of Formula (I) has the structure of Formula (XII), or a pharmaceutically acceptable salt or solvate thereof: 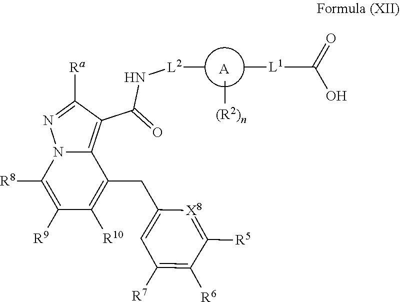
wherein,
L¹ is absent, C₁-C₄alkylene, or C₃-C₆cycloalkylene;
ring A is a phenyl or C₃-C₁₂cycloalkyl;
L² is absent, C₁-C₄alkylene, or C₃-C₆cycloalkylene;
each R² is independently selected from H, halogen, —OH, —CN, —NH₂, —NH(C₁-C₆alkyl),—N(C₁-C₆alkyl)₂, C₁-C₆alkyl, C₁-C₆alkoxy, C₁-C₆fluoroalkyl, C₁-C₆fluoroalkoxy, and C₁-C₆heteroalkyl;
R^{a} is H, halogen, C₁-C₆alkyl, C₁-C₆alkoxy, C₁-C₆fluoroalkyl, C₁-C₆fluoroalkoxy, or C₁-C₆heteroalkyl;
X⁸ is C—R⁴ or N;
each R⁴, R⁵, R⁶, and R⁷ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R⁸, R⁹, and R¹⁰ is independently selected from H, halogen, —CN, —OH, C₁-C₆alkyl, C₁-C₆alkoxy, C₁-C₆fluoroalkyl, C₁-C₆fluoroalkoxy, C₁-C₆heteroalkyl, substituted or unsubstituted C₃-C₆cycloalkyl, substituted or unsubstituted C₂-C₆heterocycloalkyl, substituted or unsubstituted phenyl, substituted unsubstituted monocyclic heteroaryl, —N(R¹³)₂, —N(R¹³)C(═O)R¹², —C(═O)N(R¹³)₂, —C(═O)R¹², —SR¹³, —S(═O)R¹², —S(═O)₂R¹², —S(═O)₂N(R¹³)₂, —OC(═O)R¹², and —CO₂R¹³;
each R¹² is independently selected from C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
each R¹³ is independently selected from H, C₁-C₆alkyl, C₁-C₆fluoroalkyl, C₁-C₆heteroalkyl, substituted or unsubstituted C₃-C₆cycloalkyl, substituted or unsubstituted C₂-C₆heterocycloalkyl, substituted or unsubstituted phenyl, and substituted unsubstituted monocyclic heteroaryl;
or two R¹³ on the same N atom are taken together with the N atom to which they are attached to form a substituted or unsubstituted N-containing C₂-C₆heterocycloalkyl;
n is 0, 1, 2, 3, or 4.

45. The compound of claim 44, or a pharmaceutically acceptable salt or solvate thereof, wherein:
L¹ is absent, —CH₂—, —CH(CH₃)—, —C(CH₃)₂—, or cyclopropyl-1,1-diyl;
ring A is phenyl, cyclobutyl, cyclopentyl, or cyclohexyl, spiro[2.2]pentanyl, spiro[3.3]heptanyl, spiro[4.3]octanyl, spiro[3.4]octanyl, spiro[3.5]nonanyl, spiro[4.4]nonanyl, spiro[4.5]decanyl, spiro[5.4]decanyl, spiro[5.5]undecanyl, bicyclo[1.1.1]pentanyl, bicyclo[2.2.2]octanyl, bicyclo[2.2.1]heptanyl, adamantyl, or decalinyl; and
L² is absent, —CH₂—, —CH(CH₃)—, —C(CH₃)₂—, or cyclopropyl-1,1-diyl.

46. The compound of claim 44 or claim 45, or a pharmaceutically acceptable salt or solvate thereof, wherein:
ring A is phenyl, cyclohexyl, spiro[3.3]heptanyl, bicyclo[1.1.1]pentanyl, or bicyclo[2.2.2]octanyl.

47. The compound of any one of claims 44-46, or a pharmaceutically acceptable salt or solvate thereof, wherein:

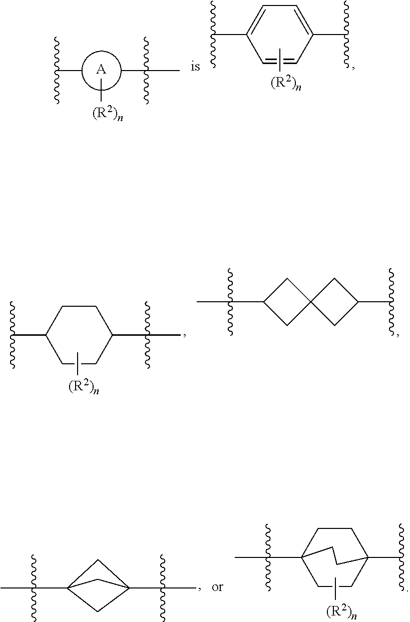

48. The compound of any one of claims 44-46, or a pharmaceutically acceptable salt or solvate thereof, wherein:

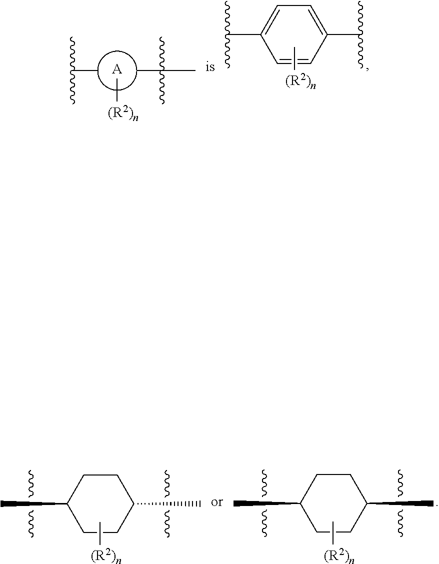

49. The compound of any one of claims 44-48, or a pharmaceutically acceptable salt or solvate thereof, wherein:
L¹ is absent.

50. The compound of any one of claims 44-49, or a pharmaceutically acceptable salt or solvate thereof, wherein:
L² is absent.

51. The compound of any one of claims 44-50, or a pharmaceutically acceptable salt or solvate thereof, wherein:
each R² is independently selected from H, F, Cl, Br, —OH, —CN, —NH₂, —NH(CH₃), —N(CH₃)₂, —CH₃, —OCH₃, —CF₃, and —OCF₃;
R^{a} is H, —CH₃, —CH₂CH₃, —CH₂CH₂CH₃, —CH(CH₃)₂, —CH₂CH(CH₃)₂, —CF₃, —CFH₂, —CHF₂, —CHCFH₂, —CH₂CH₂CFH₂;
each R⁴, R⁵, R⁶, and R⁷ is independently selected from H, H, F, Cl, Br, —OH, —CN, —CH₃, —OCH₃, —CF₃, and —OCF₃;
each R⁸, R⁹ and R¹⁰ is independently selected from H, F, Cl, Br, —OH, —CN, —NH₂, —NH(CH₃), —N(CH₃)₂, —CH₃, —OCH₃, —CF₃, and —OCF₃.

52. The compound of any one of claims 44-51, or a pharmaceutically acceptable salt or solvate thereof, wherein:
R^{a} is H; and
n is 0.

53. A compound that is: 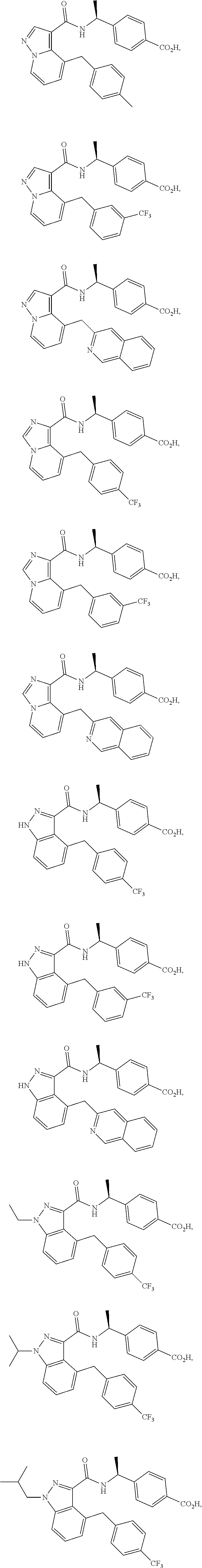 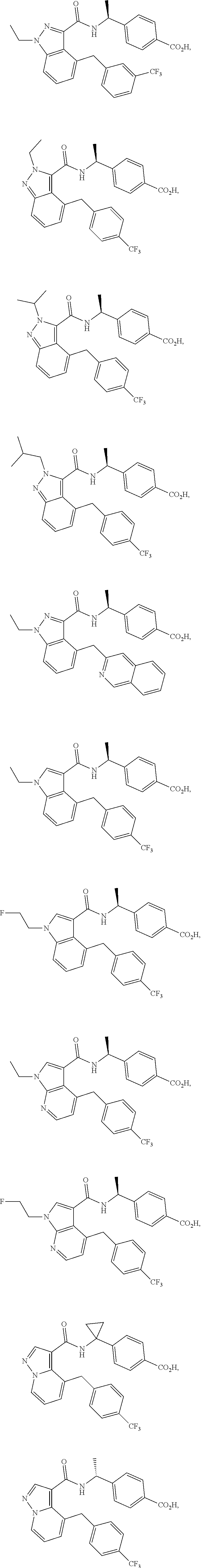 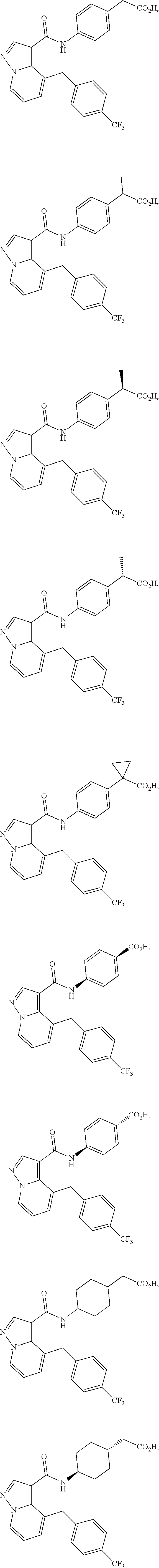 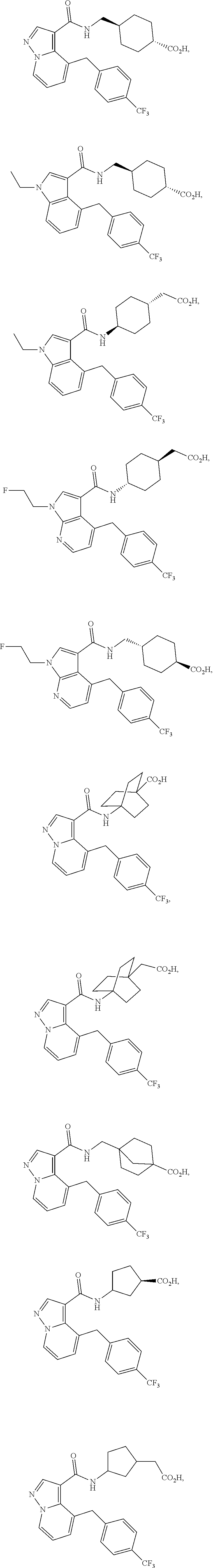 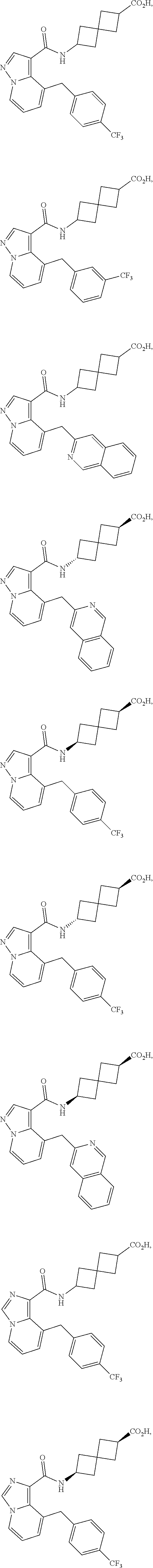 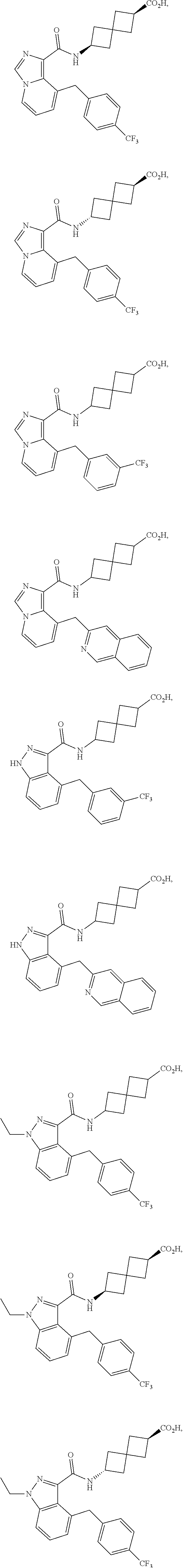 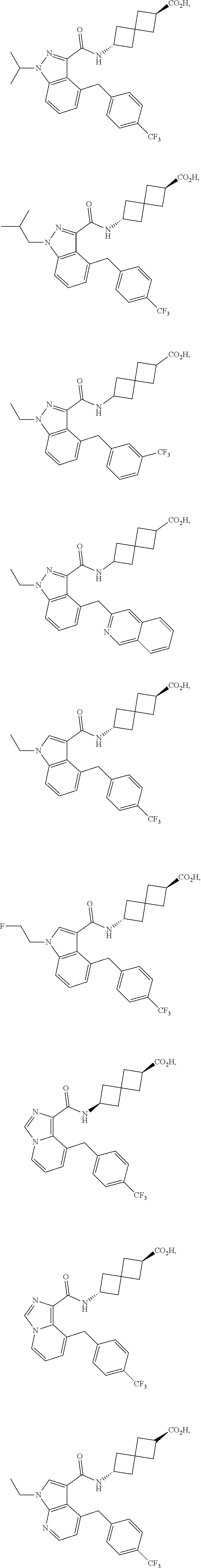 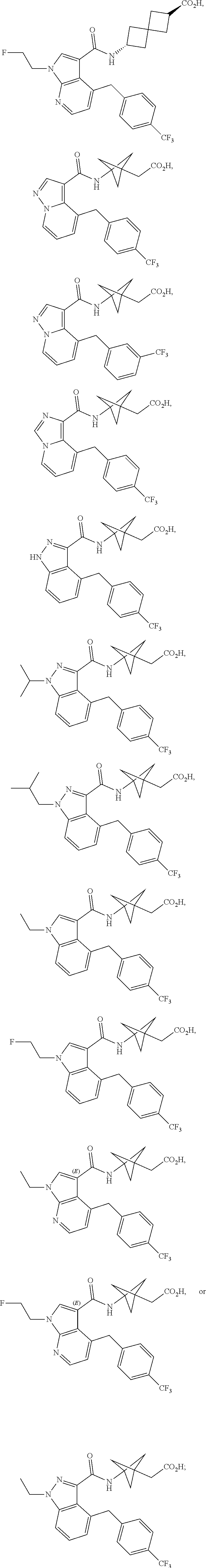
or a pharmaceutically acceptable salt or solvate thereof of any one of the preceding compounds.

54. A pharmaceutical composition comprising a compound of any one of claims 1-53, or a pharmaceutically acceptable salt or solvate thereof, and at least one pharmaceutically acceptable excipient.

55. The pharmaceutical composition of claim 54, wherein the pharmaceutical composition is formulated for administration to a mammal by oral administration, intravenous administration, or subcutaneous administration.

56. The pharmaceutical composition of claim 54, wherein the pharmaceutical composition is in the form of a tablet, a pill, a capsule, a liquid, a suspension, a dispersion, a solution, or an emulsion.

57. A method of modulating the activity of prostaglandin E2 receptor 4 (EP4) in a mammal comprising administering to the mammal a compound of any one of claims 1-53, or any pharmaceutically acceptable salt or solvate thereof.

58. A method of treating a disease or disorder in a mammal that is mediated by the action of prostaglandin E2 (PGE2) at prostaglandin E2 receptor 4 (EP4) comprising administering to the mammal a compound of any one of claims 1-53, or any pharmaceutically acceptable salt or solvate thereof.

59. The method of claim 58, wherein the disease or disorder is cancer.

60. A method for treating cancer in a mammal, the method comprising administering to the mammal a compound of any one of claims 1-53, or any pharmaceutically acceptable salt or solvate thereof.

61. The method of claim 60, wherein the cancer is a solid tumor.

62. The method of claim 60, wherein the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer.

63. The method of claim 60, wherein the cancer is prostate cancer, breast cancer, colon cancer, or lung cancer.

64. The method of claim 60, wherein the cancer is a sarcoma, carcinoma, or lymphoma.

65. The method of any one of claims 57-64, furthering comprising administering at least one additional therapy to the mammal.

66. The method of any one of claims 57-65, wherein the mammal is a human.
